SEQUENCE LISTING (25 sequences)

SEQ ID NO: 1 - DNA, 1992 bp, Homo sapiens
  REP1 gene (1)..(1992)
gatatcgaattcctgcagcccggcggcaccatggcggatactctcccttcggagtttgatgtgatcgtaatagggacgggtttgcctgaatccatcattgcagctgcatgttcaagaagtggccggagagttctgcatgttgattcaagaagctactatggaggaaactgggccagttttagcttttcaggactattgtcctggctaaaggaataccaggaaaacagtgacattgtaagtgacagtccagtgtggcaagaccagatccttgaaaatgaagaagccattgctcttagcaggaaggacaaaactattcaacatgtggaagtattttgttatgccagtcaggatttgcatgaagatgtcgaagaagctggtgcactgcagaaaaatcatgctcttgtgacatctgcaaactccacagaagctgcagattctgccttcctgcctacggaggatgagtcattaagcactatgagctgtgaaatgctcacagaacaaactccaagcagcgatccagagaatgcgctagaagtaaatggtgctgaagtgacaggggaaaaagaaaaccattgtgatgataaaacttgtgtgccatcaacttcagcagaagacatgagtgaaaatgtgcctatagcagaagataccacagagcaaccaaagaaaaacagaattacttactcacaaattattaaagaaggcaggagatttaatattgatttagtatcaaagctgctgtattctcgaggattactaattgatcttctaatcaaatctaatgttagtcgatatgcagagtttaaaaatattaccaggattcttgcatttcgagaaggacgagtggaacaggttccgtgttccagagcagatgtctttaatagcaaacaacttactatggtagaaaagcgaatgctaatgaaatttcttacattttgtatggaatatgagaaatatcctgatgaatataaaggatatgaagagatcacattttatgaatatttaaagactcaaaaattaacccccaacctccaatatattgtcatgcattcaattgcaatgacatcagagacagccagcagcaccatagatggtctcaaagctaccaaaaactttcttcactgtcttgggcggtatggcaacactccatttttgtttcctttatatggccaaggagaactcccccagtgtttctgcaggatgtgtgctgtgtttggtggaatttattgtcttcgccattcagtacagtgccttgtagtggacaaagaatccagaaaatgtaaagcaattatagatcagtttggtcagagaataatctctgagcatttcctcgtggaggacagttactttcctgagaacatgtgctcacgtgtgcaatacaggcagatctccagggcagtgctgattacagatagatctgtcctaaaaacagattcagatcaacagatttccattttgacagtgccagcagaggaaccaggaacttttgctgttcgggtcattgagttatgttcttcaacgatgacatgcatgaaaggcacctatttggttcatttgacttgcacatcttctaaaacagcaagagaagatttagaatcagttgtgcagaaattgtttgttccatatactgaaatggagatagaaaatgaacaagtagaaaagccaagaattctgtgggctctttacttcaatatgagagattcgtcagacatcagcaggagctgttataatgatttaccatccaacgtttatgtctgctctggcccagattgtggtttaggaaatgataatgcagtcaaacaggctgaaacacttttccaggaaatctgccccaatgaagatttctgtccccctccaccaaatcctgaagacattatccttgatggagacagtttacagccagaggcttcagaatccagtgccataccagaggctaactcggagactttcaaggaaagcacaaaccttggaaacctagaggagtcctctgaataa

SEQ ID NO: 2 - protein, 653 aa, Homo sapiens
  REP1 protein (1)..(653)
MADTLPSEFDVIVIGTGLPESIIAAACSRSGRRVLHVDSRSYYGGNWASFSFSGLLSWLKEYQENSDIVSDSPVWQDQILENEEAIALSRKDKTIQHVEVFCYASQDLHEDVEEAGALQKNHALVTSANSTEAADSAFLPTEDESLSTMSCEMLTEQTPSSDPENALEVNGAEVTGEKENHCDDKTCVPSTSAEDMSENVPIAEDTTEQPKKNRITYSQIIKEGRRFNIDLVSKLLYSRGLLIDLLIKSNVSRYAEFKNITRILAFREGRVEQVPCSRADVFNSKQLTMVEKRMLMKFLTFCMEYEKYPDEYKGYEEITFYEYLKTQKLTPNLQYIVMHSIAMTSETASSTIDGLKATKNFLHCLGRYGNTPFLFPLYGQGELPQCFCRMCAVFGGIYCLRHSVQCLVVDKESRKCKAIIDQFGQRIISEHFLVEDSYFPENMCSRVQYRQISRAVLITDRSVLKTDSDQQISILTVPAEEPGTFAVRVIELCSSTMTCMKGTYLVHLTCTSSKTAREDLESVVQKLFVPYTEMEIENEQVEKPRILWALYFNMRDSSDISRSCYNDLPSNVYVCSGPDCGLGNDNAVKQAETLFQEICPNEDFCPPPPNPEDIILDGDSLQPEASESSAIPEANSETFKESTNLGNLEESSE

SEQ ID NO: 3 - DNA, 248 bp, Artificial Sequence
  Recombinant synthesis
attgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgg

SEQ ID NO: 4 - DNA, 250 bp, Artificial Sequence
  Recombinant synthesis
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgg

SEQ ID NO: 5 - DNA, 509 bp, Artificial Sequence
  Recombinant synthesis
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcgggg

SEQ ID NO: 6 - DNA, 93 bp, Artificial Sequence
  Recombinant synthesis
ctctgctaaccatgttcatgccttcttctttttcctacagctcctgggcaacgtgctggttattgtgctgtctcatcattttggcaaagaatt

SEQ ID NO: 7 - DNA, 691 bp, Artificial Sequence
  Recombinant synthesis
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatc

SEQ ID NO: 8 - DNA, 941 bp, Artificial Sequence
  Recombinant synthesis
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatc

SEQ ID NO: 9 - DNA, 1992 bp, Homo sapiens; identical to SEQ ID NO: 1

SEQ ID NO: 10 - protein, 653 aa, Homo sapiens; identical to SEQ ID NO: 2

SEQ ID NO: 11 - DNA, 588 bp, Artificial Sequence
  Recombinant synthesis
atcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 12 - DNA, 270 bp, Artificial Sequence
  Recombinant synthesis
tcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggg

SEQ ID NO: 13 - DNA, 144 bp, Artificial Sequence
  Recombinant synthesis
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgatt

SEQ ID NO: 14 - DNA, 121 bp, Artificial Sequence
  Recombinant synthesis
aggaacccctagtgatggagttggccactccctctctgcgcgctcgctcgctcactgaggccgggcgaccaaaggtcgcccgacgcccgggcggcctcagtgagcgagcgagcgcgcagag

SEQ ID NO: 15 - protein, 735 aa, Artificial Sequence [sequence omitted]

SEQ ID NO: 16 - DNA, 4213 bp, Artificial Sequence
  Recombinant synthesis
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcctagcttgatatcgaattcctgcagcccggcggcaccatggcggatactctcccttcggagtttgatgtgatcgtaatagggacgggtttgcctgaatccatcattgcagctgcatgttcaagaagtggccggagagttctgcatgttgattcaagaagctactatggaggaaactgggccagttttagcttttcaggactattgtcctggctaaaggaataccaggaaaacagtgacattgtaagtgacagtccagtgtggcaagaccagatccttgaaaatgaagaagccattgctcttagcaggaaggacaaaactattcaacatgtggaagtattttgttatgccagtcaggatttgcatgaagatgtcgaagaagctggtgcactgcagaaaaatcatgctcttgtgacatctgcaaactccacagaagctgcagattctgccttcctgcctacggaggatgagtcattaagcactatgagctgtgaaatgctcacagaacaaactccaagcagcgatccagagaatgcgctagaagtaaatggtgctgaagtgacaggggaaaaagaaaaccattgtgatgataaaacttgtgtgccatcaacttcagcagaagacatgagtgaaaatgtgcctatagcagaagataccacagagcaaccaaagaaaaacagaattacttactcacaaattattaaagaaggcaggagatttaatattgatttagtatcaaagctgctgtattctcgaggattactaattgatcttctaatcaaatctaatgttagtcgatatgcagagtttaaaaatattaccaggattcttgcatttcgagaaggacgagtggaacaggttccgtgttccagagcagatgtctttaatagcaaacaacttactatggtagaaaagcgaatgctaatgaaatttcttacattttgtatggaatatgagaaatatcctgatgaatataaaggatatgaagagatcacattttatgaatatttaaagactcaaaaattaacccccaacctccaatatattgtcatgcattcaattgcaatgacatcagagacagccagcagcaccatagatggtctcaaagctaccaaaaactttcttcactgtcttgggcggtatggcaacactccatttttgtttcctttatatggccaaggagaactcccccagtgtttctgcaggatgtgtgctgtgtttggtggaatttattgtcttcgccattcagtacagtgccttgtagtggacaaagaatccagaaaatgtaaagcaattatagatcagtttggtcagagaataatctctgagcatttcctcgtggaggacagttactttcctgagaacatgtgctcacgtgtgcaatacaggcagatctccagggcagtgctgattacagatagatctgtcctaaaaacagattcagatcaacagatttccattttgacagtgccagcagaggaaccaggaacttttgctgttcgggtcattgagttatgttcttcaacgatgacatgcatgaaaggcacctatttggttcatttgacttgcacatcttctaaaacagcaagagaagatttagaatcagttgtgcagaaattgtttgttccatatactgaaatggagatagaaaatgaacaagtagaaaagccaagaattctgtgggctctttacttcaatatgagagattcgtcagacatcagcaggagctgttataatgatttaccatccaacgtttatgtctgctctggcccagattgtggtttaggaaatgataatgcagtcaaacaggctgaaacacttttccaggaaatctgccccaatgaagatttctgtccccctccaccaaatcctgaagacattatccttgatggagacagtttacagccagaggcttcagaatccagtgccataccagaggctaactcggagactttcaaggaaagcacaaaccttggaaacctagaggagtcctctgaataatctagtcgattcgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggcggcctcagtgagcgagcgagcgcgcagag

SEQ ID NO: 17 - DNA, 1962 bp, Homo sapiens
atggcggatactctcccttcggagtttgatgtgatcgtaatagggacgggtttgcctgaatccatcattgcagctgcatgttcaagaagtggccggagagttctgcatgttgattcaagaagctactatggaggaaactgggccagttttagcttttcaggactattgtcctggctaaaggaataccaggaaaacagtgacattgtaagtgacagtccagtgtggcaagaccagatccttgaaaatgaagaagccattgctcttagcaggaaggacaaaactattcaacatgtggaagtattttgttatgccagtcaggatttgcatgaagatgtcgaagaagctggtgcactgcagaaaaatcatgctcttgtgacatctgcaaactccacagaagctgcagattctgccttcctgcctacggaggatgagtcattaagcactatgagctgtgaaatgctcacagaacaaactccaagcagcgatccagagaatgcgctagaagtaaatggtgctgaagtgacaggggaaaaagaaaaccattgtgatgataaaacttgtgtgccatcaacttcagcagaagacatgagtgaaaatgtgcctatagcagaagataccacagagcaaccaaagaaaaacagaattacttactcacaaattattaaagaaggcaggagatttaatattgatttagtatcaaagctgctgtattctcgaggattactaattgatcttctaatcaaatctaatgttagtcgatatgcagagtttaaaaatattaccaggattcttgcatttcgagaaggacgagtggaacaggttccgtgttccagagcagatgtctttaatagcaaacaacttactatggtagaaaagcgaatgctaatgaaatttcttacattttgtatggaatatgagaaatatcctgatgaatataaaggatatgaagagatcacattttatgaatatttaaagactcaaaaattaacccccaacctccaatatattgtcatgcattcaattgcaatgacatcagagacagccagcagcaccatagatggtctcaaagctaccaaaaactttcttcactgtcttgggcggtatggcaacactccatttttgtttcctttatatggccaaggagaactcccccagtgtttctgcaggatgtgtgctgtgtttggtggaatttattgtcttcgccattcagtacagtgccttgtagtggacaaagaatccagaaaatgtaaagcaattatagatcagtttggtcagagaataatctctgagcatttcctcgtggaggacagttactttcctgagaacatgtgctcacgtgtgcaatacaggcagatctccagggcagtgctgattacagatagatctgtcctaaaaacagattcagatcaacagatttccattttgacagtgccagcagaggaaccaggaacttttgctgttcgggtcattgagttatgttcttcaacgatgacatgcatgaaaggcacctatttggttcatttgacttgcacatcttctaaaacagcaagagaagatttagaatcagttgtgcagaaattgtttgttccatatactgaaatggagatagaaaatgaacaagtagaaaagccaagaattctgtgggctctttacttcaatatgagagattcgtcagacatcagcaggagctgttataatgatttaccatccaacgtttatgtctgctctggcccagattgtggtttaggaaatgataatgcagtcaaacaggctgaaacacttttccaggaaatctgccccaatgaagatttctgtccccctccaccaaatcctgaagacattatccttgatggagacagtttacagccagaggcttcagaatccagtgccataccagaggctaactcggagactttcaaggaaagcacaaaccttggaaacctagaggagtcctctgaataa

SEQ ID NO: 18 - DNA, 942 bp, Artificial Sequence
  Recombinant synthesis
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcc

SEQ ID NO: 19 - DNA, 593 bp, Artificial Sequence
  Recombinant synthesis
atcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctcccc

SEQ ID NO: 20 - DNA, 269 bp, Artificial Sequence
  Recombinant synthesis
cgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggg

SEQ ID NO: 21 - DNA, 130 bp, Artificial Sequence
  Recombinant synthesis
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 22 - DNA, 121 bp, Artificial Sequence; identical to SEQ ID NO: 14
  Recombinant synthesis

SEQ ID NO: 23 - DNA, 1962 bp, Homo sapiens; identical to SEQ ID NO: 17

SEQ ID NO: 24 - DNA, 5357 bp, Artificial Sequence
  Recombinant synthesis
ctaaattgtaagcgttaatttttgttaaaattaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaagctggcgagaaaggaagggaagaaagcgaaaggagcgggcgctagggcgctggcaagtgtagcggtcacgctgcgcgtcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagctaggatcctagctgatatcgaattcctgcagcccggcggcaccatggcggatactctcccttcggagtttgatgtgatcgtaatagggacgggtttgcctgaatccatcattgcagctgcatgttcaagaagtggccggagagttctgcatgttgattcaaagaagctactatggaggaaactgggccagttttagcttttcaggactattgtcctggctaaaggaataccaggaaaacagtgacattgtaagtgacagtccagtgtggcaagaccagatccttgaaaatgaagaagccattgctcttagcaggaaggacaaaactattcaacatgtggaagtattttgttatgccagtcaggatttgcatgaagatgtcgaagaagctggtcactgcagaaaaatcatgctcttgtgacatctgcaaactccacagaagctgcagattctgcctcctgcctacggaggatgagtcattaagcactatgagctgtgaaatgctcacagaacaaactccaagcagcgatccagagaatgcgcgctagaagtaaatggtgctgaagtgacaggggaaaaagaaaaccattgtgatgataaaacttgtgtgccatcaacttacgcagaagacatgagtgaaaatgtcgctatagcagaagataccacagagcaaccaaagaaaaacagaattacttactcacaaattattaaagaaggcaggagatttaatattgatttagtatcaaagctgctgtattctcgaggattactaattgatcttctaatcaaatctaatgttagtcgatatgcagagtttaaaaatattaccaggattcttgcatttcgagaggacgagtggaacaggttccgtgttccagagccagatgtctttaatagcaaacaacttactatggtagaaagcgaatgctaatgaaatttcttacattttgtatggaatatgagaaatatctgatgaatataaaggatatgaagagatcacattttatgaatatttaaagactcaaaaattaacccccaacctccaatatattgtcatgcattcaattgcaatgacatcagagacagccagcagcaccatagatggtctccaaagctaccaaaaactttcttcactgtcttgggcggtatggcaacactccatttttgtttcctttatatggccaaggagaactcccccagtgtttctgcaggatgtgtgctgtgtttggtggaatttattgtcttcgccattcagtacagtgccttgtagtggacaaagaatccagaaaatgtaaagcaattatagatcagtttggtcagagaataatctctgagcatttcctcgtggaggacagttactttcctgagaacatgtgctcacgtgtgcaatacaggcagatctccagggcagtgctgattacagatagatctgtcctaaaaacagattcagatcaacagatttccattttgacagtgccagcagaggaaccagaacttttgctgttcgggtcattgagttatgttcttcaacgatgacatgcatgaaaggcacctatttggttcatttgacttgcacatcttctaaaacagcaagagaagatttagaatcagttgtgcagaaattgtttgttccatatactgaaatggagatagaaaatgaacaagtagaaaagccaagaattctgtgggctctttacttcaatatgagagttcgtcagacatcagcaggagctcttataatgatttaccatccaacgtttatgtctgctctggcccagattgtggtttaggaaatgataatgcagtcaaacaggctgaaacacttttccaggaaatctgccccaatgaagatttctgtccccctccaccaaatccctgaagacattatccttgatggagacagtttacagccagaggctcagaatccagtgccataccagaggctaactcggagactttcaaggaaagcacaaaccttggaaacctagaggagtcctctgaataattagagcggccaacgcgttggccgccaccgcggtggagctccagcttttgttccctttagtgagggttaattgcgcgcttggcgtaatcatggtcatagctgtttcctgtgtgaaattgttatccgctcacaattccacacaacatacgaccggaagcataaagtgtaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaagccagcaaaaggccaggaaccgtaaaaaggccgcgtgctggcgtttttccataggctccgcccccctgacgagcatcacaaaaatcgacgctcaagtcagaggtggcgaaaccgacaggactataaagataccaggcgtttccccctggaagctccctcgtgcgctctcctgttccgaccctgccgcttaccggatacctgtccgcctttctcccttcgggaagcgtggcgctttctcatagctcacgcgtaggtatctcagttcggtgtaggtcgttcgctccagctgggctgtgtgcacgaaccccccgttcagcccgaccgctgcgccttatccggtaactatctcttgagtccaacccggtaagacagacttatcgccactggcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttcgaccgaataaatacctgtgacggaagatcacttgcaaataaataaatccggttccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattagtatggcaatgaaagacggtgagctggtgatatgggatagtgttcacccttgttacaccgttttccatgagcaaactgaaacgttttcatcgctctggagtgaataccaacgagatttccggcagtttctacacatatattcgcaagatgtggcgtgttaggtgaaaacctggcctatttccctaaagggtttattgagaatatgtttttcgtctcagccaatccctgggtgagtttcaccagttttgatttaaacgtggccaatatggaaacttcttcgccccgttttcaccatgggcaaatattatacgcaaggcgacaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgctggttgctacgcctgaataatgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttcttctcaaatgcctgaggccagtttgctcaggctctccccgtggaggtaataattgacgatatgatcctttttttctgatcaaaagtgctcatcattggaaaacgttcttcggggcgaaaactctcaaggatcttaccgctgttgagatccagttgatgtaacccactcgtgcacccaactgatcttcagcatcttttactttcaccacgtttctgggtgagcaaaaacaggaaggcaaaatgccgcaaaaaagggaataagggcgacacggaaatgttgaatactcatactcttcctttttcaatattattgaagcatttatcaagggttattgtctcatgagcggatacatatttgaatgtatttagaaaaataaacaataggggttccgcgcacatttccccgaaaagtgccac

SEQ ID NO: 25 - DNA, 4213 bp, Artificial Sequence; identical to SEQ ID NO: 16
  Recombinant synthesis

(19) United States
(12) Patent Application Publication
(10) Pub. No.: US 2021/0145929 A1
(43) Pub. Date: May 20, 2021

(54) AAV COMPOSITIONS, METHODS OF MAKING AND METHODS OF USE

(71) Applicant: NightstaRx Limited, London (GB)

(72) Inventors: Valerie Girard, Boothwyn, PA (US); Richard Truran, London (GB); Tuyen Ong, London (GB)

(21) Appl. No.: 17/045,093

(22) PCT Filed: Apr. 5, 2019
(86) PCT No.: PCT/US19/26064

§ 371 Date: Oct. 2, 2020

Related U.S. Application Data
(60) Provisional application No. 62/773,975, filed on Nov. 30, 2018.
(60) Provisional application No. 62/746,980, filed on Oct. 17, 2018.
(60) Provisional application No. 62/653,139, filed on Apr. 5, 2018.

(51) Int. Cl. A61K 38/16; C12N 5/00; A61K 31/573; C12N 15/86

(52) U.S. Cl. CPC A61K 38/162; C12N 5/005; C12N 5/0031; C12N 2830/42; C12N 15/86; C12N 2750/14151; C12N 2830/48; A61K 31/573

(57) ABSTRACT

Disclosed are methods for the purification of a recombinant AAV (rAAV) particle from a mammalian host cell culture. The disclosure further provides a pharmaceutical composition produced by the methods of the disclosure, pharmaceutical compositions and method of using the pharmaceutical compositions described herein for the treatment of disease.

### RELATED APPLICATIONS

This application claims the benefit of provisional application U.S. Ser. No. 62/653,139, filed Apr. 5, 2018, U.S. Ser. No. 62/746,980, filed Oct. 17, 2018, and U.S. Ser. No. 62/773,975, filed Nov. 30, 2018, the contents of each of which are herein incorporated by reference in their entirety.

### INCORPORATION OF SEQUENCE LISTING

The contents of the text file named “NIGH-013/001WO_SeqList.txt,” which was created on Apr. 4, 2019 and is 60 KB in size, are hereby incorporated by reference in their entirety.

### FIELD OF THE DISCLOSURE

The disclosure relates to the fields of human therapeutics, biologic drug products, viral delivery of human DNA sequences and methods of manufacturing same.

### BACKGROUND

There is a long-felt and unmet need for AAV-based delivery vectors and improved methods of manufacturing these AAV-based delivery vectors.

### SUMMARY

The disclosure provides a method for the purification of an AAV (AAV) particle from a mammalian host cell culture, comprising the steps of: (a) culturing a plurality of mammalian host cells in a culture media under conditions suitable for the formation of a plurality of AAV particles, wherein the plurality of mammalian host cells have been transfected with a plasmid vector comprising an exogenous sequence, a helper plasmid vector, and a plasmid vector comprising a sequence encoding a viral Rep protein and a viral Cap protein to produce a plurality of transfected mammalian host cells; (b) harvesting the culture media comprising the plurality of transfected mammalian host cells; (c) harvesting a plurality of AAV particles from the plurality of transfected mammalian host cells; (d) concentrating the plurality of AAV particles by tangential flow filtration (TFF) to produce a concentrated plurality of AAV particles; (e) enriching the concentrated plurality of AAV particles for full AAV particles by density gradient ultracentrifugation to produce an enriched plurality of full AAV particles; (f) purifying the enriched plurality of full AAV particles by anion exchange (AEX) chromatography or affinity chromatography to produce an eluate comprising a purified and enriched plurality of full AAV particles; and (g) diafiltering and concentrating the eluate from (f) into a formulation buffer by tangential flow filtration (TFF) to produce a final composition comprising the purified and enriched plurality of full AAV particles and the formulation buffer. In some embodiments, a full AAV comprises an exogenous sequence under the control of a promoter capable of expressing the exogenous sequence in a mammalian or human cell.

The disclosure provides a method for the purification of a recombinant AAV (rAAV) particle from a mammalian host cell culture, comprising the steps of: (a) culturing a plurality of mammalian host cells in a culture media under conditions suitable for the formation of a plurality of rAAV particles, wherein the plurality of mammalian host cells have been transfected with a plasmid vector comprising an exogenous sequence, a helper plasmid vector, and a plasmid vector comprising a sequence encoding a viral Rep protein and a viral Cap protein to produce a plurality of transfected mammalian host cells; (b) harvesting the culture media comprising the plurality of transfected mammalian host cells; (c) harvesting a plurality of rAAV particles from the plurality of transfected mammalian host cells; (d) concentrating the plurality of rAAV particles by tangential flow filtration (TFF) to produce a concentrated plurality of rAAV particles; (e) enriching the concentrated plurality of rAAV particles for full rAAV particles by density gradient ultracentrifugation to produce an enriched plurality of full rAAV particles; (f) purifying the enriched plurality of full rAAV particles by anion exchange (AEX) chromatography or affinity chromatography to produce an eluate comprising a purified and enriched plurality of full rAAV particles; and (g) diafiltering and concentrating the eluate from (f) into a formulation buffer by tangential flow filtration (TFF) to produce a final composition comprising the purified and enriched plurality of full rAAV particles and the formulation buffer. In some embodiments, a full rAAV comprises an exogenous sequence under the control of a promoter capable of expressing the exogenous sequence in a mammalian or human cell.

The disclosure provides a method for the purification of a recombinant AAV (rAAV) particle from a mammalian host cell culture, comprising the steps of: (a) culturing a plurality of mammalian host cells in a culture media under conditions suitable for the formation of a plurality of rAAV-REP1 particles, wherein the plurality of mammalian host cells have been transfected with a plasmid vector comprising an exogenous sequence wherein the exogenous sequence comprises a sequence encoding a human Rab escort protein 1 (REP1) protein, a helper plasmid vector, and a plasmid vector comprising a sequence encoding a viral Rep protein and a viral Cap protein to produce a plurality of transfected mammalian host cells; (b) harvesting the culture media comprising the plurality of transfected mammalian host cells; (c) harvesting a plurality of rAAV particles from the plurality of transfected mammalian host cells; (d) concentrating the plurality of rAAV particles by tangential flow filtration (TFF) to produce a concentrated plurality of rAAV particles; (e) enriching the concentrated plurality of rAAV particles for full rAAV particles by density gradient ultracentrifugation to produce an enriched plurality of full rAAV particles; (f) purifying the enriched plurality of full rAAV particles by anion exchange (AEX) chromatography or affinity chromatography to produce an eluate comprising a purified and enriched plurality of full rAAV particles; and (g) diafiltering and concentrating the eluate from (f) into a formulation buffer by tangential flow filtration (TFF) to produce a final composition comprising the purified and enriched plurality of full rAAV particles and the formulation buffer.

In some embodiments of the methods of the disclosure, the sequence encoding the human REP1 protein comprises or consists of the nucleic acid sequence of:

(SEQ ID NO: 1)

| | |
|---|---|
| 1 | gatatcgaat tcctgcagcc cggcggcacc atggcggata |
| | ctctcccttc ggagtttgat |
| 61 | gtgatcgtaa tagggacggg tttgcctgaa tccatcattg |
| | cagctgcatg ttcaagaagt |
| 121 | ggccggagag ttctgcatgt tgattcaaga agctactatg |
| | gaggaaactg ggccagtttt |
| 181 | agcttttcag gactattgtc ctggctaaag gaataccagg |
| | aaaacagtga cattgtaagt |
| 241 | gacagtccag tgtggcaaga ccagatcctt gaaaatgaag |
| | aagccattgc tcttagcagg |
| 301 | aaggacaaaa ctattcaaca tgtggaagta ttttgttatg |
| | ccagtcagga tttgcatgaa |
| 361 | gatgtcgaag aagctggtgc actgcagaaa aatcatgctc |
| | ttgtgacatc tgcaaactcc |
| 421 | acagaagctg cagattctgc cttcctgcct acggaggatg |
| | agtcattaag cactatgagc |
| 481 | tgtgaaatgc tcacagaaca aactccaagc agcgatccag |
| | agaatgcgct agaagtaaat |
| 541 | ggtgctgaag tgacagggga aaaagaaaac cattgtgatg |
| | ataaaacttg tgtgccatca |
| 601 | acttcagcag aagacatgag tgaaaatgtg cctatagcag |
| | aagataccac agagcaacca |
| 661 | aagaaaaaca gaattactta ctcacaaatt attaaagaag |
| | gcaggagatt taatattgat |
| 721 | ttagtatcaa agctgctgta ttctcgagga ttactaattg |
| | atcttctaat caaatctaat |
| 781 | gttagtcgat atgcagagtt taaaaatatt accaggattc |
| | ttgcatttcg agaaggacga |
| 841 | gtggaacagg ttccgtgttc cagagcagat gtctttaata |
| | gcaaacaact tactatggta |
| 901 | gaaaagcgaa tgctaatgaa atttcttaca ttttgtatgg |
| | aatatgagaa atatcctgat |
| 961 | gaatataaag gatatgaaga gatcacattt tatgaatatt |
| | taaagactca aaaattaacc |
| 1021 | cccaacctcc aatatattgt catgcattca attgcaatga |
| | catcagagac agccagcagc |
| 1081 | accatagatg gtctcaaagc taccaaaaac tttcttcact |
| | gtcttgggcg gtatggcaac |
| 1141 | actccatttt tgtttccttt atatggccaa ggagaactcc |
| | cccagtgttt ctgcaggatg |
| 1201 | tgtgctgtgt ttggtggaat ttattgtctt cgccattcag |
| | tacagtgcct tgtagtggac |
| 1261 | aaagaatcca gaaaatgtaa agcaattata gatcagtttg |
| | gtcagagaat aatctctgag |
| 1321 | catttcctcg tggaggacag ttactttcct gagaacatgt |
| | gctcacgtgt gcaatacagg |
| 1381 | cagatctcca gggcagtgct gattacagat agatctgtcc |
| | taaaaacaga ttcagatcaa |
| 1441 | cagatttcca ttttgacagt gccagcagag gaaccaggaa |
| | cttttgctgt tcgggtcatt |
| 1501 | gagttatgtt cttcaacgat gacatgcatg aaaggcacct |
| | atttggttca tttgacttgc |
| 1561 | acatcttcta aaacagcaag agaagattta gaatcagttg |
| | tgcagaaatt gtttgttcca |
| 1621 | tatactgaaa tggagataga aaatgaacaa gtagaaaagc |
| | caagaattct gtgggctctt |
| 1681 | tacttcaata tgagagattc gtcagacatc agcaggagct |
| | gttataatga tttaccatcc |
| 1741 | aacgtttatg tctgctctgg cccagattgt ggtttaggaa |
| | atgataatgc agtcaaacag |
| 1801 | gctgaaacac ttttccagga aatctgcccc aatgaagatt |
| | tctgtccccc tccaccaaat |
| 1861 | cctgaagaca ttatccttga tggagacagt ttacagccag |
| | aggcttcaga atccagtgcc |
| 1921 | ataccagagg ctaactcgga gactttcaag gaaagcacaa |
| | accttggaaa cctagaggag |
| 1981 | tcctctgaat aa, |

in which the the Kozac consensus is indicated by underlining and the start codon is indicated by bold.

In some embodiments of the methods of the disclosure, the human REP1 protein comprises or consists of the amino acid sequence of

(SEQ ID NO: 2)

| | |
|---|---|
| 1 | MADTLPSEFD VIVIGTGLPE SIIAAACSRS GRRVLHVDSR |
| | SYYGGNWASF SFSGLLSWLK |
| 61 | EYQENSDIVS DSPVWQDQIL ENEEAIALSR KDKTIQHVEV |
| | FCYASQDLHE DVEEAGALQK |
| 121 | NHALVTSANS TEAADSAFLP TEDESLSTMS CEMLTEQTPS |
| | SDPENALEVN GAEVTGEKEN |
| 181 | HCDDKTCVPS TSAEDMSENV PIAEDTTEQP KKNRITYSQI |
| | IKEGRRFNID LVSKLLYSRG |
| 241 | LLIDLLIKSN VSRYAEFKNI TRILAFREGR VEQVPCSRAD |
| | VFNSKQLTMV EKRMLMKFLT |
| 301 | FCMEYEKYPD EYKGYEEITF YEYLKTQKLT PNLQYIVMHS |
| | IAMTSETASS TIDGLKATKN |
| 361 | FLHCLGRYGN TPFLFPLYGQ GELPQCFCRM CAVFGGIYCL |
| | RHSVQCLVVD KESRKCKAII |
| 421 | DQFGQRIISE HFLVEDSYFP ENMCSRVQYR QISRAVLITD |
| | RSVLKTDSDQ QISILTVPAE |
| 481 | EPGTFAVRVI ELCSSTMTCM KGTYLVHLTC TSSKTAREDL |
| | ESVVQKLFVP YTEMEIENEQ |
| 541 | VEKPRILWAL YFNMRDSSDI SRSCYNDLPS NVYVCSGPDC |
| | GLGNDNAVKQ AETLFQEICP |
| 601 | NEDFCPPPPN PEDIILDGDS LQPEASESSA IPEANSETFK |
| | ESTNLGNLEE SSE |

In some embodiments of the method of the disclosure, the culture media comprises reduced fetal bovine serum. In some embodiments, the culture media does not comprise reduced fetal bovine serum.

In some embodiments of the methods of the disclosure, the culture media comprises Dulbecco's Modified Eagle's medium (DMEM).

In some embodiments of the methods of the disclosure, the culture media comprises glycine, L-Arginine hydrochloride, L-Cysteine dihydrochloride, L-Glutamine, L-Histidine hydrochloride-H₂O, L-Isoleucine, L-Leucine, L-Lysine hydrochloride, L-Methionine, L-Phenylalanine, L-Serine, L-Threonine, L-Tryptophan, L-Tyrosine disodium salt dehydrate, L-Valine, Choline chloride, D-Calcium pantothenate, Folic Acid, Niacinamide, Pyridoxine hydrochloride, Riboflavin, Thiamine hydrochloride, i-Inositol, Calcium Chloride (CaCl₂) (anhyd.), Ferric Nitrate (Fe(NO₃)₃″9H2O), Magnesium Sulfate (MgSO₄) (anhyd.), Potassium Chloride (KCl), Sodium Bicarbonate (NaHCO₃), Sodium Chloride (NaCl), Sodium Phosphate monobasic (NaH₂PO4-H₂O), and D-Glucose (Dextrose).

In some embodiments of the methods of the disclosure, the culture media comprises a serum-free media. In some embodiments, the culture media consists of a serum-free media.

In some embodiments of the methods of the disclosure, the culture media comprises a clarified media. In some embodiments, the culture media consists of a clarified media. In some embodiments of the methods of the disclosure, the harvest media comprises a protein-free media. In some embodiments of the methods of the disclosure, the harvest media consists of a protein-free media.

In some embodiments of the method of the disclosure, the mammalian cells have been transfected with a composition comprising a PEI transduction reagent.

In some embodiments of the method of the disclosure, the plasmid vector comprising an exogenous sequence further comprises a sequence encoding a 5′ inverted terminal repeat (ITR) and a sequence encoding a 3′ ITR. In some embodiments, the sequence encoding a 5′ ITR is derived from a sequence encoding a 5′ITR of an AAV of serotype 2 (AAV2). In some embodiments, the sequence encoding a 5′ ITR comprises a sequence that is identical to a sequence encoding a 5′ITR of an AAV of serotype 2 (AAV2). In some embodiments, the sequence encoding a 5′ ITR comprises a sequence that is not identical to a sequence encoding a 5′ITR of an AAV of serotype 2 (AAV2). In some embodiments, the sequence encoding a 3′ ITR is derived from a sequence encoding a 3′ITR of an AAV of serotype 2 (AAV2). In some embodiments, the sequence encoding a 3′ ITR comprises a sequence that is identical to a sequence encoding a 3′ITR of an AAV of serotype 2 (AAV2). In some embodiments, the sequence encoding a 3′ ITR comprises a sequence that is not identical to a sequence encoding a 3′ITR of an AAV of serotype 2 (AAV2). In some embodiments, the sequence encoding a 5′ ITR or the sequence encoding a 3′ ITR comprises 145 base pairs (bp).

In some embodiments of the method of the disclosure, the plasmid vector comprising an exogenous sequence, the helper plasmid vector or the plasmid vector comprising a sequence encoding a viral Rep protein and a viral Cap protein, further comprises a sequence encoding a selection marker. In some embodiments, the plasmid vector comprising an exogenous sequence further comprises a sequence encoding a selection marker. In some embodiments, the helper plasmid vector further comprises a sequence encoding a selection marker. In some embodiments, the plasmid vector comprising a sequence encoding a viral Rep protein and a viral Cap protein further comprises a sequence encoding a selection marker. In some embodiments, the sequence encoding a selection marker conveys resistance to kanamycin.

In some embodiments of the method of the disclosure, the harvesting step (c) comprises a mechanical disruption of the plurality of transfected mammalian cells to release recombinant AAV (rAAV) particles produced by the plurality of transfected mammalian cells. In some embodiments, the mechanical disruption comprises a microfluidization.

In some embodiments of the method of the disclosure, the concentrating step further comprises (1) clarifying the concentrated plurality of rAAV particles by a depth filtration to produce a concentrated and clarified plurality of rAAV particles.

In some embodiments of the method of the disclosure, the concentrating step further comprises (2) freezing the concentrated and clarified plurality of rAAV particles at −80° C. to produce a process intermediate.

In some embodiments of the method of the disclosure, the enriching step (e) comprises an iodixanol density gradient ultracentrifugation to produce an enriched plurality of rAAV particles. In some embodiments, the density gradient is a discontinuous density gradient. In some embodiments, the iodixanol density gradient comprises one or more of an iodixanol composition having a concentration of 15%, 25%, 40% and 57%, respectively. In some embodiments, the enriched plurality of rAAV particles are isolated from an iodixanol density gradient. In some embodiments, the enriched plurality of rAAV particles are isolated from the interface of an iodixanol composition having a concentration of 40% and an iodixanol composition having a concentration of 57%. In some embodiments, the concentrated and clarified plurality of rAAV particles are applied to a density gradient of the disclosure and subsequently subjected to an ultracentrifugation step. In some embodiments, following the ultracentrifugation step, an enriched plurality of rAAV or full rAAV particles is isolated from the density gradient.

In some embodiments of the method of the disclosure, the affinity chromatography of the purifying step (f) comprises an AVB Sepharose matrix.

In some embodiments of the method of the disclosure, the formulation buffer comprises Tris, MgCl₂, and NaCl. In some embodiments, the formulation buffer comprises 20 mM Tris, 1 mM MgCl₂, and 200 mM NaCl at pH 8. In some embodiments, the formulation buffer comprises 20 mM Tris, 1 mM MgCl₂, and 200 mM NaCl at pH 8 with poloxamer 188 at 0.001%.

In some embodiments of the methods of the disclosure, the AEX Chromatography comprises the use of UnoSphere Q or Poros AEX chromatography. In some embodiments, the AEX Chromatography further comprises the steps of generating an AEX Chromatogram and selecting a peak on the AEX Chromatogram containing full rAAV particles.

In some embodiments of the methods for the purification of a recombinant AAV (rAAV) particle of the disclosure, the method further comprises a dilution step prior to step (g), wherein the dilution step comprises (1) diluting the first purified plurality of rAAV particles from step (d) by 20× prior to step (e) when the chromatography comprises contacting the first purified plurality of rAAV particles with UnoQ or (2) diluting the first purified plurality of rAAV particles from step (d) by 6× prior to step (e) when the chromatography comprises contacting the first purified plurality of rAAV particles with AVB. In some embodiments, the dilution step comprises adding a dilution buffer to the first purified plurality of rAAV particles, wherein the chromatography comprises contacting the first purified plurality of rAAV particles with UnoQ and wherein the dilution buffer comprises 10 mM Tris at pH 9. In some embodiments, the dilution step comprises adding a dilution buffer to the first purified plurality of rAAV particles, wherein the chromatography comprises contacting the first purified plurality of rAAV particles with AVB and wherein the dilution buffer comprises 20 mM Tris, 1 mM MgCl₂, and 200 mM NaCl at pH 8. In some embodiments, step (e) produces a composition comprising a second purified plurality of rAAV particles and an elution buffer. In some embodiments, the chromatography comprises UnoQ and wherein the elution buffer comprises 10 mM Tris, 650 mM NaCl at pH 9. In some embodiments, the chromatography comprises AVB and wherein the elution buffer comprises 10.8 mM NaHPO₄, 44.6 mM citric acid, 400 mM NaCl at pH 2.6. In some embodiments, the elution buffer is eluted into a neutralization buffer. In some embodiments, the neutralization buffer comprises 1M Tris at pH 8.8.

In some embodiments of the methods of the disclosure, the TFF of step (d) or step (g) is performed using a 100 kDa hollow fiber filter (HFF). In some embodiments, the TFF of step (d) or step (g) is performed using a 70 kDa HFF. In some embodiments, the TFF of step (d) or step (g) is performed using a 50 kDa HFF. In some embodiments, step (g) the method further comprises a second TFF, the TFF of step (d) and the first TFF of step (g) are performed using a 100 kDa HFF and the second TFF of step (g) is performed using a 50 kDa or a 70 kDa HFF.

In some embodiments of the methods of the disclosure, the host cell is isolated or derived from a cultured cell line. In some embodiments, the host cell is an HEK293 cell.

In some embodiments of the methods of the disclosure, the host cell is isolated or derived from a primary cell line. In some embodiments, the host cell is an immortalized cell or a stem cell.

The disclosure provides a pharmaceutical composition comprising a plurality of rAAV particles produced by a method of the disclosure.

In some embodiments of the pharmaceutical compositions of the disclosure, the pharmaceutical composition comprises (a) between 0.5 and 2.5×10¹² vector genomes (vg)/mL of replication-defective and recombinant adeno-associated virus (rAAV); (b) less than 50% empty capsids; (c) less than 4 ng/mL residual host cell protein per 1.0×10¹² vg/mL; and (d) less than 7×10⁻³ pg/ml residual host cell DNA per 1.0×10¹² vg/mL.

In some embodiments of the pharmaceutical compositions of the disclosure, the pharmaceutical composition further comprises (e) a plurality of functional vg/mL, wherein each of functional vector genomes is capable of expressing an exogenous sequence in a cell following transduction.

In some embodiments of the pharmaceutical compositions of the disclosure, following transduction of a cell with the pharmaceutical composition, the plurality of functional vg/mL express the exogenous sequence at a 2-fold increase when compared to a level of expression of a corresponding endogenous sequence in a nontransduced cell. In some embodiments, following transduction of a cell with the pharmaceutical composition, the plurality of functional vg/mL express the exogenous sequence at a 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, or any other increment fold increase in between, when compared to a level of expression of a corresponding endogenous sequence in a nontransduced cell. In some embodiments, the exogenous sequence and the corresponding endogenous sequence are identical. In some embodiments, the exogenous sequence and the corresponding endogenous sequence are not identical. In some embodiments, the exogenous sequence and the corresponding endogenous sequence are not identical, but the corresponding polypeptide is identical. In some embodiments, the exogenous sequence and the corresponding endogenous sequence have at least 70%, 75%, 80%, 85%, 90%, 95%, 97%, 99% or any percentage in between of identity. In some embodiments, the exogenous sequence is codon-optimized for expression in a mammal or a human when compared to the corresponding endogenous sequence. In some embodiments, including those wherein the exogenous sequence is codon-optimized for expression in a mammal or a human when compared to the corresponding endogenous sequence, the exogenous sequence and the corresponding endogenous sequence have at least 50%, 55%, 60%, 65%, 70%, 75%, 80%, 85%, 90%, 95%, 97%, 99% or any percentage in between of homology.

In some embodiments of the pharmaceutical compositions of the disclosure, following transduction of a cell with a pharmaceutical composition of the disclosure, the exogenous sequence encodes a protein. In some embodiments, the protein encoded by the exogenous sequence has an activity level equal to or greater than an activity level of a protein encoded by a corresponding sequence of a nontransduced cell. In some embodiments, the exogenous sequence and the corresponding endogenous sequence are identical. In some embodiments, the exogenous sequence and the corresponding endogenous sequence are not identical. In some embodiments, the exogenous sequence and the corresponding endogenous sequence are not identical, but the corresponding polypeptide is identical. In some embodiments, the exogenous sequence and the corresponding endogenous sequence have at least 70%, 75%, 80%, 85%, 90%, 95%, 97%, 99% or any percentage in between of identity. In some embodiments, including those wherein the exogenous sequence is codon-optimized for expression in a mammal or a human when compared to the corresponding endogenous sequence, the exogenous sequence and the corresponding endogenous sequence have at least 50%, 55%, 60%, 65%, 70%, 75%, 80%, 85%, 90%, 95%, 97%, 99% or any percentage in between of homology. In some embodiments, the activity comprises binding to, activating, and/or transferring one or more functional groups to a ligand or a substrate. In some embodiments, the protein comprises a REP-1 protein and the activity comprises a prenylation of REP-1 substrate.

In some embodiments of the pharmaceutical compositions of the disclosure, the pharmaceutical composition comprises (a) between 1.0 and 2.0×10¹² vector genomes (vg)/mL of replication-defective and recombinant adeno-associated virus (AAV). In some embodiments, the pharmaceutical composition comprises (a) about 1.0×10¹² vector genomes (vg)/mL of replication-defective and recombinant adeno-associated virus (AAV). In some embodiments, the pharmaceutical composition comprises (a) 1.0×10¹² vector genomes (vg)/mL of replication-defective and recombinant adeno-associated virus (AAV).

In some embodiments of the pharmaceutical compositions of the disclosure, the pharmaceutical composition comprises (b) less than 50% empty capsids. In some embodiments of the pharmaceutical compositions of the disclosure, the pharmaceutical composition comprises (b) less than 30% empty capsids.

In some embodiments of the pharmaceutical compositions of the disclosure, the replication-defective and recombinant adeno-associated virus (rAAV) contains a sequence isolated or derived from an AAV of serotype 2 (AAV2). In some embodiments, the sequence isolated or derived from an AAV2 comprises a sequence encoding an inverted terminal repeat (ITR). In some embodiments, the replication-defective and recombinant adeno-associated virus (rAAV) contains a sequence encoding a 5′ ITR and a sequence encoding a 3′ ITR. In some embodiments, the sequence encoding a 5′ ITR and the sequence encoding a 3′ ITR comprise a wild type sequence of an AAV2 ITR.

In some embodiments of the pharmaceutical compositions of the disclosure, the host cell is isolated or derived from a cultured cell line. In some embodiments, the host cell is an HEK293 cell.

In some embodiments of the pharmaceutical compositions of the disclosure, the host cell is isolated or derived from a primary cell line. In some embodiments, the host cell is an immortalized cell or a stem cell.

In some embodiments of the pharmaceutical compositions of the disclosure, each full rAAV of the plurality of full rAAVs of the final composition further comprises: a nucleic acid sequence comprising, from 5′ to 3′: (a) a sequence encoding an AAV2 5′ ITR, (b) a sequence encoding an early enhancer element, (c) a sequence encoding a promoter, (d) a sequence encoding an exon and intron, (e) a sequence encoding a splice acceptor site, (f) a sequence encoding a Rab escort protein 1 (REP1) protein, (g) a sequence encoding a post-transcriptional regulatory element (PRE), (h) a sequence encoding a polyadenylation (polyA) site, and (i) a sequence encoding an AAV2 3′ ITR. (see also, U.S. Pat. No. 9,834,788, the contents of which are incorporated herein in their entirety).

In some embodiments of the pharmaceutical compositions of the disclosure, including those wherein each full rAAV of the plurality of full rAAVs of the final composition further comprises: a nucleic acid sequence comprising, from 5′ to 3′, elements (a) through (i), the early enhancer element comprises a sequence isolated or derived from a Cytomegalovirus (CMV). In some embodiments, the early enhancer element comprises or consists of the nucleic acid sequence of

### 1 ATTGACGT CAATAATGAC GTATGTTCCC ATAGTAACGC CAATAGGGAC TTTCCATTGA

### 61 CGTCAATGGG TGGAGTATTT ACGGTAAACT GCCCACTTGG CAGTACATCA AGTGTATCAT

### 121 ATGCCAAGTA CGCCCCCTAT TGACGTCAAT GACGGTAAAT GGCCCGCCTG GCATTATGCC

### 181 CAGTACATGA CCTTATGGGA CTTTCCTACT TGGCAGTACA TCTACGTATT AGTCATCGCT

241 ATTACCATGG (SEQ ID NO: 3). In some embodiments, the early enhancer element comprises or consists of the nucleic acid sequence of

(SEQ ID NO: 4)

| | |
|---|---|
| 1 | CCATTGACGT CAATAATGAC GTATGTTCCC ATAGTAACGC |
| | CAATAGGGAC TTTCCATTGA |
| 61 | CGTCAATGGG TGGAGTATTT ACGGTAAACT GCCCACTTGG |
| | CAGTACATCA AGTGTATCAT |
| 121 | ATGCCAAGTA CGCCCCCTAT TGACGTCAAT GACGGTAAAT |
| | GGCCCGCCTG GCATTATGCC |
| 181 | CAGTACATGA CCTTATGGGA CTTTCCTACT TGGCAGTACA |
| | TCTACGTATT AGTCATCGCT |
| 241 | ATTACCATGG. |

In some embodiments of the pharmaceutical compositions of the disclosure, including those wherein each full rAAV of the plurality of full rAAVs of the final composition further comprises: a nucleic acid sequence comprising, from 5′ to 3′, elements (a) through (i), the sequence encoding the promoter comprises or consists of a sequence isolated or derived from a sequence encoding a chicken beta actin (CBA) gene. In some embodiments, the sequence encoding the promoter comprises or consists of the nucleic acid sequence of

(SEQ ID NO: 5)

| | |
|---|---|
| 1 | TCGAGGTGAG CCCCACGTTC TGCTTCACTC TCCCCATCTC |
| | CCCCCCCTCC CCACCCCCAA |
| 61 | TTTTGTATTT ATTTATTTTT TAATTATTTT GTGCAGCGAT |
| | GGGGGCGGGG GGGGGGGGGG |
| 121 | GGCGCGCGCC AGGCGGGGCG GGGCGGGGCG AGGGGCGGGG |
| | CGGGGCGAGG CGGAGAGGTG |
| 181 | CGGCGGCAGC CAATCAGAGC GGCGCGCTCC GAAAGTTTCC |
| | TTTTATGGCG AGGCGGCGGC |
| 241 | GGCGGCGGCC CTATAAAAAG CGAAGCGCGC GGCGGGCGGG |
| | AGTCGCTGCG CGCTGCCTTC |
| 301 | GCCCCGTGCC CCGCTCCGCC GCCGCCTCGC GCCGCCCGCC |
| | CCGGCTCTGA CTGACCGCGT |
| 361 | TACTCCCACA GGTGAGCGGG CGGGACGGCC CTTCTCCTCC |
| | GGGCTGTAAT TAGCGCTTGG |
| 421 | TTTAATGACG GCTTGTTTCT TTTCTGTGGC TGCGTGAAAG |
| | CCTTGAGGGG CTCCGGGAGG |
| 481 | GCCCTTTGTG CGGGGGGAGC GGCTCGGGG. |

In some embodiments of the pharmaceutical compositions of the disclosure, including those wherein each full rAAV of the plurality of full rAAVs of the final composition further comprises: a nucleic acid sequence comprising, from 5′ to 3′, elements (a) through (i), the sequence encoding the exon and intron comprises or consists of a sequence isolated or derived from a sequence encoding a chicken beta actin (CBA) gene. In some embodiments, the sequence encoding the exon and intron comprises or consists of the nucleic acid sequence of

(SEQ ID NO: 5)

| | |
|---|---|
| 1 | TCGAGGTGAG CCCCACGTTC TGCTTCACTC TCCCCATCTC |
| | CCCCCCCTCC CCACCCCCAA |
| 61 | TTTTGTATTT ATTTATTTTT TAATTATTTT GTGCAGCGAT |
| | GGGGGCGGGG GGGGGGGGGG |
| 121 | GGCGCGCGCC AGGCGGGGCG GGGCGGGGCG AGGGGCGGGG |
| | CGGGGCGAGG CGGAGAGGTG |
| 181 | CGGCGGCAGC CAATCAGAGC GGCGCGCTCC GAAAGTTTCC |
| | TTTTATGGCG AGGCGGCGGC |
| 241 | GGCGGCGGCC CTATAAAAAG CGAAGCGCGC GGCGGGCGGG |
| | AGTCGCTGCG CGCTGCCTTC |
| 301 | GCCCCGTGCC CCGCTCCGCC GCCGCCTCGC GCCGCCCGCC |
| | CCGGCTCTGA CTGACCGCGT |
| 361 | TACTCCCACA GGTGAGCGGG CGGGACGGCC CTTCTCCTCC |
| | GGGCTGTAAT TAGCGCTTGG |
| 421 | TTTAATGACG GCTTGTTTCT TTTCTGTGGC TGCGTGAAAG |
| | CCTTGAGGGG CTCCGGGAGG |
| 481 | GCCCTTTGTG CGGGGGGAGC GGCTCGGGG. |

In some embodiments of the pharmaceutical compositions of the disclosure, including those wherein each full rAAV of the plurality of full rAAVs of the final composition further comprises: a nucleic acid sequence comprising, from 5′ to 3′, elements (a) through (i), the sequence encoding the splice acceptor site comprises a sequence isolated or derived from a sequence encoding an Oryctolagus cuniculus beta globin splice acceptor site. In some embodiments, the sequence encoding the Oryctolagus cuniculus beta globin splice acceptor site comprises or consists of the nucleic acid sequence of

(SEQ ID NO: 6)

| | |
|---|---|
| 1 | CTCTGCT AACCATGTTC ATGCCTTCTT CTTTTTCCTA |
| | CAGCTCCTGG GCAACGTGCT |
| 61 | GGTTATTGTG CTGTCTCATC ATTTTGGCAA AGAATT. |

In some embodiments of the pharmaceutical compositions of the disclosure, including those wherein each full rAAV of the plurality of full rAAVs of the final composition further comprises: a nucleic acid sequence comprising, from 5′ to 3′, elements (a) through (i), the sequence comprising the early enhancer element, the sequence comprising the promoter, the sequence comprising the intron and exon and the sequence comprising the splice acceptor site, comprise or consist of the nucleic acid sequence of

(SEQ ID NO: 7)

| | |
|---|---|
| 1 | TCGAGGTGAG CCCCACGTTC TGCTTCACTC TCCCCATCTC |
| | CCCCCCCTCC CCACCCCCAA |
| 61 | TTTTGTATTT ATTTATTTTT TAATTATTTT GTGCAGCGAT |
| | GGGGGCGGGG GGGGGGGGGG |
| 121 | GGCGCGCGCC AGGCGGGGCG GGGCGGGGCG AGGGGCGGGG |
| | CGGGGCGAGG CGGAGAGGTG |
| 181 | CGGCGGCAGC CAATCAGAGC GGCGCGCTCC GAAAGTTTCC |
| | TTTTATGGCG AGGCGGCGGC |
| 241 | GGCGGCGGCC CTATAAAAAG CGAAGCGCGC GGCGGGCGGG |
| | AGTCGCTGCG CGCTGCCTTC |
| 301 | GCCCCGTGCC CCGCTCCGCC GCCGCCTCGC GCCGCCCGCC |
| | CCGGCTCTGA CTGACCGCGT |
| 361 | TACTCCCACA GGTGAGCGGG CGGGACGGCC CTTCTCCTCC |
| | GGGCTGTAAT TAGCGCTTGG |
| 421 | TTTAATGACG GCTTGTTTCT TTTCTGTGGC TGCGTGAAAG |
| | CCTTGAGGGG CTCCGGGAGG |
| 481 | GCCCTTTGTG CGGGGGGAGC GGCTCGGGGC TGTCCGCGGG |
| | GGGACGGCTG CCTTCGGGGG |
| 541 | GGACGGGGCA GGGCGGGGTT CGGCTTCTGG CGTGTGACCG |
| | GCGGCTCTAG AGCCTCTGCT |
| 601 | AACCATGTTC ATGCCTTCTT CTTTTTCCTA CAGCTCCTGG |
| | GCAACGTGCT GGTTATTGTG |
| 661 | CTGTCTCATC ATTTTGGCAA AGAATTGGAT C. |

In some embodiments of the pharmaceutical compositions of the disclosure, including those wherein each full rAAV of the plurality of full rAAVs of the final composition further comprises: a nucleic acid sequence comprising, from 5′ to 3′, elements (a) through (i), the sequence comprising the early enhancer element, the sequence comprising the promoter, the sequence comprising the intron and exon and the sequence comprising the splice acceptor site, comprise or consist of the nucleic acid sequence of

(SEQ ID NO: 8)

| | |
|---|---|
| 1 | CCATTGACGT CAATAATGAC GTATGTTCCC ATAGTAACGC |
| | CAATAGGGAC TTTCCATTGA |
| 61 | CGTCAATGGG TGGAGTATTT ACGGTAAACT GCCCACTTGG |
| | CAGTACATCA AGTGTATCAT |
| 121 | ATGCCAAGTA CGCCCCCTAT TGACGTCAAT GACGGTAAAT |
| | GGCCCGCCTG GCATTATGCC |
| 181 | CAGTACATGA CCTTATGGGA CTTTCCTACT TGGCAGTACA |
| | TCTACGTATT AGTCATCGCT |
| 241 | ATTACCATGG TCGAGGTGAG CCCCACGTTC TGCTTCACTC |
| | TCCCCATCTC CCCCCCCTCC |
| 301 | CCACCCCCAA TTTTGTATTT ATTTATTTTT TAATTATTTT |
| | GTGCAGCGAT GGGGGCGGGG |
| 361 | GGGGGGGGGG GGCGCGCGCC AGGCGGGGCG GGGCGGGGCG |
| | AGGGGCGGGG CGGGGCGAGG |
| 421 | CGGAGAGGTG CGGCGGCAGC CAATCAGAGC GGCGCGCTCC |
| | GAAAGTTTCC TTTTATGGCG |
| 481 | AGGCGGCGGC GGCGGCGGCC CTATAAAAAG CGAAGCGCGC |
| | GGCGGGCGGG AGTCGCTGCG |
| 541 | CGCTGCCTTC GCCCCGTGCC CCGCTCCGCC GCCGCCTCGC |
| | GCCGCCCGCC CCGGCTCTGA |
| 601 | CTGACCGCGT TACTCCCACA GGTGAGCGGG CGGGACGGCC |
| | CTTCTCCTCC GGGCTGTAAT |
| 661 | TAGCGCTTGG TTTAATGACG GCTTGTTTCT TTTCTGTGGC |
| | TGCGTGAAAG CCTTGAGGGG |
| 721 | CTCCGGGAGG GCCCTTTGTG CGGGGGGAGC GGCTCGGGGC |
| | TGTCCGCGGG GGGACGGCTG |
| 781 | CCTTCGGGGG GGACGGGGCA GGGCGGGGTT CGGCTTCTGG |
| | CGTGTGACCG GCGGCTCTAG |
| 841 | AGCCTCTGCT AACCATGTTC ATGCCTTCTT CTTTTTCCTA |
| | CAGCTCCTGG GCAACGTGCT |
| 901 | GGTTATTGTG CTGTCTCATC ATTTTGGCAA AGAATTGGAT C. |

In some embodiments of the pharmaceutical compositions of the disclosure, including those wherein each full rAAV of the plurality of full rAAVs of the final composition further comprises: a nucleic acid sequence comprising, from 5′ to 3′, elements (a) through (i), the sequence encoding the REP1 protein comprises a sequence isolated or derived from a mammalian REP1 sequence. In some embodiments, the mammalian REP1 sequence is isolated or derived from a mouse, a rat, a rabbit, a non-human primate or a human. In some embodiments, the mammalian REP1 sequence is isolated or derived from a human. In some embodiments, the sequence encoding the human REP1 protein comprises or consists of the nucleic acid sequence of

(SEQ ID NO: 9)

| | |
|---|---|
| 1 | gatatcgaat tcctgcagcc cggcggcacc atggcggata |
| | ctctcccttc ggagtttgat |
| 61 | gtgatcgtaa tagggacggg tttgcctgaa tccatcattg |
| | cagctgcatg ttcaagaagt |
| 121 | ggccggagag ttctgcatgt tgattcaaga agctactatg |
| | gaggaaactg ggccagtttt |
| 181 | agcttttcag gactattgtc ctggctaaag gaataccagg |
| | aaaacagtga cattgtaagt |
| 241 | gacagtccag tgtggcaaga ccagatcctt gaaaatgaag |
| | aagccattgc tcttagcagg |
| 301 | aaggacaaaa ctattcaaca tgtggaagta ttttgttatg |
| | ccagtcagga tttgcatgaa |
| 361 | gatgtcgaag aagctggtgc actgcagaaa aatcatgctc |
| | ttgtgacatc tgcaaactcc |
| 421 | acagaagctg cagattctgc cttcctgcct acggaggatg |
| | agtcattaag cactatgagc |
| 481 | tgtgaaatgc tcacagaaca aactccaagc agcgatccag |
| | agaatgcgct agaagtaaat |
| 541 | ggtgctgaag tgacagggga aaaagaaaac cattgtgatg |
| | ataaaacttg tgtgccatca |
| 601 | acttcagcag aagacatgag tgaaaatgtg cctatagcag |
| | aagataccac agagcaacca |
| 661 | aagaaaaaca gaattactta ctcacaaatt attaaagaag |
| | gcaggagatt taatattgat |
| 721 | ttagtatcaa agctgctgta ttctcgagga ttactaattg |
| | atcttctaat caaatctaat |
| 781 | gttagtcgat atgcagagtt taaaaatatt accaggattc |
| | ttgcatttcg agaaggacga |
| 841 | gtggaacagg ttccgtgttc cagagcagat gtctttaata |
| | gcaaacaact tactatggta |
| 901 | gaaaagcgaa tgctaatgaa atttcttaca ttttgtatgg |
| | aatatgagaa atatcctgat |
| 961 | gaatataaag gatatgaaga gatcacattt tatgaatatt |
| | taaagactca aaaattaacc |
| 1021 | cccaacctcc aatatattgt catgcattca attgcaatga |
| | catcagagac agccagcagc |
| 1081 | accatagatg gtctcaaagc taccaaaaac tttcttcact |
| | gtcttgggcg gtatggcaac |
| 1141 | actccatttt tgtttccttt atatggccaa ggagaactcc |
| | cccagtgttt ctgcaggatg |
| 1201 | tgtgctgtgt ttggtggaat ttattgtctt cgccattcag |
| | tacagtgcct tgtagtggac |
| 1261 | aaagaatcca gaaaatgtaa agcaattata gatcagtttg |
| | gtcagagaat aatctctgag |
| 1321 | catttcctcg tggaggacag ttactttcct gagaacatgt |
| | gctcacgtgt gcaatacagg |
| 1381 | cagatctcca gggcagtgct gattacagat agatctgtcc |
| | taaaaacaga ttcagatcaa |
| 1441 | cagatttcca ttttgacagt gccagcagag gaaccaggaa |
| | cttttgctgt tcgggtcatt |
| 1501 | gagttatgtt cttcaacgat gacatgcatg aaaggcacct |
| | atttggttca tttgacttgc |
| 1561 | acatcttcta aaacagcaag agaagattta gaatcagttg |
| | tgcagaaatt gtttgttcca |
| 1621 | tatactgaaa tggagataga aaatgaacaa gtagaaaagc |
| | caagaattct gtgggctctt |
| 1681 | tacttcaata tgagagattc gtcagacatc agcaggagct |
| | gttataatga tttaccatcc |
| 1741 | aacgtttatg tctgctctgg cccagattgt ggtttaggaa |
| | atgataatgc agtcaaacag |
| 1801 | gctgaaacac ttttccagga aatctgcccc aatgaagatt |
| | tctgtccccc tccaccaaat |
| 1861 | cctgaagaca ttatccttga tggagacagt ttacagccag |
| | aggcttcaga atccagtgcc |
| 1921 | ataccagagg ctaactcgga gactttcaag gaaagcacaa |
| | accttggaaa cctagaggag |
| 1981 | tcctctgaat aa. |

In some embodiments, the human REP1 protein comprises or consists of the amino acid sequence of

(SEQ ID NO: 10)

| | |
|---|---|
| 1 | MADTLPSEFD VIVIGTGLPE SIIAAACSRS GRRVLHVDSR |
| | SYYGGNWASF SFSGLLSWLK |
| 61 | EYQENSDIVS DSPVWQDQIL ENEEAIALSR KDKTIQHVEV |
| | FCYASQDLHE DVEEAGALQK |
| 121 | NHALVTSANS TEAADSAFLP TEDESLSTMS CEMLTEQTPS |
| | SDPENALEVN GAEVTGEKEN |
| 181 | HCDDKTCVPS TSAEDMSENV PIAEDTTEQP KKNRITYSQI |
| | IKEGRRFNID LVSKLLYSRG |
| 241 | LLIDLLIKSN VSRYAEFKNI TRILAFREGR VEQVPCSRAD |
| | VFNSKQLTMV EKRMLMKFLT |
| 301 | FCMEYEKYPD EYKGYEEITF YEYLKTQKLT PNLQYIVMHS |
| | IAMTSETASS TIDGLKATKN |
| 361 | FLHCLGRYGN TPFLFPLYGQ GELPQCFCRM CAVFGGIYCL |
| | RHSVQCLVVD KESRKCKAII |
| 421 | DQFGQRIISE HFLVEDSYFP ENMCSRVQYR QISRAVLITD |
| | RSVLKTDSDQ QISILTVPAE |
| 481 | EPGTFAVRVI ELCSSTMTCM KGTYLVHLTC TSSKTAREDL |
| | ESVVQKLFVP YTEMEIENEQ |
| 541 | VEKPRILWAL YFNMRDSSDI SRSCYNDLPS NVYVCSGPDC |
| | GLGNDNAVKQ AETLFQEICP |
| 601 | NEDFCPPPPN PEDIILDGDS LQPEASESSA IPEANSETFK |
| | ESTNLGNLEE SSE. |

In some embodiments of the pharmaceutical compositions of the disclosure, including those wherein each full rAAV of the plurality of full rAAVs of the final composition further comprises: a nucleic acid sequence comprising, from 5′ to 3′, elements (a) through (i), the sequence encoding the PRE comprises a sequence isolated or derived from a Woodchuck Hepatitis virus (WPRE). In some embodiments, the sequence encoding the WPRE comprises or consists of a nucleic acid sequence of

(SEQ ID NO: 11)

| | |
|---|---|
| 1 | atcaacctct ggattacaaa atttgtgaaa gattgactgg |
| | tattcttaac tatgttgctc |
| 61 | cttttacgct atgtggatac gctgctttaa tgcctttgta |
| | tcatgctatt gcttcccgta |
| 121 | tggctttcat tttctcctcc ttgtataaat cctggttgct |
| | gtctctttat gaggagttgt |
| 181 | ggcccgttgt caggcaacgt ggcgtggtgt gcactgtgtt |
| | tgctgacgca acccccactg |
| 241 | gttggggcat tgccaccacc tgtcagctcc tttccgggac |
| | tttcgctttc cccctcccta |
| 301 | ttgccacggc ggaactcatc gccgcctgcc ttgcccgctg |
| | ctggacaggg gctcggctgt |
| 361 | tgggcactga caattccgtg gtgttgtcgg ggaaatcatc |
| | gtcctttcct tggctgctcg |
| 421 | cctgtgttgc cacctggatt ctgcgcggga cgtccttctg |
| | ctacgtccct tcggccctca |
| 481 | atccagcgga ccttccttcc cgcggcctgc tgccggctct |
| | gcggcctctt ccgcgtcttc |
| 541 | gccttcgccc tcagacgagt cggatctccc tttgggccgc |
| | ctccccgc. |

In some embodiments of the pharmaceutical compositions of the disclosure, including those wherein each full rAAV of the plurality of full rAAVs of the final composition further comprises: a nucleic acid sequence comprising, from 5′ to 3′, elements (a) through (i), the sequence encoding a polyadenylation (polyA) site comprises a sequence isolated or derived from a mammalian gene. In some embodiments, the sequence encoding a polyadenylation (polyA) site comprises a sequence isolated or derived from a bovine growth hormone gene (BGH). In some embodiments, the sequence encoding the polyA site comprises or consists of the nucleic acid sequence of

(SEQ ID NO: 12)

| | |
|---|---|
| 1 | tcgctgatca gcctcgactg tgccttctag ttgccagcca |
| | tctgttgttt gcccctcccc |
| 61 | cgtgccttcc ttgaccctgg aaggtgccac tcccactgtc |
| | ctttcctaat aaaatgagga |
| 121 | aattgcatcg cattgtctga gtaggtgtca ttctattctg |
| | gggggtgggg tggggcagga |
| 181 | cagcaagggg gaggattggg aagacaatag caggcatgct |
| | ggggatgcgg tgggctctat |
| 241 | ggcttctgag gcggaaagaa ccagctgggg. |

In some embodiments of the pharmaceutical compositions of the disclosure, including those wherein each full rAAV of the plurality of full rAAVs of the final composition further comprises: a nucleic acid sequence comprising, from 5′ to 3′, elements (a) through (i), the sequence encoding the AAV2 5′ITR comprises or consists of the nucleic acid sequence of

(SEQ ID NO: 13)

| | |
|---|---|
| 1 | ctgcgcgctc gctcgctcac tgaggccgcc cgggcaaagc |
| | ccgggcgtcg ggcgaccttt |
| 61 | ggtcgcccgg cctcagtgag cgagcgagcg cgcagagagg |
| | gagtggccaa ctccatcact |
| 121 | aggggttcct tgtagttaat gatt. |

In some embodiments of the pharmaceutical compositions of the disclosure, including those wherein each full rAAV of the plurality of full rAAVs of the final composition further comprises: a nucleic acid sequence comprising, from 5′ to 3′, elements (a) through (i), the sequence encoding the AAV2 3′ITR comprises or consists of the nucleic acid sequence of

(SEQ ID NO: 14)

| | |
|---|---|
| 1 | AGGAACCCCT AGTGATGGAG TTGGCCACTC CCTCTCTGCG |
| | CGCTCGCTCG CTCACTGAGG |
| 61 | CCGGGCGACC AAAGGTCGCC CGACGCCCGG GCGGCCTCAG |
| | TGAGCGAGCG AGCGCGCAGA |
| 121 | G. |

In some embodiments of the pharmaceutical compositions of the disclosure, including those wherein each full rAAV of the plurality of full rAAVs of the final composition further comprises: a nucleic acid sequence comprising, from 5′ to 3′, elements (a) through (i), the nucleic acid comprising, from 5′ to 3′, elements (a) through (i) comprises or consists of a DNA sequence. In some embodiments, the nucleic acid comprising, from 5′ to 3′, elements (a) through (i) comprises or consists of a single-stranded DNA sequence.

In some embodiments of the pharmaceutical compositions of the disclosure, including those wherein each full rAAV of the plurality of full rAAVs of the final composition further comprises: a nucleic acid sequence comprising, from 5′ to 3′, elements (a) through (i), each rAAV of the plurality of full rAAV of the final composition comprises a capsid protein isolated or derived from an AAV2. In some embodiments, the AAV2 capsid protein comprises a sequence having at least 95% identity to the amino acid sequence

(SEQ ID NO: 15)

| | |
|---|---|
| 1 | MAADGYLPDW LEDTLSEGIR QWWKLKPGPP PPKPAERHKD |
| | DSRGLVLPGY KYLGPFNGLD |
| 61 | KGEPVNEADA AALEHDKAYD RQLDSGDNPY LKYNHADAEF |
| | QERLKEDTSF GGNLGRAVFQ |
| 121 | AKKRVLEPLG LVEEPVKTAP GKKRPVEHSP VEPDSSSGTG |
| | KAGQQPARKR LNFGQTGDAD |
| 181 | SVPDPQPLGQ PPAAPSGLGT NTMATGSGAP MADNNEGADG |
| | VGNSSGNWHC DSTWMGDRVI |
| 241 | TTSTRTWALP TYNNHLYKQI SSQSGASNDN HYFGYSTPWG |
| | YFDFNRFHCH FSPRDWQRLI |
| 301 | NNNWGFRPKR LNFKLFNIQV KEVTQNDGTT TIANNLTSTV |
| | QVFTDSEYQL PYVLGSAHQG |
| 361 | CLPPFPADVF MVPQYGYLTL NNGSQAVGRS SFYCLEYFPS |
| | QMLRTGNNFT FSYTFEDVPF |
| 421 | HSSYAHSQSL DRLMNPLIDQ YLYYLSRTNT PSGTTTQSRL |
| | QFSQAGASDI RDQSRNWLPG |
| 481 | PCYRQQRVSK TSADNNNSEY SWTGATKYHL NGRDSLVNPG |
| | PAMASHKDDE EKFFPQSGVL |
| 541 | IFGKQGSEKT NVDIEKVMIT DEEEIRTTNP VATEQYGSVS |
| | TNLQRGNRQA ATADVNTQGV |
| 601 | LPGMVWQDRD VYLQGPIWAK IPHTDGHFHP SPLMGGFGLK |
| | HPPPQILIKN TPVPANPSTT |
| 661 | FSAAKFASFI TQYSTGQVSV EIEWELQKEN SKRWNPEIQY |
| | TSNYNKSVNV DFTVDTNGVY |
| 721 | SEPRPIGTRY LTRNL. |

In some embodiments, the AAV2 capsid protein comprises the amino acid sequence

(SEQ ID NO: 15)

| | |
|---|---|
| 1 | MAADGYLPDW LEDTLSEGIR QWWKLKPGPP PPKPAERHKD |
| | DSRGLVLPGY KYLGPFNGLD |
| 61 | KGEPVNEADA AALEHDKAYD RQLDSGDNPY LKYNHADAEF |
| | QERLKEDTSF GGNLGRAVFQ |
| 121 | AKKRVLEPLG LVEEPVKTAP GKKRPVEHSP VEPDSSSGTG |
| | KAGQQPARKR LNFGQTGDAD |
| 181 | SVPDPQPLGQ PPAAPSGLGT NTMATGSGAP MADNNEGADG |
| | VGNSSGNWHC DSTWMGDRVI |
| 241 | TTSTRTWALP TYNNHLYKQI SSQSGASNDN HYFGYSTPWG |
| | YFDFNRFHCH FSPRDWQRLI |
| 301 | NNNWGFRPKR LNFKLFNIQV KEVTQNDGTT TIANNLTSTV |
| | QVFTDSEYQL PYVLGSAHQG |
| 361 | CLPPFPADVF MVPQYGYLTL NNGSQAVGRS SFYCLEYFPS |
| | QMLRTGNNFT FSYTFEDVPF |
| 421 | HSSYAHSQSL DRLMNPLIDQ YLYYLSRTNT PSGTTTQSRL |
| | QFSQAGASDI RDQSRNWLPG |
| 481 | PCYRQQRVSK TSADNNNSEY SWTGATKYHL NGRDSLVNPG |
| | PAMASHKDDE EKFFPQSGVL |
| 541 | IFGKQGSEKT NVDIEKVMIT DEEEIRTTNP VATEQYGSVS |
| | TNLQRGNRQA ATADVNTQGV |
| 601 | LPGMVWQDRD VYLQGPIWAK IPHTDGHFHP SPLMGGFGLK |
| | HPPPQILIKN TPVPANPSTT |
| 661 | FSAAKFASFI TQYSTGQVSV EIEWELQKEN SKRWNPEIQY |
| | TSNYNKSVNV DFTVDTNGVY |
| 721 | SEPRPIGTRY LTRNL. |

In some embodiments of the pharmaceutical compositions of the disclosure, including those wherein each full rAAV of the plurality of full rAAVs of the final composition further comprises: a nucleic acid sequence comprising, from 5′ to 3′, elements (a) through (i), the pharmaceutical composition further comprises a formulation buffer. In some embodiments, the formulation buffer comprises Tris, MgCl₂, and NaCl. In some embodiments, the formulation buffer comprises 20 mM Tris, 1 mM MgCl₂, and 200 mM NaCl at pH 8. In some embodiments, the formulation buffer comprises 20 mM Tris, 1 mM MgCl₂, and 200 mM NaCl at pH 8 with poloxamer 188 at 0.001%.

In some embodiments of the pharmaceutical compositions of the disclosure, including those wherein each full rAAV of the plurality of full rAAVs of the final composition further comprises: a nucleic acid sequence comprising, from 5′ to 3′, elements (a) through (i), the plurality of full rAAVs are at a concentration of between 1×10⁸ genome particles (gp)/mL and 1×10¹⁴ gp/mL, inclusive of the endpoints. In some embodiments, the plurality of full rAAVs are at a concentration of between 0.5×10¹⁰ gp/mL and 2.5×10¹² gp/mL, inclusive of the endpoints. In some embodiments, the plurality of full rAAVs are at a concentration of between 1×10¹¹ gp/mL and 5×10¹³ gp/mL inclusive of the endpoints. In some embodiments, the plurality of full rAAVs are at a concentration of between 1×10¹¹ gp/mL and 2×10¹² gp/mL, inclusive of the endpoints. In some embodiments, the plurality of full rAAVs are at a concentration of 1×10¹² gp/mL. In some embodiments, the plurality of full rAAVs are at a concentration of 1×10¹¹ gp/mL. In some embodiments, the concentration of the plurality of full rAAVs is measured using qPCR. In some embodiments, the qPCR uses a supercoiled plasmid vector as a standard. In some embodiments, the qPCR uses a linearized plasmid vector as a standard.

The disclosure provides a delivery device comprising the pharmaceutical composition of the disclosure. In some embodiments, the delivery device comprises one or more of a syringe, a catheter and a needle. In some embodiments, the delivery device is suitable for administering the pharmaceutical composition by injection. In some embodiments, the delivery device is suitable for administering the pharmaceutical composition by infusion. In some embodiments, the delivery device is suitable for administering the pharmaceutical composition by a subretinal route. In some embodiments, the delivery device is suitable for administering the pharmaceutical composition by a suprachoroidal route.

The disclosure provides a method of treating a disease or disorder in a subject in need thereof, comprising administering to the subject a therapeutically effective amount of a pharmaceutical composition of the disclosure.

In some embodiments of the methods of treating a disease or disorder of the disclosure, the disease or disorder is a retinal disease or disorder. In some embodiments, the disease or disorder is Choroideremia.

In some embodiments of the methods of treating a disease or disorder of the disclosure, the therapeutically effective amount comprises an amount between a minimally effective amount and a maximally tolerable amount of the pharmaceutical composition.

In some embodiments of the methods of treating a disease or disorder of the disclosure, the minimally effective amount comprises an amount of the pharmaceutical composition sufficient to transduce at least one neuron of a retina or a target portion thereof. In some embodiments, the minimally effective amount comprises an amount of the pharmaceutical composition sufficient to transduce at least 1%, 5%, 10%, 15%, 20%, 25%, 30%, 35%, 40%, 45%, 50%, 55%, 60%, 65%, 70%, 75%, 80%, 85%, 90%, 95%, 97%, 99% or any percentage in between of the neurons of a retina or a target portion thereof. In some embodiments, the minimally effective amount comprises an amount of the pharmaceutical composition sufficient to improve visual acuity of the subject. In some embodiments, the minimally effective amount comprises an amount of the pharmaceutical composition sufficient to reduce a sign or symptom of a retinal disease. In some embodiments, the retinal disease is Choroideremia.

In some embodiments of the methods of treating a disease or disorder of the disclosure, the maximally tolerable amount comprises an amount of the pharmaceutical composition sufficient to induce an adverse event. In some embodiments, the adverse effect comprises an immune response to the pharmaceutical composition. In some embodiments, the immune response comprises inflammation. In some embodiments, the inflammation is systemic. In some embodiments, the inflammation is local. In some embodiments, the adverse event is severe. In some embodiments, the adverse event cannot be prevented, reduced or controlled by administering a secondary medical treatment to the subject. In some embodiments, the secondary medical treatment comprises a suppressant of the immune system. In some embodiments, the suppressant comprises an anti-inflammatory agent. In some embodiments, the anti-inflammatory agent comprises a corticosteroid. In some embodiments, the corticosteroid comprises prednisone or prednisolone.

In some embodiments of the methods of treating a disease or disorder of the disclosure, the therapeutically effective amount of the pharmaceutical composition comprises an amount having a multiplicity of infection (MOI) of between 10⁴ and 10⁷, inclusive of the endpoints. In some embodiments, the therapeutically effective amount of the pharmaceutical composition comprises an amount having a multiplicity of infection (MOI) of between 1×10⁶ and 9×10⁶, inclusive of the endpoints. In some embodiments, the therapeutically effective amount of the pharmaceutical composition comprises an amount having a multiplicity of infection (MOI) of between 10⁴ and 10⁵, inclusive of the endpoints. In some embodiments, the therapeutically effective amount of the pharmaceutical composition comprises an amount having a multiplicity of infection (MOI) of 10⁵.

In some embodiments of the methods of treating a disease or disorder of the disclosure, the therapeutically effective amount comprises between 1×10⁸ gp and 1×10¹³ gp, inclusive of the endpoints. In some embodiments, the therapeutically effective amount comprises between 6×10⁹ gp and 1×10¹³ gp, inclusive of the endpoints. In some embodiments, the therapeutically effective amount comprises between 6×10⁹ gp and 7×10¹² gp, inclusive of the endpoints. In some embodiments, the therapeutically effective amount comprises between 6×10⁹ gp and 5×10¹² gp, inclusive of the endpoints. In some embodiments, the therapeutically effective amount comprises between 1×10¹⁰ gp and 1×10¹² gp, inclusive of the endpoints. In some embodiments, the therapeutically effective amount comprises or consists of 1×10¹⁰ gp. In some embodiments, the therapeutically effective amount comprises or consists of 1×10¹¹ gp. In some embodiments, the therapeutically effective amount comprises or consists of 1×10¹² gp.

In some embodiments of the methods of treating a disease or disorder of the disclosure, the therapeutically effective amount comprises or consists of a volume between 10 μL and 200 μL, inclusive of the endpoints. In some embodiments, the therapeutically effective amount comprises or consists of a volume between 10 μL and 50 μL, between 50 μL and 100 μL, between 100 μL and 150 μL or between 150 μL and 200 μL, inclusive of the endpoints, for each range. In some embodiments, the therapeutically effective amount comprises or consists of a volume between 70 μL and 120 μL, inclusive of the endpoints. In some embodiments, the therapeutically effective amount comprises or consists of a volume a volume of 100 μL.

In some embodiments of the methods of treating a disease or disorder of the disclosure, the therapeutically effective amount comprises or consists of at least one injection of a volume between 10 μL and 200 μL, inclusive of the endpoints. In some embodiments, the therapeutically effective amount comprises or consists of at least 2, 3, 4, 5, 6, 7, 8, 9, 10 injections of a volume between 10 μL and 200 μL, inclusive of the endpoints. In some embodiments, two or more injections are made into a subretinal space during the same medical procedure on the same eye. In some embodiments, two or more injections are made into two or more distinct subretinal spaces during the same medical procedure on the same eye. In some embodiments, the area of the subretinal space contacted by the therapeutically-effective amount of the vector comprises or consists of between 5 and 20 mm². In some embodiments, the area of the subretinal space contacted by the therapeutically-effective amount of the vector comprises or consists of 10 mm². In some embodiments, one or more injections are made into at least 2, 3, 4, 5, 6, 7, 8, 9, or 10 distinct areas of the retina of an eye. In some embodiments, one or more injections are made into at least 2, 3, 4, 5, 6, 7, 8, 9, or 10 distinct areas of the retina of an eye during a single procedure. In some embodiments, one or more injections are made into at least 2, 3, 4, 5, 6, 7, 8, 9, or 10 distinct areas of the retina of an eye over the course of two or more procedures.

In some embodiments of the methods of treating a disease or disorder of the disclosure, the therapeutically effective amount comprises or consists of at least one injection of a volume between 10 μL and 200 μL, inclusive of the endpoints, administered from the same device. In some embodiments of the methods of treating a disease or disorder of the disclosure, the therapeutically effective amount is delivered by the same device as a split dose, divided between one or more injections. The split dose may be administered to the same subretinal space or to two or more distinct subretinal spaces within the same eye.

In some embodiments of the methods of treating a disease or disorder of the disclosure, the administering step comprises an injection or an infusion. In some embodiments, the administering step comprises a subretinal, a suprachoroidal or an intravitreal route. In some embodiments, the administering step comprises a subretinal injection or infusion. In some embodiments, the subretinal injection or infusion comprises a 2 step subretinal injection. In some embodiments, the administering step comprises a suprachoroidal injection or infusion.

In some embodiments of the methods of treating a disease or disorder of the disclosure, the subject is male. In some embodiments, the subject is at least 18 years of age. In some embodiments, the subject has a genetically confirmed diagnosis of choroideremia. In some embodiments, the subject has been identified as having a mutation in the REP1 gene. In some embodiments, the subject presents a clinical sign of choroideremia in a macula of at least one eye. In some embodiments, the subject has a Best Corrected Visual Acuity (BCVA) score of 34-73 letters in at least one eye. In some embodiments, the subject has mild or early stage choroideremia. In some embodiments, the subject has advanced or severe choroideremia.

In some embodiments of the methods of treating a disease or disorder of the disclosure, the method comprises treating 10 mm² of a retina of at least one eye. In some embodiments, the method comprises treating between 5 mm² and 10 mm², inclusive of the endpoints, of a retina of at least one eye. In some embodiments, the method comprises treating between 2 mm² and 15 mm², inclusive of the endpoints, of a retina of at least one eye.

In some embodiments of the methods of treating a disease or disorder of the disclosure, the pharmaceutical composition is administered to one eye of the subject. In some embodiments, the pharmaceutical composition is administered to both eyes of the subject. In some embodiments, the eyes of the subject are treated simultaneously. In some embodiments, both eyes of the subject are treated sequentially. In some embodiments, at least one eye of the subject had been treated for choroideremia prior to administration of the pharmaceutical composition to the subject.

In some embodiments of the methods of treating a disease or disorder of the disclosure, the method comprises administering between 1 and 12 doses per eye, inclusive of the endpoints. In some embodiments, the method comprises administering at least one dose at least once per day, once per week, once per month, once every three months, once every 6 months, or once per year. In some embodiments, the method comprises administering multiple doses and wherein each dose comprises the same amount of the pharmaceutical composition. In some embodiments, the method comprises administering multiple doses and wherein each dose does not comprise the same amount of the pharmaceutical composition. In some embodiments, the method comprises administering multiple doses and wherein each successive dose comprises a greater number of full rAAV than the previous dose. In some embodiments, the method comprises administering multiple doses and wherein each successive dose comprises a lesser number of full rAAV than the previous dose.

In some embodiments of the methods of treating a disease or disorder of the disclosure, the method comprises administering between 1 and 12 doses per eye, inclusive of the endpoints. In some embodiments, the doses are provided following a period of recovery. In some embodiments, the period of recovery is at least 1, 5, 10, 15, 20, 25, 30, 35, 40, 45, 50, 55, 60 or any number of minutes in between. In some embodiments, the period of recovery is at least 1, 2, 3, 4, 5, 6, 7, 8, 9, 10, 11, 12, 13, 14, 15, 16, 17, 18, 19, 20, 21, 22, 23, or 24 hours. In some embodiments, the period of recovery is at least 1, 2, 3, 4, 5, 6, or 7 days. In some embodiments, the period of recovery is at least 1, 2, 3, or 4 weeks. In some embodiments, the period of recovery is at least 1, 2, 3, 4, 5 6, 7, 8, 9, 10, 11, or 12 months. In some embodiments, the period of recovery is at least 1, 2, 3, 4, 5 or 6 years.

In some embodiments of the methods of treating a disease or disorder of the disclosure, including those wherein the method comprises administering between 1 and 12 doses per eye, inclusive of the endpoints, the subject has experienced an adverse event following a therapeutically effective dose and the subsequent dose comprises a lesser number of full rAAV than the previous dose that induced the adverse event. In some embodiments, the subject recovers from the adverse event and a subsequent dose of the pharmaceutical composition is administered to the subject. In some embodiments, the therapeutically effective dose that induced the adverse event and the subsequent dose contain an equal number of full rAAV. In some embodiments, the therapeutically effective dose that induced the adverse event and the subsequent dose does not contain an equal number of full rAAV.

In some embodiments of the methods of treating a disease or disorder of the disclosure, the method further comprises administering an amount of a plurality of placebo rAAVs to the subject prior to administration of a therapeutically effective amount of the pharmaceutical composition, wherein each placebo rAAV is an empty rAAV. In some embodiments, the empty rAAV does not contain either a promoter to express an exogenous sequence or an exogenous sequence. In some embodiments, administration of the amount of a plurality of placebo rAAVs is systemic. In some embodiments, administration of the amount of a plurality of placebo rAAVs is local. In some embodiments, the method further comprises (a) determining whether the plurality of placebo rAAVs induced an immune response in the subject and/or (b) determining whether the subject developed an immune tolerance to the plurality of placebo rAAVs, thereby indicating that administration of a therapeutically effective amount of the pharmaceutical composition should not induce an immune-mediated adverse event in the subject.

In some embodiments of the methods of treating a disease or disorder of the disclosure, the method further comprises administering to the subject a suppressant of an immune response. In some embodiments, the suppressant comprises an anti-inflammatory agent. In some embodiments, the anti-inflammatory agent comprises a corticosteroid. In some embodiments, the corticosteroid comprises prednisone or prednisolone. In some embodiments, the administration of the suppressant of an immune response is systemic. In some embodiments, the suppressant of an immune response is administered orally. In some embodiments, the administration of the suppressant of an immune response is local. In some embodiments, the suppressant of an immune response is administered to the eye treated with the pharmaceutical composition. In some embodiments, the pharmaceutical composition and the suppressant of an immune response are administered simultaneously. In some embodiments, the pharmaceutical composition and the suppressant of an immune response are administered on the same day. In some embodiments, the pharmaceutical composition and the suppressant of an immune response are administered sequentially. In some embodiments, the administration of the suppressant precedes the administration of the pharmaceutical composition by at least one day. In some embodiments, the administration of the pharmaceutical composition precedes the administration of the suppressant by at least one day.

In some embodiments of the methods of treating a disease or disorder of the disclosure, the method further comprises determining an initial severity of choroideremia-mediated damage in at least one eye of the subject. In some embodiments, the method further comprises determining a subsequent severity of choroideremia-mediated damage in the at least one eye of the subject following administration of the pharmaceutical composition to the at least one eye. In some embodiments, the initial or subsequent severity of choroideremia-mediated damage is determined by determining a Best Corrected Visual Acuity (BCVA) test score, measuring an area or a volume of viable retinal tissue, measuring a preserved ellipsoid zone, measuring retinal sensitivity, measuring contrast sensitivity, measuring color vision, measuring low luminance visual acuity, measuring speed reading or any combination thereof. In some embodiments, the BCVA test utilizes an (Early Treatment of Diabetic Retinopathy Study) ETDRS chart. In some embodiments, the BCVA test comprises an assessment of one or more of finger counting, hand movement, light perception and a combination thereof. In some embodiments, the viable retinal tissue comprises fundus autofluorescence and wherein measuring viable retinal tissue comprises detecting a level or pattern of fundus autofluorescence. In some embodiments, the measuring a preserved ellipsoid zone comprises Spectral Domain Optical Coherence Tomography (SD-OCT). In some embodiments, the measuring retinal sensitivity comprises microperimetry.

In some embodiments of the methods of treating a disease or disorder of the disclosure, the administration of the therapeutically effective amount of the pharmaceutical composition inhibits or reduces progression of a sign or a symptom of choroideremia. In some embodiments, the administration of the therapeutically effective amount of the pharmaceutical composition reduces a sign or a symptom of choroideremia. In some embodiments, a sign or a symptom of choroideremia comprises a loss of photoreceptor cells, a loss of RPE cells, a decreased visual acuity, decreased low luminescence visual acuity, total area of preserved autofluorescence (AF), a low score on a BCVA test, a decreased area of preserved ellipsoid zone, decreased retinal sensitivity, decreased contrast sensitivity, decreased or faded color vision, decreased rates of speed reading or any combination thereof. In some embodiments, the severity of the sign or symptom of choroideremia is determined relative to a healthy retina. In some embodiments, the healthy retina belongs to an age-matched control subject.

The disclosure provides a method of determining a therapeutically effective amount of a pharmaceutical composition of the disclosure, the method comprising: (a) measuring an area of a retina of a subject to be treated, (b) determining whether the area of (a) is in the central 0.5 mm² foveal area or in the macula, (c) calculating the number of rods, cones and retinal pigment epithelial (RPE) cells within the area of (a), and (d) multiplying the total number of cells by a multiplicity of infection (MOI) of 1×10⁵ to calculate a number of genome particles (gp) to be included in the therapeutically effective amount, wherein the maximal area of the retinal to be treated is 10 mm², wherein the density of RPE cells in the retina is 5,000 cells per mm², wherein the density of rods in the retina is 75,000 rods per mm² exclusive of the central 0.5 mm² foveal area, wherein the density of cones in the retina is 150,000 cones per mm² in the central 0.5 mm² foveal area and the density of cones in the retina is 25,000 per mm² in the macula outside the central 0.5 mm² foveal area.

The disclosure provides a pharmaceutical composition of the disclosure for use in treating a disease or a disorder in a subject in need thereof.

The disclosure provides a vector of the disclosure for use in treating a disease or a disorder in a subject in need thereof.

The disclosure provides an AAV or a rAAV of the disclosure for use in treating a disease or a disorder in a subject in need thereof.

### BRIEF DESCRIPTION OF THE DRAWINGS

FIG. 1 is an overview of an exemplary AAV2-Construct Drug Substance manufacturing method (Process 2)

FIG. 2 is a flow diagram corresponding to the overview provided in FIG. 1.

FIG. 3 is a diagram depicting a structural organization of an exemplary AAV2-Construct.

FIG. 4 is an overview of HEK293 MCB production.

FIG. 5 is an overview of an AAV2-Construct Drug Substance Manufacturing Method (Original Process).

FIG. 6 is a flow diagram corresponding to the overview provided in FIG. 5.

FIG. 7 is a flow diagram depicting an AAV2-Construct Drug Product Manufacturing Method (Original Process).

FIG. 8 is a pair of flow diagrams depicting a comparison of Original and Improved Methods—Cell Culture Phases.

FIG. 9 is a graph depicting a comparison of Original and Improved Methods—Cell Culture Phases.

FIG. 10 is a graph depicting a comparison of Original and Improved Methods—Cell Culture Phases.

FIG. 11 is a flow diagram depicting a downstream method transferred from Original to Improved Process.

FIG. 12 is a graph depicting a comparison of Yield Analysis AVG DRP/cm² for AmpR (left bar) and KanR (right bar) Plasmids.

FIG. 13 is a graph depicting a Mean Fluorescence Intensity of a detectable label elucidating Construct expression from each cell following transduction with AAV2-Construct.

FIG. 14 is a graph depicting a percentage of GFP-positive cells, as determined by flow cytometry, indicating a percentage of cells expressing the Construct protein from following transduction with AAV2-Construct.

FIG. 15 is a graph depicting the average yield results for AAV2-Construct Yield Analysis.

FIG. 16 is a photograph of a gel electrophoresis depicting purity data for an AAV2-Construct (non-GMP) composition (from Improved Process).

FIG. 17 is a photograph of a gel electrophoresis depicting Construct expression and activity following in vitro transduction of HEK293 cells with equivalent MOI of AAV2-Construct Vector from (non-GMP) compositions produced by Original and Improved Processes.

FIG. 18 is a map of the pAAV-REP1-Kan Plasmid.

FIG. 19 is a full sequence of the pBC-hREP1 vector (SEQ ID NO: 24).

FIG. 20 is a graph depicting visual acuity in treated and untreated eyes at 2 years after gene therapy (Example 13). Visual acuity measured with the Early Treatment for Diabetic Retinopathy Study (ETDRS) chart at 2 years in 12 choroideremia patients who received subretinal gene therapy without complications (horizontal line represents median, box plot represents interquartile range, and whiskers represent limits). Treated eyes are shown in blue with untreated eyes in green (left bar and right bar, respectively, at each time point). The interim data at 1 year are also shown. There was an improvement in visual acuity in the treated eyes 2 years after gene therapy (p=0.020). The treated eyes improved relative to the untreated eyes over this period (p=0.003).

FIG. 21 is a graph depicting macular function in treated and untreated eyes 2 years after gene therapy. Retinal sensitivity measured with microperimetry includes points all over the macula and increases with improved retinal function. There was a drop in retinal sensitivity in the untreated eyes from baseline by 2 years (p=0.004), but no significant change in the treated eyes over this period. The interim data at 1 year are also shown. Treated eyes are shown in blue and untreated eyes in green (left bar and right bar, respectively, at each time point)—error bars represent standard error of the mean.

FIG. 22 is a schematic drawing depicting the design of the GEMINI trial described in Example 14.

FIG. 23A-B is a schematic drawing depicting a vitrectomy (A) and a sub-retinal Injection (B) of AAV2 Vector. (A) A standard vitrectomy through the BIOM operating system to remove the vitreous gel is followed by (B) 2 step procedure: 1) retinal detachment by injection of BSS; 2) injection of a volume of 0.1 mL vector suspension through a 41-gauge cannula into the sub-retinal space.

FIG. 24 is a schematic drawing depicting the design of the STAR trial described in Example 15.

FIG. 25 is a graph showing AAV titer as determined by PCR. On the X axis are samples at an initial titer of 1×10¹² DNase resistant particles (DRP)/mL, 1×10¹¹ DRP/mL and 1×10¹¹ DRP/mL in balanced saline solution (BSS). On the Y axis, is shown titer measured after samples were treated as described to the right of the graph.

FIG. 26 is a series of 3 photographs of Western blots showing the prenylation activity of rAAV2.REP-1 in a compatibility study using AAV2.REP1.ENG1014-A vector at a high dose of 1×10¹² DRP/mL and an MOI of 10,000. From top to bottom are shown: hREP1 (83 KDa), Actin (42 KDa) and biotinylated Rab6a (24 KDa). Protein sizes are indicated at left, from top to bottom, as 180, 135, 100, 75, 63, 48, 35, 25, 20, 17 and 11 KDa. Samples, from left to right, in triplicate, are: untransduced control, cells transduced with baseline vector, with vector held 6 hours at 4° C., with vector held 6 hours at 4° C. and injected after 180 minutes, with vector held 6 hours at 4° C. and 180 minutes in a syringe, and fish REP1 as a positive control (single sample).

FIG. 27 is a series of 3 photographs of Western blots showing the prenylation activity of rAAV2.REP-1 in a compatibility study using AAV2.REP1.ENG1014-A vector at a low dose of 1×10¹¹ DRP/mL and an MOI of 10,000. From top to bottom are shown: hREP1 (83 KDa), Actin (42 KDa) and biotinylated Rab6a (24 KDa). Protein sizes are indicated at left, from top to bottom, as 180, 135, 100, 75, 63, 48, 35, 25, 20, 17 and 11 KDa. Samples, from left to right, in triplicate, are: untransduced control, cells transduced with baseline vector, with vector held 6 hours at 4° C., with vector held 6 hours at 4° C. and injected after 180 minutes, with vector held 6 hours at 4° C. and 180 minutes in a syringe, and fish REP1 as a positive control (single sample).

FIG. 28A-B are a pair of plots showing semi quantification of Western blots of prenylation activity of rAAV2.REP-1 in a compatibility study using AAV2.REP1.ENG1014-A vector. (A) Shows normalized REP1. Band density values (a.u.) are on the y-axis and AAV2-REP1 at a high dose of 1×10¹² DRP/mL and a low dose of 1×10¹¹ DRP/mL are on the x-axis. (B) Shows normalized biotinylated Rab6a. Band density values (a.u.) are on the y-axis and AAV2-REP1 at a high dose of 1×10¹² DRP/mL and a low dose of 1×10¹¹ DRP/mL are on the x-axis. In (A) and (B), bars for each dose, from left to right, indicate untransduced cells, cells transduced with baseline vector, with vector held 6 hours at 4° C., +6 hours at 4° C. and injected after 180 minutes at 20° C., with vector 6 hours at 4° C. and 180 minutes in a syringe at 20° C.

FIG. 29 is an exemplary construct of the disclosure comprising or consisting of a nucleotide sequence comprising from 5′ to 3′, a sequence encoding a 5′ inverted terminal repeat (ITR), a sequence encoding a CAG promoter, a sequence encoding a human REP1 protein, a sequence encoding a mutated WPRE signal, a sequence encoding polyadenylation signal of the bovine growth hormone, and a sequence encoding a 3′ ITR.

### DETAILED DESCRIPTION

The disclosure provides methods for the purification of a recombinant AAV (rAAV) particle of the disclosure. The disclosure provides pharmaceutical compositions comprising rAAV particles produced by the methods of the disclosure. In some embodiments, the pharmaceutical compositions comprising rAAV particles produced by the methods of the disclosure comprise (a) between 0.5 and 2.5×10¹² vector genomes (vg)/mL of replication-defective and recombinant adeno-associated virus (rAAV); (b) less than 50% empty capsids; (c) less than 4 ng/mL residual host cell protein per 1.0×10¹² vg/mL; and (d) less than 7×10⁻³ pg/ml residual host cell DNA per 1.0×10¹² vg/mL.

The disclosure provides a method of treating a disease or disorder in a subject in need thereof, comprising administering to the subject a therapeutically effective amount of a pharmaceutical composition of the disclosure. In some embodiments, the disease or disorder is a retinal disease or disorder. In some embodiments, the disease or disorder is Choroideremia.

The disclosure provides a pharmaceutical composition comprising: (a) between 0.5 and 2.5×10¹² vector genomes (vg)/mL of replication-defective and recombinant adeno-associated virus (rAAV); (b) less than 50% empty capsids; (c) less than 4 ng/mL residual host cell protein per 1.0×10¹² vg/mL; and (d) less than 7×10⁻³ pg/ml residual host cell DNA per 1.0×10¹² vg/mL.

In some embodiments, the pharmaceutical composition comprises: (a) between 0.5 and 2.5×10¹² vector genomes (vg)/mL of replication-defective and recombinant adeno-associated virus (rAAV); (b) less than 50% empty capsids; (c) less than 4 ng/mL residual host cell protein per 1.0×10¹² vg/mL; (d) less than 7×10⁻³ pg/ml residual host cell DNA per 1.0×10¹² vg/mL; and (e) a plurality of functional vg/mL, wherein each of functional vector genomes is capable of expressing an exogenous sequence in a cell following transduction. In some embodiments, following transduction of a cell with a pharmaceutical composition of the disclosure, the plurality of functional vg/mL express the exogenous sequence at a 2-fold increase when compared to a level of expression of a corresponding endogenous sequence in a nontransduced cell. In some embodiments, following transduction of a cell with a pharmaceutical composition of the disclosure, the plurality of functional vg/mL express the exogenous sequence at a 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, or any other increment fold increase in between, when compared to a level of expression of a corresponding endogenous sequence in a nontransduced cell. In some embodiments, the exogenous sequence and the corresponding endogenous sequence are identical. In some embodiments, the exogenous sequence and the corresponding endogenous sequence are not identical. In some embodiments, the exogenous sequence and the corresponding endogenous sequence have at least 70%, 75%, 80%, 85%, 90%, 95%, 97%, 99% or any percentage in between of identity.

In some embodiments, the pharmaceutical composition comprises: (a) between 0.5 and 2.5×10¹² vector genomes (vg)/mL of replication-defective and recombinant adeno-associated virus (rAAV); (b) less than 50% empty capsids; (c) less than 4 ng/mL residual host cell protein per 1.0×10¹² vg/mL; (d) less than 7×10⁻³ pg/ml residual host cell DNA per 1.0×10¹² vg/mL; and (e) a plurality of functional vg/mL, wherein each of functional vector genomes is capable of expressing an exogenous sequence in a cell following transduction. In some embodiments, following transduction of a cell with a pharmaceutical composition of the disclosure, the exogenous sequence encodes a protein. In some embodiments, the protein encoded by the exogenous sequence has an activity level equal to or greater than an activity level of a protein encoded by a corresponding sequence of a nontransduced cell. In some embodiments, the exogenous sequence and the corresponding endogenous sequence are identical. In some embodiments, the exogenous sequence and the corresponding endogenous sequence are not identical. In some embodiments, the exogenous sequence and the corresponding endogenous sequence have at least 70%, 75%, 80%, 85%, 90%, 95%, 97%, 99% or any percentage in between of identity. In some embodiments, the activity comprises binding to, activating, and/or transferring one or more functional groups to a ligand or a substrate. In some embodiments, the protein comprises a REP-1 protein and the activity comprises prenylation of REP-1 substrate.

### AAV Compositions

Compositions of the disclosure comprise a therapeutic Construct suitable for systemic or local administration to a mammal, and preferable, to a human. Exemplary Constructs of the disclosure comprise a sequence encoding a gene or a portion thereof. Preferably, Constructs of the disclosure comprise a sequence encoding a human gene or a portion thereof. Exemplary Constructs of the disclosure may further comprise one or more sequence(s) encoding regulatory elements to enable or to enhance expression of the gene or a portion thereof. Exemplary regulatory elements include, but are not limited to, promoters, introns, enhancer elements, response elements (including post-transcriptional response elements or post-transcriptional regulatory elements), polyadenosine (polyA) sequences, and a gene fragment to facilitate efficient termination of transcription (including a β-globin gene fragment and a rabbit β-globin gene fragment).

In some embodiments of the compositions of the disclosure, the Construct comprises a human gene or a portion thereof corresponding to a human Rab Escort Protein type 1 (REP-1) protein or a portion thereof. In some embodiments of the compositions of the disclosure, the Construct comprises a human gene or a portion thereof comprising a codon-optimized sequence. In some embodiments, the sequence is codon-optimized for expression in mammals. In some embodiments, the sequence is codon-optimized for expression in humans.

In some embodiments of the compositions of the disclosure, wherein the Construct comprises a human REP-1 protein or a portion thereof, the Construct is referred to as “AAV2-REP1 or AAV2.REP1” and the International Nonproprietary Name (INN) is timrepigene emparvovec. In some embodiments, the Construct comprises or consists of the sequence of:

CTGCGCGCTC GCTCGCTCAC TGAGGCCGCC CGGGCAAAGC CCGGGCGTCG GGCGACCTTT

61 GGTCGCCCGG CCTCAGTGAG CGAGCGAGCG CGCAGAGAGG GAGTGGCCAA CTCCATCACT

121 AGGGGTTCCT TGTAGTTAAT GATTAACCCG CCATGCTACT TATCTACGTA GCCATGCTCT

181 AGGTACCATT GACGTCAATA ATGACGTATG TTCCCATAGT AACGCCAATA GGGACTTTCC

241 ATTGACGTCA ATGGGTGGAG TATTTACGGT AAACTGCCCA CTTGGCAGTA CATCAAGTGT

301 ATCATATGCC AAGTACGCCC CCTATTGACG TCAATGACGG TAAATGGCCC GCCTGGCATT

361 ATGCCCAGTA CATGACCTTA TGGGACTTTC CTACTTGGCA GTACATCTAC GTATTAGTCA

421 TCGCTATTAC CATGGTCGAG GTGAGCCCCA CGTTCTGCTT CACTCTCCCC ATCTCCCCCC

481 CCTCCCCACC CCCAATTTTG TATTTATTTA TTTTTTAATT ATTTTGTGCA GCGATGGGGG

541 CGGGGGGGGG GGGGGGGCGC GCGCCAGGCG GGGCGGGGCG GGGCGAGGGG CGGGGCGGGG

601 CGAGGCGGAG AGGTGCGGCG GCAGCCAATC AGAGCGGCGC GCTCCGAAAG TTTCCTTTTA

661 TGGCGAGGCG GCGGCGGCGG CGGCCCTATA AAAAGCGAAG CGCGCGGCGG GCGGGAGTCG

721 CTGCGCGCTG CCTTCGCCCC GTGCCCCGCT CCGCCGCCGC CTCGCGCCGC CCGCCCCGGC

781 TCTGACTGAC CGCGTTACTC CCACAGGTGA GCGGGCGGGA CGGCCCTTCT CCTCCGGGCT

841 GTAATTAGCG CTTGGTTTAA TGACGGCTTG TTTCTTTTCT GTGGCTGCGT GAAAGCCTTG

901 AGGGGCTCCG GGAGGGCCCCT TTGTGCGGGG GGAGCGGCTC GGGGCTGTCC GCGGGGGGAC

961 GGCTGCCTTC GGGGGGGACG GGGCAGGGCG GGGTTCGGCT TCTGGCGTGT GACCGGCGGC

1021 TCTAGAGCCT CTGCTAACCA TGTTCATGCC TTCTTCTTTT TCCTACAGCT CCTGGGCAAC

1081 GTGCTGGTTA TTGTGCTGTC TCATCATTTT GGCAAAGAAT TGGATCCTAG CTTGATATCG

1141 AATTCCTGCA GCCCGGCGGC ACCATGGCGG ATACTCTCCC TTCGGAGTTT GATGTGATCG

1201 TAATAGGGAC GGGTTTGCCT GAATCCATCA TTGCAGCTGC ATGTTCAAGA AGTGGCCGGA

1261 GAGTTCTGCA TGTTGATTCA AGAAGCTACT ATGGAGGAAA CTGGGCCAGT TTTAGCTTTT

1321 CAGGACTATT GTCCTGGCTA AAGGAATACC AGGAAAACAG TGACATTGTA AGTGACAGTC

1381 CAGTGTGGCA AGACCAGATC CTTGAAAATG AAGAAGCCAT TGCTCTTAGC AGGAAGGACA

1441 AAACTATTCA ACATGTGGAA GTATTTTGTT ATGCCAGTCA GGATTTGCAT GAAGATGTCG

1501 AAGAAGCTGG TGCACTGCAG AAAAATCATG CTCTTGTGAC ATCTGCAAAC TCCACAGAAG

1561 CTGCAGATTC TGCCTTCCTG CCTACGGAGG ATGAGTCATT AAGCACTATG AGCTGTGAAA

1621 TGCTCACAGA ACAAACTCCA AGCAGCGATC CAGAGAATGC GCTAGAAGTA AATGGTGCTG

1681 AAGTGACAGG GGAAAAAGAA AACCATTGTG ATGATAAAAC TTGTGTGCCA TCAACTTCAG

1741 CAGAAGACAT GAGTGAAAAT GTGCTATAG CAGAAGATAC CACAGAGCAA CCAAAGAAAA

1801 ACAGAATTAC TTACTCACAA ATTATTAAAG AAGGCAGGAG ATTTAATATT GATTTAGTAT

1861 CAAAGCTGCT GTATTCTCGA GGATTACTAA TTGATCTTCT AATCAAATCT AATGTTAGTC

1921 GATATGCAGA GTTTAAAAAT ATTACCAGGA TTCTTGCATT TCGAGAAGGA CGAGTGGAAC

1981 AGGTTCCGTG TTCCAGAGCA GATGTCTTTA ATAGCAAACA ACTTACTATG GTAGAAAAGC

2041 GAATGCTAAT GAAATTTCTT ACATTTTGTA TGGAATATGA GAAATATCCT GATGAATATA

2101 AAGGATATGA AGAGATCACA TTTTATGAAT ATTTAAAGAC TCAAAAATTA ACCCCCAACC

2161 TCCAATATAT TGTCATGCAT TCAATTGCAA TGACATCAGA GACAGCCAGC AGCACCATAG

2221 ATGGTCTCAA AGCTACCAAA AACTTTCTTC ACTGTCTTGG GCGGTATGGC AACACTCCAT

2281 TTTTGTTTCC TTTATATGGC CAAGGAGAAC TCCCCAGTG TTTCTGCAGG ATGTGTGCTG

2341 TGTTTGGTGG AATTTATTGT CTTCGCCATT CAGTACAGTG CCTTGTAGTG GACGAAAGAAT

2401 CCAGAAAATG TAAAGCAATT ATAGATCAGT TTGGTCAGAG AATAATCTCT GAGCATTTCC

2461 TCGTGGAGGA CAGTTACTTT CCTGAGAACA TGTGCTCACG TGTGCAATAC AGGCAGATCT

2521 CCAGGGCAGT GCTGATTACA GATAGATCTG TCCTAAAAAC AGATTCAGAT CAACAGATTT

2581 CCATTTTGAC AGTGCCAGCA GAGGAACCAG GAACTTTTGC TGTTCGGGTC ATTGAGTTAT

2641 GTTCTTCAAC GATGACATGC ATGAAAGGCA CCTATTTGGT TCATTTGACT TGCACATCTT

2701 CTAAAACAGC AAGAGAAGAT TTAGAATCAG TTGTGCAGAA ATTGTTTGTT CCATATACTG

2761 AAATGGAGAT AGAAAATGAA CAAGTAGAAA AGCCAAGAAT TCTGTGGGCT CTTTACTTCA

2821 ATATGAGAGA TTCGTCAGAC ATCAGCAGGA GCTGTTATAA TGATTTACCA TCCAACGTTT

2881 ATGTCTGCTC TGGCCCAGAT TGTGGTTTAG GAAATGATAA TGCAGTCAAA CAGGCTGAAA

2941 CACTTTTCCA GGAAATCTGC CCCAATGAAG ATTTCTGTCC CCCTCCACCA AATCCTGAAG

3001 ACATTATCCT TGATGGAGAC AGTTTACAGC CAGAGGCTTC AGAATCCAGT GCCATACCAG

3061 AGGCTAACTC GGAGACTTTC AAGGAAAGCA CAAACCTTGG AAACCTAGAG GAGTCCTCTG

3121 AATAATCTAG TCGATTCGAA TTCGATATCA AGCTTATCGA TAATCAACCT CTGGATTACA

3181 AAATTTGTGA AAGATTGACT GGTATTCTTA ACTATGTTGC TCCTTTTACG CTATGTGGAT

3241 ACGCTGCTTT AATGCCTTTG TATCATGCTA TTGCTTCCCG TATGGCTTTC ATTTTCTCCT

3301 CCTTGTATAA ATCCTGGTTG CTGTCTCTTT ATGAGGAGTT GTGGCCCGTT GTCAGGCAAC

3361 GTGGCGTGGT GTGCACTGTG TTTGCTGACG CAACCCCCAC TGGTTGGGGC ATTGCCACCA

3421 CCTGTCAGCT CCTTTCCGGG ACTTTCGCTT TCCCCCTCCC TATTGCCACG GCGGAACTCA

3481 TCGCCGCCTG CCTTGCCCGC TGCTGGACAG GGGCTCGGCT GTTGGGCACT GACAATTCCG

3541 TGGTGTTGTC GGGGAAATCA TCGTCCTTTC CTTGGCTGCT CGCCTGTGTT GCCACCTGGA

3601 TTCTGCGCGG GACGTCCTTC TGCTACGTCC CTTCGGCCCT CAATCCAGCG GACCTTCCTT

3661 CCCGCGGCCT GCTGCCGGCT CTGCGGCCTC TTCCGCGTCT TCGCCTTCGC CCTCAGACGA

3721 GTCGGATCTC CCTTTGGGCC GCCTCCCCGC ATCGATACCG TCGACTCGCT GATCAGCCTC

3781 GACTGTGCCT TCTAGTTGCC AGCCATCTGT TGTTTGCCCC TCCCCCGTGC CTTCCTTGAC

3841 CCTGGAAGGT GCCACTCCCA CTGTCCTTTC CTAATAAAAT GAGGAAATTG CATCGCATTG

3901 TCTGAGTAGG TGTCATTCTA TTCTGGGGGG TGGGGTGGGG CAGGACAGCA AGGGGGAGGA

3961 TTGGGAAGAC AATAGCAGGC ATGCTGGGGA TGCGGTGGGC TCTATGGCTT CTGAGGCGGA

4021 AAGAACCAGC TGGGGCTCGA CTAGAGCATG GCTACGTAGA TAAGTAGCAT GGCGGGTTAA

4081 TCATTAACTA CAAGGAACCC CTAGTGATGG AGTTGGCCAC TCCCTCTCTG CGCGCTCGCT

4141 CGCTCACTGA GGCCGGGCGA CCAAAGGTCG CCCGACGCCC GGGCGGCCTC AGTGAGCGAG

4201 CGAGCGCGCA GAG (SEQ ID NO: 16). In some embodiments, the Construct comprises or consists of a sequence having at least 50%, 55%, 60%, 65%, 70%, 75%, 80%, 85%, 90%, 95%, 99%, or any percentage in between of identify to the sequence of:

(SEQ ID NO: 16)

| | |
|---|---|
| 1 | CTGCGCGCTC GCTCGCTCAC TGAGGCCGCC CGGGCAAAGC |
| | CCGGGCGTCG GGCGACCTTT |
| 61 | GGTCGCCCGG CCTCAGTGAG CGAGCGAGCG CGCAGAGAGG |
| | GAGTGGCCAA CTCCATCACT |
| 121 | AGGGGTTCCT TGTAGTTAKT GATTAACCCG CCATGCTACT |
| | TATCTACGTA GCCATGCTCT |
| 181 | AGGTACCATT GACGTCAATA ATGACGTATG TTCCCATAGT |
| | AACGCCAATA GGGACTTTCC |
| 241 | ATTGACGTCA ATGGGTGGAG TATTTACGGT AAACTGCCCA |
| | CTTGGCAGTA CATCAAGTGT |
| 301 | ATCATATGCC AAGTACGCCC CCTATTGACG TCAATGACGG |
| | TAAATGGCCC GCCTGGCATT |
| 361 | ATGCCCAGTA CATGACCTTA TGGGACTTTC CTACTTGGCA |
| | GTACATCTAC GTATTAGTCA |
| 421 | TCGCTATTAC CATGGTCGAG GTGAGCCCCA CGTTCTGCTT |
| | CACTCTCCCC ATCTCCCCCC |
| 481 | CCTCCCCACC CCCAATTTTG TATTTATTTA TTTTTTAATT |
| | ATTTTGTGCA GCGATGGGGG |
| 541 | CGGGGGGGGG GGGGGGGCGC GCGCCAGGCG GGGCGGGGCG |
| | GGGCGAGGGG CGGGGCGGGG |
| 601 | CGAGGCGGAG AGGTGCGGCG GCAGCCAATC AGAGCGGCGC |
| | GCTCCGAAAG TTTCCTTTTA |
| 661 | TGGCGAGGCG GCGGCGGCGG CGGCCCTATA AAAAGCGAAG |
| | CGCGCGGCGG GCGGGAGTCG |
| 721 | CTGCGCGCTG CCTTCGCCCC GTGCCCCGCT CCGCCGCCGC |
| | CTCGCGCCGC CCGCCCCGGC |
| 781 | TCTGACTGAC CGCGTTACTC CCACAGGTGA GCGGGCGGGA |
| | CGGCCCTTCT CCTCCGGGCT |
| 841 | GTAATTAGCG CTTGGTTTAA TGACGGCTTG TTTCTTTTCT |
| | GTGGCTGCGT GAAAGCCTTG |
| 901 | AGGGGCTCCG GGAGGGCCCT TTGTGCGGGG GGAGCGGCTC |
| | GGGGCTGTCC GCGGGGGGAC |
| 961 | GGCTGCCTTC GGGGGGGACG GGGCAGGGCG GGGTTCGGCT |
| | TCTGGCGTGT GACCGGCGGC |
| 1021 | TCTAGAGCCT CTGCTAACCA TGTTCATGCC TTCTTCTTTT |
| | TCCTACAGCT CCTGGGCAAC |
| 1081 | GTGCTGGTTA TTGTGCTGTC TCATCATTTT GGCAAAGAAT |
| | TGGATCCTAG CTTGATATCG |
| 1141 | AATTCCTGCA GCCCGGCGGC ACCATGGCGG ATACTCTCCC |
| | TTCGGAGTTT GATGTGATCG |
| 1201 | TAATAGGGAC GGGTTTGCCT GAATCCATCA TTGCAGCTGC |
| | ATGTTCAAGA AGTGGCCGGA |
| 1261 | GAGTTCTGCA TGTTGATTCA AGAAGCTACT ATGGAGGAAA |
| | CTGGGCCAGT TTTAGCTTTT |
| 1321 | CAGGACTATT GTCCTGGCTA AAGGAATACC AGGAAAACAG |
| | TGACATTGTA AGTGACAGTC |
| 1381 | CAGTGTGGCA AGACCAGATC CTTGAAAATG AAGAAGCCAT |
| | TGCTCTTAGC AGGAAGGACA |
| 1441 | AAACTATTCA ACATGTGGAA GTATTTTGTT ATGCCAGTCA |
| | GGATTTGCAT GAAGATGTCG |
| 1501 | AAGAAGCTGG TGCACTGCAG AAAAATCATG CTCTTGTGAC |
| | ATCTGCAAAC TCCACAGAAG |
| 1561 | CTGCAGATTC TGCCTTCCTG CCTACGGAGG ATGAGTCATT |
| | AAGCACTATG AGCTGTGAAA |
| 1621 | TGCTCACAGA ACAAACTCCA AGCAGCGATC CAGAGAATGC |
| | GCTAGAAGTA AATGGTGCTG |
| 1681 | AAGTGACAGG GGAAAAAGAA AACCATTGTG ATGATAAAAC |
| | TTGTGTGCCA TCAACTTCAG |
| 1741 | CAGAAGACAT GAGTGAAAAT GTGCCTATAG CAGAAGATAC |
| | CACAGAGCAA CCAAAGAAAA |
| 1801 | ACAGAATTAC TTACTCACAA ATTATTAAAG AAGGCAGGAG |
| | ATTTAATATT GATTTAGTAT |
| 1861 | CAAAGCTGCT GTATTCTCGA GGATTACTAA TTGATCTTCT |
| | AATCAAATCT AATGTTAGTC |
| 1921 | GATATGCAGA GTTTAAAAAT ATTACCAGGA TTCTTGCATT |
| | TCGAGAAGGA CGAGTGGAAC |
| 1981 | AGGTTCCGTG TTCCAGAGCA GATGTCTTTA ATAGCAAACA |
| | ACTTACTATG GTAGAAAAGC |
| 2041 | GAATGCTAAT GAAATTTCTT ACATTTTGTA TGGAATATGA |
| | GAAATATCCT GATGAATATA |
| 2101 | AAGGATATGA AGAGATCACA TTTTATGAAT ATTTAAAGAC |
| | TCAAAAATTA ACCCCCAACC |
| 2161 | TCCAATATAT TGTCATGCAT TCAATTGCAA TGACATCAGA |
| | GACAGCCAGC AGCACCATAG |
| 2221 | ATGGGTCTAA AGCTACCAAA AACTTTCTTC ACTGTCTTGG |
| | GCGGTATGGC AACACTCCAT |
| 2281 | TTTTGTTTCC TTTATATGGC CAAGGAGAAC TCCCCCAGTG |
| | TTTCTGCAGG ATGTGTGCTG |
| 2341 | TGTTTGGTGG AATTTATTGT CTTCGCCATT CAGTACAGTG |
| | CCTTGTAGTG GACAAAGAAT |
| 2401 | CCAGAAAATG TAAAGCAATT ATAGATCAGT TTGGTCAGAG |
| | AATAATCTCT GAGCATTTCC |
| 2461 | TCGTGGAGGA CAGTTACTTT CCTGAGAACA TGTGCTCACG |
| | TGTGCAATAC AGGCAGATCT |
| 2521 | CCAGGGCAGT GCTGATTACA GATAGATCTG TCCTAAAAAC |
| | AGATTCAGAT CAACAGATTT |
| 2581 | CCATTTTGAC AGTGCCAGCA GAGGAACCAG GAACTTTTGC |
| | TGTTCGGGTC ATTGAGTTAT |
| 2641 | GTTCTTCAAC GATGACATGC ATGAAAGGCA CCTATTTGGT |
| | TCATTTGACT TGCACATCTT |
| 2701 | CTAAAACAGC AAGAGAAGAT TTAGAATCAG TTGTGCAGAA |
| | ATTGTTTGTT CCATATACTG |
| 2761 | AAATGGAGAT AGAAAATGAA CAAGTAGAAA AGCCAAGAAT |
| | TCTGTGGGCT CTTTACTTCA |
| 2821 | ATATGAGAGA TTCGTCAGAC ATCAGCAGGA GCTGTTATAA |
| | TGATTTACCA TCCAACGTTT |
| 2881 | ATGTCTGCTC TGGCCCAGAT TGTGGTTTAG GAAATGATAA |
| | TGCAGTCAAA CAGGCTGAAA |
| 2941 | CACTTTTCCA GGAAATCTG CCCAATGAAG ATTTCTGTCC |
| | CCCTCCACCA AATCCTGAAG |
| 3001 | ACATRATCCT TGATGGAGAC AGTTTACAGC CAGAGGCTTC |
| | AGAATCCAGT GCCATACCAG |
| 3061 | AGGCTAACTC GGAGACTTTC AAGGAAAGCA CAAACCTTGG |
| | AAACCTAGAG GAGTCCTCTG |
| 3121 | AATAATCTAG TCGATTCGAA TTCGATATCA AGCTTATCGA |
| | TAATCAACCT CTGGATTACA |
| 3181 | AAATTTGTGA AAGATTGACT GGTATTCTTA ACTATGTTGC |
| | TCCTTTTACG CTATGTGGAT |
| 3241 | ACGCTGCTTT AATGCCTTTG TATCATGCTA TTGCTTCCCG |
| | TATGGCTTTC ATTTTCTCCT |
| 3301 | CCTTGTATAA ATCCTGGTTG CTGTCTCTTT ATGAGGAGTT |
| | GTGGCCCGTT GTCAGGCAAC |
| 3361 | GTGGCGTGGT GTGCACTGTG TTTGCTGACG CAACCCCCAC |
| | TGGTTGGGGC ATTGCCACCA |
| 3421 | CCTGTCAGCT CCTTTCCGGG ACTTTCGCTT TCCCCCTCCC |
| | TATTGCCACG GCGGAACTCA |
| 3481 | TCGCCGCCTG CCTTGCCCGC TGCTGGACAG GGGCTCGGCT |
| | GTTGGGCACT GACAATTCCG |
| 3541 | TGGTGTTGTC GGGGAAATCA TCGTCCTTTC CTTGGCTGCT |
| | CGCCTGTGTT GCCACCTGGA |
| 3601 | TTCTGCGCGG GACGTCCTTC TGCTACGTCC CTTCGGCCCT |
| | CAATCCAGCG GACCTTCCTT |
| 3661 | CCCGCGGCCT GCTGCCGGCT CTGCGGCCTC TTCCGCGTCT |
| | TCGCCTTCGC CCTCAGACGA |
| 3721 | GTCGGATCTC CCTTTGGGCC GCCTCCCCGC ATCGATACCG |
| | TCGACTCGCT GATCAGCCTC |
| 3781 | GACTGTGCCT TCTAGTTGCC AGCCATCTGT TGTTTGCCCC |
| | TCCCCCGTGC CTTCCTTGAC |
| 3841 | CCTGGAAGGT GCCACTCCCA CTGTCCTTTC CTAATAAAAT |
| | GAGGAAATTG CATCGCATTG |
| 3901 | TCTGAGTAGG TGTCATTCTA TTCTGGGGGG TGGGGTGGGG |
| | CAGGACAGCA AGGGGGAGGA |
| 3961 | TTGGGAAGAC AATAGCAGGC ATGCTGGGGA TGCGGTGGGC |
| | TCTATGGCTT CTGAGGCGGA |
| 4021 | AAGAACCAGC TGGGGCTCGA CTAGAGCATG GCTACGTAGA |
| | TAAGTAGCAT GGCGGGTTAA |
| 4081 | TCATTAACTA CAAGGAACCC CTAGTGATGG AGTTGGCCAC |
| | TCCCTCTCTG CGCGCTCGCT |
| 4141 | CGCTCACTGA GGCCGGGCGA CCAAAGGTCG CCCGACGCCC |
| | GGGCGGCCTC AGTGAGCGAG |
| 4201 | CGAGCGCGCA GAG. |

In some embodiments, the Construct comprises or consists of a sequence encoding a human REP1 sequence having the sequence of

1 ATGGCGGATA CTCTCCCTTC GGAGTTTGAT GTGATCGTAA TAGGGACGGG TTTGCCTGAA

61 TCCATCATTG CAGCTGCATG TTCAAGAAGT GGCCGGAGAG TTCTGCATGT TGATTCAAGA

121 AGCTACTATG GAGGAAACTG GGCCAGTTTT AGCTTTTCAG GACTATTGTC CTGGCTAAAG

181 GAATACCAGG AAAACAGTGA CATTGTAAGT GACAGTCCAG TGTGGCAAGA CCAGATCCTT

241 GAAAATGAAG AAGCCATTGC TCTTAGCAGG AAGGACAAAA CTATTCAACA TGTGGAAGTA

301 TTTTGTTATG CCAGTCAGGA TTTGCATGAA GATGTCGAAG AAGCTGGTGC ACTGCAGAAA

361 AATCATGCTC TTGTGACATC TGCAAACTCC ACAGAAGCTG CAGATTCTGC CTTCCTGCCT

421 ACGGAGGATG AGTCATTAAG CACTATGAGC TGTGAAATGC TCACAGAACA AACTCCAAGC

481 AGCGATCCAG AGAATGCGCT AGAAGTAAAT GGTGCTGAAG TGACAGGGGA AAAAGAAAAC

541 CATTGTGATG ATAAAACTTG TGTGCCATCA ACTTCAGCAG AAGACATGAG TGAAAATGTG

601 CCTATAGCAG AAGATACCAC AGAGCAACCA AAGAAAAACA GAATTACTTA CTCACAAATT

661 ATTAAAGAAG GCAGGAGATT TAATATTGAT TTAGTATCAA AGCTGCTGTA TTCTCGAGGA

721 TTACTAATTG ATCTTCTAAT CAAATCTAAT GTTAGTCGAT ATGCAGAGTT TAAAAATATT

781 ACCAGGATTC TTGCATTTCG AGAAGGACGA GTGGAACAGG TTCCGTGTTC CAGAGCAGAT

841 GTCTTTAATA GCAAACAACT TACTATGGTA GAAAAGCGAA TGCTAATGAA ATTTCTTACA

901 TTTTGTATGG AATATGAGAA ATATCCTGAT GAATATAAAG GATATGAAGA GATCACATTT

961 TATGAATATT TAAAGACTCA AAAATTAACC CCCAACCTCC AATATATTGT CATGCATTCA

1021 ATTGCAATGA CATCAGAGAC AGCCAGCAGC ACCATAGATG GTCTCAAAGC TACCAAAAAC

1081 TTTCTTCACT GTCTTGGGCG GTATGGCAAC ACTCCATTTT TGTTTCCTTT ATATGGCCAA

1141 GGAGAACTCC CCCAGTGTTT CTGCAGGATG TGTGCTGTGT TTGGTGGAAT TTATTGTCTT

1201 CGCCATTCAG TACAGTGCCT TGTAGTGGAC AAAGAATCCA GAAAATGTAA AGCAATTATA

1261 GATCAGTTTG GTCAGAGAAT AATCTCTGAG CATTTCCTCG TGGAGGACAG TTACTTTCCT

1321 GAGAACATGT GCTCACGTGT GCAATACAGG CAGATCTCCA GGGCAGTGCT GATTACAGAT

1381 AGATCTGTCC TAAAAACAGA TTCAGATCAA CAGATTTCCA TTTTGACAGT GCCAGCAGAG

1441 GAACCAGGAA CTTTTGCTGT TCGGGTCATT GAGTTATGTT CTTCAACGAT GACATGCATG

1501 AAAGGCACCT ATTTGGTTCA TTTGACTTGC ACATCTTCTA AAACAGCAAG AGAAGATTTA

1561 GAATCAGTTG TGCAGAAATT GTTTGTTCCA TATACTGAAA TGGAGATAGA AAATGAACAA

1621 GTAGAAAAGC CAAGAATTCT GTGGGCTCTT TACTTCAATA TGAGAGATTC GTCAGACATC

1681 AGCAGGAGCT GTTATAATGA TTTACCATCC AACGTTTATG TCTGCTCTGG CCCAGATTGT

1741 GGTTTAGGAA ATGATAATGC AGTCAAACAG GCTGAAACAC TTTTCCAGGA AATCTGCCCC

1801 AATGAAGATT TCTGTCCCCC TCCACCAAAT CCTGAAGACA TTATCCTTGA TGGAGACAGT

1861 TTACAGCCAG AGGCTTCAGA ATCCAGTGCC ATACCAGAGG CTAACTCGGA GACTTTCAAG

1921 GAAAGCACAA ACCTTGGAAA CCTAGAGGAG TCCTCTGAAT AA (SEQ ID NO: 17) or

a sequence having at least 50%, 55%, 60%, 65%, 70%, 75%, 80%, 85%, 90%, 95%, 99%, or any percentage in between of identify to the sequence of

(SEQ ID NO: 17)

| | |
|---|---|
| 1 | ATGGCGGATA CTCTCCCTTC GGAGTTTGAT GTGATCGTAA |
| | TAGGGACGGG TTTGCCTGAA |
| 61 | TCGATCATTG CAGCTGCATG TTCAAGAAGT GGCCGGAGAG |
| | TTCTGCATGT TGATTCAAGA |
| 121 | AGCTACTATG GAGGAAACTG GGCCAGTTTT AGCTTTTCAG |
| | GACTATTGTC CTGGCTAAAG |
| 181 | GAATACCAGG AAAACAGTGA CATTGTAAGT GACAGTCCAG |
| | TGTGGCAAGA CCAGATCCTT |
| 241 | GAAAATGAAG AAGCCATTGC TCTTAGCAGG AAGGACAAAA |
| | CTATTCAACA TGTGGAAGTA |
| 301 | TTTTGTTATG CCAGTCAGGA TTTGCATGAA GATGTCGAAG |
| | AAGCTGGTGC ACTGCAGAAA |
| 361 | AATCATGCTC TTGTGACATC TGCAAACTCC ACAGAAGCTG |
| | CAGATTCTGC CTTCCTGCCT |
| 421 | ACGGAGGATG AGTCATTAAG CACTATGAGC TGTGAAATGC |
| | TCACAGAACA AACTCCAAGC |
| 481 | AGCGATCCAG AGAATGCGCT AGAAGTAAAT GGTGCTGAAG |
| | TGACAGGGGA AAAAGAAAAC |
| 541 | CATTGTGATG ATAAAACTTG TGTGGCATCA ACTTCAGCAG |
| | AAGACATGAG TGAAAATGTG |
| 601 | CCTATAGCAG AAGATACCAC AGAGCAACCA AAGAAAAACA |
| | GAATTACTTA CTCACAAATT |
| 661 | ATTAAAGAAG GCAGGAGATT TAATATTGAT TTAGTATCAA |
| | AGCTGCTGTA TTCTCGAGGA |
| 721 | TTACTAATTG ATCTTCTAAT CAAATCTAAT GTTAGTCGAT |
| | ATGCAGAGTT TAAAAATATT |
| 781 | ACCAGGATTC TTGCATTTCG AGAAGGACGA GTGGAACAGG |
| | TTCCGTGTTC CAGAGCAGAT |
| 841 | GTCTTTAATA GCAAACAACT TACTATGGTA GAAAAGCGAA |
| | TGCTAATGAA ATTTCTTACA |
| 901 | TTTTGTATGG AATATGAGAA ATATCCTGAT GAATATAAAG |
| | GATATGAAGA GATCACATTT |
| 961 | TATGAATATT TAAAGACTCA AAAATTAACC CCCAACCTCC |
| | AATATATTGT CATGCATTCA |
| 1021 | ATTGCAATGA CATCAGAGAC AGCCAGCAGC ACCATAGATG |
| | GTCTCAAAGC TACCAAAAAC |
| 1081 | TTTCTTCACT GTCTTGGGCG GTATGGCAAC ACTCCATTTT |
| | TGTTTCCTTT ATATGGCCAA |
| 1141 | GGAGAACTCC CCCAGTGTTT CTGCAGGATG TGTGCTGTGT |
| | TTGGTGGAAT TTATTGTCTT |
| 1201 | CGCCATTCAG TACAGTGCCT TGTAGTGGAC AAAGAATCCA |
| | GAAAATGTAA AGCAATTATA |
| 1261 | GATCAGTTTG GTCAGAGAAT AATCTCTGAG CATTTCCTCG |
| | TGGAGGACAG TTACTTTCCT |
| 1321 | GAGAACATGT GCTCACGTGT GCAATACAGG CAGATCTCCA |
| | GGGCAGTGCT GATTACAGAT |
| 1381 | AGATCTGTCC TAAAAACAGA TTCAGATCAA CAGATTTCCA |
| | TTTTGACAGT GCCAGCAGAG |
| 1441 | GAACCAGGAA CTTTTGCTGT TCGGGTCATT GAGTTATGTT |
| | CTTCAACGAT GACATGCATG |
| 1501 | AAAGGCACCT ATTTGGTTCA TTTGACTTGC ACATCTTCTA |
| | AAACAGCAAG AGAAGATTTA |
| 1561 | GAATCAGTTG TGCAGAAATT GTTTGTTCCA TATACTGAAA |
| | TGGAGATAGA AAATGAACAA |
| 1621 | GTAGAAAAGC CAAGAATTCT GTGGGCTCTT TACTTCAATA |
| | TGAGAGATTC GTCAGACATC |
| 1681 | AGCAGGAGCT GTTATAATGA TTTACCATCC AACGTTTATG |
| | TCTGCTCTGG CCCAGATTGT |
| 1741 | GGTTTAGGAA ATGATAATGC AGTCAAACAG GCTGAAACAC |
| | TTTTCCAGGA AATCTGCCCC |
| 1801 | AATGAAGATT TCTGTCCCCC TCCACCAAAT CCTGAAGACA |
| | TTATCCTTGA TGGAGACAGT |
| 1861 | TTACAGCCAG AGGCTTCAGA ATCCAGTGCC ATACCAGAGG |
| | CTAACTCGGA GACTTTCAAG |
| 1921 | GAAAGCACAA ACCTTGGAAA CCTAGAGGAG TCCTCTGAAT |
| | AA. |

In some embodiments, the Construct comprises or consists of a sequence encoding a CAG promoter having the sequence of

1 CCATTGACGT CAATAATGAC GTATGTTCCC ATAGTAACGC CAATAGGGAC TTTCCATTGA

61 CGTCAATGGG TGGAGTATTT ACGGTAAACT GCCCACTTGG CAGTACATCA AGTGTATCAT

121 ATGCCAAGTA CGCCCCCTAT TGACGTCAAT GACGGTAAAT GGCCCGCCTG GCATTATGCC

181 CAGTACATGA CCTTATGGGA CTTTCCTACT TGGCAGTACA TCTACGTATT AGTCATCGCT

241 ATTACCATGG TCGAGGTGAG CCCCACGTTC TGCTTCACTC TCCCCATCIC CCCCCCCTCC

301 CCACCCCCAA TTTTGTATTT ATTTATTTTT TAATTATTTT GTGCAGCGAT GGGGGCGGGG

361 GGGGGGGGGG GGCGCGCGCC AGGCGGGGCG GGGCGGGGCG AGGGGCGGGG CGGGGCGAGG

421 CGGAGAGGTG CGGCGGCAGC CAATCAGAGC GGCGCGCTCC GAAAGTTTCC TTTTATGGCG

481 AGGCGGCGGC GGCGGCGGCC CTATAAAAAG CGAAGCGCGC GGCGGGCGGG AGTCGCTGCG

541 CGCTGCCTTC GCCCCGTGCC CCGCTCGGCC GCCGCCTCGC GCCGCCCGCC CCGGCTCTGA

601 CTGACCGCGT TACTCCCACA GGTGAGCGGG CGGGACGGCC CTTCTCCTCC GGGCTGTAAT

661 TAGCGCTTGG TTTAATGACG GCTTGTTTCT TTTCTGTGGC TGCGTGAAAG CCTTGAGGGG

721 CTCCGGGAGG GCCCTTTGTG CGGGGGGAGC GGCTCGGGGC TGTCCGCGGG GGGACGGCTG

781 CCTTCGGGGG GGACGGGGCA GGGCGGGGTT CGGCTTCTGG CGTGTGACCG GCGGCTCTAG

841 AGCCTCTGCT AACCATGTTC ATGCCTTCTT CTTTTTCCTA CAGCTCCTGG GCAACGTGCT

901 GGTTATTGTG CTGTCTCATC ATTTTGGCAA AGAATTGGAT CC (SEQ ID NO: 18) or

a sequence having at least 50%, 55%, 60%, 65%, 70%, 75%, 80%, 85%, 90%, 95%, 99%, or any percentage in between of identify to the sequence of

(SEQ ID NO: 18)

| | |
|---|---|
| 1 | CCATTGACGT CAATAATGAC GTATGTTCCC ATAGTAACGC |
| | CAATAGGGAC TTTCCATTGA |
| 61 | CGTCAATGGG TGGAGTATTT ACGGTAAACT GCCCACTTGG |
| | CAGTACATCA AGTGTATCAT |
| 121 | ATGCCAAGTA CGCCCCCTAT TGACGTCAAT GACGGTAAAT |
| | GGCCCGCCTG GCATTATGCC |
| 181 | CAGTACATGA CCTTATGGGA CTTTCCTACT TGGCAGTACA |
| | TCTACGTATT AGTCATCGCT |
| 241 | ATTACCATGG TCGAGGTGAG CCCCACGTTC TGCTTCACTC |
| | TCCCCATCTC CCCCCCCTCC |
| 301 | CCACCCCCAA TTTTGTATTT ATTTATTTTT TAATTATTTT |
| | GTGCAGCGAT GGGGGCGGGG |
| 361 | GGGGGGGGGG GGCGCGCGCC AGGCGGGGCG GGGCGGGGCG |
| | AGGGGCGGGG CGGGGCGAGG |
| 421 | CGGAGAGGTG CGGCGGCAGC CAATCAGAGC GGCGCGCTCC |
| | GAAAGTTTCC TTTTATGGCG |
| 481 | AGGCGGCGGC GGCGGCGGCC CTATAAAAAG CGAAGCGCGC |
| | GGCGGGCGGG AGTCGCTGCG |
| 541 | CGCTGCCTTC GCCCCGTGCC CCGCTCCGCC GCCGCCTCGC |
| | GCCGCCCGCC CCGGCTCTGA |
| 601 | CTGACCGCGT TACTCCGACA GGTGAGCGGG CGGGACGGCC |
| | CTTCTCCTCC GGGCTGTAAT |
| 661 | TAGCGCTTGG TTTAATGACG GCTTGTTTCT TTTCTGTGGC |
| | TGCGTGAAAG CCTTGAGGGG |
| 721 | CTCCGGGAGG GCCCTTTGTG CGGGGGGAGC GGCTCGGGGC |
| | TGTCCGCGGG GGGACGGCTG |
| 781 | CCTTCGGGGG GGACGGGGCA GGGCGGGGTT CGGCTTCTGG |
| | CGTGTGACCG GCGGCTCTAG |
| 841 | AGCCTCTGCT AACCATGTTC ATGCCTTCTT CTTTTTCCTA |
| | CAGCTCCTGG GCAACGTGCT |
| 901 | GGTTATTGTG CTGTCTCATC ATTTTGGCAA AGAATTGGAT |
| | CC. |

In some embodiments, the Construct comprises or consists of a sequence encoding a mutated WPRE signal having the sequence of

1 ATCGATAATC AACCTCTGGA TTACAAAATT TGTGAAAGAT TGACTGGTAT TCTTAACTAT

61 GTTGCTCCTT TTACGCTATG TGGATACGCT GCTTTAATGC CTTTGTATCA TGCTATTGCT

121 TCCCGTATGG CTTTCATTTT CTCCTCCTTG TATAAATCCT GGTTGCTGTC TCTTTATGAG

181 GAGTTGTGGC CCGTTGTCAG GCAACGTGGC GTGGTGTGCA CTGTGTTTGC TGACGCAACC

241 CCCACTGGTT GGGGCATTGC CACCACCTGT CAGCTCCTTT CCGGGACTTT CGCTTTCCCC

301 CTCCCTATTG CCACGGCGGA ACTCATCGCC GCCTGCCTTG CCCGCTGCTG GACAGGGGCT

361 CGGCTGTTGG GCACTGACAA TTCCGTGGTG TTGTCGGGGA AATCATCGTC CTTTCCTTGG

421 CTGCTCGCCT GTGTTGCCAC CTGGATTCTG CGCGGGACGT CCTTCTGCTA CGTCCCTTCG

481 GCCCTCAATC CAGCGGACCT TCCTTCCCGC GGCCTGCTGC CGGCTCTGCG GCCTCTTCCG

541 CGTCTTCGCC TTCGCCCTCA GACGAGTCGG ATCTCCCTTT GGGCCGCCTC CCC (SEQ ID NO: 19) or a sequence having at least 50%, 55%, 60%, 65%, 70%, 75%, 80%, 85%, 90%, 95%, 99%, or any percentage in between of identify to the sequence of

(SEQ ID NO: 19)

| | |
|---|---|
| 1 | ATCGATAATC AACCTCTGGA TTACAAAATT TGTGAAAGAT |
| | TGACTGGTAT TCTTAACTAT |
| 61 | GTTGCTCCTT TTACGCTATG TGGATACGCT GCTTTAATGC |
| | CTTTGTATCA TGCTATTGCT |
| 121 | TCCCGTATGG CTTTCATTTT CTCCTCCTTG TATAAATCCT |
| | GGTTGCTGTC TCTTTATGAG |
| 181 | GAGTTGTGGC CCGTTGTCAG GCAACGTGGC GTGGTGTGCA |
| | CTGTGTTTGC TGACGCAACC |
| 241 | CCCACTGGTT GGGGCATTGC CACCACCTGT CAGCTCCTTT |
| | CCGGGACTTT CGCTTTCCCC |
| 301 | CTCCCTATTG CCACGGCGGA ACTGATCGCC GCCTGCCTTG |
| | CCCGCTGCTG GACAGGGGCT |
| 361 | CGGCTGTTGG GCACTGACAA TTCCGTGGTG TTGTCGGGGA |
| | AATCATCGTC CTTTCCTTGG |
| 421 | CTGCTCGCCT GTGTTGCCAC CTGGATTCTG CGCGGGACGT |
| | CCTTCTGCTA CGTCCCTTCG |
| 481 | GCCCTCAATC CAGCGGACCT TCCTTCCCGC GGCCTGCTGC |
| | CGGCTCTGCG GCCTCTTCCG |
| 541 | CGTCTTCGCC TTCGCCCTCA GACGAGTCGG ATCTCCCTTT |
| | GGGCCGCCTC CCC |

In some embodiments, the Construct comprises or consists of a sequence encoding a polyadenylation signal of the bovine growth hormone having the sequence of

1 CGCTGATCAG CCTCGACTGT GCCTTCTAGT TGCCAGCCAT CTGTTGTTTG CCCCTCCCCC

61 GTGCCTTCCT TGACCCTGGA AGGTGCCACT CCCACTGTCC TTTCCTAATA AAATGAGGAA

121 ATTGCATCGC ATTGTCTGAG TAGGTGTCAT TCTATTCTGG GGGGTGGGGT GGGGCAGGAC

181 AGCAAGGGGG AGGATTGGGA AGACAATAGC AGGCATGCTG GGGATGCGGT GGGCTCTATG

241 GCTTCTGAGG CGGAAAGAAC CAGCTGGGG (SEQ ID NO 20) or a sequence having at least 50%, 55%, 60%, 65%, 70%, 75%, 80%, 85%, 90%, 95%, 99%, or any percentage in between of identify to the sequence of

(SEQ ID NO: 20)

| | |
|---|---|
| 1 | CGCTGATCAG CCTCGACTGT GCCTTCTAGT TGCCAGGCAT |
| | CTGTTGTTTG CCCCTCCCCC |
| 61 | GTGCCTTCCT TGACCCTGGA AGGTGCCACT CCCACTGTCC |
| | TTTCCTAATA AAATGAGGAA |
| 121 | ATTGCATCGC ATTGTCTGAG TAGGTGTCAT TCTATTCTGG |
| | GGGGTGGGGT GGGGCAGGAC |
| 181 | AGCAAGGGGG AGGATTGGGA AGACAATAGC AGGCATGCTG |
| | GGGATGCGGT GGGCTCTATG |
| 241 | GCTTCTGAGG CGGAAAGAAC CAGCTGGGG. |

In some embodiments, the Construct comprises or consists of a sequence encoding a 5′ITR having the sequence of

1 CTGCGCGCTC GCTCGCTCAC TGAGGCCGCC CGGGCAAAGC CCGGGCGTCG GGCGACCTTT

61 GGTCGCCCGG CCTCAGTGAG CGAGCGAGCG CGCAGAGAGG GAGTGGCCAA CTCCATCACT

121 AGGGGTTCCT (SEQ ID NO: 21) or a sequence having at least 50%, 55%, 60%, 65%, 70%, 75%, 80%, 85%, 90%, 95%, 99%, or any percentage in between of identity to the sequence of

(SEQ ID NO: 21)

| | |
|---|---|
| 1 | CTGCGCGCTC GCTCGCTCAC TGAGGCCGCC CGGGCAAAGC |
| | CCGGGCGTCG GGCGACCTTT |
| 61 | GGTCGCCCGG CCTCAGTGAG CGAGCGAGCG CGCAGAGAGG |
| | GAGTGGCCAA CTCCATCACT |
| 121 | AGGGGTTCCT. |

In some embodiments, the Construct comprises or consists of a sequence encoding a 3′ITR having the sequence of

1 AGGAACCCCT AGTGATGGAG TTGGCCACTC CCTCTCTGCG CGCTCGCTCG CTCACTGAGG

61 CCGGGCGACC AAAGGTCGCC CGACGCCCGG GCGGCCTCAG TGAGCGAGCG AGCGCGCAGA

121 G (SEQ ID NO: 22) or a sequence having at least 50%, 55%, 60%, 65%, 70%, 75%, 80%, 85%, 90%, 95%, 99%, or any percentage in between of identify to the sequence of

(SEQ ID NO: 22)

| | |
|---|---|
| 1 | AGGAACCCCT AGTGATGGAG TTGGCCACTC CCTCTCTGCG |
| | CGCTCGCTCG CTCACTGAGG |
| 61 | CCGGGCGACC AAAGGTCGCC CGACGCCCGG GCGGCCTCAG |
| | TGAGCGAGCG AGCGCGCAGA |
| 121 | G. |

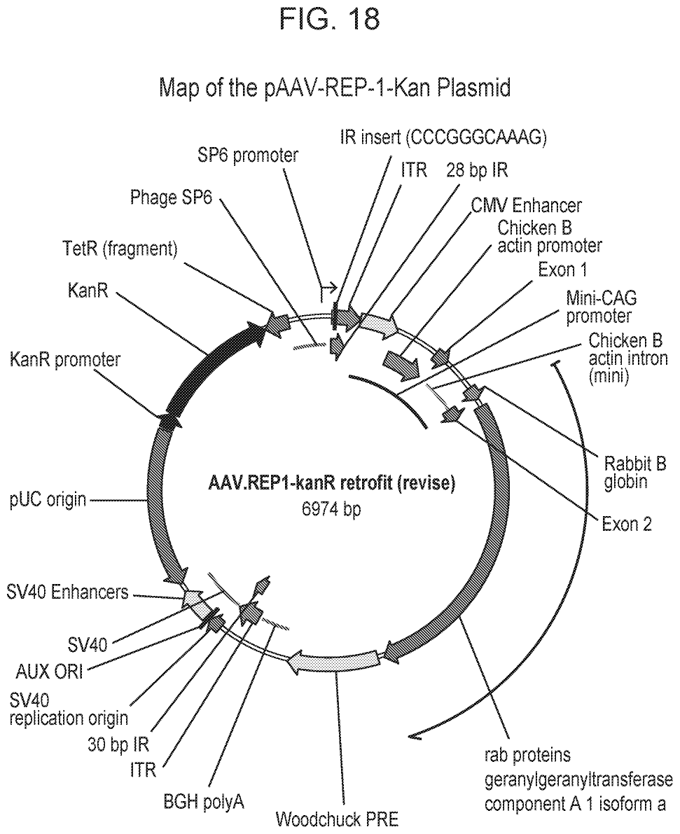

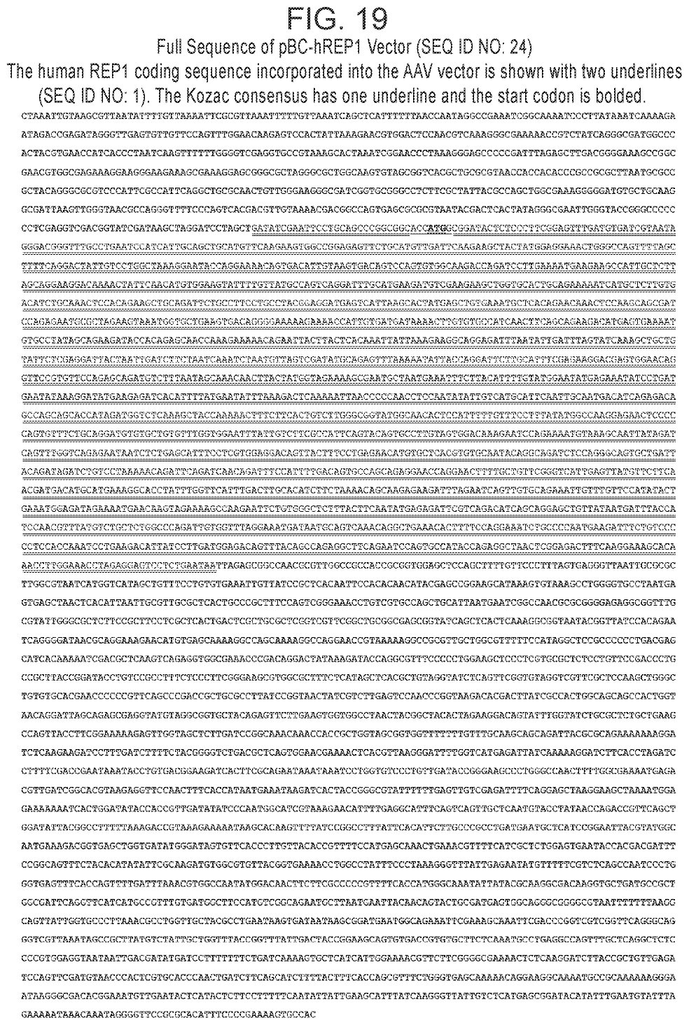

In some embodiments of the compositions of the disclosure, wherein the Construct comprises a human REP-1 protein or a portion thereof, the Construct is referred to as “AAV2-REP1 or AAV2.REP1” and the International Nonproprietary Name (INN) is timrepigene emparvovec. In some embodiments of the compositions of the disclosure, wherein the Construct comprises a human REP-1 protein or a portion thereof, the AAV2-REP1 product consists of a purified recombinant serotype 2 adeno-associated viral vector (rAAV) encoding the cDNA of human Rab escort protein type 1 (REP1). In some embodiments, each 20 nm AAV virion contains a single stranded DNA insert sequence of 4173 bp in length (plus short cloning sites flanking each element) comprising: a 177 bp 5′ inverted terminal repeat (ITR), a 934 bp Cytomegalovirus enhancer/chicken-beta actin (CBA) hybrid promoter, a 1962 bp human REP1 cDNA, a 589 bp Woodchuck hepatitis virus post-transcriptional regulatory element (WPRE), a 242 bp Bovine growth hormone polyadenylation sequence (BGH-polyA), and a 165 bp 3′ ITR. In some embodiments, a pAAV.REP-1-Kan plasmid used to generate the AAV2-REP1 vector is shown in FIG. 18. In some embodiments, the 1962 bp human REP1 cDNA comprises the nucleic acid sequence of:

(SEQ ID NO: 23)

| | |
|---|---|
| 1 | atggcggata ctctcccttc ggagtttgat gtgatcgtaa |
| | tagggacggg tttgcctgaa |
| 61 | tccatcattg cagctgcatg ttcaagaagt ggccggagag |
| | ttctgcatgt tgattcaaga |
| 121 | agctactatg gaggaaactg ggccagtttt agcttttcag |
| | gactattgtc ctggctaaag |
| 181 | gaataccagg aaaacagtga cattgtaagt gacagtccag |
| | tgtggcaaga ccagatcctt |
| 241 | gaaaatgaag aagccattgc tcttagcagg aaggacaaaa |
| | ctattcaaca tgtggaagta |
| 301 | ttttgttatg ccagtcagga tttgcatgaa gatgtcgaag |
| | aagctggtgc actgcagaaa |
| 361 | aatcatgctc ttgtgacatc tgcaaactcc acagaagctg |
| | cagattctgc cttcctgcct |
| 421 | acggaggatg agtcattaag cactatgagc tgtgaaatgc |
| | tcacagaaca aactccaagc |
| 481 | agcgatccag agaatgcgct agaagtaaat ggtgctgaag |
| | tgacagggga aaaagaaaac |
| 541 | cattgtgatg ataaaacttg tgtgccatca acttcagcag |
| | aagacatgag tgaaaatgtg |
| 601 | cctatagcag aagataccac agagcaacca aagaaaaaca |
| | gaattactta ctcacaaatt |
| 661 | attaaagaag gcaggagatt taatattgat ttagtatcaa |
| | agctgctgta ttctcgagga |
| 721 | ttactaattg atcttctaat caaatctaat gttagtcgat |
| | atgcagagtt taaaaatatt |
| 781 | accaggattc ttgcatttcg agaaggacga gtggaacagg |
| | ttccgtgttc cagagcagat |
| 841 | gtctttaata gcaaacaact tactatggta gaaaagcgaa |
| | tgctaatgaa atttcttaca |
| 901 | ttttgtatgg aatatgagaa atatcctgat gaatataaag |
| | gatatgaaga gatcacattt |
| 961 | tatgaatatt taaagactca aaaattaacc cccaacctcc |
| | aatatattgt catgcattca |
| 1021 | attgcaatga catcagagac agccagcagc accatagatg |
| | gtctcaaagc taccaaaaac |
| 1081 | tttcttcact gtcttgggcg gtatggcaac actccatttt |
| | tgtttccttt atatggccaa |
| 1141 | ggagaactcc cccagtgttt ctgcaggatg tgtgctgtgt |
| | ttggtggaat ttattgtctt |
| 1201 | cgccattcag tacagtgcct tgtagtggac aaagaatcca |
| | gaaaatgtaa agcaattata |
| 1261 | gatcagtttg gtcagagaat aatctctgag catttcctcg |
| | tggaggacag ttactttcct |
| 1321 | gagaacatgt gctcacgtgt gcaatacagg cagatctcca |
| | gggcagtgct gattacagat |
| 1381 | agatctgtcc taaaaacaga ttcagatcaa cagatttcca |
| | ttttgacagt gccagcagag |
| 1441 | gaaccaggaa cttttgctgt tcgggtcatt gagttatgtt |
| | cttcaacgat gacatgcatg |
| 1501 | aaaggcacct atttggttca tttgacttgc acatcttcta |
| | aaacagcaag agaagattta |
| 1561 | gaatcagttg tgcagaaatt gtttgttcca tatactgaaa |
| | tggagataga aaatgaacaa |
| 1621 | gtagaaaagc caagaattct gtgggctctt tacttcaata |
| | tgagagattc gtcagacatc |
| 1681 | agcaggagct gttataatga tttaccatcc aacgtttatg |
| | tctgctctgg cccagattgt |
| 1741 | ggtttaggaa atgataatgc agtcaaacag gctgaaacac |
| | ttttccagga aatctgcccc |
| 1801 | aatgaagatt tctgtccccc tccaccaaat cctgaagaca |
| | ttatccttga tggagacagt |
| 1861 | ttacagccag aggcttcaga atccagtgcc ataccagagg |
| | ctaactcgga gactttcaag |
| 1921 | gaaagcacaa accttggaaa cctagaggag tcctctgaat |
| | aa. |

In some embodiments of the compositions of the disclosure, the Construct further comprises a sequence corresponding to a 5′ inverted terminal repeat (ITR) and a sequence corresponding to a 3′ inverted terminal repeat (ITR). In some embodiments, the sequence encoding the 5′ ITR and the sequence encoding the 3′ITR are identical. In some embodiments, the sequence encoding the 5′ ITR and the sequence encoding the 3′ITR are not identical. In some embodiments, the sequence encoding the 5′ ITR and the sequence encoding the 3′ITR are isolated or derived from an adeno-associated viral vector of serotype 2 (AAV2). In some embodiments, the sequence encoding the 5′ ITR and the sequence encoding the 3′ITR comprise a wild type sequence. In some embodiments, the sequence encoding the 5′ ITR and the sequence encoding the 3′ITR comprise a truncated wild type AAV2 sequence. In some embodiments, the sequence encoding the 5′ ITR and the sequence encoding the 3′ITR comprise a variation when compared to a wild type sequence of the same AAV serotype. In some embodiments, the variation comprises a substitution, an insertion, a deletion, an inversion, or a transposition. In some embodiments, the variation comprises a truncation or an elongation of a wild type or a variant sequence.

In some embodiments of the compositions of the disclosure, an AAV comprises a sequence corresponding to a 5′ inverted terminal repeat (ITR) and a sequence corresponding to a 3′ inverted terminal repeat (ITR). In some embodiments, the sequence encoding the 5′ ITR and the sequence encoding the 3′ITR are identical. In some embodiments, the sequence encoding the 5′ ITR and the sequence encoding the 3′ITR are not identical. In some embodiments, the sequence encoding the 5′ ITR and the sequence encoding the 3′ITR are isolated or derived from an adeno-associated viral vector of serotype 2 (AAV2). In some embodiments, the sequence encoding the 5′ ITR and the sequence encoding the 3′ITR comprise a wild type sequence. In some embodiments, the sequence encoding the 5′ ITR and the sequence encoding the 3′ITR comprise a truncated wild type AAV2 sequence. In some embodiments, the sequence encoding the 5′ ITR and the sequence encoding the 3′ITR comprise a variation when compared to a wild type sequence of the same AAV serotype. In some embodiments, the variation comprises a substitution, an insertion, a deletion, an inversion, or a transposition. In some embodiments, the variation comprises a truncation or an elongation of a wild type or a variant sequence.

In some embodiments of the compositions of the disclosure, an AAV comprises a viral sequence essential for formation of a replication-deficient AAV. In some embodiments, the viral sequence is isolated or derived from an AAV of the same serotype as one or both of the sequence encoding the 5′ITR or the sequence encoding the 3′ITR. In some embodiments, the viral sequence, the sequence encoding the 5′ITR or the sequence encoding the 3′ITR are isolated or derived from an AAV2. In some embodiments, the viral sequence, the sequence encoding the 5′ITR and the sequence encoding the 3′ITR are isolated or derived from an AAV2.

In some embodiments of the compositions of the disclosure, an AAV comprises a viral sequence essential for formation of a replication-deficient AAV, a sequence encoding the 5′ITR and a sequence encoding the 3′ITR, but does not comprise any other sequence isolated or derived from an AAV. In some embodiments, the AAV is a recombinant AAV (rAAV), comprising a viral sequence essential for formation of a replication-deficient AAV, a sequence encoding the 5′ITR, a sequence encoding the 3′ITR, and a sequence encoding a Construct of the disclosure.

In some embodiments, a plasmid DNA used to create the rAAV in a host cell comprises a selection marker. Exemplary selection markers include, but are not limited to, antibiotic resistance genes. Exemplary antibiotic resistance genes include, but are not limited to, ampicillin and kanamycin. Exemplary selection markers include, but are not limited to, drug or small molecule resistance genes. Exemplary selection markers include, but are not limited to, dapD and a repressible operator including but not limited to a lacO/P construct controlling or suppressing dapD expression, wherein plasmid selection is performed by administering or contacting a transformed cell with a plasmid capable of operator repressor titration (ORT). Exemplary selection markers include, but are not limited to, a ccd selection gene. In some embodiments, the ccd selection gene comprises a sequence encoding a ccdA selection gene that rescues a host cell line engineered to express a toxic ccdB gene. Exemplary selection markers include, but are not limited to, sacB, wherein an RNA is administered or contacted to a host cell to suppress expression of the sacB gene in sucrose media. Exemplary selection markers include, but are not limited to, a segregational killing mechanism such as the parAB+ locus composed of Hok (a host killing gene) and Sok (suppression of killing).

### AAV-Construct Structure

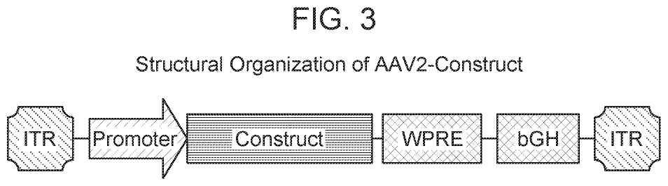

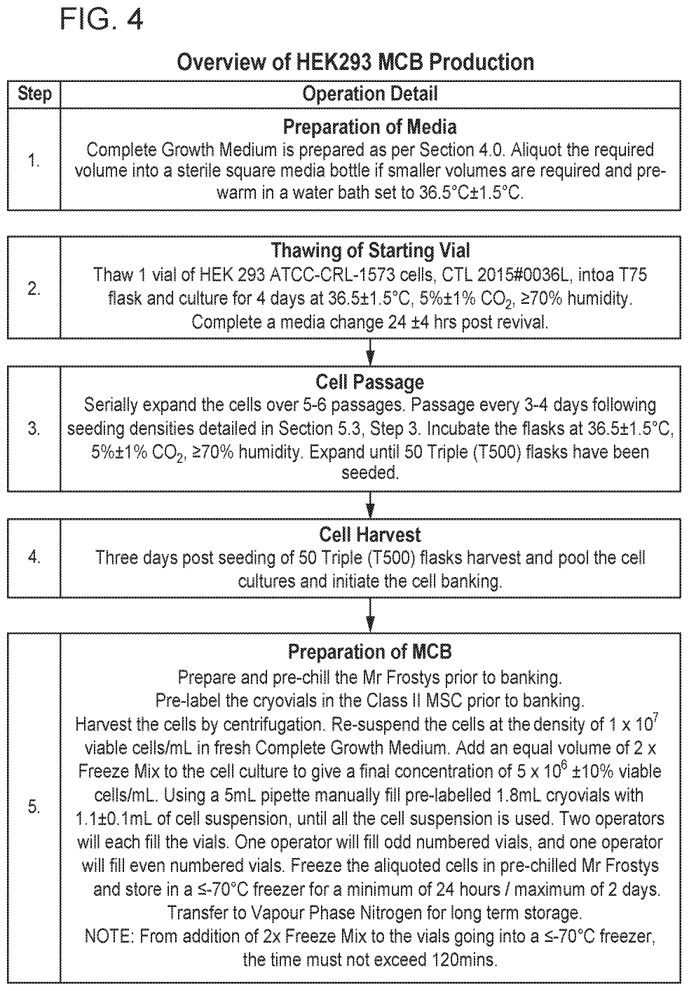

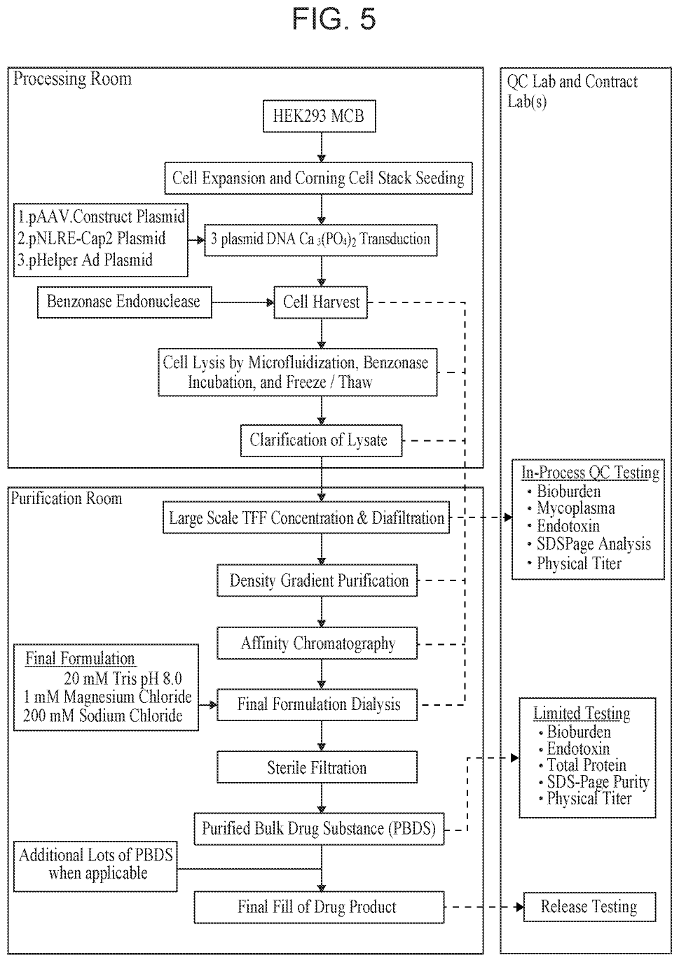

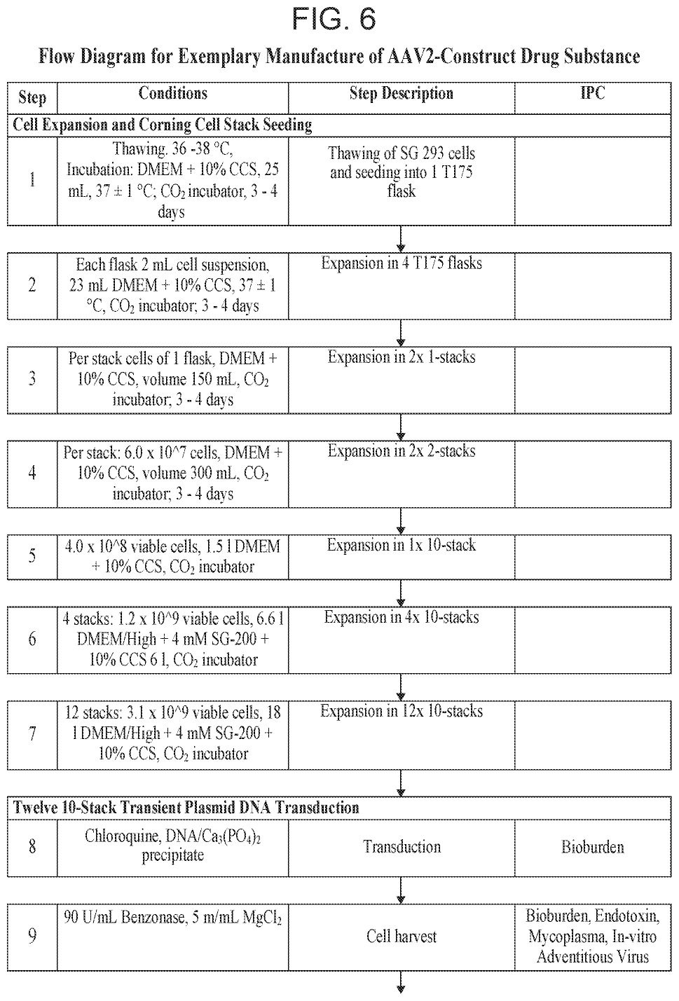

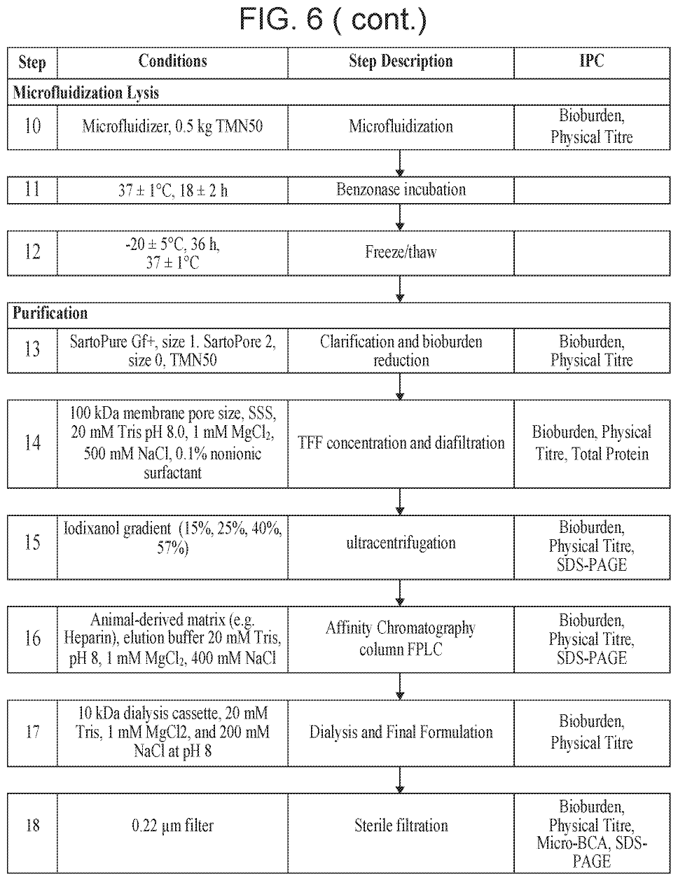

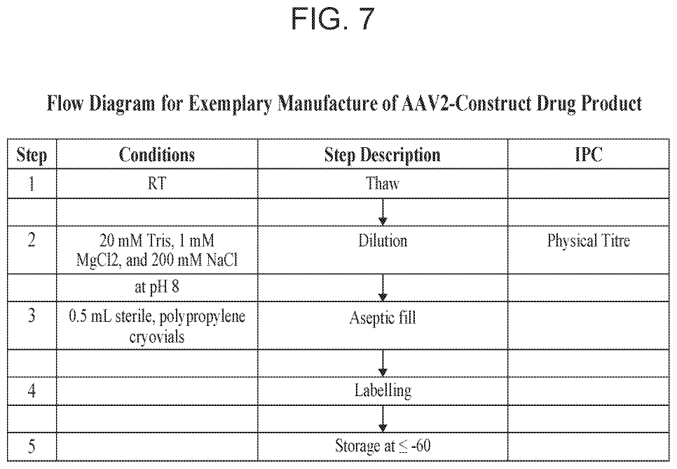

The AAV2-Construct product consists of a purified recombinant serotype 2 adeno-associated viral vector (rAAV) encoding the cDNA encoding a therapeutic construct. An exemplary diagram is provided in FIG. 3.

In some embodiments, the AAV2-Construct comprises one or more of a sequence encoding a 5′ ITR, a sequence encoding a 3′ ITR and a sequence encoding a capsid protein that is isolated and/or derived from a serotype 2 adeno-associated viral vector (AAV2). In some embodiments, the AAV2-Construct comprises a sequence encoding a 5′ ITR, a sequence encoding a 3′ ITR and a sequence encoding a capsid protein that is isolated and/or derived from a serotype 2 adeno-associated viral vector (AAV2). In some embodiments, the AAV2-Construct comprises a truncated sequence encoding a 5′ ITR and a sequence encoding a 3′ ITR that is isolated and/or derived from a serotype 2 adeno-associated viral vector (AAV2). In some embodiments, the AAV2-Construct comprises wild type AAV2 ITRs (a wild type 5′ ITR and a wild type 3′ ITR).

In some embodiments, each 20 nm AAV virion contains a single stranded DNA insert sequence (plus short cloning sites flanking each element) comprising: (a) a 5′ inverted terminal repeat (ITR), (b) a promoter suitable for expression in mammalian cells, (c) a cDNA encoding a therapeutic construct, and (d) a 3′ ITR.

In some embodiments, each 20 nm AAV virion contains a single stranded DNA insert sequence (plus short cloning sites flanking each element) comprising: (a) a 145 bp 5′ inverted terminal repeat (ITR), (b) a promoter suitable for expression in mammalian cells, (c) a cDNA encoding a therapeutic construct, and (d) a 145 bp 3′ ITR. In some embodiments, each 20 nm AAV virion contains a single stranded DNA insert sequence (plus short cloning sites flanking each element) comprising: (a) a 5′ inverted terminal repeat (ITR), (b) a promoter suitable for expression in mammalian cells, (c) a cDNA encoding a therapeutic construct, and (d) a 3′ ITR, wherein the 5′ ITR or the 3′ ITR comprises or consists of 134, 135, 136, or 137 bp.

In some embodiments, each 20 nm AAV virion contains a single stranded DNA insert sequence (plus short cloning sites flanking each element) comprising: (a) a 145 bp5′ inverted terminal repeat (ITR), (b) a promoter suitable for expression in mammalian cells, (c) a cDNA encoding a therapeutic construct, (e) a polyadenylation sequence (polyA), and (f) a 145 bp3′ ITR. In some embodiments, each 20 nm AAV virion contains a single stranded DNA insert sequence (plus short cloning sites flanking each element) comprising: (a) a 5′ inverted terminal repeat (ITR), (b) a promoter suitable for expression in mammalian cells, (c) a cDNA encoding a therapeutic construct, (e) a polyadenylation sequence (polyA), and (f) a 3′ ITR, wherein the 5′ ITR or the 3′ ITR comprises or consists of 134, 135, 136, or 137 bp.

In some embodiments, each 20 nm AAV virion contains a single stranded DNA insert sequence (plus short cloning sites flanking each element) comprising: (a) a 145 bp 5′ inverted terminal repeat (ITR), (b) a promoter suitable for expression in mammalian cells, (c) a cDNA encoding a therapeutic construct, (d) a post-transcriptional regulatory element (PRE), (e) a polyadenylation sequence (polyA), and (f) a 145 bp 3′ ITR. In some embodiments, each 20 nm AAV virion contains a single stranded DNA insert sequence (plus short cloning sites flanking each element) comprising: (a) a 5′ inverted terminal repeat (ITR), (b) a promoter suitable for expression in mammalian cells, (c) a cDNA encoding a therapeutic construct, (d) a post-transcriptional regulatory element (PRE), (e) a polyadenylation sequence (polyA), and (f) a 3′ ITR, wherein the 5′ ITR or the 3′ ITR comprises or consists of 134, 135, 136, or 137 bp.

In some embodiments, each 20 nm AAV virion contains a single stranded DNA insert sequence (plus short cloning sites flanking each element) comprising: (a) a 145 bp 5′ inverted terminal repeat (ITR), (b) a promoter, optionally, a 934 bp Cytomegalovirus enhancer/chicken-beta actin (CBA) hybrid promoter, (c) a cDNA encoding a therapeutic construct, (d) a 589 bp Woodchuck hepatitis virus post-transcriptional regulatory element (WPRE), (e) a 242 bp Bovine growth hormone polyadenylation sequence (BGH-polyA), and (f) a 145 bp 3′ ITR. In some embodiments, each 20 nm AAV virion contains a single stranded DNA insert sequence (plus short cloning sites flanking each element) comprising: (a) a 5′ inverted terminal repeat (ITR), (b) a promoter, optionally, a 934 bp Cytomegalovirus enhancer/chicken-beta actin (CBA) hybrid promoter, (c) a cDNA encoding a therapeutic construct, (d) a 589 bp Woodchuck hepatitis virus post-transcriptional regulatory element (WPRE), (e) a 242 bp Bovine growth hormone polyadenylation sequence (BGH-polyA), and (f) a 3′ ITR, wherein the 5′ ITR or the 3′ ITR comprises or consists of 134, 135, 136, or 137 bp.

AAVs or Constructs of the disclosure may comprise a sequence encoding a promoter capable of expression in a mammalian cell. Preferably, AAVs or Constructs of the disclosure may comprise a sequence encoding a promoter capable of expression in a human cell. Exemplary promoters of the disclosure include, but are not limited to, constitutively active promoters, cell-type specific promoters, viral promoters, mammalian promoters, and hybrid or recombinant promoters. In some embodiments of the compositions of the disclosure, the therapeutic Construct of an AAV2-Construct is under the control of a chicken beta-actin promoter (CBA) Promoter. In some embodiments of the compositions of the disclosure, the CBA promoter comprises a sequence encoding a cytomegalovirus (CMV) enhancer and a sequence encoding a chicken beta-actin promoter (variously termed CBA or CAG).

AAVs or Constructs of the disclosure may comprise a sequence encoding a post-transcriptional regulatory element (PRE). Exemplary PREs of the disclosure include, but are not limited to, a Woodchuck hepatitis virus post-transcriptional regulatory element (WPRE). In some embodiments of the compositions of the disclosure, the AAV comprises a 589 bp WPRE, originating from the 3′ region of the viral S transcript, directly downstream of the cDNA encoding a therapeutic Construct of the disclosure. This WPRE is important for high-level expression of native mRNA transcripts, acting to enhance mRNA processing and transport of intronless genes. In some embodiments of the compositions of the disclosure, the WPRE has been modified to prevent expression of the viral X antigen by ablation of the translation initiation site. This has been achieved by deleting the We2 promoter/enhancer and mutating the We1 promoter.

AAVs or Constructs of the disclosure may comprise a polyadenosine (polyA) sequence. Exemplary polyA sequences of the disclosure include, but are not limited to, a bovine growth hormone polyadenylation (BGH-polyA) sequence. The BGH-polyA sequence is used to enhance gene expression and has been shown to yield three times higher expression levels than other polyA sequences such as SV40 and human collagen polyA. This increased expression is largely independent of the type of upstream promoter or transgene. Increasing expression levels using both BGH-polyA and WPRE sequences allows a lower overall dose of AAV or plasmid vector to be injected, which is less likely to generate a host immune response.

In one illustrative embodiment, the pBC-hREP1 vector comprises or consists of the nucleic acid sequence set forth in SEQ ID NO:24.

In some embodiments of the compositions of the disclosure, the composition comprises a Drug Substance. As used herein, a Drug Substance comprises a rAAV of the disclosure comprising a Construct of the disclosure.

In some embodiments of the compositions of the disclosure, the composition comprises a Drug Product. As used herein, a Drug Product comprises a drug substance, formulated for administration to a subject for the treatment or prevention of a disease or disorder.

The components of an exemplary Drug Product of the disclosure, their functions and specifications are listed in Table 1.

TABLE 1
Composition of AAV2-Construct Drug Product

| Name of | | | Quantity/ |
|---|---|---|---|
| Ingredient | Function | Grade | Concentration |
| AAV2- | Active | GMP | 1.0 × 10\hat{ }12 DRP/mL |
| Construct | | | |
| Tris, pH 8.0 | Buffer | EP, BP, USP, JPC | 20 mM |
| MgCl₂ | Enhance vector | EP, BP, USP, JPC, | 1 mM |
| | stability | FCC | |
| NaCl | Enhance vector | EP, BP, USP, JP | 200 mM |
| | stability and | | |
| | prevent vector | | |
| | aggregation | | |
| Poloxamer | | EP, USP | 0.001% |
| 188 | | | |
| Water for | Diluent | EP, USP | QS to 125 mL |
| Injections | | | |

### Dosage Form

Compositions of the disclosure may be formulated for systemic or local administration.

Compositions of the disclosure may be formulated as a Suspension for Injection or Infusion.

Compositions of the disclosure may be formulated for injection or infusion by any route, including but not limited to, an intravitreous injection or infusion, a subretinal injection or infusion, or a suprachoroidal injection or infusion.

Compositions of the disclosure may be formulated at a concentration of between 1.0×10\hat{ }10 DRP/mL and 1.0×10\hat{ }14 DRP/mL, inclusive of the endpoints. In some embodiments, compositions of the disclosure may be formulated at a concentration of about 1.0×10\hat{ }12 DRP/mL. In some embodiments, compositions of the disclosure may be formulated at a concentration of 1.0×10\hat{ }12 DRP/mL. In some embodiments, compositions of the disclosure may be formulated at a concentration of between 0.1×10\hat{ }12 DRP/mL and 10.0×10\hat{ }12 DRP/mL, inclusive of the endpoints. In some embodiments, compositions of the disclosure may be formulated at a concentration of between 0.1×10\hat{ }12 DRP/mL and 5.0×10\hat{ }12 DRP/mL, inclusive of the endpoints. In some embodiments, compositions of the disclosure may be formulated at a concentration of between 0.1×10\hat{ }12 DRP/mL and 2.0×10\hat{ }12 DRP/mL, inclusive of the endpoints. In some embodiments, compositions of the disclosure may be formulated at a concentration of between 0.5×10\hat{ }12 DRP/mL and 1.5×10\hat{ }12 DRP/mL, inclusive of the endpoints. In some embodiments, compositions of the disclosure may be formulated at a concentration of between 0.7×10\hat{ }12 DRP/mL and 1.3×10\hat{ }12 DRP/mL, inclusive of the endpoints. In some embodiments, compositions of the disclosure may be formulated at a concentration of between 0.8×10\hat{ }12 DRP/mL and 1.2×10\hat{ }12 DRP/mL, inclusive of the endpoints. In some embodiments, compositions of the disclosure may be formulated at a concentration of between 0.9×10\hat{ }12 DRP/mL and 1.1×10\hat{ }12 DRP/mL, inclusive of the endpoints.

Compositions of the disclosure may be diluted prior to administration using a using a diluent of the disclosure. In some embodiments, the diluent is identical to a formulation buffer used for preparation of the AAV2-Construct Drug Product. In some embodiments, the diluent is not identical to a formulation buffer used for preparation of the AAV2-Construct Drug Product.

Compositions of the disclosure, including the AAV2-Construct Drug Product described in Table 1, may be formulated as a Suspension for Injection containing 1.0×10\hat{ }12 DRP/mL. If required by the protocol, AAV2-Construct Drug Product may be diluted in the clinic (i.e. by a medical professional) before administration using a diluent of the disclosure. In some embodiments, this diluent is the same formulation buffer used for preparation of the AAV2-Construct Drug Product.

### Pharmaceutical Formulations

Compositions of the disclosure may comprise a Drug Substance. In some embodiments, the Drug Substance comprises or consists of an AAV2-Construct. In some embodiments, the Drug Substance comprises or consists of an AAV2-Construct and a formulation buffer. In some embodiments, the formulation buffer comprises 20 mM Tris, 1 mM MgCl₂, and 200 mM NaCl at pH 8. In some embodiments, the formulation buffer comprises 20 mM Tris, 1 mM MgCl₂, and 200 mM NaCl at pH 8 with poloxamer 188 at 0.001%.

### Excipients

Compositions of the disclosure may comprise a Drug Product. In some embodiments, the Drug Product comprises or consists of a Drug Substance and a formulation buffer. In some embodiments, the Drug Product comprises or consists of a Drug Substance diluted in a formulation buffer. In some embodiments, the Drug Product comprises or consists of an AAV2-Construct Drug Substance diluted to a final Drug Product AAV2-Construct vector genome (vg) concentration in a formulation buffer.

### Ocular Formulations

Compositions of the disclosure may be formulated to comprise, consist essentially of or consist of an AAV2-Construct Drug Substance at an optimal concentration for ocular injection or infusion.

Compositions of the disclosure may comprise one or more buffers that increase or enhance the stability of an AAV of the disclosure. In some embodiments, compositions of the disclosure may comprise one or more buffers that ensure or enhance the stability of an AAV2 of the disclosure. Alternatively, or in addition, compositions of the disclosure may comprise one or more buffers that prevent, decrease, or minimize AAV particle aggregation. In some embodiments, compositions of the disclosure may comprise one or more buffers that prevent, decrease, or minimize AAV2 particle aggregation.

Compositions of the disclosure may comprise one or more components that induce or maintain a neutral or slightly basic pH. In some embodiments, compositions of the disclosure comprise one or more components that induce or maintain a neutral or slightly basic pH of between 7 and 9, inclusive of the endpoints. In some embodiments, compositions of the disclosure comprise one or more components that induce or maintain a pH of about 8. In some embodiments, compositions of the disclosure comprise one or more components that induce or maintain a pH of between 7.5 and 8.5. In some embodiments, compositions of the disclosure comprise one or more components that induce or maintain a pH of between 7.7 and 8.3. In some embodiments, compositions of the disclosure comprise one or more components that induce or maintain a pH of between 7.9 and 8.1. In some embodiments, compositions of the disclosure comprise one or more components that induce or maintain a pH of 8.

Following contact of a composition of the disclosure and a cell, the AAV2-Construct expresses a gene or a portion thereof, resulting in the production of a product encoded by the gene or a portion thereof. In some embodiments, the cell is a target cell. In some embodiments, the target cell is a retinal cell. In some embodiments, the retinal cell is a neuron. In some embodiments, the neuron is a photoreceptor. In some embodiments, the cell is in vivo, in vitro, ex vivo or in situ. In some embodiments, including those wherein the cell is in vivo, the contacting occurs following administration of the composition to a subject. In some embodiments, the AAV2-Construct expresses a gene or a portion thereof, results in the production of a product encoded by the gene or a portion thereof at a therapeutically-effective level of expression of the gene product. In some embodiments, the gene product is a protein.

### Batch Formulations

Compositions of the disclosure may be manufactured at a scale of between 1 to 1000 vials per batch, inclusive of the endpoints. In some embodiments of the compositions of the disclosure, a composition, Drug Substance, or Drug Product may be manufactured at a scale of between 50 to 500 vials per batch, inclusive of the endpoints. In some embodiments of the compositions of the disclosure, a composition, Drug Substance, or Drug Product may be manufactured at a scale of between 100 to 415 vials per batch, inclusive of the endpoints.

Exemplary batches of the disclosure may comprise between 0.01 mL and 5 mL, inclusive of the endpoints, of a composition, Drug Substance, or Drug Product of the disclosure. In some embodiments, a batch of the disclosure may comprise between 0.01 mL and 1 mL, inclusive of the endpoints, of a composition, Drug Substance, or Drug Product of the disclosure. In some embodiments, a batch of the disclosure may comprise between 0.1 mL and 1 mL, inclusive of the endpoints, of a composition, Drug Substance, or Drug Product of the disclosure. In some embodiments, a batch of the disclosure may comprise between 0.1 mL and 5 mL, inclusive of the endpoints, of a composition, Drug Substance, or Drug Product of the disclosure. In some embodiments, a batch of the disclosure may comprise between 0.25 mL and 0.35 mL, inclusive of the endpoints, of a composition, Drug Substance, or Drug Product of the disclosure. In some embodiments, a batch of the disclosure may comprise about 0.3 mL of a composition, Drug Substance, or Drug Product of the disclosure. In some embodiments, a batch of the disclosure may comprise 0.3 mL of a composition, Drug Substance, or Drug Product of the disclosure.

TABLE 2
Exemplary Batch Formula for a vial of AAV2-Construct Drug Product

| Component | Quantity | Reference to Standard |
|---|---|---|
| AAV2- | 1.25 × 10\hat{ }14 DRP | In-house, GMP |
| Construct | | |
| Tris, pH 8.0 | 20 mM | EP, BP, USP, JPC |
| MgCl₂ | 1 mM | EP, BP, USP, JPC, FCC |
| (anhydrous) | | |
| NaCl | 200 mM | EP, BP, USP, JP |
| Poloxamer 188 | 0.001% | EP, USP |
| Water For | QS to 125 mL | EP, USP |
| Injections | | |

In some embodiments of the methods of the disclosure for preparation of the Drug Product, a Drug Substance is thawed at +35±2° C., and diluted as required in sterile formulation buffer to the target concentration (1.0×10\hat{ }12 DRP/mL).

In some embodiments of the compositions of the disclosure, the target final DRP titre of the AAV2-Construct Drug Product is 1×10\hat{ }12 DRP/mL, the minimum and maximum acceptable titre is 0.8×10\hat{ }12 DRP/mL and 1.5×10\hat{ }12 DRP/mL, respectively. The AAV2-Construct Drug Product is sterile filtered and filled into 3 mL 2R Type I glass vials with bromobutyl rubber stoppers at volumes of 0.3 mL per vial. The vials are then frozen and stored at ≤−60° C. For labelling and storage prior to QP release and distribution to site, the Drug Product is transferred to the qualified clinical distributor. The Drug Product is stored at ≤−60° C. in a temperature monitored freezer until QP release and distribution.

### Storage of Compositions

In some embodiments of the compositions of the disclosure, including those wherein the composition comprises a Drug Product and the composition is supplied in a sterile vial, the composition may be stored at below zero (° C.). In some embodiments, the compositions may be thawed and frozen without loss of efficacy of the Drug Product or integrity to the sterile packaging. In some embodiments, the compositions may undergo multiple rounds of thawing and freezing without loss of efficacy of the Drug Product or integrity to the sterile packaging.

In some embodiments of the compositions of the disclosure, including those wherein the composition comprises a Drug Product and the composition is supplied in a sterile vial, the composition may be stored at room temperature.

### Organic Materials

Starting materials used in the preparation of buffers and media of the disclosure are certified as free from material of animal origin.

### Compendial Materials: See Table 3.

TABLE 3

| | Exemplary | |
|---|---|---|
| | Supplier/Catalog | |
| Component | Number | Grade |
| Hydrochloric Acid, Concentrated | Merck/1370072500 | NF |
| Magnesium Chloride, Hexahydrate | Merck/1058321000 | USP |
| Potassium Chloride | Merck/1049355000 | USP |
| Sodium Chloride | Merck/1162245000 | USP |
| Sodium Hydroxide, Pellets | Merck/1064821000 | NF |
| Tris Base | Merck/1083861000 | USP |
| Ethanol, absolute | Merck/1009862500 | USP |
| Water for Injection | | EP, USP |
| Citric acid, monohydrate | Merck/137003 | EP, USP |
| H3PO4 | Merck/100563 | EP, USP |
| Na2HPO4•2H2O | Merck/106580 | EP, USP |
| Poloxamer 188 | Merck/137065 | EP, USP |

NF = National Forumulatory;
USP = US Pharmacopeia;
EP = European Pharmacopeia

### Non-Compendial Materials: see Table 4.

TABLE 4

| | Exemplary | |
|---|---|---|
| | Supplier/Catalog | Manufacturers |
| Component | Number | Testing/Specification |
| FBS IRR | Hyclone/ | Certificate and CEP |
| (30-45 kGy) | SH30406.03IR | |
| FBS IRR | Hyclone/ | Certificate and CEP |
| (30-45 kGy) | SH30406.02IR | |
| FBS, irradiated | Life technologies/ | Certificate and CEP |
| | 10101 | |
| TrypLE TM Select | Gibco/12563-029 | pH 7.0-7.4, Identity: |
| | | HPLC |
| PBS w/o Ca | Gibco/10010-031 | pH 7.3-7.5 |
| & Mg | | |
| HBSS w/o Ca | Gibco/14170-138 | pH 6.7-7.8, Sterile |
| & Mg w/o RP | | |
| Benzonase | Merck/1060950001 | Purity ≥99.0%, |
| | | Endotoxin <0.25 EU/ |
| | | 1000 U, Identity: Pass |
| DMEM glutamax | Gibco/61965-026 | pH 6.8-7.2. Sterile |
| BTL | | |
| DMEM glutamax | Gibco/10566-016 | Sterile |
| PCH | | |
| PEI pro | PolyPlus/ | Sterile, Endotoxin |
| | PPLU115-375 | <0.5 EU/mL |
| pAAV.Construct-Kan | custom made | Certificate of Analysis |
| plasmid DNA | | |
| pNLRep-Cap2-Kan | custom made | Certificate of Analysis |
| plasmid DNA | | |
| pHELP-Kan | custom made | Certificate of Analysis |
| plasmid DNA | | |
| HEK293 Master | Cobra Biologies | |
| Cell Bank | | |
| Clarification filter, | Sartorius/ | Sterilized at Novasep |
| 0.65 μm | 5551305P1-- | before use |
| | SS | |
| Clarification filter, | Sartorius/ | Sterile - autoclaved, |
| 0.2 μm | 5445307H9-- | Endotoxins <0.18 |
| | FF—A | EU/mL, Meets USP |
| | | class VI plastics |
| UFDF filter, | Spectrum labs/N02- | Sanitized before use, |
| 100 kDa | E100-05-N or equivalent | LAL test performed |
| | lower effective filtration | after equilibration |
| | area (EFA) filter | |
| Ultracentrifuge | Beckman/342414 | Complies with the FDA |
| tubes | | Code of Federal |
| | | Regulations, Title 21 |
| | | §177.1520 (c) 3.1a & |
| | | 3.2a and the U.S. |
| | | Pharmacopeia criteria |
| | | for Class VI plastics |
| Sterile filtration | Sartorius/5445307HS-- | Endotoxins <0.18 |
| filter. 0.2 μm | FF | EU/mL, Meets USP |
| | or equivalent lower | class VI plastics |
| | EFA filter | |
| Ion exchange | Biorad/156-0101 | Sanitized with 0.5M |
| medium | | NaOH, LAL test |
| | | performed after |
| | | equilibration |
| Column | Merck Millipore/ | Sanitized with 0.5M |
| | 96100250 | NaOH, Endotoxin on |
| | | the final rinse with |
| | | WFI <0.25 EU/mL). |
| Column | Merck Vantage-L | Sanitized with 0.17M |
| | VL22×250 column | H3PO4, 1M NaCl and |
| | | conditioned before use. |
| | | pH and conductivity |
| | | tested after |
| | | equilibration |
| UFDF/Dialysis | Spectrum labs/S02- | Sanitized before use, |
| Filter, 100 kDa | E100-05-N or equivalent | LAL test performed |
| | lower EFA filter | after equilibration |
| UnoQ resin | Bio-Rad | Sanitized with 0.5M |
| | | NaOH, Endotoxin on |
| | | the final rinse with |
| | | WFI <0.25 EU/mL) |
| AVB Sepharose | GE Healthcare | Sanitized with 0.17M |
| resin | | H3PO4, 1M NaCl and |
| | | conditioned before use. |
| | | pH and conductivity |
| | | tested after |
| | | equilibration |

CEP = Certificate of Suitability

### Raw Materials of Animal Origin

Foetal bovine sera are of animal origin. Source, manufacturer and usage of these raw materials is summarized in Table 5.

TABLE 5

| Component | Source | Manufacturer | Use | CEP | Source Documentation |
|---|---|---|---|---|---|
| Foetal Bovine | Bovine | HyClone | HEK293 cell | R1-CEP | Certificate and CEP, |
| Serum, irradiated | | New Zealand | culture, | 2000-211-Rev | New Zealand sourced |
| | | | clinical lot | 01 | |
| | | | manufacture | | |
| Foetal Bovine | Bovine | Life Technologies | HEK293 | R1-CEP | Certificate and CEP, |
| Serum, irradiated | | | MCB | 2001-024-Rev | Australian sourced |
| | | | production | 02 | |

### Filters and Chromatographic Matrices

Filters used for the filtration of the Drug Substance and Drug Product are Sartopore 0.45 μm and 0.2 μm filters. The filters are non-sterile when purchased and are sterilized in house at the contract manufacturer by autoclaving. They are integrity tested by bubble point testing at 3.2 Bars.

All chromatographic materials are released on a Certificate of Analysis prior to use. Columns are purchased prepacked and are sanitized prior to use.

### Methods of Manufacture of AAV2-Construct Drug Product

### “Original Process” (Process 1)

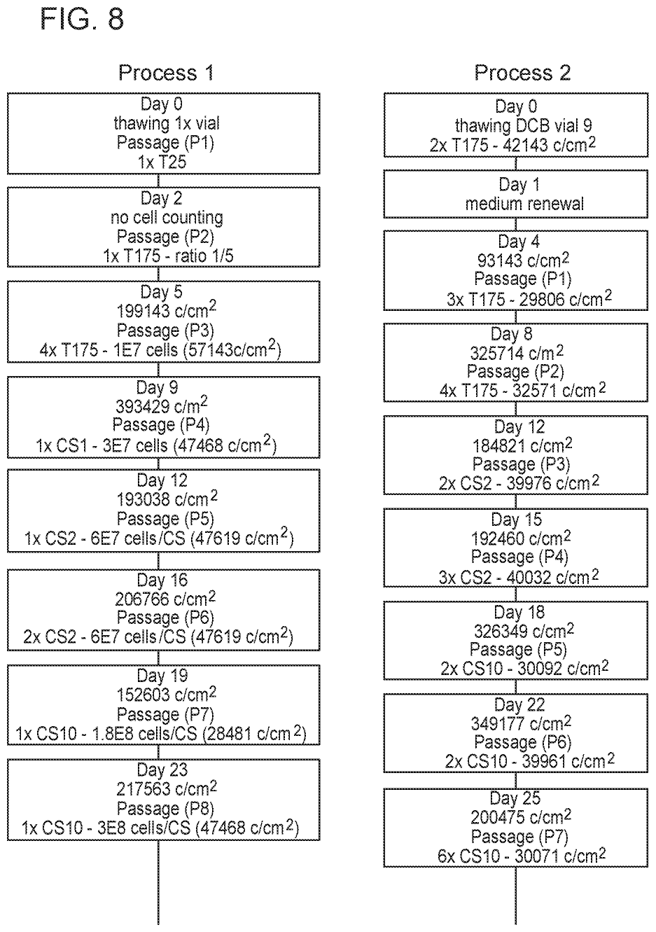

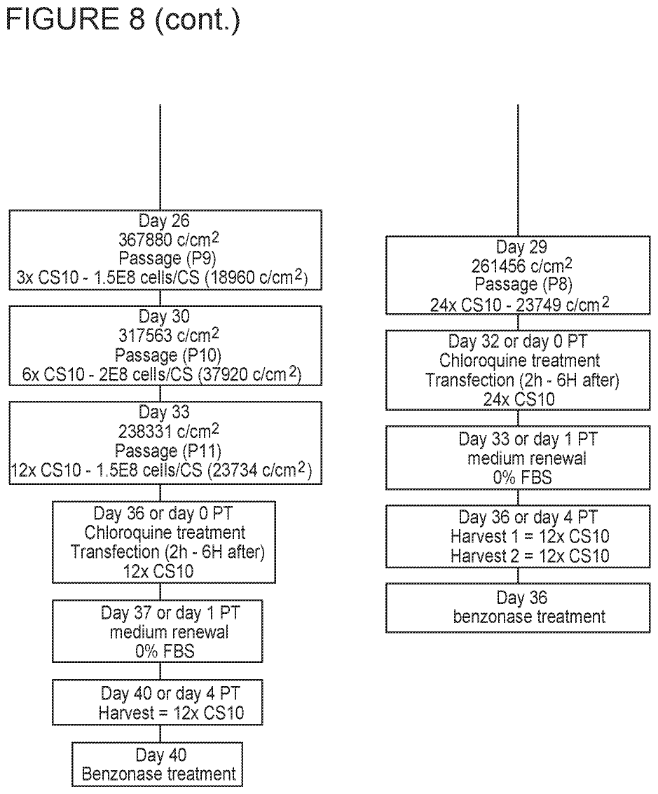

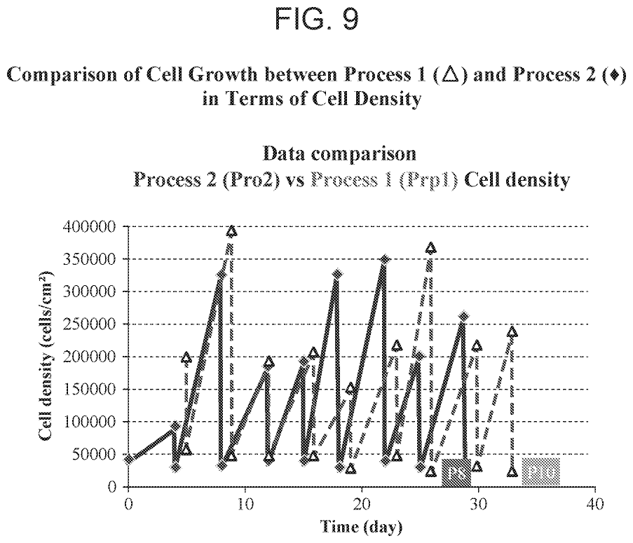

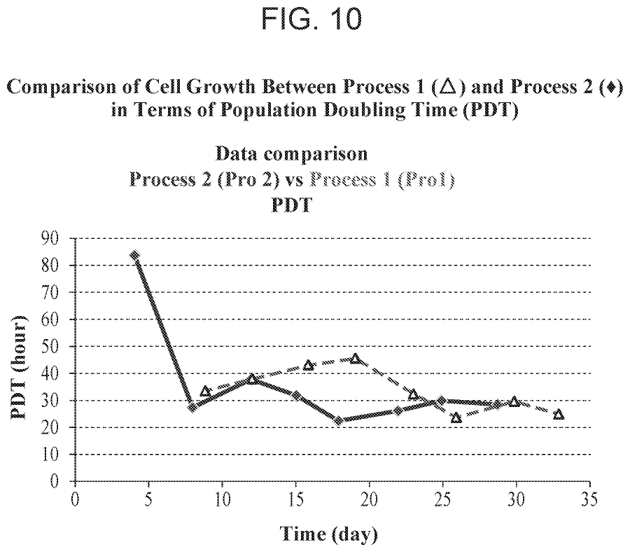

An overview of an exemplary manufacturing process for AAV2-Construct Drug Substance is illustrated in FIG. 8. Flow diagrams corresponding to this process are provided in FIGS. 9 and 10. The manufacturing process described in FIGS. 8, 9 and 10 may be referred to herein as “Process 1.”

Exemplary changes to Process 1 are provided in Table 6 below. Some embodiments of Process 2 may include the changes described in Table 6.

TABLE 6

| Process Step/Materials | Process 1 | Process 2 | Notes |
|---|---|---|---|
| Grade of Manufacture | GMP clean rooms | Non-GMP | |
| | | laboratories | |
| HEK293 Cells | Original Process 1 | ATCC CRL-1573 | To assess changes to source of |
| | | development cell | cells |
| | | bank | |
| Plasmids | Ampicillin | Kanamycin plasmids | To assess changes to the plasmid |
| | plasmids | | backbone (switch from AmpR to |
| | | | KanR gene) |
| Raw Materials | GMP grade | Research grade | |
| Timing from Re-feed | 22-24 h | 72-73 h | Harvesting up to 96 h post |
| (following plasmid | | | transduction does not adversely |
| transduction) to Harvest | | | affect yields of cells + media |
| | | | compared to harvesting earlier |

### Optimization of Process 1 (Upstream Process)

The starting material for Process 1 was initiated from a single vial of HEK293 cells from a development cell bank (DCB). This DCB was produced using a single vial of ATCC HEK293 cells. CS10 were used to culture the HEK293 cells but the scale of this culture was increased from 12 CS10 to 24 CS10 to generate additional material for subsequent process development that was being performed concurrently. The procedure proceeded with 2×12 CS10 up until the point of transduction, at which time, the 24 CS10 were treated within the same operation.

Cell Culture Vessels and Raw Materials: Table 7 highlights key differences between the raw materials used for two different manufacturing processes and provides notes regarding any significance of the change.

TABLE 7

| | Development of New | | |
|---|---|---|---|
| | Process | | |
| | (incorporating changes | Original Process | |
| | to Process 1 and | (corresponding to | |
| Raw Material | Process 2) | Process 1) | Notes |
| Serum | FBS | Cosmic Calf serum | Cosmic calf serum not suitable |
| | Hyclone- | Hyclone-SH30087- | for late clinical phase |
| | SH30406.03IR | 04IR | manufacture. Change in serum |
| | | | could impact cell growth/ |
| | | | impurity profile but needs to be |
| | | | changed for GMP manufacture. |
| | | | FBS used routinely in the |
| | | | improved process. |
| Cell | HyQtase | Lonza-Tiypzean | Clinical batch used HyQtase |
| Dissociation | Hyclone-ref: | | (original process). |
| Reagent | SV30030.01 | | |
| Medium | L-glutamine 200 mM | SG-200 Stable | Preferred supplier for improved |
| Supplement | Life technology-ref: | dipeptide of L-Alanyl | process. |
| | 25030 | L-Glutamine | |
| | | (200 mM) | |
| | | Hyclone-ref: SH30590 | |
| Chloroquine | Sigma Aldrich- | Spectrum chemical- | Preferred supplier for improved |
| | ref: C6628 | C1448 | process. |
| Dextrose | Roquette-ref: 361409 | Sigma Aldrich-ref: | Preferred supplier for improved |
| | | D9434 | process. |
| Plasmids | pAAV.Construct | pAAV.Construct | All plasmids manufactured are |
| | pHELP (Kan) | pHELP (Kan) | sequenced in their entirety to |
| | | | ensure homology. Although lot #s |
| | pNLRep-Cap2 (Kan) | pNLRep-Cap2 (Kan) | may change, the sequences |
| | | | remain the identical between |
| | | | original process and improved |
| | | | process. |

Cell Culture: The source of cells used for the improved process were from a single vial of the DCB HEK293 cells. As this was a different bank used to produce the batch of the original process, albeit the same original source of ATCC cells, the scale of culture was increased (12 CS10 to 24 CS10) and a different source of serum was used to support cell growth during the cell amplification, this stage was adapted accordingly. The pre-culture cell growth of the improved process included 8 passages compared to 10 passages for the batch produced by the original process. In addition, cell densities post passage were variable according to the original process but fixed according to the improved process. FIG. 8 illustrates a comparison between both sites for the cell culture phase of the process.

Cell growth was assessed between the original and improved processes using cell density measurements. Results indicated that the cells produced by the improved processes grew faster. This could be potentially attributed to a different source of serum and/or the cell passaging regime based on fixed seeding densities of the improved processes. For both processes, the population doubling time was shown to be 20-30 hr once cell growth had stabilized (FIG. 10).

In some embodiments of Process 2, including those utilizing the raw materials listed in Table 7 (with the exception of the serum), the cell culture and/or cell growth media comprises or consists of a serum-free media.

Transduction: Transduction as part of the improved process was performed using the same method as the original process.

Cell Harvest: According to the improved process, the harvest was split into 2 harvests of 12 CS10 to be representative of the original process (1×12 CS10). Following harvest, the cells were disrupted using a PANDA device and the titre of the vector determined. The PANDA device used during the improved process was a different manufacturer compared to the cell disrupter used during the original process (see Table 8 below), but has the capability to perform cell lysis at the same pressure as the cell disrupter of the original process (80 bar).

TABLE 8
Comparison of Cell Disrupter Equipment Used in original
versus improved processes.

| Process | Cell Disrupter Equipment Used |
|---|---|
| Original Process | Microfluidics International HC-8000 Microfluidizer |
| | with 200 μm ceramic Z channel module |
| Improved Process | PANDA 2K homogenizer GEA/NiroSoavi |

Vector Titre Post Upstream Processing: The titre of the upstream process vector material from 1×12 CS10 (6.4×10¹⁵ DRP/batch) (according to improved process) was comparable to that generated from the same scale of upstream process (1.44×10¹⁵ DRP/batch) (according to the original process).

### Optimization of Process 1 (Downstream Process)

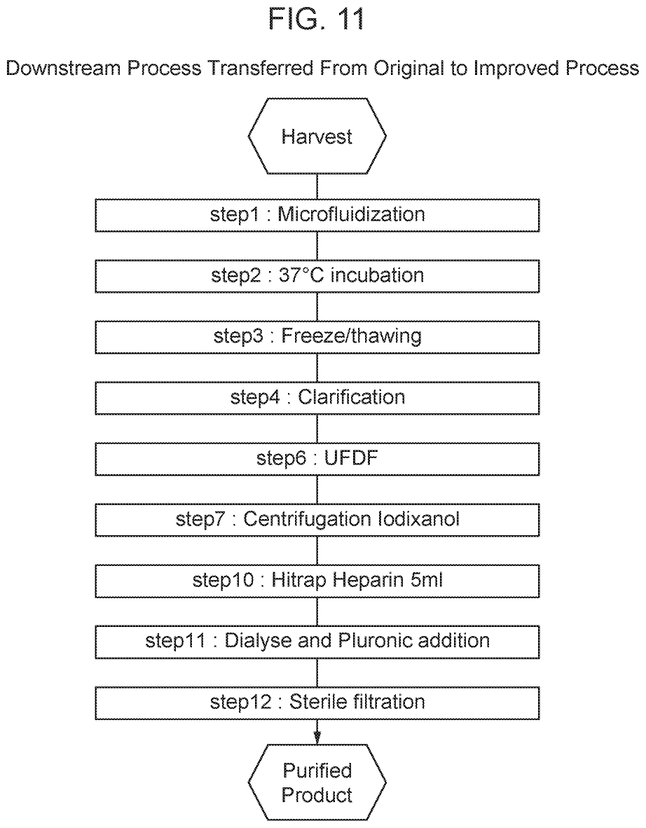

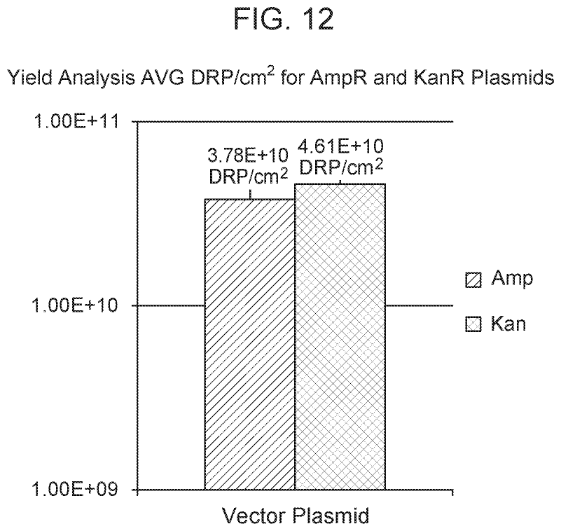

The downstream process that was transferred from the original to the improved process is illustrated in FIG. 11.

Experimental and Raw Materials: Where possible, the same equipment and raw materials and suppliers were selected for use in the downstream “improved” process as those used in the original process.

Downstream Performance: Observations at individual process stages are summarized below.

TABLE 9
Cell Disruption/Lysis

| | Original Process | Improved Process | Notes |
|---|---|---|---|
| Inlet Product Appearance | Red; particles | Red; particles | No Significant Change |
| Duration (min) | 60 | 65 | |
| Outlet Product Appearance | Red; particles | Red; particles | |

TABLE 10
Freeze/Thaw Step

| | Original Process | Improved Process | Notes |
|---|---|---|---|
| Inlet Product Appearance | Red & Particles | Red & Particles | No Significant Change |
| Incubation | 37° C. | 37° C. | |
| Duration | 19 | 19 | |
| Freezing | −20° C. | −20° C. | |
| Thawing Duration | 4 h | 4.5 h | |
| Outlet Product Appearance | Red & Particles | Red & Particles | |

TABLE 11
Clarification

| | Original Process | Improved Process | Notes |
|---|---|---|---|
| Inlet Product Appearance | Red & particles | Red & particles | No Significant Change |
| Flow Rate (mL/min) | 750 | 750 | No Significant Change |
| Duration (min) | 20 | 2 | |
| Maximum Inlet Pressure | 0.9 | 1.7 | The acceptance |
| (psi) | | | criterion was pressure |
| | | | below 10 psi. |
| Outlet Product Appearance | Red | Red | No Significant Change |

TABLE 12
Concentration and Diafiltration

| | Original Process | Improved Process | Notes |
|---|---|---|---|
| Inlet Product Appearance | Red | Red | No Significant Change |
| % (v/v) Volume of SSS | 10 | 10 | |
| Addition | | | |
| Recirculation Flow Rate | 2.76 | 2.76 | |
| (L/min) | | | |
| Duration | 1 h 30 min | 1 h 25 min | |
| Diafiltration Rate | 4 | 4 | |
| TMP (psi) | 6-7 | 5.4-6.0 | A range from 3 to 6 psi |
| | | | for TMP was also used |
| | | | Process 2 and may be |
| | | | used in a |
| | | | manufacturing process |
| | | | of the disclosure |
| Outlet Product Appearance | Pink | Pink Cloudy | No Significant Change |
| Volume (mL) | 230 | 220 | No Significant Change |

Heparin Dialysis, Diafiltration and Filtration Steps: The product was limpid and colorless from the Iodixanol gradient step to the final product. No unexpected observations were evident during these steps.

### “Improved Process” (Process 2)

A batch of product is prepared from a single production campaign consisting of 24 Corning 10-stack vessels containing plasmid DNA transfected HEK293 cells that produce the AAV2-Construct biologic product. The cell mass and cell culture media is harvested and pooled from the 24 Corning 10-stack vessels and followed by a single purification process. A batch of product prepared from a single campaign is expected to produce ≥5×10¹³ viral particles (vp; also referred to herein as vector particles) of Drug Substance.

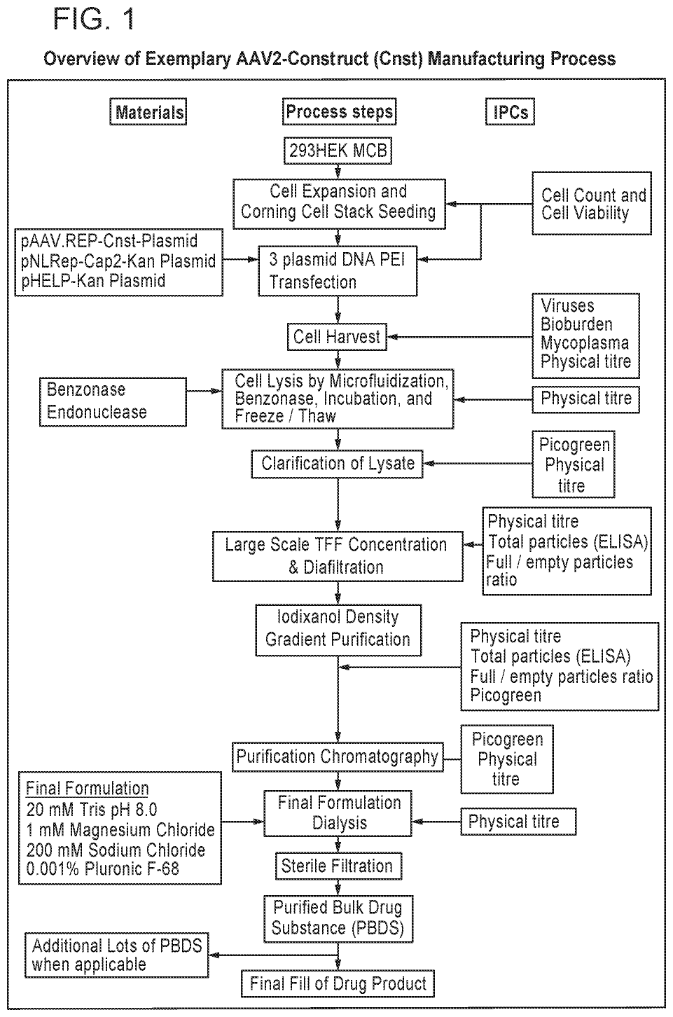

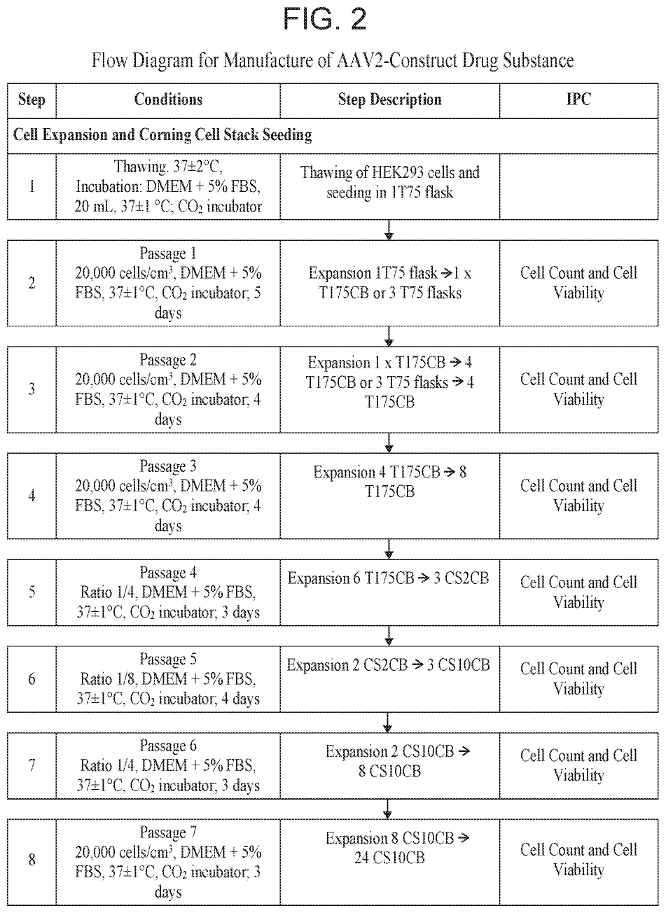

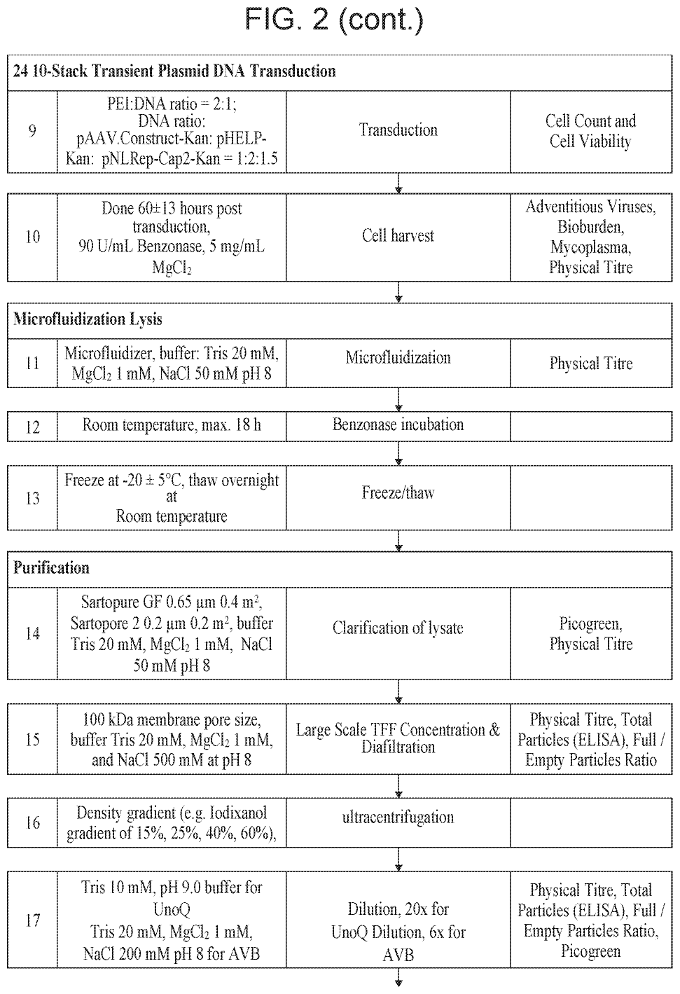

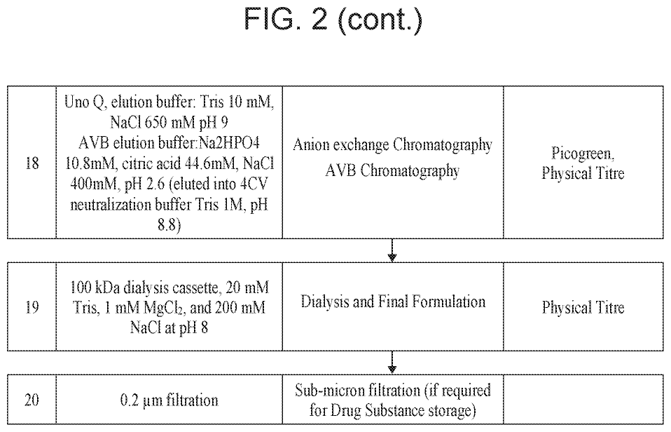

An overview of an exemplary manufacturing process for AAV2-Construct Drug Substance is illustrated in FIG. 1. The flow diagram corresponding to this process is provided in FIG. 2. The manufacturing process described in this example may be referred to herein as “Process 2.”

In some embodiments of Process 2, the cell culture media harvested and pooled from the 24 Corning 10-stack vessels comprises a serum free media. In some embodiments of Process 2, the cell culture media harvested and pooled from the 24 Corning 10-stack vessels consists of a serum-free media.

### Cell Expansion and Corning Cell Stack Seeding

See FIG. 2 for a schematic diagram corresponding to the detailed description of an exemplary manufacturing process provided below.

Step 1: Thawing and seeding of cells used to produce AAV2-Construct Drug Substance. Temperatures, durations, spin speed, and volumes below may be adjusted for optimal results depending upon the cell type used. For the exemplary embodiment described below, conditions were optimized for the use of HEK293 Cells.

Thawing of HEK293 Cells and Seeding into 1 T75 flask: A vial of the HEK293 MCB is thawed at 37±2° C. Cells are transferred in a 15 mL tube containing 9 mL of cold growth medium (DMEM media with 5% FBS). The vial is rinsed with 1 mL of growth medium. The suspension is centrifuged at 4° C. for 5 minutes at 300 g, the supernatant is discarded and cells are suspended in 10 mL of pre-warmed growth medium. Cell density is determined and the cell suspension is transferred to a T75 cm² flask containing 10.5 mL of growth medium to obtain a final volume of 20 mL. The T75 cm² flask is transferred to an incubator at 37±1° C. under humidified 4-6% CO₂ atmosphere. 24±4 hours after thawing, the growth medium is replaced by 20 mL of pre-warmed growth medium.

In some embodiments of the steps of thawing of HEK293 Cells and seeding into 1 T75 flask, cells are transferred in a 15 mL tube containing 9 mL of cold growth medium. In some embodiments, the growth medium comprises or consists of glycine, L-Arginine hydrochloride, L-Cysteine dihydrochloride, L-Glutamine, L-Histidine hydrochloride-H₂O, L-Isoleucine, L-Leucine, L-Lysine hydrochloride, L-Methionine, L-Phenylalanine, L-Serine, L-Threonine, L-Tryptophan, L-Tyrosine disodium salt dehydrate, L-Valine, Choline chloride, D-Calcium pantothenate, Folic Acid, Niacinamide, Pyridoxine hydrochloride, Riboflavin, Thiamine hydrochloride, i-Inositol, Calcium Chloride (CaCl₂)) (anhyd.), Ferric Nitrate (Fe(NO₃)₃″9H2O), Magnesium Sulfate (MgSO₄) (anhyd.), Potassium Chloride (KCl), Sodium Bicarbonate (NaHCO₃), Sodium Chloride (NaCl), Sodium Phosphate monobasic (NaH₂PO4-H₂O), and D-Glucose (Dextrose). In some embodiments, the growth medium comprises or consists of a serum-free media. In some embodiments, the growth medium comprises or consists of a clarified media.

Step 2: Expansion of cells used to produce AAV2-Construct Drug Substance. Temperatures, durations, and volumes below may be adjusted for optimal results depending upon the cell type used. For the exemplary embodiment described below, conditions were optimized for the use of HEK293 Cells.

Expansion 1 T75 Flask to 1×T175CB or 3 T75 Flasks: Media is discarded and cells washed with pre-warmed PBS. The cells are loosened with TrypLE cell dissociation reagent. The T-flasks or Cell Stacks are incubated 5 to 10 minutes in an incubator set at 37±1° C. and the cells are fully dislodged by gently tapping the vessel. Growth medium is added to inhibit the TrypLE. The volumes of growth medium, PBS and TrypLE used for different supports are presented in Table 14. All cell suspensions are then pooled.

TABLE 14
Volume of Media and Solutions

| | | | | Inhibition | Working |
|---|---|---|---|---|---|
| | | | | (Growth | Culture |
| Supports | Surface | PBS | TrypLE | Medium) | Volume |
| T75 | 75 cm² | 5 mL | 2 mL | 6 mL | 20 mL |
| T175CB | 175 cm² | 10 mL | 4 mL | 12 mL | 50 mL |
| CS2 CB | 1260 cm² | 100 mL | 40 mL | 120 mL | 400 mL |
| CS10 CB | 6320 cm² | 400 mL | 140 mL | 420 mL | 2 L |

Cell count and cell viability is determined and the cells are seeded, incubated and passaged in accordance with Table 15.

TABLE 15
Process Parameters for Passages

| Pa Passage | | | Seeding Density/Ratio | | Incubation Time |
|---|---|---|---|---|---|
| P1 | 1T75 | → xT175CB | 20000 | cells/cm² | 5 days |
| | Or 1T75 | → 3T75 | | | |
| P2 | xT1T75 | → 4T175CB | 20000 | cells/cm² | 4 days |
| | Or 3T75 | → 4T175CB | | | |
| P3 | 4T175CB | → 8T175CB | 20000 | cells/cm² | 4 days |
| P4 | 6T175CB | → 3CS2CB | Ration | 1/4 | 3 days |
| P5 | 2CS2CB | → 3CS10CB | Ratio | 1/8 | 4 days |
| P6 | 2CS10CB | → 8CS10CB | Ratio | 1/4 | 3 days |
| P7 | 8CS10CB | → 24CS10CB | 20000 | cells/cm² | 3 days |

In Process Controls (IPCs): Cell Count and Cell Viability

Step 3: Expansion 1 T175CB to 4 T175CB or 3 T75 Flasks to 4 T175CB: See Step 2.

IPCs: Cell Count and Cell Viability.

Step 4: Expansion 4 T175CB to 8 T175CB: See Step 2.

IPCs: Cell Count and Cell Viability.

Step 5: Expansion 6 T175CB to 3 CS2CB: See Step 2.

IPCs: Cell Count and Cell Viability.

Step 6: Expansion 2 CS2CB to 3 CS10CB: See Step 2.

IPCs: Cell Count and Cell Viability.

Step 7: Expansion 2 CS10CB to 8 CS10CB: See Step 2.

IPCs: Cell Count and Cell Viability.

Step 8: Expansion 8 CS to 24 CS10CB. See step 2.

IPCs: Cell Count and Cell Viability

### Plasmid DNA Transfection

Step 9: Transfection of cells used to produce AAV2-Construct Drug Substance. Temperatures, durations, spin speed, and volumes below may be adjusted for optimal results depending upon the cell type used. For the exemplary embodiment described below, conditions were optimized for the use of HEK293 Cells.

The HEK293 cells are transfected with three plasmids using Polyethylenimine (PEI).

The three plasmids (pAAV.Construct-Kan, pHELP-Kan, and pNLRep-Cap2-Kan) are added in a sequential way with a specific ratio.

TABLE 16
Transfection Conditions
Transduction Conditions

| Working Volume | | 2125 mL/CS10 CB |
|---|---|---|
| DNA + | Growth Medium (Solution 1) | 40 mL/CS10 CB |

| Total DNA Quantity | 1.02 mg/CS10 CB | |
|---|---|---|
| DNA Ratio | pAAV.Construct-Kan | 1 |
| | pHELP-Kan | 2 |
| | pNLRep-Cap2-Kan | 1.5 |

| | | |
|---|---|---|
| PEI + | Growth Medium (Solution 2) | 40 mL/CS10 CB |

| | |
|---|---|
| PEI: DNA Ration | 2:1 |

The plasmids and the PEI are prepared with growth medium.

The required amount of plasmid DNA is presented in Table 17.

TABLE 17
Plasmid DNA Amounts

| | | Quantity (mg) for | Total Quantity (mg) for |
|---|---|---|---|
| Plasmid | Ratio | 1CB10 CB | 24CS10 CB |
| pAAV.Construct-Kan | 1 | 0.427 mg | 10.248 mg |
| pHELP-Kan | 2 | 0.854 mg | 20.496 mg |
| pNLRep-Cap-Kan | 1.5 | 0.640 mg | 15.360 mg |

The required amount of PEI mix is transferred in the DNA mix to form the PEI-DNA complex, shake for 10 seconds and incubate at room temperature. The PEI-DNA complex is transferred into 8.2 L growth medium. The mixture of growth medium and PEI-DNA complex is homogenized, the PEI-DNA complex bottle is rinsed and the mixture of the growth medium and the PEI-DNA complex is homogenized again. The medium is drained from the CS10CB and 2 L of growth medium/PEI-DNA complex mixture is transferred to the CS10CB. The transfected CS10CB are transferred into an incubator set at 37±1° C. under humidified 4-6% CO₂ atmosphere.

The transfection mixture is left on the cells for 24±1 hours and then the media is replaced with DMEM Glutamax.

IPCs: Cell Count and Cell Viability.

Step 10: Harvest of cells used to produce AAV2-Construct Drug Substance. Temperatures, durations, spin speed, and volumes below may be adjusted for optimal results depending upon the cell type used. For the exemplary embodiment described below, conditions were optimized for the use of HEK293 Cells.

The harvest is done 60±13 hours post transfection.

The product is harvested by the following steps:

First half of medium is transferred to the harvest bag.

The CS10CB are shaken and the remaining medium and cells are harvested.

200 mL of HBSS-EDTA are transferred to the CS10CB and incubated at room temperature for 10 minutes.

At the end of the incubation time, the buffer is drained to the harvest bag.

This harvesting process is repeated with all vessels.

After the harvest, MgCl₂ 1M is added to a final concentration of 5 mM and benzonase is added to a final concentration of 90 U/mL.

IPC: Adventitious Viruses, Bioburden, Mycoplasma, Physical titre.

Step 11: Microfluidization Lysis.

The PANDA disrupter is equilibrated with minimum 3 L of Tris 20 mM, MgCl₂ 1 mM, NaCl 50 mM pH 8 buffer. The bag containing the harvest treated with Benzonase is connected to the lysis assembly. The product is loaded on to the PANDA system at a pressure of 80±10 bars and collected in a new bag. After loading the product, the system is rinsed with 2×200 mL of Tris 20 mM, MgCl₂ 1 mM, NaCl 50 mM pH 8 buffer. Both rinses are pooled with the lysed product.

IPC: Physical Titre

Step 12: Benzonase Incubation:

The microfluidized cells are incubated overnight at room temperature for a maximal duration of 18 hours for benzonase endonuclease activity.

Step 13: Freeze/Thaw:

The final lysis step is a freeze at −15 to −25° C. followed by a thaw overnight at room temperature to promote aggregation of cellular debris to facilitate the clarification step.

### Purification

Step 14: Clarification of Microfluidized Lysate.

The filtration process utilizes a pre-filter (Sartopure GF 0.65 μm 0.4 m²) followed by a filter for bioburden reduction (Sartopore 2, 0.2 μm, 0.2 m²) and Tris 20 mM, MgCl₂ 1 mM, NaCl 50 mM pH 8 buffer for flushing.

IPCs: Picogreen, Physical Titre

Step 15: Large Scale TFF Concentration and Diafiltration.

This process achieves volume reduction and an initial purification using the principle of Tangential Flow Filtration (TFF). The volume reduction can be as large as a 100 fold. Salt and surfactant solution (SSS, 4.14 M NaCl+nonionic surfactant) is added to the clarified lysate with 1/10 of the lysate weight. The purification is achieved by diafiltration (100 kDa) of the concentrate with 20 mM Tris pH 8.0, 1 mM MgCl2, 500 mM NaCl, 0.1% nonionic surfactant to “wash” smaller molecular weight impurities from the sample. This part is required in order to achieve high levels of concentration without protein/AAV particle precipitation.

IPCs: Physical Titre, Total Particles (ELISA), Full/Empty Particles Ratio.

Step 16: Iodixanol Purification.

This process is designed to purify rAAV using a four layer discontinuous iodixanol gradient (15%, 25%, 40%, 57%). This orthogonal purification step greatly enriches the preparation for DNA containing rAAV particles, while removing the bulk of rAAV particles that are devoid of DNA (empty particles) based on the differential buoyant density of these particles in the iodixanol gradient medium following ultracentrifugation. The density of DNA containing rAAV results in the migration of rAAV into the 40% iodixanol layer, while the majority of contaminating cellular proteins migrate to the 25%/40% interface. Aggregation of rAAV with proteins and DNA can alter the particles buoyant density, so inclusion of 1M NaCl in the 15% iodixanol layer minimizes this aggregation.

Separation is achieved by a discontinuous gradient of iodixanol (Optiprep 57% solution) that is subjected to high g forces in an ultracentrifuge. The 40% fraction is harvested until reaching the impurity band (˜3 mL).

The collected fractions are pooled and stored at +2/+8° C. until subsequent purification steps.

Step 17: Dilution:

The fraction obtained at the end of the purification step is diluted 20 times with Tris 10 mM, pH 9.0 buffer for purification of GMP lot G214/REP1/FC001 by anion exchange chromatography. For purification by AVB affinity, the pooled fraction at the end of the Purification (density gradient) step is diluted 6 fold with 20 mM Tris, 1 mM MgCl₂, and 200 mM NaCl at pH 8 buffer.

IPCs: Physical Titre, Total Particles (ELISA), Full/Empty Particles Ratio, Picogreen

Step 18: Anion Exchange Chromatography.

The Anion exchange chromatography is performed at room temperature with a Unosphere Q (UnoQ) media (Biorad) packed at a flow rate of 14 mL/min with Tris 10 mM, NaCl 200 mM pH 9 in a Vantage VL 11×250 column (packed volume ˜9.5 mL). The column is conditioned first with Tris 10 mM, NaCl 1 M pH 9 buffer and second with Tris 10 mM, pH 9 buffer to obtain a pH equal to pH 9.0±0.2 and a conductivity equal to the Tris 10 mM, pH 9.0 Conductivity ±10%.

The diluted product is filtered through a Sartoscale 0.2 μm 17 cm² filter.

It is loaded on the column and the column is rinsed with equilibration buffer. After a second wash with Tris 10 mM, NaCl 45 mM pH 9 buffer, the product is eluted with minimum 30 column volumes of Tris 10 mM, NaCl 650 mM pH 9 buffer.

The eluted product is stored at +2/+8° C. until the next step.

IPCs: Picogreen, Physical Titre

Step 18 with AVB Sepharose Affinity Chromatography.

The affinity chromatography is performed at room temperature with AVB Sepharose HP (GE Healthcare) packed in a Vantage-L VL22×250 column (8±1 cm bed height) packed and flushed in Tris 20 mM, MgCl₂ 1 mM, NaCl 200 mM pH 8 buffer and then is sanitized with H₃PO₄ 0.17M, NaCl 1 M for 30 minutes. Following sanitization, the column is equilibrated in Tris 20 mM, MgCl₂ 1 mM, NaCl 200 mM pH 8 buffer prior to loading product (pooled fractions from step 16) diluted in Tris 20 mM, MgCl₂ 1 mM, NaCl 200 mM pH 8. The diluted product is filtered through a Sartoscale 0.2 μm 17 cm² filter prior to column loading. Once loaded, the column is washed with approximately 35 column volumes of Tris 20 mM, MgCl₂ 1 mM, NaCl 200 mM pH 8 buffer and then eluted with approximately 20 column volumes of Na₂HPO₄ 10.8 mM, citric acid 44.6 mM, NaCl 400 mM pH 2.6 buffer. Immediately following elution, the eluate is neutralized with approximately 4 column volumes of Tris 1 M pH8 buffer.

The eluted product is stored at +2/+8° C. until the next step.

IPCs: Picogreen, Physical Titre

Step 19: Dialysis and Final Formulation.

This process utilizes dialysis to formulate the AAV product into the desired Final Formulation buffer (20 mM Tris, pH 8, 1 mM MgCl₂, 200 mM NaCl, optionally with poloxamer at 0.001%).

Tangential flow filtration of the Elution product is conducted using a hollow fiber cartridge with a molecular weight cut-off of 100 kDa (Spectrum). The cartridge and the system are equilibrated with Tris 20 mM, MgCl₂ 1 mM, NaCl 200 mM pH 8 buffer to obtain a pH8.0±0.2 on the Permeate side.

The product is concentrated to the minimum volume before the diafiltration in continuous mode against minimum 6 volumes of Formulation Buffer. The retentate is collected. The system is rinsed with Formulation Buffer. This rinse is collected in a different vessel.

IPCs: Physical Titre.

Step 20: Submicron Filtration of Purified Bulk Drug Substance.

If required for longer term storage (>60 days) the product is submicron filtered using a 0.2 μm filter. Once the Drug Substance is completely filtered, the filter is rinsed with the final formulation buffer.

After QC sampling, the purified bulk Drug Substance is stored at <−60° C.

Method of Making a Drug Product from a Drug Substance

The disclosure provides several exemplary processes/methods of making a Drug Product from a Drug Substance (as a starting material). Two of these exemplary methods are Process 1 (the original process) and Process 2 (the improved process). As described herein, Process 2 includes changes and/or improvements made to Process 1.

In both Process 1 and Process 2, the Drug Substance is thawed, diluted, sterile filtered and filled into the primary packaging containers. However, the improved process (Process 2) introduced the following changes to the original procedure (the in-process controls remain unchanged):

Change in thawing temperature from room temperature to +35±2° C.

Change in the primary packaging from 0.5 mL polypropylene cryovials to 3 mL type I glass vials and bromobutyl rubber stoppers

Change in filling size per single vial from 0.1 mL/vial to 0.3 mL/vial

Compositions of the disclosure may be supplied as liquids. In some embodiments of the compositions of the disclosure, including those wherein the composition comprises a Drug Product, the Drug Product is supplied in sterile glass vials. In some embodiments, the sterile glass vials are sterile clear glass vials. In some embodiments, the sterile glass vials are capped with stoppers. In some embodiments, the stoppers are plastic. In some embodiments, the sterile glass vials are capped and further enclosed with overseals.

### Optimization of Manufacturing Process

Following review of the original manufacturing process (Process 1), opportunities to improve the quality of future clinical and commercial product and/or to make a more robust process were identified and implemented as Process 2. The process development activities undertaken fall into two categories: (1) Process Changes; (2) Process Optimization. A summary of the process development strategy and rationale is provided in Table 18 and each category is discussed in more detail below.

TABLE 18
Summary of Changes to Process 1

| Topic | Intended Change | Rationale |
|---|---|---|
| Process Changes | | |
| HEK293 Master Cell Bank | Source of cells from which MCB is | Improved traceability of cells to |
| | generated | increase reassurance of product |
| | | quality and safety |
| Vector Plasmids | Replacement of ampicillin resistance | Removal of theoretical risk of |
| | gene with kanamycin resistance gene | ampicillin/ampicillin resistance |
| | | gene transfer to patients |
| Heparin Affinity | Replacement of animal-derived matrix | Reduction of biosafety risks |
| Chromatography | with non-animal derived matrix | associated with animal-derived |
| | | materials |
| Final Product Container and | Change from screw-capped, | Improved final Drug Substance |
| Container Closure | polypropylene to stoppered, glass | integrity |
| | vials | |
| Scale Up of Upstream Process | Increase scale of cell culture phase | To increase yield of final product |
| Steps | | |
| HEK293 Cell Transfection | Change from calcium phosphate to | Improved robustness, removal of |
| | PEI | need for chloroquine (endosomal |
| | | escape reagent) |
| Process Optimization | | |
| HEK293 Cell Culture and | Optimization of seeding density and | Improved process control and |
| Expansion | cell culture performance. | reproducibility to ensure process |
| Plasmid Triple Transfection | Development studies to optimise | consistency and robustness |
| and Harvest | transduction | |
| Microfluidization for Cell | Optimisation of operating conditions | |
| Disruption | with equipment (improved process) | |
| Density Gradient | Increase robustness of purification | |
| Ultracentrifugation | process step | |
| Ultrafiltration/Diafiltration | Optimisation of buffer exchange steps | Ensure process consistency and |
| | | robustness, and suitability of |
| | | product contact materials |

MCB = Master Cell Bank

In summary, to generate Process 2, changes were made to the source of cells from which the MCB is generated, the antibiotic selection gene of the three plasmids, the scale of the upstream processing step, the transduction reagent, the affinity chromatography step and the final product container and closure.

HEK293 Master Cell Bank

Process 1: The MCB used to produce the first clinical batch was generated from cells optimized AAV2 production. There is limited traceability of the history of this cell line both in terms of how the cells were optimized and exposure to materials of animal origin.

Change to Process 1: An original vial of HEK293 cells deposited at ATCC (the precursor cell bank from which the Process 1 were generated) was used to initiate manufacture of a new GMP MCB. This MCB, now characterized and tested, is used to support clinical AAV vector manufacture and can be used for commercial manufacture. Full traceability of cell passage number, materials used, methods followed and cell bank characterization studies performed provides increased reassurance of product quality, traceability and safety. A working cell bank was subsequently generated from a single vial of the GMP MCB under conditions of Good Manufacturing Process (GMP) to form a GMP WCB. This WCB, now characterized and tested, can be used for commercial manufacture. Full traceability of cell passage number, materials used, methods followed and cell bank characterization studies performed provides increased reassurance of product quality, traceability and safety.

The ATCC sourced HEK293 cells were assessed for comparability following a defined protocol whereby vector production, as assessed by genomic titre, was directly compared with that from the original Stratagene cells. Vector titre was assessed in triplicate and concurrently from cells at comparable passage limits, including both low and high passage. Cells were also assessed for growth kinetics and viability. In addition cells were treated with a green fluorescent protein (GFP) expression vector to assess transduction efficacy.

The ATCC sourced cells were also assessed as part of a full-scale non-GMP engineering run in combination with the plasmid changes (see below) following the same batch manufacturing protocol as the original clinical batch. This batch was tested against the full panel of in-process control tests, bulk harvest, Drug Substance and Drug Product tests (with the exception of those tests for bioburden, endotoxin, sterility and adventitious agents) and compared against the same test results, which were previously obtained from the clinical batch (original process).

Once suitability of the ATCC HEK293 cells was established, a GMP MCB was prepared and fully tested for adventitious agents, identity, purity and safety. The GMP MCB was QA released for subsequent GMP manufacture.

Vector Plasmids

Process 1: The three plasmid constructs used to produce the AAV2-Construct vector containing a gene encoding ampicillin resistance, used for selection during plasmid production. Although ampicillin, a β-lactam antibiotic, is not used in the manufacturing process of the AAV2-Construct vector, removal of the AmpR is preferable to avoid the potential for any carry-over of residual ampicillin from the plasmid production process to the AAV2-Construct production process and also removes the potential for the gene encoding ampicillin resistance to be present in the final packaged product.

Change to Process 1: Each plasmid was modified to replace the ampicillin resistance gene with a gene encoding kanamycin resistance. The remainder of the plasmid backbone was not changed. During plasmid manufacture, ampicillin used for plasmid selection could be carried over into the AAV2-Construct production process and pose a potential risk for patients with sensitivity to β-lactam antibiotics. In addition, the presence of an ampicillin resistance gene from the plasmid material could end up packaged into the final vector product and theoretically confer or transfer antibiotic resistance to recipients. Although the likelihood of either risk is considered low, it is understood that where possible the use of β-lactams should be avoided. Therefore, the safety profile of the product was improved by replacing the AmpR with a KanR.

The new plasmids were fully sequenced and this sequencing provided confirmation that only the resistance gene had changed.

A comparability study to evaluate AAV2-Construct production using the new plasmids was conducted following a defined protocol. Both AmpR (old) and KanR (new) plasmids were used to transfect HEK293 cells concurrently and vector production compared by assessing genomic titre. Triplicate testing was performed and the comparison of vector titre reported.

Following demonstration of acceptable titre, the modified plasmids were also assessed as part of a full-scale non-GMP production run in combination with the cell changes (detailed above) following the same batch manufacturing protocol as the original clinical batch. The batch was tested against the full panel of bulk harvest, drug substance and drug product tests (with the exception of those tests for bioburden, endotoxin, sterility and adventitious agents) and compared against the same test results obtained from the clinical batch (original process). Once data from this technical run had been generated and considered acceptable, manufacture of plasmid suitable for use in GMP product manufacture was implemented.

Heparin Chromatography

Process 1: An affinity chromatography column was used to selectively purify AAV2-Construct. The animal-derived matrix used within the column (e.g. heparin) can be supplied as GMP material suitable for use in the manufacture of human therapeutic products. Whilst the source, manufacture and supply of the animal-derived matrix is accredited from a GMP and human use perspective, a viral safety risk remains with the use of any animal derived raw materials.

Change to Process 1: Alternative, non-animal derived chromatography matrices were explored as a replacement for the current affinity column filled with an animal-derived matrix (e.g. heparin). An ion exchange chromatography resin was initially used to replace the animal-derived matrix for the first GMP batch according to the improved process and subsequently a non-animal derived affinity chromatography resin was introduced. This change was implemented to achieve a reduction in the biosafety risks associated with animal derived raw materials.

Different chromatography matrices were assessed and suitability was based on the purity/impurity profile, titre and potency of the final product.

Final Product Container and Container Closure

Original Process: The Drug Product was filled into 0.5 mL sterile, polypropylene, screw-capped, gasketed vials at volumes of 0.1 mL per vial.

Change to Process 1: Filling of 0.3 mL Drug Product into Type 1 borosilicate glass injection vials, with a bromobutyl stopper and crimped (tamper evident) vial overseal closure. This change was implemented to achieve improved integrity of final Drug Product, improved containment during use, improved pharmaceutical acceptability of the primary container product contacting materials, ease of visual inspection and safety (security provided by tamper evident overseal).

Assessment of compatibility of Drug Product in glass vials was performed by product recovery and product stability.

Scale Up of Upstream Process Steps

Process 1: Expansion and scale-up of a single vial of HEK293 MCB, through serial passaging, to 12 ten-layer cell stacks.

Change to Process 1: Expansion and scale-up of a single vial of HEK293 MCB, through serial passaging, to 24 ten-layer cell stacks. This change was implemented to achieve increased scale of upstream process to allow additional material for in process testing and controls as well as increased Drug Substance volume for testing and fill finish. Furthermore, this change was implemented to achieve increased Drug Product availability.

Analysis of cell viability, growth rate and cell yield were assessed to determine optimal and consistent performance.

Process Optimization

A number of process steps from the original manufacturing process remained unchanged in terms of physical and chemical definition; however they were further evaluated in order to improve process understanding and characterization and to ensure optimal process performance in terms of process consistency and robustness.

HEK293 Cell Culture and Expansion

Process 1: Expansion and scale-up of a single vial of HEK293 MCB, through serial passaging, to 12 ten-layer cell factories.

Process Optimization: Concurrent with the change in cell culture scale from 12 CS10 to 24 CS10, studies to optimize seeding density and assess growth kinetics for optimal cell culture performance were performed. Operational (non-process) changes were optimized to improve control and reproducibility of process steps to ensure process consistency and robustness. Specifically, this aspect of the process was optimized to increase from 12 CS10 to 24 CS10 to improve yield.

Analysis of cell viability, growth rate and cell yield was performed to determine optimal and consistent performance.

Plasmid Triple Transfection and Harvest

Process 1: The three plasmid transfection of HEK293 cells was performed by calcium phosphate DNA precipitation following chloroquine pre-treatment of cells.

Process Optimization: Optimize transfection, including cell seeding conditions, comparison of calcium phosphate and polyethylenimine. Optimize plasmid concentration/ratio. Determine optimal day post-transfection for medium exchange and harvest. Operational (non-process) changes were optimized to improve control and reproducibility of process steps to ensure process consistency and robustness. Chloroquine was removed from the process to improve safety.

Further optimizations were performed at small scale, including cell seeding density, media for cell growth, FBS concentration and DNA:PEI ratio. Once an optimized transfection process had been established, the process was performed at full scale using PEI and compared to the same process using calcium phosphate to demonstrate comparability.

Microfluidization for Cell Disruption

Process 1: This process uses mechanical lysis of cells to release intracellular virus using a microfluidizer.

Process Optimization: No changes to this process step were performed. Development studies focused on optimization and definition of operating conditions using a sanitary microfludizer suitable for GMP manufacture and containment._Operational (non-process) changes were implemented to improve control and reproducibility of process steps and to improve process understanding and characterization to ensure process consistency and robustness._Consistency of in-process control tests for product purity and vector titre were determined.

Density Gradient Ultracentrifugation

Process 1: Density gradient purification to reduce process impurities.

Process optimization: As the current gradient purification method is manual, it is possible that it could contribute to process variability. Whilst the principles of the purification method remain unchanged, opportunities to make this process step more robust and semi-automated were assessed._Operational (non-process) changes were implemented to improve control and reproducibility of process steps and to improve process understanding and characterization to ensure process consistency and robustness._Consistency of tests for product purity and vector titre were determined.

Ultrafiltration (UF)/Diafiltration (DF)

Process 1: Buffer exchange through UF/DF and dialysis.

Process Optimization: Each process step involving buffer exchange through UF/DF and dialysis was optimized to establish the critical process parameters, as well as ensure acceptability of contact materials for product quality. This change was implemented to ensure process consistency and robustness, to improve process understanding and characterization and to ensure suitability of product contact materials. The virus yield and impurity profile was assessed.

### Switch in Plasmid Encoded Antibiotic Resistance

The AAV2-Construct clinical vector expressing a therapeutic construct is produced via a transient transduction platform utilizing three plasmids; a vector plasmid containing the therapeutic construct/transgene and promoter with inverted terminal repeat sequences, an AAV helper plasmid that contains the AAV2-Rep and Cap sequences and finally a helper plasmid that encodes the essential Adenovirus genes E2A, E4 and VA. All three plasmids used for the production of the AAV2-Construct clinical vector produced according to the original process contained an ampicillin (Amp) selectable marker. To avoid concerns over patient sensitivity to residual ampicillin and a theoretical transfer of the AmpR gene to the final vector the plasmids were re-engineered to swap the Amp selectable marker sequence for a kanamycin (Kan) resistance sequence. Full sequencing was performed to confirm that the remainder of the plasmid backbone remains the same for all three plasmids. These plasmids were evaluated for their yield performance in producing rAAVs in small scale in vitro studies to establish their suitability for use in cGMP production. This experimental analysis of the plasmids was via a head to head comparison of yield with a control consisting of the plasmids previously used for production of the AAV2-Construct clinical vector and is described below. Once plasmid suitability was determined in small scale in vitro studies, the KanR plasmids were assessed in a full scale, non-GMP process.

### Change in Master Cell Bank Cell Line

The production of the AAV2-Construct clinical vector from the original process utilized an HEK293 cell line that was derived, tested and banked by the GMP facility (original process 1) from an HEK293 cell line that is commercially available through Stratagene. This cell bank was fully tested and characterized but a full documented history of the cell bank was not available. To ensure full traceability of the HEK293 cells, new cells were sourced from the ATCC, evaluated for comparability against the original process 1 cells and used for GMP MCB production. Three cell lines (two HEK293 cell lines and one 293T cell line) were screened in a head to head comparison of the following parameters to determine the optimal cell line for the production of AAV2-Construct vector: 1) Generic transduction efficiency via GFP Mean Fluorescence Intensity & Percent GFP using an AAV-based eGFP plasmid, 2) Specific transduction efficiency for AAV2-Construct product assessing yield of AAV2-Construct via DRP (DNase Resistant Particle) analysis, 3) Growth kinetics by cell counts and percent of viable cells, and 4) Continued passage and retest for yield and transduction efficiency to compare transduction efficiency with cells at a later passage.

### Improved Robustness of the AAV2 Purification Chromatography

Animal derived (e.g. heparin) affinity column step in the original Phase 1/2 manufacturing process was replaced with a non-animal derived ion exchange (AEX) column, UnoQ, to improve potential safety concerns (potential of adventitious contaminants from animal derived matrix). FIG. 2 shows a flow diagram of the process highlighting the key process steps and the final column chromatography step (Step 18). Prior to the final UnoQ purification step a low salt dilution step reduces conductivity and supports efficient AAV2-Construct binding to the column. The acceptable salt concentration (conductivity) range for successful performance of this stage was demonstrated to be very narrow. If conductivity was too high, the product did not bind to the AEX column and was lost in the flow-through. If conductivity was too low, product aggregation could occur resulting in loss of product during the purification. This impacted product yield and concentration and as a result batch success—a low yielding batch would not be a viable in terms of material required to support the clinical studies. The UnoQ step did not however have any negative impact on product efficacy or safety as demonstrated by successful production of a non-clinical AAV2-Construct batch and the GMP batch using this improved process.

To address the narrow salt range required prior to AEX, an alternative non-animal derived affinity chromatography column, AVB Sepharose from GE Healthcare, was assessed. Following a comprehensive development phase to ensure maintenance of product comparability as described below the affinity column (with an animal-derived matrix) was replaced with AVB for manufacture of subsequent GMP batches.

AVB Sepharose is an affinity medium with affinity for adeno associated viruses used in AAV2 purification to enable high purity and yield production. The affinity ligand is a 14 kDa fragment from a single chain antibody, expressed in yeast. The AVB column allows for good binding and elution of AAV2-Construct and can be performed in higher salt (conductivity). Thus, the AVB column represents a robust process step that minimizes product losses. AVB is non-animal derived so its use poses no risk to safety from adventitious contaminants.

### Control of Drug Substances of the Disclosure

Exemplary Drug Substances are characterized by the tests listed in Table 19.

TABLE 19

| Test | Test Method | Acceptance Criteria |
|---|---|---|
| Vector Characterization | | |
| Physical Titre | qPCR Based DNase Resistant Particle (DRP) | >0.7 × 10\hat{ }12 |
| | Assay | |
| Infectious Unit (IU) Titre | Infection of RC32 cells followed by detection | >1.24 × 10\hat{ }8 IU/mL |
| | of AAV2 by qPCR | |
| DRP:IU Ratio - Calculation | n/a | <5645:1 |
| Total Particles | Commercial anti-AAV2 particle ELISA | >0.49 × 10\hat{ }12 |
| | | particles/mL |
| Full:Empty Ratio | Transmission electron microscopy | <55% empty particles |
| Identity | | |
| Vector Identity (DNA) | Purification of viral DNA with DNA | Matches the expected |
| | sequencing of both strands | sequence with the |
| | | exception of the ITR |
| | | regions |
| Purity and Impurities | | |
| Total Protein | Micro-BCA Protein Quantification | >5 μg/mL |
| Purity | SDS-PAGE Assay with impurities estimated by | Purity >90% with no |
| | intensity analysis | single impurity >2%. |
| | | Three bands |
| | | corresponding to AAV2 |
| | | VP1, VP2 and VP3 |
| | | should be evident. |
| Replication Competent AAV | HEK293 Cell Based Amplification and qPCR | <LOD of assay |
| HEK293 Host Cell Protein | Commercial ELISA Kit | <80 ng/mL |
| Total DNA | Picogreen assay | Report result |
| HEK293 Host Cell DNA | qPCR assay | <2.66 × 10\hat{ }7 pg/mL |
| | Method res DNA SEQ Human (Life | |
| | Technologies kit) | |
| Residual BSA | Commercial ELISA Kit | <126 ng/mL |
| Residual Benzonase | Commercial ELISA Kit | <5 ng/mL |
| Residual AVB | Commercial ELISA kit | Report result |
| Safety | | |
| Bioburden Assay | Membrane Filtration | <1 CFU/1% PB |
| Endotoxin Assay | Quantitative kinetic-chromogenic method. | <5 EU/mL |

n/a: Not Applicable

Analytical Procedures

Physical Titre: Genomic titre is determined using qPCR. This method allows quantification of genomic copy number. Samples of the vector stock are diluted in buffer. The samples are DNase treated and the viral capsids lysed with proteinase K to release the genomic DNA. A dilution series is then made. Replicates of each sample are subjected to qPCR using a Taqman based Primer/Probe Set specific for the CAG sequence. A standard curve is produced by taking the average for each point in the linear range of the standard plasmid dilution series and plotting the log copy number against the average C_{T} value for each point. The titre of the rAAV vector can be calculated from the standard curve and is expressed as DNase Resistant Particles (DRP)/mL.

Infectious Unit (IU) Titre: This assay quantifies the number of infectious particles of AAV. Quantification is performed by infecting RC32 cells (HeLa expressing AAV2 Rep/Cap) with serial dilutions of the vector sample and uniform concentrations of wild type adenovirus to provide helper function. Several days post infection, the cells are lysed diluted to reduce PCR inhibitors and assayed by qPCR in the same manner as described in the physical titre assay above, except that the DNase and Proteinase K digestion is omitted and only the qPCR portion is performed. Individual wells are scored as Positive or Negative for AAV amplification. The scored wells are used to determine the TCID₅₀ in IU/mL using the Karber Method.

Total Particles: The assay uses an ELISA technique (AAV2 Titration ELISA KIT). A monoclonal antibody specific for a conformational epitope on assembled AAV2 capsids is coated onto microtitre strips and is used to capture AAV2 particles from the specimen. Captured AAV particles are detected in two steps. First a biotin-conjugated monoclonal antibody to AAV2 is bound to the immune complex. In the second step streptavidin peroxidase conjugate reacts with the biotin molecules. Addition of substrate solution results in a color reaction which is proportional to the amount of specifically bound viral particles. The absorbance is measured photometrically at 450 nm.

Full:empty Ratio (Transmission Electron Microscopy): The full:empty ratio of AAV2 particles may be determined using negative staining transmission electron microscopy (TEM). Samples are applied to a grid fixed. Samples are visualized using a transmission electron microscope and counts are performed of the full (i.e. containing DNA) and empty AAV2 capsid particles based on their morphology. The ratio of full:empty particles is calculated from the particle counts.

Full:empty Ratio (Analytical Ultracentrifugation): The full:empty ratio of AAV2 particles may be determined using analytical ultracentrifugation (AUC). AUC has an advantage over other methods of being non-destructive, meaning that samples may be recovered following AUC for additional testing. Samples comprising empty and full AAV2 particles are applied to a liquid composition through which the AAV2 move during an ultracentrifugation. A measurement of sedimentation velocity of one or more AAV2 particles provides hydrodynamic information about the size and shape of the AAV particles. A measure of sedimentation equilibrium provides thermodynamic information about the solution molar masses, stoichiometries, association constants, and solution nonideality of the AAV2 particles. Exemplary measurements acquired during AUC are radial concentration distributions, or “scans”. In some embodiments, scans are acquired at intervals ranging from minutes (for velocity sedimentation) to hours (for equilibrium sedimentation). The scans of the methods of the disclosure may contain optical measurements (e.g. light absorbance, interference and/or fluorescence). Ultracentrifugation speeds may range from between 10,000 rotations per minute (rpm) and 75,000 rpm, inclusive of the endpoints. As full AAV2 particles and empty AAV2 particles demonstrate distinct measurements by AUC, the full/empty ratio of a sample may be determined using this method.

Vector Identity (DNA): This assay provides a confirmation of the viral DNA sequence. The assay is performed by digesting the viral capsid and purifying the viral DNA. The DNA is sequenced with a minimum of 2 fold coverage both forward and reverse where possible (some regions, e.g., ITRs are problematic to sequence). The DNA sequencing contig is compared to the expected sequences to confirm identity.

Total Protein: This assay quantifies the total amount of protein present in the test article by using a Micro-BCA kit. In order to eliminate matrix effects of the formulation buffer samples are precipitated with acetone and the precipitated protein re-suspended in an equal volume of water prior to analysis. The protein concentration determination is performed by mixing test article or diluted test article with a Micro-BCA reagent provided in the kit. The same is performed using dilutions of a Bovine Serum Albumin (BSA) Standard. The mixtures are incubated at 60° C. and the absorbance measured at 562 nm. A standard curve is generated from the standard absorbance and the known concentrations using a linear regression fit. The unknown samples are quantified according to the linear regression.

Purity: This assay provides a semi-quantitative determination of AAV purity. Based on the results of the AAV2 capsid particle ELISA, samples are concentrated by SpeedVac and either 4×10\hat{ }10 or 1×10\hat{ }11 particles are loaded and the capsid proteins are separated on an SDS-PAGE gel. Densitometry analysis of the SYPRO Orange stained gels allows calculation of the approximate impurity levels relative to the capsid proteins (Vp1, Vp2 and Vp3).

Replication Competent AAV: Test article is used to transduce HEK293 cells in the presence or the absence of wild type adenovirus. Three successive rounds of cell amplification will be conducted and total genomic DNA is extracted at each amplification step.

The rcAAV2 are detected by real-time quantitative PCR. Two sequences are isolated genomic DNA; one specific to the AAV2 Rep gene and one specific to an endogenous gene of the HEK293 cells (human albumin). The relative copy number of the Rep gene per cell is determined. The positive control is the wild type AAV virus serotype 2 tested alone or in the presence of the rAAV vector preparation.

The limit of detection of the assay is challenged for each tested batch. The limit of detection is 10 rcAAV per 1×10\hat{ }8, or 1×10\hat{ }10, genome copies of test sample. If a test sample is negative for Rep sequence, the result for this sample will be reported as: NO REPLICATION, <10 rcAAV per 1×10\hat{ }8 (or 1×10\hat{ }10) genome copies of test sample. If a test sample is positive for Rep sequence, the result for this sample will be reported as: REPLICATION.

HEK293 Host Cell Protein: The HEK293 host cell protein (HCP) assay is an immunoenzymetric assay. Samples of purified virus are reacted in microtitre strips coated with an affinity purified capture antibody. A secondary horseradish peroxidase (HRP) conjugated enzyme is reacted simultaneously, resulting in the formation of a sandwich complex of solid phase antibody-HCP-enzyme labelled antibody. The microtitre strips are washed to remove any unbound reactants. The quantity of HEK293 HCPs is detected by the addition of 3,3′,5,5′ tetramethyl benzidine peroxidase, an HRP substrate, to each well. The amount of hydrolyzed substrate is read on a plate reader and is directly proportional to the concentration of HEK293 HCPs present.

Total DNA: Picogreen reagent is an ultra-sensitive fluorescent nucleic acid stain that binds double-stranded DNA and forms a highly luminescent complex (λexcitation=480 nm-λemission=520 nm). This fluorescence emission intensity is proportional to dsDNA quantity in solution. Using a DNA standard curve with known concentrations, DNA content in test samples is obtained by converting measured fluorescence.

HEK293 Host Cell DNA: The original process measured size and quantity of 3 different amplicons whereas the improved process measures total hcDNA including high molecular weight and sheared DNA. The qualification data the improved process demonstrates that the assay is specific and sufficiently sensitive to meet the requirements in assessing hcDNA per dose of <10 ng/dose (WHO Expert Committee on Biological Standardization, 2013).

Residual BSA: Residual BSA is quantified using a commercially available ELISA kit manufactured and marketed by Bethyl. The scientific principle to the ELISA kit is very similar to that specified for the Host Cell Protein ELISA.

Residual Benzonase: This assay uses purified polyclonal antibodies specific to Benzonase endonuclease to detect residual Benzonase in the test sample by sandwich ELISA. Accurate measurement is achieved by comparing the signal of the sample to the Benzonase endonuclease standards assayed at the same time.

Bioburden Assay: This procedure is used to determine quantitatively (if detectable) the amount of bioburden present in a sample. The method used involves membrane filtration of half of the sample onto each of two membranes. The membranes are placed onto separate agar media plates which are incubated in aerobic and anaerobic conditions sequentially at 20-25° C. and 30-35° C. At the conclusion of incubation; aerobe, anaerobe, and fungal counts are expressed as CFU/mL of sample.

Endotoxin Assay: This assay is used to determine if bacterial endotoxins are present in the test article. A quantitative procedure is performed by the kinetic-chromogenic method. Known amounts of endotoxin are tested in parallel with the test article for an accurate determination of the level of bacterial endotoxin. The potential for interference by the test article is examined by spiking the test article plus LAL reagent with specified levels of endotoxin. Following the inhibition/enhancement test, the endotoxin content of the test article is determined.

Residual AVB: Residual AVB analysis is performed using a commercial ELISA kit, CaptureSelect™ AVB Sepharose HP Ligand Leakage ELISA, manufactured by Life Technologies. The sandwich ELISA principle involves coating microtitre plates with anti-affinity ligand polyclonal goat antibodies to capture AVB present in the sample. Detection is via a biotinylated affinity-purified anti-AVB ligand polyclonal goat antibodies and streptavidin horseradish peroxidase conjugate. Concentration of residual AVB in the sample is determined by measurement against a standard curve.

### Stability of AAV Compositions

Compositions of the disclosure maintain long term stability when stored at <−60° C. For example, compositions of the disclosure maintain long term stability when stored at temperature between −80° C. and 40° C. (approximately human body temperature), inclusive of the endpoints. For example, compositions of the disclosure maintain long term stability when stored at temperature between −80° C. and 5° C., inclusive of the endpoints. For example, compositions of the disclosure maintain long term stability when stored at −80° C., −20° C. or 5° C. In some embodiments, compositions of the disclosure are formulated as liquids or suspensions, aliquoted into one or more containers (e.g. vials), and stored at <−60° C. In some embodiments, compositions of the disclosure are formulated as liquids or suspensions, aliquoted into one or more containers (e.g. vials), and stored at −80° C., −20° C. or 5° C.

Compositions of the disclosure may be provided in a container with an optimal surface area to volume ratio for maintaining long term stability when stored at <−60° C. Compositions of the disclosure may be provided in a container with an optimal surface area to volume ratio for maintaining long term stability when stored at −80° C., −20° C. or 5° C. In some embodiments, compositions of the disclosure are formulated as liquids or suspensions, aliquoted into one or more containers (e.g. vials), and stored in one or more containers with a surface area to volume ratio as large as possible when all storage requirements are considered.

Compositions of the disclosure maintain long term stability when stored at ambient relative humidity.

### Clinical Dose Design

Choroideremia (CHM) is a hereditary X-linked retinal dystrophy first described in the 19th century. A deletion or mutation of the CHM gene, encoding REP1 leads to degeneration of the choroid, RPE and retina. Choroideremia is characterized by progressive chorioretinal degeneration in affected males and milder signs in carrier females. Symptoms in affected males may evolve from night blindness to peripheral visual field loss, with central vision preserved until late in life. Although carrier females are generally asymptomatic, signs of chorioretinal degeneration can be observed with careful fundus examination. These signs become more readily apparent after the second decade.

CHM is caused by mutations in the CHM gene (Xq21), which encodes component A of Rab geranyl-geranyl-transferase, referred to as REP1. REP1 is required for intra-cellular trafficking, and is therefore essential for normal retinal function. When REP1 is deficient, as is the case in CHM sufferers, there is a gradual loss of function and atrophy of the retinal pigment epithelium, photoreceptors and the choroid, which ultimately leads to blindness. Prior to the development of the pharmaceutical compositions of the disclosure, no treatment was available for CHM.

Retinal gene therapy dosing using an adeno-associated viral (AAV) vector involves the calculation of a single dose which may give lifelong effects. In traditional systemic drug dosing, the half-life of the drug is measured in the blood and this determines the frequency of repeat doses administered throughout the day, so that the peak levels are non-toxic and the trough levels are sufficient to maintain pharmacodynamic activity. With traditional dosing regimens it is possible to use healthy volunteers to work out drug clearances and hence calculate the half-life of the drug and thereby estimate the required plasma levels in order to calculate the dose and frequency of administration. Adjustments can be made—for instance, dosing may be reduced for drugs metabolized by the liver in cases of liver failure. In pediatric cases, dosing can be adjusted according to weight which recognizes the smaller blood volume and target organs. These traditional methods of actively titrating the dosage present challenges with ophthalmic gene therapy administration because, in preferred embodiments of the methods of treatment and uses of the pharmaceutical compositions of the disclosure, the aim of the treatment is to give only one dose that will achieve lifelong therapeutic effects.

In some embodiments of the methods of the disclosure, the pharmaceutical composition is administered to the eye via a sub-retinal injection to target RPE and photoreceptor cell layers. As part of the surgical procedure for a sub-retinal injection, a vitrectomy is performed on the eye to be treated prior to the sub-retinal rejection. This vitrectomy is followed by a sub-retinal infusion of balanced salt solution to form a hemispherical bleb. In some embodiments of the disclosure, the hemispherical bleb is approximately 10 mm² in area or the hemispherical bleb is 10 mm² in area. In some embodiments of the disclosure, a pharmaceutical composition of the disclosure is formulated in a final volume of 0.1 mL and injected into the preformed bleb, allowing the pharmaceutical composition to evenly distribute itself across the retinal surface (e.g. across the 10 mm² retinal surface). Thus, in those embodiments in which a hemispherical bleb is formed into which the pharmaceutical composition may be injected, the bleb facilitates an even distribution of full rAAVs of the composition across the RPE cell layer of the retina. Following the injection of the pharmaceutical composition, the bleb resolves within a few hours post-surgery.

A potential complication of the vitrectomy/sub-retinal surgery is reduced visual acuity (VA) immediately post-surgery. Another potential and longer-term complication is the formation of cataracts. Both these factors affect the vision when a VA test is performed, and therefore, these factors are considered when determining the outcome of dose-ranging, especially when VA is the sole predictor of the effective dose determination.

Choosing the appropriate dose in gene therapy trials is different to traditional ways of assessing dose-ranging. The goal is to administer the highest efficacious dose possible that is known to be safe. The reasons for this are primarily driven by the fact that the multiplicity of infection (MOI) for retinal photoreceptors is very high in the order of 10⁵ genome particles (gp) per retinal cell. Given that, in some embodiments, and depending upon the biology of the individual subject being treated, the maximum area of retina that can be treated is approximately 10 mm², and that the area of retina that is treatable is around the macula, the total number of rods, cones and retinal pigment epithelium (RPE) cells in this area amount to approximately 1×10⁶ cells, the low dose cohort (10¹⁰ gp) in the study (described in Example 13) provides an MOI of 1×10⁴ gp only, whereas the high dose arm (10¹¹ gp) in the same study provides an MOI of 1×10⁵ gp.

Phase I/II data are provided in Example 13. The high dose of this study (10¹¹ gp) has been shown to be safe and efficacious. In certain embodiments, a steroid therapy regime may be used to address any immune response of the subject to the AAV of the pharmaceutical composition. As described in Example 13, a dose of 10¹¹ gp of the pharmaceutical composition of the disclosure results in an MOI in a preferred range of 10⁵ gp.

### Assessing the Therapeutic Dose in Choroideremia

One parameter to measure is an improvement in visual function. This assumes that the visual function parameter is adversely affected by the genetic defect and might therefore be reversible to some extent so the improvement can be measured following successful gene transfer. In some embodiments of methods of treating CHM, visual function parameters are measured, including, but not limited to visual acuity (VA), retinal sensitivity and measurements of dark adapted vision. For some subjects, however, depending on the severity of the defect and the stage of the degeneration, some of the above visual function parameters may be considered normal. In some embodiments of the methods of treatment of the disclosure, the visual function parameter of a subject improves following treatment and the improvement results in visual function parameter measurements that are equal to those measurements obtained from a healthy subject (who does not have CHM) and who did not receive the treatment. In some embodiments, the healthy subject is an age-matched individual, for example, to account for natural age-related vision deterioration that is independent of CHM. Moreover, as men and women can differ in their color vision acuity, in some embodiments, the healthy subject is an individual having the same XY chromosome composition. In some embodiments of the methods of treatment of the disclosure, including those embodiments in which the subject has already experienced significant retinal damage (neuronal loss) or in which the unaffected areas of the retina are small and/or discontinuous, a full recovery of visual function may not be expected. In those embodiments in which only a partial recovery is possible, an improvement of visual function may be apparent when the improvement is compared to the subject's baseline function (the function assessed in this subject and in the treated eye prior to administration of the pharmaceutical composition). Even in the embodiments in which only a partial recovery is possible, the treatment raises the baseline function of the eye and the subject's vision function such that CHM and/or age-related retinal degeneration are postponed or reduced, thereby prolonging the lifetime of useful vision for the subject.

### Anatomical Density of Retinal Cells in Target Zone

In order to calculate the MOI for clinical treatments in vivo, the total number of vector particles and the number of target cells in the region to be treated are determined. These number depend on the area of retina exposed to vector and the density of each of the different cell types in that region.

Pharmaceutical compositions of the disclosure are administered in CHM patients via a sub-retinal injection following an induced retinal detachment. For example, if it is assumed that the retinal detachment has linear dimensions of approximately 2-3 disc diameters, this area of retinal detachment is equivalent to a circle with a diameter of 3-4 mm, or an area of approximately 10 mm².

The disclosure provides exemplary cellular densities for exemplary dose calculations, however, the doses of the pharmaceutical compositions of the disclosure are not bound by these exemplary calculations. In the central macula, the density of RPE cells in normal human subjects is 5,000 cells per mm² (based on post-mortem studies in patients in the 40-50 year age group). In the central macula, the density of rod cells in normal human subjects is 75,000 cells per mm² but excludes central foveal area of approx. 0.5 mm². In the central macula, the density of cone cells in normal human subjects is 150,000 per mm² in the central 0.5 mm² fovea (75,000 total)+25,000 per mm² in the macula outside the foveal area. Thus, in some embodiments of the disclosure that are based on these exemplary cell densities, the total number of each of the three cell types exposed to rAAV of the pharmaceutical compositions of the disclosure in a standard 10 mm² detachment can be calculated from the above as follows: (a) RPE: 5,000 cells per mm²—TOTAL=5×10⁴ in 10 mm²; (b) Rods: 75,000 cells per mm², but excluding the central 0.5 mm² fovea—TOTAL=7.1×10⁵ in 10 mm²; and (c) Cones: 150,000 cells per mm² in central 0.5 mm² fovea (75,000), but only 25,000 per mm² extra-foveal—TOTAL=3.1×10⁵ in 10 mm². The density of RPE, rods and cones differs in the central 10 mm² of the macula and varies by about one log unit, with the lowest density (and hence highest MOI) seen in relation to the RPE.

### List of Abbreviations

AAV Adeno-Associated Viral

AmpR Ampicillin Resistance

ATCC American Type Culture Collection

AVG Average

BCA Bicinchoninic Acid

BGH-polyA Bovine Growth Hormone Polyadenylation Sequence

bp Base Pairs

BSA Bovine Serum Albumin

BSE Bovine Spongiform Encephalopathy

BSS Balanced Salt Solution

° C. Degrees Centigrade

CBA Cytomegalovirus Enhancer/Chicken-Beta Actin

CEP Certificates of Suitability

c.f. Conferre (Compare)

CFU Colony Forming Units

CI Confidence Interval

cm Centimetre

C_{T} Cycle Threshold

CV Coefficient of Variation

cGMP current Good Manufacturing Practice

CHM Choroideraemia

CMV Cytomegalovirus

CNS Central Nervous System

DCB Development Cell Bank

DF Diafiltration

DLS Dynamic Light Scattering

DMEM Dulbecco's Modified Eagle Medium

DNA Deoxyribonucleic Acid

DP Drug Product

DRP DNase Resistant Particle Assay

dsDNA Double Stranded DNA

EDTA Ethylenediaminetetraacetic Acid

EF Elongation Factor

EFS Elongation Factor 1 Alpha Short

ERA Effective Filtration Area

EGFP Enhanced Green Fluorescent Protein

ELISA Enzyme-Linked Immunosorbant Assay

EP European Pharmacopoeia

et al. et alii (and others)

EtBr Ethidium Bromide

FACS Fluorescence-Activated Cell Sorting

FBS Fetal Bovine Serum

FC Final Container

FTM Fluid Thioglycollate Medium

g x gravity

GAD Glutamic Acid Decarboxylase

GGPP Geranylgeranylpyrophosphate

GF Gel Filtration

GFP Green Fluorescent Protein

GLP Good Laboratory Practice

GMP Good Manufacturing Practice

gp Genome Particles

gp/mL Genome Particles per mL

h Hour

HBSS Hanks' Balanced Salt Solution

HCP Host Cell Protein

HEK Human Embryonic Kidney

HI-FBS Heat Inactivated FBS

HPLC High Performance Liquid Chromatography

HRP Horse Radish Peroxidase

HSA Human Serum Albumin

INN International Non-Proprietary Name

IPC In Process Control

IST Investigator Sponsored Trial

ITR Inverted Terminal Repeat

IU Infectious Units

KanR Kanomycin Resistance

kb Kilobase

kDa Kilodaltons

kg Kilogram

kGy Kilogray

L Litre

LOQ Limit of Quantitation

LAL Limulus Amoebocyte Lysate

LOD Limit of Detection

μL Microlitres

μm Micrometre

m Metre

M Molar

mbars Millibars

MCB Master Cell Bank

mDa Mega Daltons

mg Milligrams

MIA (IMP) Manufacturer/Importer Authorisation

min Minute

mL Millilitre

mm Millimetre

mM Millimolar

mOsm Milliosmoles

MSC Microbiological Safety Cabinet

N/A Not Applicable

ND Not Determined

NEAA Non-Essential Amino Acids

ng Nanogram

NF National Formulary

nm Nanometre

No. Number

OCT Optimal Cutting Temperature

OD Optical Density

OD₆₀₀ Optical Density Measured at 600 nm

ONL Outer Nuclear Layer

p Probability

Pa Pascals

PAGE Polyacrylamide Gel Electrophoresis

PB Purified Bulk

PBDS Purified Bulk Drug Substance

PCR Polymerase Chain Reaction

PDA Photodiode Array Detector

PEI Polyethylenimine

PETG Polyethylene Terephthalate Glycol

pfu Plaque Forming Units

pg Picograms

ppm Parts per Million

PSG Pure Steam Generator

psi Pounds per Square Inch

PTC Points to Consider

QC Quality Control

QP Qualified Person

QFPERT Quantitative Fluorescent Product Enhanced Reverse Transcriptase

qPCR Quantitative Polymerase Chain Reaction

qs Quantum Sufficit

rAAV Recombinant AAV

R&D Research and Development

rcAAV Replication Competent AAV

rDNA Recombinant DNA

REP1 Rab Escort Protein Type 1

RH Relative Humidity

RO Reverse Osmosis

RPE Retinal Pigment Epithelium

RPE65 Retinal Pigment Epithelium 65 Protein

rpm Revolutions per Minute

s Second

SCDM Soybean-Casein Digest Medium

SDA Sabouraud Dextrose Agar

SDS Sodium Dodecyl Sulphate

SLO Scanning-Laser Ophthalmoscopy

SOP Standard Operating Procedure

SSS Salt and Surfactant Solution

ST DEV Standard Deviation

TAMC Total Aerobic Microbial Count

TBA To Be Assigned

TCID₅₀ Tissue Culture Infectious Dose 50

TFF Tangential Flow Filtration

TSA Trypticase Soy Agar

TT Technology Transfer

TYMC Total Yeast/Mould Count

U Units

UF Ultrafiltration

USP United States Pharmacopoeia

UV Ultraviolet

vg Vector Genome

vp Viral Particles

v/v Volume to Volume

WFI Water for Injection

WPRE Woodchuck Hepatitis Virus Post-Transcriptional Regulatory Element

WT Wild Type

μl Microlitre

μm Micrometer

### EXAMPLES

### Example 1: Comparative Analytical Testing Data from Original and Improved Processes

Samples from the original process were provided for use in data analysis of the improved process so that data could be generated from both sets of batches using the same methods (where applicable) to allow for a direct relative comparison of data generated, unless stated otherwise. For the data provided below, the measurements are taken from one or more batches of AAV2-Construct, wherein the Construct is identical in all batches.

The intermediate products generated by the improved process were analyzed for physical titre (qPCR), total protein (Micro-BCA), total DNA (Picogreen) and purity (SDS-PAGE/Sypro Orange stain).

The genomic titre data for samples from the original process were taken from the testing documentation provided.

The total protein and total DNA data from the original in-process intermediate samples presented here were generated according to the improved process from the starting material produced from the original process.

The Electron Microscopy (EM) analysis used for full to empty particle ratio determination was not fully established at the time that the process was transferred from the original to the improved process. The original process did not involve any testing at the time of manufacture to determine the ratio of full to empty particles within the clinical batch, either to assess the purification (density gradient) step or to measure final Drug Product, therefore no data was available for comparison of the purification (density gradient) process step from the improved process with that from the original process. Therefore, the full to empty particle ratio was not used as an assessment of comparability. Instead, determination of physical titre (or genome particles) as measured by qPCR was considered a suitable determination of successful transfer of the entire process and of successful transfer of individual process steps as this is specific to the vector product and therefore representative of full capsids. However, for the original process, a method based on analytical ultracentrifugation (AUC) was used to assess the full to empty ratio for information only. The result of this test was presented in Table 33.

EM is the preferred method and has been performed on all batches manufactured to date. Additionally, EM analysis has been used retrospectively to test both batches from the original process to give an understanding of the full to empty ratio. This testing has demonstrated that the improved process is capable of generating a comparable ratio of full to empty capsids as shown in Table 20.

TABLE 20
Electron Microscopy Analysis

| | Empty Capsids as |
|---|---|
| Batch | Estimated by EM |
| (Original Process) Drug Product* | 11% |
| (Original Process) Drug Substance | 11% |
| (Improved Process) Drug Substance - batch 1 | 15% |
| (Improved Process) Drug Substance - batch 2 | 24% |
| (Improved Process) Drug Substance - batch 3 | 51%** |

*No DS available for testing. Testing performed on DP for information only, however there is limited processing between the Drug Substance and Drug Product therefore we believe that this would be representative of a Drug Substance result.
**Note this value is slightly higher than expected. According to the improved process, the higher ratio of empty particles observed in this batch appears to be due to an operational manipulation where a greater volume of material was removed from the centrifuge tubes during fraction collection/selection for full capsids. A corrective action has been implemented to address this for future batches, and batch 2, which was manufactured after this investigation, had the expected lower number of empty capsids.

The analytical results from the starting material and the reference material (both from the original process), are summarized in Table 21.

TABLE 21

| | Original Process | | | | Improved process—batch 2 | | | |
|---|---|---|---|---|---|---|---|---|
| | | | | Total | | | | Total |
| | Recovery | Titer | DNA | Protein | Recovery | Titer | DNA | Protein |
| | % | (DRP/mL) | (ng/mL) | (ug/mL) | % | (DRP/mL) | (ng/mL) | (ug/mL) |
| Post Lysis | NA | 2.1 × 10\hat{ }9 | ND | 4655 | NA | 1.3 × 10\hat{ }11 | 505 | 7624 |
| Post Clarification | NA | 8.4 × 10\hat{ }10 | ND | ND | 110% | 1.3 × 10\hat{ }11 | ND | 3226 |
| Post TFF | 118% | 7.4 × 10\hat{ }12 | ND | ND | 104% | 9.5 × 10\hat{ }12 | ND | ND |
| Post Purification | 53% | 2.3 × 10\hat{ }13 | 6 | 821 | 28% | 1.6 × 10\hat{ }13 | 7 | 1223 |
| Post Affinity | 88% | 8.5 × 10\hat{ }12 | ND | ND | 105% | 6.8 × 10\hat{ }12 | ND | ND |
| Chromatography | | | | | | | | |
| (animal-derived | | | | | | | | |
| matrix) | | | | | | | | |
| Final Product | 82% | 6.7 × 10\hat{ }12 | 7 | 32 | 105% | 7.1 × 10\hat{ }12 | 9 | 36 |
| Global Recovery | 45% | | | | 36% | | | |
| % | | | | | | | | |

The harvest material produced by the improved process contained a higher total protein concentration than the harvest material produced by the original process. However, the purity profiles of the final Drug Substance were similar between the original and improved processes with no major impurities detected. The titre recovery between the original and improved processes was also similar.

With regards to the acceptance criteria defined for a successful technology transfer of the process, the improved process was controlled and the performance of the individual process steps of the improved process were similar to those observed during the original process. The process recovery (vector titre) from the improved process was within 20% of that obtained by the original process. The final product purity (DNA/protein content) from the improved process was within 20% of those obtained by the original process, with similar SDS-PAGE profile.

### Example 2: Optimization of Manufacturing Process

For the data provided below, the measurements are taken from one or more batches of AAV2-Construct, wherein the Construct is identical in all batches.

Plasmid Triple Transfection and Harvest

Process 1: The three plasmid transfection of HEK293 cells was performed by Calcium phosphate DNA precipitation following chloroquine pre-treatment of cells.

Process Optimization: Optimize transfection, including cell seeding conditions, comparison of Calcium phosphate and polyethylenimine. Optimize plasmid concentration/ratio. Determine optimal day post-transfection for medium exchange and harvest. Operational (non-process) changes were optimized to improve control and reproducibility of process steps to ensure process consistency and robustness. Chloroquine was removed from the process to improve safety.

An analysis of vector titre yield was performed. Polyethylenimine (PEI) transfection reagent was compared with Calcium phosphate (used for Process 1) to determine if the former could be used to replace Calcium phosphate as a more robust transfection agent. The requirement for a pre-treatment with chloroquine was also evaluated. All parameters were assessed by determining the physical titre by qPCR using a non-validated method.

Table 22 presents the initial results where the titre of vector following transfection with PEI was compared directly with the titre obtained using Calcium phosphate, both in the presence and absence of chloroquine. All studies were performed with DMEM media containing Glutamax, which is the media used for all upstream process steps.

TABLE 22
Comparison of Transfection Reagents-Physical Titre

| | | Physical Titre Measured |
|---|---|---|
| Transfection | Chloroquine | Post-Transfection |
| Reagent | Pre-Treatment | Using qPCR (DRP/mL) |
| Calcium phosphate | Yes | 1.90 × 10\hat{ }8 DRP/mL |
| Calcium phosphate | No | 6.90 × 10\hat{ }7 DRP/mL |
| PEI | Yes | 1.70 × 10\hat{ }6 DRP/mL |
| PEI | No | 5.60 × 10\hat{ }6 DRP/mL |

This work demonstrated successful transduction using PEI, albeit with lower physical titre results when compared to transfection using Calcium phosphate. This work also showed that when PEI was used as a transfection reagent the chloroquine pre-treatment was not required and could be removed from the process.

Based on this result, further optimizations were performed at small scale, including cell seeding density, media for cell growth, FBS concentration and DNA:PEI ratio. Once an optimized transfection process had been established, the process was performed at full scale using PEI and compared to the same process using Calcium phosphate to demonstrate comparability.

Table 23 shows the data obtained from the full-scale manufacture, which demonstrates the suitability and comparability of PEI as a transfection reagent. Based on this data PEI was selected as the transfection reagent of choice to allow a more robust process step when compared to Calcium phosphate.

TABLE 23
Comparison of Transfection Reagents from
Full Scale Manufacture-Physical Titre

| Transfection Reagent | Titre Post-Transfection |
|---|---|
| PEI | 11.9 × 10\hat{ }14 DRP/flask |
| | (5.41 × 10\hat{ }11 DRP/mL) |
| Calcium | 6.644 × 10\hat{ }14 DRP/flask |
| phosphate | (3.02 × 10\hat{ }11 DRP/mL) |

Furthermore, the overall productivity and process yield of batches produced by the improved process are comparable to those produced by the original process (Table 24) demonstrating the suitability of PEI as a transfection reagent.

TABLE 24
Comparison of Overall Productivity and Process Yield-Batches from
Original and Improved Processes

| Manufacturer | Original Process − | Improved Process − | Improved Process − |
|---|---|---|---|
| and Batch | GMP | non-GMP | GMP |
| Virus Produced | 3.4 × 10\hat{ }13 DRP total | 6.7 × 10\hat{ }13 DRP total | 4.7 × 10\hat{ }13 DRP total |
| Scale of Manufacture | 12 Cell factory vessels | 24 Cell factory vessels | 22 Cell factory vessels |
| Virus Yield/ | 2.83 × 10\hat{ }12 DRP/vessel | 2.79 × 10\hat{ }12 DRP/vessel | 2.14 × 10\hat{ }12 |
| Cell Factory | | | DRP/vessel |

### Example 3: Switch in Plasmid Encoded Antibiotic Resistance—from AmpR to KanR

For the data provided below, the measurements are taken from one or more batches of AAV2-Construct, wherein the Construct is identical in all batches.

Study Materials and Procedure

The ampicillin containing plasmids used for production of the clinical batch produced by the original process served as the control for analysis of AAV2. Construct vector yield in terms of DNase resistant particles (DRP)/cm². The experiments were performed from the same cell thaw event and all cells received the same treatments, media, reagents and growth conditions throughout the experiment.

Plasmids Tested: pAAV.Construct (KAN); pNLREP-CAP2 (KAN); pHELP (KAN); pAAV.Construct, CN1055CM (Original Process; Amp version, used as control); pNLREP-CAP2, CN1054CM (Original Process; Amp version, used as control), pHELP, CN2291CM (Original Process; Amp version, used as control).

Triplicate testing was performed for each set of plasmids. Three 1-Stacks were transfected with pAAV.Construct (KAN), pNLREP-CAP2 (KAN) and pHELP (KAN). Three 1-Stacks were transfected with the equivalent AmpR containing plasmids. Cells were re-fed at 16 to 26 hours post transduction, and the media and cells harvested at 44 to 52 hours post transduction, and the yield of DRP determined for each 1-Stack determined.

Results

The average DRP/cm² was determined among the 3 replicates tested via qPCR. The yields from both the media and the cell lysate were analyzed separately then added to determine the total vector yield per replicate which was then divided by the number of square centimetres.

TABLE 25
Yield Analysis DRP/cm² for AmpR and KanR Plasmids

| | Total Yield | | AVG | |
|---|---|---|---|---|
| Plasmid | (Media + Lysate) | DRP/cm² | DRP/cm² | ST DEV |
| Kan 1 | 3.04 × 10\hat{ }13 | 4.79 × 10\hat{ }10 | 4.61 × 10\hat{ }10 | 1.57 × 10\hat{ }9 |
| Kan 2 | 2.88 × 10\hat{ }13 | 4.52 × 10\hat{ }10 | | |
| Kan 3 | 2.87 × 10\hat{ }13 | 4.51 × 10\hat{ }10 | | |
| Amp 1 | 2.50 × 10\hat{ }13 | 3.93 × 10\hat{ }10 | 3.78 × 10\hat{ }10 | 4.68 × 10\hat{ }9 |
| Amp 2 | 2.64 × 10\hat{ }13 | 4.15 × 10\hat{ }10 | | |
| Amp 3 | 2.07 × 10\hat{ }13 | 3.25 × 10\hat{ }10 | | |

The acceptance criteria for the re-engineered (Kan) plasmids used for manufacture of the Drug Substance and Drug Product was defined as being no less than fourfold of the yield of the control plasmids. The re-cloned Kan versions of the plasmids met the acceptance criteria. Based on the similar yield of DRP obtained and the sequence confirmation, the KanR containing plasmids are considered suitable for production of clinical batches of AAV2-Construct.

### Example 4: Change in Master Cell Bank Cell Line to ATCC HEK-293

For the data provided below, the measurements are taken from one or more batches of AAV2-Construct, wherein the Construct is identical in all batches.

Study Materials and Procedures

The Original Process 1 cell line used for production of the GMP batch by the original process served as the control for transfection efficiency and yield of AAV2-Construct vector. Continued passage and re-testing for yield and transfection efficiency had not formerly been assessed for the Original Process 1 used cells and testing is for information only. The experiments for transfection efficiency, yield analysis and growth kinetics were performed from the same cell thaw event(s) and all cells received the same treatments, media, reagents and growth conditions throughout the screening experiment.

Cell Lines Tested: ATCC=[HEK-293] (ATCC® CRL-1573TM), Bioreliance=293 Cells (G95001 2), Process 1 cell line, and 293T=293T/17 [HEK 293T/17] (ATCC® CRL-1 1268TM). Of note, the 293T Cell line was included as an alternate source.

For transfection efficiency and yield analysis, monolayers of each cell line were transfected with the relevant plasmids and re-fed at 16 to 26 hours post transfection. Harvesting of cells and media for GFP or AAV2-Construct DRP analysis was conducted at 44 to 52 hours post transduction.

Growth kinetics was evaluated by seeding T175 flasks with either 10\hat{ }7 or 2.5×10\hat{ }6 cells/flask and the cell health, confluency, cell count and viability recorded at days 1, 2, 3, 4 and 7 post seeding. This was repeated at a passage ≥15.

Results

Mean Fluorescence Intensity: Mean fluorescence intensity was analyzed via flow cytometry to measure levels of GFP protein expression coupled with number of cells transfected as an indicator of overall gene expression in correlation with viral yield.

TABLE 26
Results for Mean Fluorescence Intensity

| | Mean GFP/Sample | | | Mean |
|---|---|---|---|---|
| Cell Line | Rep 1 | Rep 2 | Rep 3 | GFP |
| ATCC Experiment 1 | 40,935 | 39,347 | 35,525 | 38,602 |
| Bioreliance Experiment 1 | 24,934 | 24,569 | 23,033 | 24,179 |
| MCB Control Experiment 1 | 34,217 | 33,151 | 37,010 | 34,793 |
| 293T | 30,508 | 29,768 | 33,665 | 31,314 |
| ATCC Experiment 2 | 32,722 | 36,068 | 32,656 | 33,815 |
| Bioreliance Experiment 2 | 24,224 | 23,060 | 22,072 | 23,119 |
| MCB Control Experiment 2 | 45,604 | 50,686 | 44,817 | 47,036 |

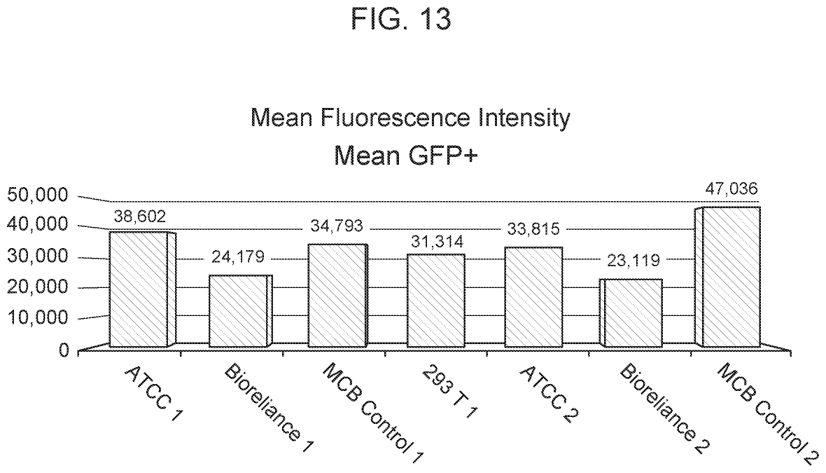

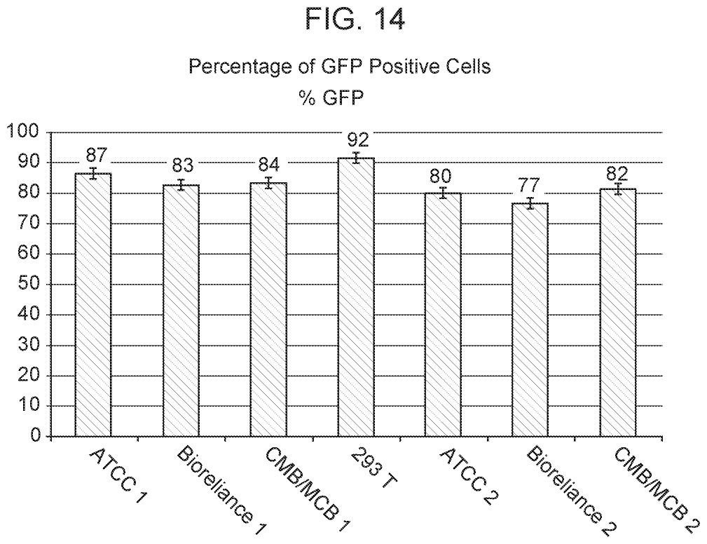

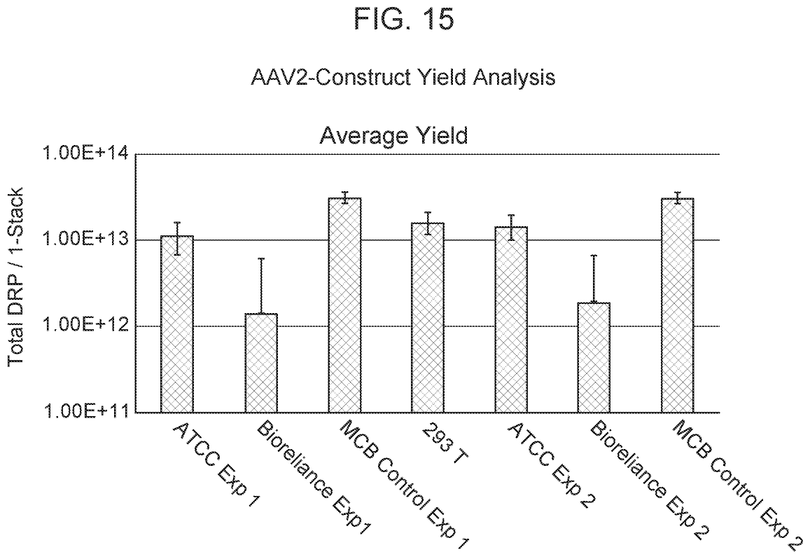

Percent GFP: The percentage of GFP positive cells as determined by Flow Cytometry is shown in FIGS. 13 and 14.

AAV2-Construct Yield Analysis: DRP titre assay results were generated for head to head comparative analysis of AAV2-Construct yield in the harvested cells+media for each treatment (1-Stack) per qPCR analysis.

The acceptance criterion for a cell line to be suitable for use in clinical manufacture was deemed to be a difference of no more than fourfold of the control (Original Process 1 cells) per treatment. Experiment 1 (Exp1) was carried out at cell passage 7 and Experiment 2 (Exp2) at passage 15.

TABLE 27
Results for AAV2-Construct Yield Analysis

| | | | | | | | Fold |
|---|---|---|---|---|---|---|---|
| | | | | | | | Difference |
| | Yield DRPs | | | | Avg | | from |
| Cell Line | Rep 1 | Rep 2 | Rep 3 | Avg Yield | DRP/cm² | Range | Control |
| ATCC Exp1 | 1.23 × 10\hat{ }13 | 1.17 × 10\hat{ }13 | 1.01 × 10\hat{ }13 | 1.14 × 10\hat{ }13 | 1.79 × 10\hat{ }10 | 2.28 × 10\hat{ }12 | 2.77 |
| Bioreliance | 1.70 × 10\hat{ }12 | 1.33 × 10\hat{ }12 | 1.21 × 10\hat{ }12 | 1.41 × 10\hat{ }12 | 2.22 × 10\hat{ }09 | 4.85 × 10\hat{ }11 | 22.27 |
| Exp1 | | | | | | | |
| MCB Control | 2.84 × 10\hat{ }13 | 2.60 × 10\hat{ }13 | 4.01 × 10\hat{ }13 | 3.15 × 10\hat{ }13 | 4.95 × 10\hat{ }10 | 1.41 × 10\hat{ }13 | N/A |
| Exp1 | | | | | | | |
| 293T | 1.96 × 10\hat{ }13 | 2.21 × 10\hat{ }13 | 7.29 × 10\hat{ }12 | 1.63 × 10\hat{ }13 | 2.56 × 10\hat{ }10 | 1.48 × 10\hat{ }13 | 1.93 |
| ATCC Exp2 | 1.50 × 10\hat{ }13 | 1.46 × 10\hat{ }13 | 1.40 × 10\hat{ }13 | 1.45 × 10\hat{ }13 | 2.29 × 10\hat{ }10 | 9.70 × 10\hat{ }11 | 2.16 |
| Bioreliance | 1.74 × 10\hat{ }12 | 1.98 × 10\hat{ }12 | 2.06 × 10\hat{ }12 | 1.93 × 10\hat{ }12 | 3.03 × 10\hat{ }9 | 3.12 × 10\hat{ }11 | 16.30 |
| Exp2 | | | | | | | |
| MCB Control | 3.99 × 10\hat{ }13 | 1.94 × 10\hat{ }13 | 3.49 × 10\hat{ }13 | 3.14 × 10\hat{ }13 | 4.94 × 10\hat{ }10 | 2.05 × 10\hat{ }13 | N/A |
| Exp 2 | | | | | | | |

Growth Kinetics: Growth kinetics were evaluated by recording cell counts and viability of T175 flasks, seeded at two densities simultaneously, on days 1, 2, 3, 4 & 7 post seeding. Each experiment consisted of two replicates on consecutive passages. Experiment 1 was carried out at passage 7 & repeated at passage 8 from thaw. Experiment 2 was carried out at passage 15 & repeated at passage 16 from thaw. The quantity and percentage of viable cells was determined by Trypan Blue staining.

TABLE 28
Results for Growth Kinetics
Cell Counts Experiment 1 Passage 7

| Days | | | | | | | | | | |
|---|---|---|---|---|---|---|---|---|---|---|
| Post | | | | | | | | | | |
| Seed | Day 1 | Day 2 | Day 3 | Day 4 | Day 7 | Day 1 | Day 2 | Day 3 | Day 4 | Day 7 |
| Seed | 2.50 × 10\hat{ }6 | | | | | 1.00 × 10\hat{ }7 | | | | |
| Density | | | | | | | | | | |
| ATCC | 4.20 × | 2.10 × | 1.49 × | 1.90 × | 3.80 × | 1.90 × | 3.30 × | 5.53 × | 1.60 × | 9.20 × |
| | 10\hat{ }5 | 10\hat{ }5 | 10\hat{ }6 | 10\hat{ }6 | 10\hat{ }6 | 10\hat{ }6 | 10\hat{ }6 | 10\hat{ }6 | 10\hat{ }6 | 10\hat{ }6 |
| MCB | 2.70 × | 2.70 × | 2.47 × | 3.08 × | 4.00 × | 3.10 × | 4.80 × | 4.35 × | 3.35 × | 8.70 × |
| | 10\hat{ }5 | 10\hat{ }5 | 10\hat{ }6 | 10\hat{ }6 | 10\hat{ }6 | 10\hat{ }6 | 10\hat{ }6 | 10\hat{ }6 | 10\hat{ }6 | 10\hat{ }6 |
| Bioreliance | 1.40 × | 1.10 × | 1.16 × | 2.68 × | 3.70 × | 1.30 × | 1.40 × | 5.40 × | 2.05 × | 6.70 × |
| | 10\hat{ }5 | 10\hat{ }5 | 10\hat{ }6 | 10\hat{ }6 | 10\hat{ }6 | 10\hat{ }6 | 10\hat{ }6 | 10\hat{ }6 | 10\hat{ }6 | 10\hat{ }6 |
| 293T | 5.00 × | 2.90 × | 3.52 × | 5.20 × | 6.10 × | 1.80 × | 3.60 × | 6.40 × | 9.70 × | 9.30 × |
| | 10\hat{ }5 | 10\hat{ }5 | 10\hat{ }6 | 10\hat{ }6 | 10\hat{ }6 | 10\hat{ }6 | 10\hat{ }6 | 10\hat{ }6 | 10\hat{ }6 | 10\hat{ }6 |

Cell Counts Experiment 1 Passage 8

| Days | | | | | | | | | | |
|---|---|---|---|---|---|---|---|---|---|---|
| Post | | | | | | | | | | |
| Seed | Day 1 | Day 2 | Day 3 | Day 4 | Day 7 | Day 1 | Day 2 | Day 3 | Day 4 | Day 7 |
| Seed | 2.50 × 10\hat{ }6 | | | | | 1.00 × 10\hat{ }7 | | | | |
| Density | | | | | | | | | | |
| ATCC | 9.50 × | 1.60 × | 1.60 × | 4.40 × | 2.10 × | 7.45 x | 4.60 × | 9.30 × | 1.30 × | 2.50 × |
| | 10\hat{ }4 | 10\hat{ }5 | 10\hat{ }5 | 10\hat{ }5 | 10\hat{ }6 | 10\hat{ }5 | 10\hat{ }5 | 10\hat{ }5 | 10\hat{ }6 | 10\hat{ }6 |
| MCB | 3.70 × | 9.30 × | 2.70 × | 4.60 × | 4.10 × | 1.90 × | 2.70 × | 5.10 × | 4.10 × | 4.70 × |
| | 10\hat{ }5 | 10\hat{ }5 | 10\hat{ }6 | 10\hat{ }6 | 10\hat{ }6 | 10\hat{ }6 | 10\hat{ }6 | 10\hat{ }6 | 10\hat{ }6 | 10\hat{ }6 |
| Bioreliance | 1.40 × | 1.20 × | 5.90 × | 2.20 × | 5.30 × | 7.60 × | 1.50 × | 3.40 × | 4.90 × | 6.00 × |
| | 10\hat{ }5 | 10\hat{ }5 | 10\hat{ }5 | 10\hat{ }6 | 10\hat{ }6 | 10\hat{ }6 | 10\hat{ }6 | 10\hat{ }6 | 10\hat{ }6 | 10\hat{ }6 |
| 293T | 3.60 × | 1.10 × | 3.00 × | 6.00 × | 7.30 × | 1.80 × | 3.40 × | 7.80 × | 1.10 × | 7.10 × |
| | 10\hat{ }5 | 10\hat{ }6 | 10\hat{ }6 | 10\hat{ }6 | 10\hat{ }6 | 10\hat{ }6 | 10\hat{ }6 | 10\hat{ }6 | 10\hat{ }7 | 10\hat{ }6 |

Cell Counts Experiment 2 Passage 15

| Days | | | | | | | | | | |
|---|---|---|---|---|---|---|---|---|---|---|
| Post | | | | | | | | | | |
| Seed | Day 1 | Day 2 | Day 3 | Day 4 | Day 7 | Day 1 | Day 2 | Day 3 | Day 4 | Day 7 |
| Seed | 2.50 × 10\hat{ }6 | | | | | 1.00 × 10\hat{ }7 | | | | |
| Density | | | | | | | | | | |
| ATCC | 2.90 × | 3.10 × | 2.10 × | 1.50 × | 4.30 × | 1.20 × | 2.80 × | 3.10 × | 6.50 × | 7.80 × |
| | 10\hat{ }5 | 10\hat{ }5 | 10\hat{ }5 | 10\hat{ }6 | 10\hat{ }6 | 10\hat{ }6 | 10\hat{ }6 | 10\hat{ }6 | 10\hat{ }6 | 10\hat{ }6 |
| MCB | 5.10 × | 7.00 × | 3.20 × | 4.40 × | 4.20 × | 1.70 × | 5.60 × | 3.60 × | 4.40 × | 5.40 × |
| | 10\hat{ }5 | 10\hat{ }5 | 10\hat{ }6 | 10\hat{ }6 | 10\hat{ }6 | 10\hat{ }6 | 10\hat{ }6 | 10\hat{ }6 | 10\hat{ }6 | 10\hat{ }6 |
| Bioreliance | 3.80 × | 3.50 × | 1.50 × | 2.10 × | 5.70 × | 1.30 × | 2.90 × | 5.50 × | 6.20 × | 8.20 × |
| | 10\hat{ }5 | 10\hat{ }5 | 10\hat{ }6 | 10\hat{ }6 | 10\hat{ }6 | 10\hat{ }6 | 10\hat{ }6 | 10\hat{ }6 | 10\hat{ }6 | 10\hat{ }6 |

Cell Counts Experiment 2 Passage 16

| Days | | | | | | | | | | |
|---|---|---|---|---|---|---|---|---|---|---|
| Post | | | | | | | | | | |
| Seed | Day 1 | Day 2 | Day 3 | Day 4 | Day 7 | Day 1 | Day 2 | Day 3 | Day 4 | Day 7 |
| Seed | 2.50 × 10\hat{ }6 | | | | | 1.00 × 10\hat{ }7 | | | | |
| Density | | | | | | | | | | |
| ATCC | 1.50 × | 1.20 × | 2.20 × | 4.00 × | 7.00 × | 1.10 × | 2.50 × | 5.60 × | 5.40 × | 6.80 × |
| | 10\hat{ }6 | 10\hat{ }6 | 10\hat{ }6 | 10\hat{ }6 | 10\hat{ }6 | 10\hat{ }6 | 10\hat{ }6 | 10\hat{ }6 | 10\hat{ }6 | 10\hat{ }6 |
| MCB | 4.80 × | 2.20 × | 3.40 × | 2.20 × | 4.90 × | 2.70 × | 8.80 × | 4.20 × | 3.30 × | 5.00 × |
| | 10\hat{ }5 | 10\hat{ }6 | 10\hat{ }6 | 10\hat{ }6 | 10\hat{ }6 | 10\hat{ }6 | 10\hat{ }6 | 10\hat{ }6 | 10\hat{ }6 | 10\hat{ }6 |
| Bioreliance | 1.40 × | 3.00 × | 2.00 × | 3.40 × | 8.90 × | 1.40 × | 2.80 × | 3.80 × | 7.20 × | 1.10 × |
| | 10\hat{ }5 | 10\hat{ }5 | 10\hat{ }6 | 10\hat{ }6 | 10\hat{ }6 | 10\hat{ }6 | 10\hat{ }6 | 10\hat{ }6 | 10\hat{ }6 | 10\hat{ }7 |

Percent Viability Experiment 1 Passage 7

| Days | | | | | | | | | | |
|---|---|---|---|---|---|---|---|---|---|---|
| Post | | | | | | | | | | |
| Seed | Day 1 | Day 2 | Day 3 | Day 4 | Day 7 | Day 1 | Day 2 | Day 3 | Day 4 | Day 7 |
| Seed | 2.50 × 10\hat{ }6 | | | | | 1.00 × 10\hat{ }7 | | | | |
| Density | | | | | | | | | | |
| ATCC | 94% | 100% | 98% | 100% | 99% | 87% | 99% | 98% | 99% | 99% |
| CMF/MCB | 96% | 95% | 99% | 89% | 99% | 97% | 100% | 97% | 86% | 100% |
| Bioreliance | 93% | 96% | 100% | 94% | 100% | 91% | 99% | 97% | 89% | 99% |
| 293T | 96% | 98% | 99% | 97% | 100% | 91% | 100% | 98% | 97% | 100% |

Percent Viability Experiment 1 Passage 8

| Days | | | | | | | | | | |
|---|---|---|---|---|---|---|---|---|---|---|
| Post | | | | | | | | | | |
| Seed | Day 1 | Day 2 | Day 3 | Day 4 | Day 7 | Day 1 | Day 2 | Day 3 | Day 4 | Day 7 |
| Seed | 2.50 × 10\hat{ }6 | | | | | 1.00 × 10\hat{ }7 | | | | |
| Density | | | | | | | | | | |
| ATCC | 95% | 94% | 100% | 95% | 94% | 99% | 97% | 95% | 93% | 94% |
| CMF/MCB | 100% | 98% | 92% | 96% | 96% | 100% | 100% | 96% | 92% | 94% |
| Bioreliance | 100% | 100% | 95% | 96% | 99% | 100% | 98% | 99% | 94% | 99% |
| 293T | 100% | 99% | 95% | 96% | 99% | 100% | 100% | 97% | 98% | 98% |

Percent Viability Experiment 2 Passage 15

| Days | | | | | | | | | | |
|---|---|---|---|---|---|---|---|---|---|---|
| Post | | | | | | | | | | |
| Seed | Day 1 | Day 2 | Day 3 | Day 4 | Day 7 | Day 1 | Day 2 | Day 3 | Day 4 | Day 7 |
| Seed | 2.50 × 10\hat{ }6 | | | | | 1.00 × 10\hat{ }7 | | | | |
| Density | | | | | | | | | | |
| ATCC | 95% | 98% | 73% | 91% | 87% | 98% | 98% | 67% | 89% | 85% |
| CMF/MCB | 96% | 100% | 90% | 86% | 82% | 93% | 96% | 55% | 83% | 90% |
| Bioreliance | 100% | 100% | 95% | 97% | 84% | 96% | 99% | 90% | 92% | 92% |

Percent Viability Experiment 2 Passage 16

| Days | | | | | | | | | | |
|---|---|---|---|---|---|---|---|---|---|---|
| Post | | | | | | | | | | |
| Seed | Day 1 | Day 2 | Day 3 | Day 4 | Day 7 | Day 1 | Day 2 | Day 3 | Day 4 | Day 7 |
| Seed | 2.50 × 10\hat{ }6 | | | | | 1.00 × 10\hat{ }7 | | | | |
| Density | | | | | | | | | | |
| ATCC | 98% | 81% | 90% | 96% | 82% | 89% | 90% | 97% | 87% | 76% |
| CMF/MCB | 96% | 83% | 93% | 96% | 85% | 94% | 83% | 92% | 78% | 81% |
| Bioreliance | 94% | 67% | 95% | 98% | 93% | 96% | 91% | 91% | 97% | 89% |

All four cell lines expressed GFP at levels consistent with those expected for good transduction efficiency based on previous experience relative to the correlating yield analysis. The two cell lines from ATCC, HEK293 (CRL-1573) and 293T (CRL-11268) exhibited good cell growth and expansion from thaw. It is notable that the Bioreliance cell line had a slower initial growth and expansion with poorer overall cell health relative to the other cell lines and control. The Bioreliance cell line also failed to meet the acceptance criteria of yielding within fourfold of the control for both experiments where both of the ATCC cell lines fell within the minimum acceptance criteria.

For further production of the AAV2-Construct vector, the ATCC cell line HEK293 (CRL-1573) was chosen based on the results presented above for exemplary embodiments of the manufacturing processes of the disclosure.

### Example 5: Analytical Development of Manufacturing Processes

For the data provided below, the measurements are taken from one or more batches of AAV2-Construct, wherein the Construct is identical in all batches.

As part of the strategy for transferring from the original to an improved manufacturing process, an analytical strategy was developed in parallel. Where possible, methods developed for the original process were transferred to the improved process or to next-generation process.

TABLE 29
Summary of Analytical Methods

| Tests | Original | Improved | Summary of Comparability |
|---|---|---|---|
| Performed | Process | Process | of Methods |
| General Tests | | | |
| Appearance | Color, appearance | Color, appearance and | Methods not significantly different |
| | and clarity. | clarity. EP/USP | |
| | EP/USP | compendial method | |
| | compendial | | |
| | method | | |
| pH | pH meter. | pH meter. EP/USP | Methods not significantly different |
| | EP/USP | compendial method | |
| | compendial | | |
| | method | | |
| Osmolality | Freezing point | Freezing point | Methods not significantly different |
| | depression. | depression. EP/USP | |
| | EP/USP | compendial method | |
| | compendial | | |
| | method | | |
| Content | | | |
| Physical Titre | qPCR using | qPCR using supercoiled | Methods not significantly different. |
| | supercoiled | plasmid vector used as | |
| | plasmid vector | standard. | |
| | used as standard. | | |
| Infectious Titre | Cell culture with | Cell culture with qPCR | Methods not significantly different. |
| | qPCR endpoint. | endpoint. | |
| Physical: Infectious | NA | NA | NA |
| Titre Ratio | | | |
| Total Particles | NA | Commercial ELISA | NA—not determined on clinical material |
| | | kit—Progen, Cat# PRATV | from original process |
| Full: Empty Ratio | NA | Transmission electron | NA—not determined on clinical material |
| | | microscopy | from original process |
| Identity | | | |
| Identity DNA | DNA sequencing | DNA sequencing | Methods not significantly different. |
| Potency | | | |
| Construct Protein | NA | Cell transduction with | NA—not determined on clinical material |
| Expression and | | Construct expression | from original process |
| Activity | | and substrate activity | |
| | | measured by Western | |
| | | Blot. ELISAs for | |
| | | Construct and the | |
| | | substrate. | |
| Purity and Impurities | | | |
| Total Protein | Micro-BCA | Micro-BCA | Matrix effect observed in purified bulk |
| | assay—everything | assay—following protein | buffer and final product buffer during |
| | quantified against | precipitation to remove | validation of the improved process. Protein |
| | BSA calibration in | matrix effect. | precipitation (acetone) step introduced into |
| | PBS. | | the improved assay to address the matrix |
| | | | effect |
| Purity | SDS-PAGE | SDS-PAGE | Methods not significantly different. Gel |
| | | | loaded by total particles as measured by a |
| | | | validated ELISA method instead of total |
| | | | particles as measured by non-validated |
| | | | Micro-BCA to improve quantitation. |
| rcAAV | qPCR | qPCR | Methods not significantly different. |
| Host Cell Protein | Commercial | Commercial ELISA | Methods not significantly different. |
| | ELISA | kit—Cygnus Cat # F650 | |
| | kit—Cygnus Cat # | | |
| | F650 | | |
| Total DNA | N/A | Pico green assay | NA—not determined on clinical material |
| | | | from original process |
| Host Cell DNA | Custom Method | Life Technologies assay | Measured size and quantity of 3 different |
| | | kit | amplicons whereas the improved process |
| | | | measures total hcDNA including high |
| | | | molecular weight and sheared DNA. The |
| | | | qualification data from the improved |
| | | | process demonstrates that the assay is |
| | | | specific and sufficiently sensitive to meet |
| | | | the requirements in assessing hcDNA per |
| | | | dose of <10 ng/dose. |
| Residual BSA | Commercial ELISA | Commercial ELISA | Methods not significantly different. Both |
| | kit—Bethy1 | kit—Alpha Diagnostic, Cat | kits have an appropriate level of sensitivity |
| | Laboratories, Inc., | # 8100 | and specificity to meet product |
| | Cat # E10-113 | | specifications |
| Residual Benzonase | Commercial | Commercial ELISA | Methods not significantly different. |
| | ELISA kit—EMD | kit—Merck, Cat # | |
| | Millipore (Merck), | 1.01681.0001 | |
| | Cat. # | | |
| | 1.01681.0001 | | |
| Residual AVB | NA | Commercial ELISA kit, | New IPC added. NA—not determined on |
| | | Life Technologies | clinical material from original process |
| Safety | | | |
| Sterility | EP/USP | EP/USP compendial | Methods not significantly different. |
| | compendial | method | |
| | method | | |
| Bioburden | EP/USP | EP/USP compendial | Methods not significantly different. |
| | compendial | method | |
| | method | | |
| Endotoxin | EP/USP | EP/USP compendial | Methods not significantly different. |
| | compendial | method | |
| | method | | |

NA = Not applicable

Both Original and Improved rcAAV assays utilize human embryonic kidney cells (HEK293) that are infected with dilutions of the AAV2-construct or wild type (wt) AAV as a control. The cells are then co-infected with adenovirus serotype 5 (Ad5) to provide helper functions for rcAAV (if present). At maximum cytopathic effect (CPE) the cells are lysed. This constitutes the end of passage 0 (P0) of the vector. In passage 1 (amplification 1), a portion of the P0 lysates are used to inoculate HEK293 cells co-infected with and without Ad5. The cells without Ad5 serve to determine the input level of DNA into the cells while the cells with Ad5 permit amplification of the rcAAV2-Construct, or wt AAV in the case of the controls. In the Original assay, when the Ad5 positive samples reach maximum CPE after the first amplification the cells are analyzed by PCR. In the Improved assay, the cell lysis/amplification step is performed a total of 3 times prior to PCR analysis.

The assay has been validated as a limit test of the improved process. Neither the Original clinical nor the engineering batch were analyzed as part of this validation so a direct comparison of the results obtained is not possible. The Original assay was not validated. In conclusion, the two methods provide results that are not significantly different.

The hcDNA measurements from the Original and Improved process both use qPCR on the Taqman instrument. The Original assay measured three different amplicons (102, 401 and 765 bp) with a sensitivity (LOD) of 20 pg/mL for each amplicon. The assay used by the improved process has a single amplicon and a sensitivity (LOQ) of 1.3156 ng/mL. A head-to-head comparison of the methods was not conducted, however, the hcDNA method has subsequently been qualified and demonstrated fit for purpose. Thus, there is not an adverse impact on the comparability assessment.

Residual BSA was measured by the Original Process using a commercially available ELISA kit from Bethyl Laboratories, Cat #E10-113. The sensitivity of this kit, used in the Original process, was 12.6 ng/mL. A commercially available ELISA kit, Alpha Diagnostics Cat #8100, is used in the improved process. This kit has not been validated but the sensitivity is defined as the lowest calibration standard i.e. 1 ng/mL. The manufacturers of the primary antibodies used in both kits assert that the primary antibodies are specific for the detection of human BSA. A head-to-head comparison of the methods was not conducted; however, all batches tested to data have demonstrated very low levels of residual BSA (below the sensitivity of the method).

Therefore, it was concluded that all batches contain comparably low levels of residual BSA.

In addition, because BSA can cause allergic reactions in humans the World Health Organization (WHO) has set a guidance of 50 ng or less residual BSA per vaccine dose. For example, for a dose of AAV2-Construct of 100 μL, the concentration limit of BSA would be 500 ng/mL. Both ELISA kit assays used in the Original and Improved processes have levels of sensitivity below this required limit and are therefore considered comparable in measuring residual BSA within acceptable limits.

Infectious Titre

The infectious titre assay was transferred from the Original to the improved process. Both the cell culture part of the assay and the PCR were performed as part of this transfer following the procedure used in the Original Process. AAV2-Construct (non-GMP) (by Original process) was analyzed on 3 occasions during the assay transfer and the results obtained were compared with the infectious titre, 2.90×10¹⁰ IU/mL. The data obtained are provided in Table 30.

TABLE 30
Infectious Titre Assay

| Sample | Result (IU/mL) | Pass/Fail |
|---|---|---|
| Occasion 1 | 4.31 × 10\hat{ }10 | Pass |
| Occasion 2 | 9.28 × 10\hat{ }10 | Pass |
| Occasion 3 | 3.56 × 10\hat{ }10 | Pass |
| Mean | 5.72 × 10\hat{ }10 | — |
| Std. dev. | 3.11 × 10\hat{ }10 | — |
| Precision (% CV) | 54.4 | — |

The data obtained from the assay transfer study are not significantly different, i.e. within a 4-fold range of the result obtained from the Original process.

The AAV-Construct (non-GMP) produced by the Original process was analyzed on 3 occasions according to the Improved process and the results are provided in Table 31 relative to the reported titre (4.95×10\hat{ }12 DRP/mL) (Original Process). Data was considered acceptable if it was within a 3-fold range of the result reported according to the Original process.

TABLE 31
Physical Titre Assay − Comparability Data

| Sample | Result (IU/mL) | Pass/Fail |
|---|---|---|
| Occasion 1 | 9.57 × 10\hat{ }12 | Pass |
| Occasion 2 | 7.99 × 10\hat{ }12 | Pass |
| Occasion 3 | 7.75 × 10\hat{ }12 | Pass |
| Mean | 8.44 × 10\hat{ }12 | — |
| Std. dev. | 9.888 × 10\hat{ }11 | — |
| Precision (% CV) | 10.3 | — |

The assay has subsequently been validated and the additional values in Table 32 were obtained for the AAV-Construct (non-GMP) (from Original process) material (1000 and 10,000 dilution results only).

TABLE 32
Physical Titre Results (Post-Assay Validation) for
AAV-Construct (Non -GMP) (Original Process)

| Validation Assay | Result (IU/mL) | Pass/Fail |
|---|---|---|
| 2a | 6.11 × 10\hat{ }12 | Pass |
| 2b | 1.50 × 10\hat{ }13 | Pass |
| 7 | 1.72 × 10\hat{ }12 | Pass |
| 10 | 6.93 × 10\hat{ }12 | Pass |
| | 4.60 × 10\hat{ }12 | |
| | 4.98 × 10\hat{ }12 | |
| 11 | 3.57 × 10\hat{ }12 | Pass |
| | 3.47 × 10\hat{ }12 | |
| | 3.15 × 10\hat{ }12 | |
| 12 | 7.99 × 10\hat{ }12 | Pass |
| | 8.55 × 10\hat{ }12 | |
| | 8.66 × 10\hat{ }12 | |
| Mean | 6.23 × 10\hat{ }12 | — |
| Std. dev. | 3.574 × 10\hat{ }12 | — |
| Precision (% CV) | 57.4 | — |

The AAV2-Construct (non-GMP) material (from Original process) is routinely analyzed in DS and DP sample analysis as an internal reference control (primary reference) with system suitability acceptance criteria set as within 3-fold difference from the nominal titre (4.95×10\hat{ }12 DRP/mL) (determined by Original process) to ensure continued comparable performance of the method.

### Example 6: Comparison of Compositions Generated by Improved Process

For the data provided below, the measurements are taken from one or more batches of AAV2-Construct, wherein the Construct is identical in all batches.

To demonstrate that the change process changes/optimizations (to Process 1 to generate Process 2) have not affected the critical quality attributes of the vector material produced, a Comparability Assessment was performed.

Once the process changes were made to the cells, plasmids, transfection conditions, affinity chromatography column, scale and final container, and the optimization work had been completed with respect to Process 1, a new process for manufacture of Clinical Phase 3 material was defined and a full scale ‘engineering’ batch performed to measure process performance and equivalence of Drug Substance and Drug Product to the material produced by the Original process.

The analyses below compare the data generated from the full scale engineering batch and GMP batches from the improved process to the engineering batch and clinical material generated by the Original process.

An engineering batch produced by the Original process (AAV2-Construct (non-GMP)), for which additional testing was performed (see Table 33 below) to provide information for comparison to the engineering batch produced by the Improved process.

TABLE 33
Comparison of AAV2-Construct (non-GMP) Batches produced by Original
and Improved Processes

| | | Improved | Improved | | |
|---|---|---|---|---|---|
| | | Process: | Process: | Original | |
| | Improved | GMP lot | GMP lot | Process: | |
| | Process: | G214/ | G214/ | Clinical | |
| | AAV2-Construct | (AAV2- | (AAV2- | Lot | Original Process: |
| | (non- | Construct) | Construct) | (AAV2- | AAV2-Construct |
| Tests Performed | GMP) | batch 1 | batch 2 | Construct) | (non-GMP) |
| Bioburden | TAMC 0 | 0 CFU/ | 0 CFU/ | TAMC <8.0 | TAMC <2.01 |
| | CFU/mL* | 0.410 mL | 0.552 mL | CFU/mL* | CFU/mL |
| | TYMC 0 | (formulated | (formulated | TYMC <8.0 | TYMC <2.01 |
| | CFU/mL* | bulk pre- | bulk pre- | CFU/mL* | CFU/mL |
| | | filtration) | filtration) | | |
| Endotoxin | 0.79 EU/mL | <1 EU/mL | <1 EU/mL | <0.054 EU/mL | <0.050 EU/mL |
| Physical Titre*** | 1.86 × | 1.06 × | 9.57 × | 8.71 × 10\hat{ }11 | 4.95 × 10\hat{ }12 |
| | 10\hat{ }11 | 10\hat{ }12 | 10\hat{ }11 | DRP/mL | DRP/mL |
| | DRP/mL [symbol] | DRP/mL | DRP/mL | | |
| Infectious Titre | 2.00 × 10\hat{ }9 | 4.3 × 10\hat{ }9 | 5.2 × 10\hat{ }9 | 2.4 × 10'10 | 2.9 × 10\hat{ }10 IU/mL |
| | IU/mL | IU/mL | IU/mL | IU/mL | |
| Physical: Infectious | 93:1 | 247:1 | 184:1 | 36:1 | 171:1 |
| Titre Ratio | | | | | |
| Total Particles | 2.7 × 10\hat{ }11 | 1.9 × 10\hat{ }12* | 1.9 × 10\hat{ }12* | ND | ND |
| | capsids/mL | capsids/mL | capsids/mL | | |
| Full: Empty Ratio | 5.7:1* | 0.96:1* | 8.1:1* | 9:1** | 25:1 [symbol] [symbol] |
| Total Protein | <LOQ | 22 μg/mL* | 16 μg/mL* | 28.979 μg/mL | 29.434 μg/mL |
| Purity | No | Purity = 100% | Purity = 100% | Purity = 100% | Purity = 93.238% |
| | detectable | No | No | No detectable | Impurity 1: 52.33 kDa |
| | impurities | detectable | detectable | impurities | and 1.656% |
| | (Sample | impurities | impurities | | Impurity 2: 47.05 kDa |
| | presents the | | | | and 1.805% |
| | specific | | | | Impurity 3: 45.01 kDa |
| | bands of | | | | and <1% |
| | VP1, VP2 | | | | Impurity 4: 35.14 kDa |
| | and VP3 | | | | and 1.693% |
| | proteins) | | | | |
| rcAAV | <10 IU of | <LOD of | <10 IU of | <10 IU of | No detectable rcAAV |
| | rcAAV2 in | Assay | rcAAV in | rcAAV in 1.0 × | in 1 × 10 DRP with |
| | 1 × 10\hat{ }10 | | 1.0 × 10\hat{ }10 | 10\hat{ }11 DRP | LOD of 3.3 IU |
| | DRP**** | | DRP | | |
| Identity DNA | Sequence | Matches | DNA sequence | DNA sequence | DNA sequence |
| | of cDNA | Expected | matches the | available | available matches the |
| | encoding | sequence | correct | matches the | correct sequence (full |
| | Construct | | sequence | correct | sequence not |
| | matches the | | | sequence (full | obtained) |
| | expected | | | sequence not | |
| | sequence | | | obtained) | |
| | (full | | | | |
| | sequence | | | | |
| | not | | | | |
| | available)* | | | | |
| Host cell protein | <4 ng/mL | <LOD | <LOD (<10 | <8 ng/mL | <8 ng/mL |
| | | <4 ng/mL | ng/mL*) | | |
| Host cell DNA | 0.53 ng/mL | <6.58 × | <6.58 × | 102 bp: 4.41 × | 102 bp: 1612 pg/1 × |
| | | 10\hat{ }3 | 10\hat{ }3 | 10\hat{ }4 pg/mL | 10\hat{ }9 DRP |
| | | Pg/mL* | pg/mL* | 401 bp: 3.79 × | 401 bp: 1204 pg/1 × |
| | | | | 10\hat{ }4 pg/mL | 10\hat{ }9 DRP |
| | | | | 765 bp: 2.74 × | 765 bp: 832 pg/1 × |
| | | | | 10\hat{ }4 pg/mL | 10\hat{ }9 DRP |
| Host cell DNA | 530 pg/mL | <LOQ | <LOQ | 102 bp: 4.41 × | 102 bp: 7.98 × 10\hat{ }6 |
| (normalized units | | | | 10\hat{ }4 pg/mL | pg/mL |
| to pg/mL) | | (<6.58 × | (<6.58 × | 401 bp: 3.79 × | 401 bp: 5.96 × 10\hat{ }6 |
| | | 10\hat{ }3 | 10\hat{ }3 | 10\hat{ }4 pg/mL | pg/mL |
| | | pg/mL)* | pg/mL)* | 765 bp: 2.74 × | 765 bp: 4.12 × 10\hat{ }6 |
| | | | | 10\hat{ }4 pg/mL | pg/mL |
| Residual BSA | 4 ng/mL | <LOD | <LOD | <12.6 ng/mL | <12.6 ng/mL |
| | | (<1 ng/m)L)* | (<1 ng/mL)* | | |
| Residual | <LOD | | | | |
| benzonase | <0.2 ng/mL | (<4 ng/mL)* | <0.4 ng/mL* | <0.5 ng/mL | <0.5 ng/mL |
| Osmolality | 408 | 402 | 408 | 400 mOsm/kg | 408 mOsm/kg H₂O |
| | mOsm/kg | mOsm/kg | mOsm/kg | H₂O | |
| | H₂O [symbol] | H₂O | H₂O | | |
| pH | 8.0 [symbol] | pH 8.0 | pH 7.9 | pH 7.9 | pH 8.0 |
| Appearance | Clear | Clear | Clear | Clear, | Clear, |
| | colorless | colorless | colorless | colorless | colorless liquid |
| | liquid | liquid | liquid | liquid free | free from visible |
| | without | | without | from visible | particulates |
| | visible | | visible | particulates | |
| | particulates | | particulates | | |
| Construct protein | Construct | Construct | Construct | Construct | Construct protein |
| expression | protein | protein | protein | protein | expression detected** |
| | expression | expression | expression | expression | |
| | detected [symbol] ** | detected** | detected** | detected** | |
| Residual AVB | n/a | n/a | <0.088 | n/a | n/a |
| ligand | | | ng/mL | | |
| | | | (LOD)* | | |

[symbol]

Testing performed on Drug Product (final container) [symbol] [symbol]
Testing performed by analytical ultracentrifugation, not electron microscopy
*Testing performed on purified bulk (Drug Substance)
** Testing performed using non GMP analysis
***Physical Titre reported was performed according to Original process
****Analyzed in validation study according to improved process

Additional Analytical Information and Data

Physical Titre: The target physical titre for the filled drug product clinical material (GMP product) was 1×10\hat{ }12 DRP/mL (produced by Original process). For the engineering batch manufactured by the Original process (non-GMP product), there was no target titre and AAV2-Construct (non-GMP) represented Drug Substance that would be diluted to the target fill concentration had this batch been progressed to Drug Product. For AAV2-Construct (non-GMP) produced by the Improved process, the target concentration was 1-2×10\hat{ }12 DRP/mL. The reported titre of 1.87×10\hat{ }11 DRP/mL for AAV2-Construct (non-GMP) is lower than expected, but this is due to the minimum hold up volume in the final tangential flow ultrafiltration step (TFF2) limiting the extent to which the final Drug Substance could be concentrated. Specifically, this incomplete concentration resulted in the batch being 6-fold less concentrated. If the physical and infectious titre is corrected for this 6-fold dilution then the physical titre, infectious titre and physical:infectious titre ratios are similar between the batches, as summarized in Table 34. The use of a lower hold-up volume (smaller) TFF cartridge has been evaluated to achieve a higher concentration factor.

TABLE 34
Comparison of Physical Titre, Infectious Titre and Physical:Infectious Titre
Ratio

| | Improved Process: | | | | | |
|---|---|---|---|---|---|---|
| | AAV2-Construct | | Improved | | | |
| | (non-GMP)—batch 2 | | Process: | | Original | Original |
| | | Result | AAV2- | Improved | Process: | Process: |
| | | corrected | Construct | Process: | Clinical | AAV2- |
| | | for 6- | (non- | GMP Lot | Lot | Construct |
| | Uncorrected | fold | GMP)- | (AAV2- | (AAV2- | (non- |
| Tests Performed | result | dilution | batch 1 | Construct) | Construct) | GMP) |
| Physical Titre | 1.86 × 10\hat{ }11 | 1.11 × 10\hat{ }12 | 1.06 × 10\hat{ }12 | 9.57 × 10\hat{ }11 | 8.71 × 10\hat{ }11 | 4.95 × 10\hat{ }12 |
| | DRP/mL | DRP/mL | DRP/mL | DRP/mL | DRP/mL | DRP/mL |
| Infectious Titre | 2.00 × 10\hat{ }9 | 1.2 x 10\hat{ }10 | 4.3 × 10\hat{ }9 | 5.2 × 10\hat{ }9 | 2.4 × 10\hat{ }10 | 2.9 × 10\hat{ }10 |
| | IU/mL | IU/mL | IU/mL | IU/mL | IU/mL | IU/mL |
| Physical:Infectious | 93:1 | 93:1 | 247:1 | 184:1 | 36:1 | 171:1 |
| Titre Ratio | | | | | | |

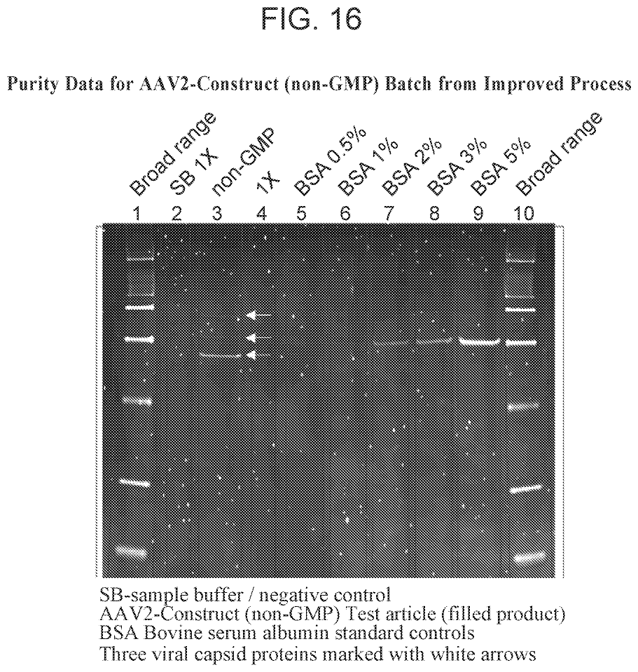

Purity: Purity is measured by analysis with Sypro Orange stained, reduced SDS polyacrylamide gel electrophoresis. FIG. 16 shows the purity profile generated for the AAV2-Construct (non-GMP) batch produced by the improved process. Data shows presence of the 3 viral capsid (VP1, VP2 and VP3) proteins with no additional impurities detected.

Construct Protein Expression: Prior to the introduction of a quantitative assay for Construct protein expression (via an ELISA method), preliminary analysis was performed using a qualitative non-GMP assay. This cell based, in vitro assay, described below, is capable of measuring both Construct expression and biological activity of the expressed Construct in a human cell line. The assay has 3 components:

Cell culture and viral vector transduction

Western blot for the detection of Construct protein expression

Assay for the detection of Construct protein activity

Cell culture is performed using HEK293 cells which are transduced with a known and comparable physical titre of AAV2-Construct vector. These cells express Construct protein at a level below or at the limit of that detected within this assay, allowing for a comparative qualitative assessment of increased Construct protein expression due to cell transduction and expression from the AAV2-Construct vector. Following transduction and incubation, cell lysates are collected and the cytosolic fraction is used in Western blot and in an in vitro assay to determine Construct protein expression and activity, respectively. Protein detection is performed using a monoclonal antibody specific to the Construct protein and expression levels are normalized against, for example, intracellular B-actin expression to provide a semi-quantitative analysis.

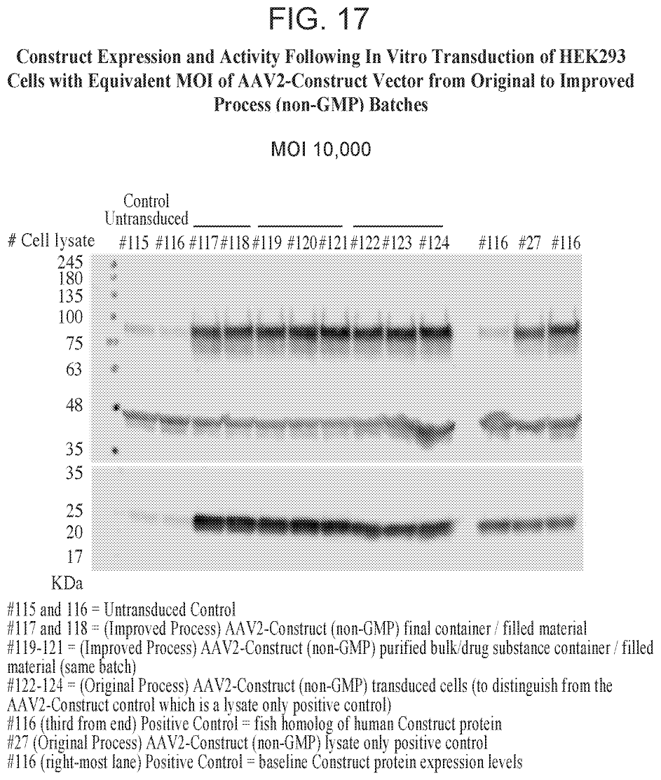

FIG. 17 shows the data illustrating the results for Construct expression and functional activity following transduction of cultured HEK293 cells with the engineering lots from the Original and Improved processes. Staining for the Construct protein is positive for both lots, and indicates increased levels of expression of the Construct protein compared to baseline Construct protein expression levels seen in untransduced control 293 cells. Detection of a substrate of the Construct protein is also positive for both lots and indicates increased levels of activity with the substrate compared to baseline levels seen in untransduced control 293 cells. Furthermore, levels of Construct expression and substrate activity appear comparable for both Original and improved engineering runs demonstrating comparable potency for these batches. Data from GMP batches also shows increase in Construct expression and activity.

Product of Optimized Process:

Safety: Bioburden and endotoxin data indicated no issues with safety relating to introduction of new contaminants. The batches from Original and Improved processes are not significantly different in this regard.

Impurities: Levels of process related impurities (HCP, residual benzonase, BSA, Total Protein) were not significantly different for all batches.

Some differences in host cell DNA (hcDNA) levels are noted. The difference between the AAV2-Construct (non-GMP) and AAV2-Construct (GMP), both produced by the Original process may be due to the fact that AAV2-Construct (non-GMP) was produced in non-GMP laboratories whereas AAV2-Construct (GMP) was produced in GMP clean rooms where there is more control of process steps, process operations, sampling, testing and raw materials, allowing better and more reproducible results to be obtained. In addition, a longer incubation time following plasmid transduction of cells was used to produce AAV2-Construct (non-GMP); 72 hours from cell feeding until harvest compared to 23 hours for the cells used to produce AAV2-Construct (GMP). The longer incubation can allow more cell growth and more hcDNA to be released by those cells.

These differences can account for the differences observed in hcDNA levels between the 2 lots of material produced by the Original process.

Conversely, for the AAV2-Construct (non-GMP) and the 2 GMP lots, all of which were produced by the Improved process, the hcDNA levels are much lower (530 pg/mL or <LOD respectively). In the process used to produce these batches there is a chromatography purification and polishing step which is capable of removing residual DNA.

Product Characteristics: Appearance, pH and osmolality were not significantly different for all batches.

Vector Characteristics: The non-GMP and GMP batches produced by the improved process show comparable Construct expression and biological activity when assessed in a non-quantitative assay. These batches also had comparable purity to batches generated by the Original process with no detectable impurities.

Data on the ratio of full to empty vector capsids are similar, and data from the Construct expression and activity presented above confirm that the ratio of full to empty vector capsids in the (non-GMP) batch produced by the Improved process is equivalent to GMP batches produced by the Original process and does not negatively impact the ability of vector to transduce human cells and demonstrate biological activity.

Data on the identity (vector sequence) confirms homology across the full sequence for all batches with no mutations introduced. Identity is also confirmed by ability to detect in vitro Construct expression and biological activity, both of which rely on an intact and functional Construct protein flanked by AAV ITRs.

Replication competent AAV has not been detected in any batch. Presence of an rcAAV is unlikely due to the nature of the plasmid and vector genetics but will continue to be assessed for any GMP batch prior to release for clinical studies to assure patient safety.

Process Yield:

Table 35 summarizes the overall process yield of the Improved process. From twice the number of 10 stack cell factories (24 c.f. 12 for the Original process), twice the number of virus particles (DRP) are made (6.7×10\hat{ }13 c.f. 3.4×10\hat{ }13 for the Original process). Overall process recovery is lower but this is partly due to the additional in-process and quality control testing performed during the improved process development that was not included during the Original batch production.

TABLE 35
Relative Process Yields and Recovery

| | Original process: | Improved Process: |
|---|---|---|
| Manufacturer | AAV2- | AAV2- |
| and Batch | Construct (Clinical) | Construct (non-Clinical) |
| Virus Produced | 3.4 × 10\hat{ }13 | 6.7 × 10\hat{ }13 |
| (Physical Titre Total)* | DRP total | DRP total |
| Scale of Manufacture | 12 Cell factories | 24 Cell factories |
| Overall Process Yield | 28% yield | 14% yield** |

*Physical Titre reported was performed using the Original process
**~10% is lost in samples (IPC's, QC)

Based on the data presented above the AAV2-Construct non-clinical batch produced from the new optimized process is considered comparable to the non-clinical and clinical batches generated by the Original process. This process was used to produce the first GMP batch according to the improved process.

### Example 7: Improved Robustness of the AAV2 Purification Chromatography

All testing parameters relating to product quality and efficacy were assessed following introduction of AVB and there was no detectable change in the quality or efficacy profile of the AVB chromatography derived product, as detailed in Table 36 to Table 41.

In addition, a new method for the detection of residual AVB ligand was introduced and demonstrated no detectable AVB ligand in any of the material manufactured using the AVB Sepharose column.

Seven AVB engineering runs performed with AAV2-Construct harvest material (FIG. 2, Step 13) to evaluate aggregation prevention and step yield, 5 runs at small scale (1.5 to 7 L) and 2 runs at larger scale (16 L). The AVB step performed well in all experiments with good step recovery of 40-78% yield (and with an average yield of 64%). No significant aggregation was detected in any experiments. Following AVB chromatography levels of potential impurities or process contaminants were all low and in-line with levels previously demonstrated and the final vector product showed good potency and infectious titres. In conclusion from the scaled-down runs with the AVB Sepharose purification step, all assay results (Table 36) show good comparability to Original Phase 1/2 process (employing the animal-derived matrix filled affinity chromatography) and the initial “Improved” process for AAV2-Construct GMP clinical material (UnoQ anion exchange process).

TABLE 36
AVB Sepharose Purification Step—Assay Results

| AVB Experiment | | | | | | | |
|---|---|---|---|---|---|---|---|
| ID Code | 10a | 10b | 11 | 12 | 13 | 14 | 15 |
| Physical Titre | 1.4 × 10\hat{ }12 | 1.6 × 10\hat{ }12 | 1.87 × 10\hat{ }12 | 1 × 10\hat{ }12 | 0.7 × 10\hat{ }12 | 0.8 × 10\hat{ }12 | 2.2 × 10\hat{ }12 |
| (DRP/mL) | | | | (2.0 × 10\hat{ }12 | (1.3 × 10\hat{ }12 | (1.6 × 10\hat{ }12 | |
| | | | | at scale)* | at scale)* | at scale)* | |
| Global Process | 12% | 13% | 11% | 8% | 6.5%** | 9% | 6% |
| Recovery (%) | | | | | | | |
| Capsid Titre | 9.7 × 10\hat{ }11 | 1.1 × 10\hat{ }12 | 1.4 × 10\hat{ }12 | 4.3 × 10\hat{ }11 | 2.8 × 10\hat{ }11 | 3.9 × 10\hat{ }11 | 1.1 × 10\hat{ }12 |
| (cap/mL) | | | | | | | |
| Residual HCP | All results below the assay limit of quantitation (<LOQ) | | | | | | |
| (ng/mL) | | | | | | | |
| Residual AVB | | | | | | | |
| ligand (ng/mL) | | | | | | | |
| DLS*** (% volume) | 98.1 | 99.2 | 96.1 | 99.2 | 99.2 | 98.6 | 99.4 |

*Titre equivalent at full scale
**Yield corrected for loss of one centrifuge tube
***R&D method to assess aggregation

TABLE 37
AVB Sepharose Purification Step-Assay Results

| AVB | | | | | |
|---|---|---|---|---|---|
| Experiment | Physical | Potency (Fold Increase vs | | | |
| ID | Titre | Non-Transduced Cells) | | Infectious | Full Capsids |
| Code | (DRP/Ml) | Construct | Substrate | Titre | (%) |
| Exp11 | 1.87 × 10\hat{ }12 | 5.0 | 4.3 | 2.4 × 10\hat{ }10 | 82% full |
| Exp12 | 1.0 × 10\hat{ }12 | 4.8 | 16.9 | Not tested | 87% full |
| Exp13 | 0.7 × 10\hat{ }12* | 3.4 | 9.3 | Not tested | 78% full |
| Exp14 | 0.8 × 10\hat{ }12 | 3.1 | 11.5 | 7.7 × 10\hat{ }9 | No data |
| Exp15 | 2.2 × 10\hat{ }12 | 4.7 | 15.4 | 8.4 × 10\hat{ }9 | 70% full |
| Specification | >0.7 × 10\hat{ }12 | >2-fold | >2-fold | >1.4 × 10\hat{ }8 | <55% empty |
| | | increase | increase | | (>45% full |
| | | compared to | compared to | | capsids) |
| | | non-transduced | non-transduced | | |

*sample volume did not allow concentration to final titre.

Following demonstration of product comparability and improved process robustness by small scale development runs and scaled up production lots using the AVB affinity chromatography step, AAV2-Construct GMP was manufactured using the AVB process. All analytical test results for this GMP batch have met specification and expectation.

The preliminary analytical results for AAV2-Construct GMP drug substance and drug product (produced by the improved process) are summarized below in Table 38 and Table 39. All tests meet specification and expectation.

TABLE 38
Preliminary Release Test Results for AAV2-Construct
GMP (from Improved process)

| Test(s) | Specifications | Result | Conclusion |
|---|---|---|---|
| HC DNA | <2.66 × 10\hat{ }7 pg/mL | <6.58 × 10\hat{ }3 pg/mL | Pass |
| Safety, Bacterial | <5 IU/mL | <1 IU/mL | Pass |
| Endotoxins | | (<0.05 IU/mL) | |
| Physical Titre | >0.7 × 10\hat{ }12 DRP/mL | 4.30 × 10'12 | Pass |
| | | DRP/mL | |
| Infectious Titre | >1.24 × 10\hat{ }10 IU/mL | 1.1 × 10\hat{ }10 IU/mL | Pass |
| DRP: IU Ratio- | <5645:1 | 391 | Pass |
| Calculation | | | |
| Purity by | Purity >90% with no | 100% | Pass |
| SD S-PAGE | single impurity >2%. | | |
| | Three bands | | |
| | corresponding to AAV2 | | |
| | VP1, VP2 and VP3 | | |
| | should be evident. | | |
| AAV Identity | Matches the Expected | Matches the | Pass |
| DNA | Sequence with the | Expected Sequence | |
| | exception of the | with the exception | |
| | ITR regions | of the ITR regions | |
| rcAAV | < LOD of assay | < LOD of assay | Pass |
| | | (<10 rcAAV per 1 × | |
| | | 10\hat{ }10 vg) | |

TABLE 39
Preliminary Characterization Test Results forAAV2-
Construct GMP Drug Substance from Improved process
Characterization Testing

| Test(s) | Result |
|---|---|
| Total Protein | 16 μg/mL |
| HCP | <LOQ = <10 ng/mL |
| Residual BSA | <1 ng/mL |
| Residual Benzonase | <0.4 ng/mL |
| Residual AVB Ligand | <0.088 ng/mL (LOD) |
| Full/Empty Particles Ratio | 89:11 |
| Total Particles | 1.9 x 10\hat{ }12 particles/mL |
| Total DNA | 328 ng/mL |

The purification process improvement has resulted in comparable AAV2-Construct drug substance and improved process robustness when compared to the process and product from the Original process.

During manufacture by the improved process, the ratio of full to empty particles is determined as a characterization test.

Potential product-related impurities are given in Table 40.

TABLE 40

| Potential Product | |
|---|---|
| Related Impurities | Control |
| Replication competent | Replication competent |
| AAV | AAV Assay* |
| Empty AAV particle | Majority removed by |
| | gradient purification |

*Drug Substance release testing.

Potential process-related impurities and their control are given in Table 41.

TABLE 41

| | Control (Drug Substance |
|---|---|
| Potential Process Related Impurities | Release Testing) |
| Benzonase | Residual Benzonase ELISA |
| Endotoxin | Endotoxin Assay-Kinetic LAL |
| Bovine Serum Albumin | Residual BSA ELISA |
| Host Cell Protein | HEK293 Host Cell Protein ELISA |
| Host Cell DNA | HEK293 Host Cell DNA qPCR |
| Total DNA | Picogreen residual double |
| | stranded DNA analysis |
| AVB Ligand | Residual AVB ELISA |

### Example 8: Control of Drug Substance

For the data provided below, the measurements are taken from one or more batches of AAV2-Construct, wherein the Construct is identical in all batches.

Validation of Analytical Procedures: Summaries of method validation results, where available, are presented below.

Physical Titre: This assay has been validated to fulfil the following criteria (see Table 42). The “Actual Value” is a value for an exemplary AAV2-Construct of the disclosure.

TABLE 42

| Test Parameter | Acceptance Criteria | Actual Value | Pass or Fail |
|---|---|---|---|
| Precision | The % CV using replicate C_{T} values | <3.87% | Pass |
| | of each calibration curve dilution is ≤5% | | |
| | for both inter and intra assay precision | | |
| | (inter-assay precision value detailed | | |
| | is based on the calibration standard CT | | |
| | values. The precision based on the analysis | | |
| | of the assay trending control (AAV2. | | |
| | Construct GMP), using the mean of | | |
| | 1 in 1000 and 1 in 10,000 dilutions, | | |
| | was 57.4% (n = 13 analyzed in 7 runs). | | |
| Specificity and | Test article is within 3.3 (1 log) C_{T} values | No inhibition | Pass |
| accuracy | of the appropriate standard | observed in the | |
| | | test article in the | |
| | | presence of 1 μg | |
| | | human DNA | |
| Linearity and range | r² ≥0.98. The target slope of the | r² 0.993 to 0.994 | Pass |
| | calibration curve for each assay should be | and a slope of | |
| | between −3.0 to −3.8, indicating a QPCR | −3.21 to −3.48 for | |
| | efficiency of 83% to 115%. | assays 1, 2b and 6 | |
| | | r² 0.988 to 0.997 | |
| | | and a slope of | |
| | | −3.17 to −3.56 for | |
| | | assays 7, 4 and 5 | |
| Co-linearity | Amplification efficiencies of the | Comparable | Pass |
| | supercoiled plasmid vector and viral DNA | across the range | |
| | are comparable | of 5 × 10\hat{ }5 | |
| | | copies/2.5 μL to 5 | |
| | | × 10\hat{ }2 copies/2.5 | |
| | | μL, 100 | |
| | | copies/2.5 μL and | |
| | | 50 copies/2.5 μL | |
| Limit of Detection | Report result | 168.2 copies/2.5 | Pass |
| | | μL | |
| Limit of | Report result | Valid over the | Pass |
| Quantification | | ranges of 5 × | |
| | | 10\hat{ }6 | |
| | | copies/2.5 μL to 5 | |
| | | × 10\hat{ }2 copies/2.5 | |
| | | μL, 100 | |
| | | copies/2.5 μL and | |
| | | 50 copies/2.5 μL | |
| Robustness | % CV using replicate C_{T} values of each | Reagents: 0.24% | Pass |
| | calibration curve dilution is ≤5%. | to 2.78% with | |
| | | respect to | |
| | | variation in | |
| | | different batches | |
| | | of reagent. | |
| | | Operator: | |
| | | <3.06% | |
| Reaction Mix | % CV using replicate C_{T} values of each | <1.91% | Pass |
| Preparation | dilution is ≤5% for the 2 batches of | | |
| | reaction mix prepared. | | |

DRP:IU Ratio—Calculation: No validation required. Value determined by calculation from physical titre and infectious unit titre assay data.

Total Particles: The total particles assay has been validated. A summary of the validation results is presented in Table 43. The “Actual Value” is a value measured for an exemplary AAV2-Construct of the disclosure.

TABLE 43
Total Particles Assay Validation Summary

| | Acceptance | | |
|---|---|---|---|
| Test Parameter | Criteria | Actual Value | Pass or Fail |
| Precision | CV≤20% | CV ≤14% | Pass |
| (Intra-Assay) | | (n = 4) | |
| Precision | CV≤30% | CV ≤28% | Pass |
| (Intermediate) | | (n = 4) | |
| Accuracy | Recovery 70- | Recovery between | Pass |
| | 130% | 106% and 115% for results obtained at | |
| | | dilution 2 to 16-fold of the standard | |
| Specificity | Blank sample | Blank sample does not show the | Pass |
| | must not show the | presence of particles (≤0.2 OD) | |
| | presence of | (n = 20) | |
| | particles (≤0.3 | | |
| | OD) | | |
| Range | Report result | From 1.88 × 10\hat{ }8 capsids/mL to 14.5 × | Pass |
| | | 10\hat{ }8 capsids/mL | |
| | | (n = 1) | |
| Linearity | r² ≥0.98 | r² ≥0.99 | Pass |
| | | (n = 1) | |
| Limit of | Report result | The LOD corresponds to the low plate | Pass |
| Detection | | of the calibration curve | |
| | | (n = 4) | |
| Limit of | Report result | 0.7 × 10\hat{ }8 capsid/mL | Pass |
| Quantification | | (n = 4) | |

Full:Empty Ratio: Drug specific assay validation not performed. Counting of full and empty AAV2 particles on transmission electron microscope images performed in GMP compliant facility.

Vector Identity (DNA): Drug specific assay validation not performed. DNA sequencing is performed using qualified method and equipment in a GMP compliant facility.

Total Protein Assay: This assay is validated to fulfil the following criteria (see Table 44).

TABLE 44
Total Protein Assay Validation Criteria

| Test Parameter | Acceptance Criteria |
|---|---|
| Precision | CV ≤20% |
| (Intra-Assay) | |
| Precision (Intermediate) | CV ≤30% |
| Accuracy | Recovery 70-130% |
| Range | Report result |
| Linearity | r² ≥0.98 |
| Limit of Detection | Report result |
| Limit of Quantification | Report result |

Purity Assay (SDS-PAGE): The purity assay has been validated. A summary of the validation results is presented (in Table 45). The “Actual Value” is a value measured for an exemplary AAV2-Construct of the disclosure.

TABLE 45
Purity Assay Validation Summary

| Test Parameter | Acceptance Criteria | Actual Value | Pass or Fail |
|---|---|---|---|
| Precision | CV ≤10% | ≤4% | Pass |
| (Intra-Assay) | | | |
| Precision (Intermediate) | CV ≤15% | ≤4% | Pass |
| Linearity | r² ≥0.98 | ≥0.99 | Pass |
| Limit of Quantification and | Report result | Limit of quantification | Pass |
| Range | | (by extrapolation) is | |
| | | 1.4%. | |
| | | Loading range from | |
| | | 1 × 10\hat{ }10 to | |
| | | 1 × 10\hat{ }11 | |
| | | particles/lane | |
| Specificity | No bands should be | No bands are detected | Pass |
| | detected in the lane | in formulation buffer | |
| | where the formulation | | |
| | buffer will be loaded | | |
| Robustness | A variation in | No effect on results | Pass |
| | temperature does not | | |
| | affect the result | | |

Replication Competent AAV: The replication competent AAV assay has been validated to fulfil the relevant validation requirements as specified in ICH Q2(R1) for a limit test for impurities (see Table 46). A summary of the validation results is presented below. The “Actual Value” is a measurement of an exemplary AAV2-Construct of the disclosure.

TABLE 46
Replication Competent AAV Assay Validation Summary

| Test | | | |
|---|---|---|---|
| Parameter | Acceptance Criteria | Actual Value | Pass or Fail |
| Specificity | Positive signal obtained only if | Positive signal obtained when | Pass |
| | adenovirus and replicative AAV are | adenovirus and replicative AAV | |
| | present | are present | |
| Detection | ≤10 rcAAV2 in 1 × 10\hat{ }10 DRP | ≤10 rcAAV2 in 1 × 10\hat{ }10 DRP | Pass |
| limit | AAV vector | | |

Total DNA: The total DNA assay has been validated. A summary of the validation results is presented (see Table 47). The “Actual Value” provided is a measurement of an exemplary AAV2-Construct of the disclosure.

TABLE 47
Total DNA Assay Validation Summary

| Test Parameter | Acceptance Criteria | Actual Value | Pass or Fail |
|---|---|---|---|
| Precision | CV ≤15% | CV ≤9.0% (n = 6) | Pass |
| (Intra-Assay) | | | |
| Precision | CV ≤20% | CV ≤12.1% (n = 4) | Pass |
| (Intermediate) | | | |
| Accuracy (Trueness) | Spike recovery within 50-200% | 66-83% (n = 4) | Pass |
| Range | Report result | 0.1 to 1.0 ng/mL | Pass |
| Linearity | r² ≥0.95 | r² ≥0.99 (n = 4) | Pass |
| Limit of Detection | Report result | 0.042 ng/mL (n = 4) | Pass |
| Limit of Quantification | Report result | 0.127 ng/mL (n = 4) | Pass |

HEK293 Host Cell DNA: The HEK293 Host Cell DNA assay has been qualified (see Table 48). A summary of the qualification results is presented below. The “Actual Value” provided is a measurement of an exemplary AAV2-Construct of the disclosure.

TABLE 48
Host Cell DNA Assay Qualification Summary

| Test Parameter | Acceptance Criteria | Actual Value | Pass or Fail |
|---|---|---|---|
| Precision | Report result | 24.81% | Pass |
| (Intra-Assay) | | | |
| Precision (Intermediate) | Report result | 27.84% | Pass |
| Accuracy Profile | Range within ±50% | Recoveries 77.87- | Pass |
| | | 107.6% | |
| Linearity | r² ≥0.97 | 1.00 | Pass |
| Limit of Quantification and | Report result | 0.6578-300.0 pg/μL | Pass |
| Range | | | |
| Specificity | No amplification | No amplification | Pass |
| | detected with E. coli or | detected with E. coli | |
| | if >5-fold difference in | | |
| | C_{T} value at the same | | |
| | qualification standard | | |
| | concentration | | |
| Trueness | Report result | 0.03-300 pg/μL | Pass |

Bioburden Assay: This is a compendial method harmonized to meet EP 2.6.12 and USP <61> requirements. Qualification/validation is conducted in accordance with the relevant pharmacopoeial monographs.

Endotoxin Assay: This is a compendial method harmonized to meet EP 2.6.14 and USP <85> requirements. Qualification/validation is conducted in accordance with the relevant pharmacopoeia monographs.

### Example 9: Batch Analysis Results for Compositions Manufactured Using Process 2

For the data provided below, the measurements are taken from one or more batches of AAV2-Construct, wherein the Construct is identical in all batches.

Batch analysis data from 2 non-GMP and 2 GMP batches are presented below in Table 50.

TABLE 50
Quality Control Testing Results for AAV2-Construct Drug Substance
Manufactured according to Process 2

| | Lot Number | | | |
|---|---|---|---|---|
| | AAV2- | AAV2- | AAV2- | AAV2- |
| | Construct | Construct | Construct | Construct |
| | (non-GMP)— | (non-GMP)— | (GMP)— | (GMP)— |
| | batch 1 | batch 2 | batch 1 | batch 2 |
| | Use | | | |
| | Process | | | |
| Acceptance | Development | Stability | Clinical | Clinical |

| | | | |
|---|---|---|---|
| Test | Test Method | Criteria | Results |

Vector Characterization

| | | | | | | |
|---|---|---|---|---|---|---|
| Physical | qPCR Based DNase | >0.7 × 10\hat{ }12 | 4.22 × 10\hat{ }11 | 1.11 × 10\hat{ }12 | 1.82 × 10\hat{ }12 | 4.30 × 10\hat{ }12 |
| Titre (DRP) | Resistant Particle | DRP/mL | DRP/mL^{*c} | DRP/mL | DRP/mL | DRP/mL |
| Assay | (DRP) Assay | | | | | |
| Infectious | Infection of RC32 | >1.24 × 10\hat{ }8 | 6.32 × 10\hat{ }9 | No result***** | 1.70 × 10\hat{ }10 | 1.1 × 10\hat{ }10 |
| Unit Titre | cells followed by | IU/mL | IU/mL | | IU/mL | IU/mL |
| Assay (IU) | detection of AAV2 | | | | | |
| | by qPCR | | | | | |
| DRP:IU | n/a | <5645:1 | 67:1 | n/a | 107:1 | 391:1 |
| Ratio— | | | | | | |
| Calculation | | | | | | |
| Total | Commercial anti- | >0.49 × 10\hat{ }12 | 2.7 × 10\hat{ }11 | 1.2 × 10\hat{ }12 | 3.2 × 10\hat{ }12 | 1.9 × 10\hat{ }12 |
| Particles | AAV2 particle | particles/mL | capsids/mL*** | particles/mL | particles/mL | particles/mL |
| | ELISA | | | | | |
| Full:Empty | Transmission | <55% empty | 85:15 | 76:24 | 49:51 | 89:11 |
| Ratio | electron microscopy | particles | | | | |

Identity

| | | | | | | |
|---|---|---|---|---|---|---|
| Vector | Purification of viral | Matches the | Sequence of | Not | Matches the | Matches the |
| identity | DNA with DNA | Expected | Construct | performed** | Expected | Expected |
| (DNA) | sequencing of both | Sequence with | matches the | | Sequence with | Sequence with |
| | strands | the exception | expected | | the exception | the exception |
| | | of the ITR | sequence | | of the ITR | of the ITR |
| | | regions | | | regions | regions |

Purity and Impurities

| | | | | | | |
|---|---|---|---|---|---|---|
| Total | Micro-BCA Protein | >5 μg/mL | <LOQ*** | 14 μg/mL | 22 μg/mL | 16 μg/mL |
| Protein | Quantification | | | | | |
| Purity | SDS-PAGE Assay | Purity >90% | Sample | 100% | 100% | 100% |
| | with impurities | with no single | presents me | | | |
| | estimated by | impurity >2%. | specific bands | | | |
| | intensity analysis | Three bands | of VP1, VP2 | | | |
| | | corresponding | and VP3 | | | |
| | | to AAV2 VP1, | proteins | | | |
| | | VP2 and VP3 | | | | |
| | | should be | | | | |
| | | evident. | | | | |
| Replication | HEK293 Cell Based | <LOD of | <LOD of | Not performed** | <LOD of | <LOD of |
| Competent | Amplification and | assay | assay**** | | assay | assay |
| AAV | qPCR | | | | | |
| HEK293 | Commercial ELISA | <80 ng/mL | <4 ng/mL | 10.5 ng/mL | <4 ng/mL | <LOD (<10 |
| Host Cell | Kit | | | | | ng/mL) |
| Protein | | | | | | |
| Total DNA | Picogreen assay | Report result | 130 ng/mL | 487 ng/mL | 575 ng/mL | 328 ng/mL |
| HEK293 | qPCR assay, Method | <2.66 × 10\hat{ }7 | 5.30 × 10\hat{ }2 | <6.58 × 10\hat{ }3 | <6.58 × 10\hat{ }3 | <6.58 × 10\hat{ }3 |
| Host Cell | res DNA SEQ | pg/mL | pg/mL | pg/mL | pg/mL | pg/mL |
| DNA (Qty | Human | | | | | |
| & Size) | | | | | | |
| Residual | Commercial ELISA | <126 ng/mL | 4 ng/mL | <1 ng/mL | <1 ng/mL | <1 ng/mL |
| BSA | Kit | | | | | |
| Residual | Commercial ELISA | <5 ng/mL | <0.2 ng/mL | <0.2 ng/mL | <0.4 ng/mL | <0.4 ng/mL |
| Benzonase | Kit | | | | | |

Safety

| | | | | | | |
|---|---|---|---|---|---|---|
| Bioburden | Membrane Filtration | <1 CFU/1% | TAMC: | Not performed | 0 CFU/0.410 | 0 CFU/0.552 |
| Assay | | PB | 0 CFU/mL | | mL | mL |
| | | | TYMC: | | (formulated | (formulated |
| | | | 0 CFU/mL | | bulk pre- | bulk pre- |
| | | | | | filtration) | filtration) |
| Endotoxin | Quantitative kinetic- | <5 EU/mL | 0.79 EU/mL | Not performed | <1 EU/mL | <1 EU/mL |
| Assay | chromogenic | | | | | |
| | method. | | | | | |

Footnotes:
*Analysis conducted according to Process 1.
**Not performed to conserve material for stability studies.
***Result fails to meet specification; specification set after results were obtained.
****Result obtained as part of validation of assay. Titre determined by Process 2 using plasmid DNA as assay standard (1.87 × 10\hat{ }11 DRP/mL) used instead of physical titre value reported.
*****Assay failed to meet validity criteria. Insufficient sample for re-analysis.

### Example 10: Batch Analysis Results for Compositions Manufactured Using Process 1

For the data provided below, the measurements are taken from one or more batches of AAV2-Construct, wherein the Construct is identical in all batches.

One clinical GMP batch has been manufactured according to Process 1. Batch analysis data from this batch of Drug Substance, is provided in Table 51.

TABLE 51
Quality Control Testing Results

| Test | Acceptance Criteria | Results |
|---|---|---|
| Physical Titre (DRP) Assay | Report result | 1.46 × 10\hat{ }12 DRP/mL |
| | | (Day 1) |
| | | 1.92 × 10\hat{ }12 DRP/mL |
| | | (Day 2) |
| Micro-BCA Protein Quantification | Report result | 35.350 μg/mL |
| AAV Purity | Purity >90% with no | Purity = 100% |
| Determination by SDS- | single impurity >2% | No detectable impurities |
| Polyacrylamide Gel | | |
| Electrophoresis | | |
| In-vitro assay for viral | None detected | None detected |
| contaminants¹ | | |
| Bioburden Assay | <1 CFU/1% of PBDS | <8.0 CFU/mL Total Yeast and |
| | (0.5% in each media) | Mold Count |
| | | <8.0 CFU/mL Total Aerobic |
| | | Microbial Count |
| Endotoxin Assay | <5 EU/mL | 0.234 EU/mL |
| Mycoplasma Assay¹ | None detected | Negative |

¹Results from IPC testing.

### Example 11: Stability of AAV Compositions

Aliquots of AAV2-Construct Drug Substance are analyzed for long term stability to support the AAV2-Construct Drug Substance storage condition of <−60° C. This study augments the ongoing Drug Product real time and accelerated stability studies at −80° C., −20° C. and 5° C.

AAV2-Construct Drug Substance is aliquoted into small containers (5 mL PETG screw cap bottles) representative of the full size AAV2-Construct Drug Substance containers to ensure sufficient containers are available for stability testing over time. These containers have the same product contacting materials and are chosen to best represent the final AAV2-Construct Drug Substance containers, with the exception of surface area to volume ratio. The smaller volume and larger surface area to volume ratio in the small container aliquots is believed to represent a ‘worst case scenario’ with respect to long term stability of the AAV2-Construct Drug Substance.

The aliquoted AAV2-Construct Drug Substance is placed at −80±10° C. with ambient relative humidity.

If insufficient aliquots of AAV2-Construct Drug Substance are available to complete the full 36 Month study, the study will be truncated at an earlier time point.

A reduced supportive stability study has been initiated using material from a small-scale AAV2-Construct (non-GMP) batch. This study uses 100 μl aliquots in 2 mL polypropylene cryotubes stored at <−60° C. The smaller fill volume and larger surface area to volume ratio in the small container aliquots are believed to represent a “worst case scenario” with respect to long term stability of the AAV2-Construct Drug Substance. The study will assess Drug Substance stability at 2, 6 and 12-month time points. Additional details of this study are presented below.

Test Requirements

Assays have been selected for the primary stability protocol based on the following rationale (see Table 52)

TABLE 52
Tests used for Primary Drug Substance Stability Protocol

| Test | Test Type | Objective and Rationale/Justification |
|---|---|---|
| Appearance | Physico-chemical | To accumulate data on the appearance during the stability |
| | (Visual) | program. Changes in appearance can result from a change |
| | | in the product, for example precipitation. |
| pH | Physico-chemical | To accumulate data about the pH during the stability |
| | (measurement | program. Changes of the pH can result from a change in |
| | with electrode) | the product, for example chemical degradation. |
| Purity by SDS- | Purity | To accumulate data about the purity during |
| PAGE | | the stability program. A decrease of the purity |
| | | could indicate product degradation. |
| Aggregation by | Integrity | To accumulate data about possible aggregation |
| DLS | | (particles size profile) during the stability program. |
| Physical Titre | Quantification | To accumulate data about the quantity of rAAV viral |
| (DRP) by qPCR | | vector during the stability program. |
| Construct | Activity | To measure and accumulate data about the expression of |
| Expression and | | Construct during the stability program. |
| Activity | | |
| Container Closure | Container Integrity | To verify the integrity of the container closure at |
| | | the end of the stability program. |

Assays have been selected for the supportive stability protocol to include physical titre, purity, total particles and potency as indicated in Table 53. These stability tests and time points (Table 54 and Table 57) were considered the most appropriate for the amount of material available to perform the study. An assessment of vector aggregation was not included in this study as the method is currently under development for AAV2 vector material. Container integrity was also excluded from the study since this is a reduced supportive stability study.

TABLE 53
Tests Used for Supportive Drug Substance Stability Protocol

| Test | Test Type | Objective and Rationale/Justification |
|---|---|---|
| Physical Titre (DRP) | Quantification | To accumulate data about the quantity of rAAV |
| by qPCR | | viral vector during the stability program. |
| Total Particles | Quantification | To accumulate data about the total quantity of |
| | | viral vector particles during the stability program. |
| Purity by SDS-PAGE | Purity | To accumulate data about the purity |
| | | during the stability program. A decrease of the |
| | | purity could indicate product degradation. |
| Construct Expression | Activity | To measure and accumulate data about |
| and Activity | | the expression of Construct during |
| | | the stability program. |
| Construct Activity | Activity | To measure and accumulate data |
| | | about the activity of expressed Construct |
| | | during the stability program. |

TABLE 54
Stability Protocol and Shelf Life Specifications for AAV2-Construct
Primary Drug Substance Stability Study, Storage at −80° C. ± 10° C.

| | | Time-Point (Months) | | | | | | | |
|---|---|---|---|---|---|---|---|---|---|
| | | T0 | T3 | T6 | T9 | T12 | T18 | T24 | T36 |
| Test | Specification | −80° C. | −80° C. | −80° C. | −80° C. | −80° C. | −80° C. | −80° C. | −80° C. |
| Appearance | Report result | X | — | X | — | X | X | X | X |
| pH | Report result | X | — | X | — | X | X | X | X |
| Purity by | Report result | X | — | X | — | X | X | X | X |
| SDS-PAGE | | | | | | | | | |
| Aggregation | Report result | X | X | X | X | X | X | X | X |
| by DLS | | | | | | | | | |
| Physical | Report result | X | — | X | — | X | X | X | X |
| Titre (DRP) | | | | | | | | | |
| by qPCR | | | | | | | | | |
| Construct | Report result | X | X | X | X | X | X | X | X |
| Expression | | | | | | | | | |
| and Activity | | | | | | | | | |
| Container | Conforms | — | — | — | — | — | — | — | X |
| Closure | | | | | | | | | |

TABLE 55
Stability Protocol and Shelf Life Specifications for AAV2-Construct
Supportive Drug Substance Stability Study, Storage at <−60° C.

| | | Time-Point (Months) | | | |
|---|---|---|---|---|---|
| | | T0 | T2 | T6 | T12 |
| | | <− | <− | <− | <− |
| Test | Specification | 60° C. | 60° C. | 60° C. | 60° C. |
| Physical Titre | >0.7 × 10\hat{ }12 DRP/mL | X | X | X | X |
| (DRP) by qPCR | | | | | |
| Total Particles | 90% with no single impurity >2%. | X | X | X | X |
| | Three bands corresponding to VP1, | | | | |
| | VP2 and VP3 should be evident | | | | |
| Purity by SDS- | >0.49 × 10\hat{ }12 particles/mL | X | X | X | X |
| PAGE | | | | | |
| Construct | >2-fold increase in band density of | X | X | X | X |
| Expression and | Construct protein in transduced cells | | | | |
| Activity | compared to non-transduced cells | | | | |
| Construct | >2-fold increase in band density of | X | X | X | X |
| Activity | Construct substrate in transduced cells | | | | |
| | compared to non-transduced cells | | | | |

TABLE 56
Stability Data for AAV2-Construct Primary Drug Substance Stability
Study, Storage at −80° C. ± 10° C.

| | | Time-Point (Months) | | | | | | | |
|---|---|---|---|---|---|---|---|---|---|
| | | T0 | T3 | T6 | T9 | T12 | T18 | T24 | T36 |
| Test | Specification _ | −80° C. | −80° C. | −80° C. | −80° C. | −80° C. | −80° C. | −80° C. | −80° C. |
| Appearance | Report result | X | — | X | — | X | X | X | X |
| pH | Report result | X | — | X | — | X | X | X | X |
| Purity by | Report result | X | — | X | — | X | X | X | X |
| SD STAGE | | | | | | | | | |
| Aggregation | Report result | X | X | X | X | X | X | X | X |
| by DLS | | | | | | | | | |
| Physical | Report result | X | — | X | — | X | X | X | X |
| Titre (DRP) | | | | | | | | | |
| by qPCR | | | | | | | | | |
| Construct | Report result | X | X | X | X | X | X | X | X |
| Expression | | | | | | | | | |
| and Activity | | | | | | | | | |
| Container | Conforms | — | — | — | — | — | — | — | X |
| Closure | | | | | | | | | |

TABLE 57
Stability Data for AAV2-Construct Supportive Drug Substance Stability
Study, Storage at <−60° C.

| | | Time-Point (Months) | | | |
|---|---|---|---|---|---|
| | | T0 | T2 | T6 | T12 |
| Test | Specification | <−60° C. | <−60° C. | <−60° C. | <−60° C. |
| Physical | >0.7 × 10\hat{ }12 DRP/mL | 1.8 × 10\hat{ }12 | 1.8 × 10\hat{ }12 | X | X |
| Titre | | DRP/mL | DRP/mL | | |
| (DRP) by | | | | | |
| qPCR | | | | | |
| Total | >0.49 × 10\hat{ }12 | 1.4 × 10\hat{ }12 | 1.2 × 10\hat{ }12 | X | X |
| Particles | particles/mL | capsids/mL | capsids/mL | | |
| Purity by | >90% with no single | Purity 100%. | Purity 100%. | X | X |
| SDS- | impurity >2%. Three | Three bands | Three bands | | |
| PAGE | bands corresponding to | corresponding | corresponding | | |
| | VP1, VP2 and VP3 should | to VP1, VP2 | to VP1, VP2 | | |
| | be evident | and VP3 | and VP3 | | |
| | | evident | evident | | |
| Construct | >2-fold increase in band | 5.0 fold | 3.1 fold | X | X |
| Expression | density of Construct | increase in | increase in | | |
| and | protein in transduced cells | band density | band density | | |
| Activity | compared to non- | of Construct | of Construct | | |
| | transduced cells | protein in | protein in | | |
| | | transduced | transduced | | |
| | | cells | cells | | |
| | | compared to | compared to | | |
| | | non- | non- | | |
| | | transduced | transduced | | |
| | | cells | cells | | |
| Construct | >2-fold increase in band | 4.3 fold | 8.3 fold | X | X |
| Activity | density of substrate in | increase in | increase in | | |
| | transduced cells compared | band density | band density | | |
| | to non-transduced cells | of substrate in | of substrate in | | |
| | | transduced | transduced | | |
| | | cells | cells | | |
| | | compared to | compared to | | |
| | | non- | non- | | |
| | | transduced | transduced | | |
| | | cells | cells | | |

X: Denotes future stability time point.

### Example 12: Analysis of Clinical Drug Product

For the data provided below, the measurements are taken from one or more batches of AAV2-Construct, wherein the Construct is identical in all batches.

The analytical procedures which are performed for release testing of AAV2-Construct Drug Product are detailed in Table 58, in addition to the respective specifications.

TABLE 58
Drug Product Specifications

| | | |
|---|---|---|
| Test Parameter | Test Method | Acceptance Criteria |

General Tests

| | | |
|---|---|---|
| Appearance | Color, Visible Particles and Clarity | Clear to Slightly Opaque, |
| | | Colorless to Faint White Solution |
| pH | pH Meter | 8 ± 0.5 |
| Osmolality | Freezing Point Depression | 388 − 426 mOsm/kg H₂O |

Content

| | | |
|---|---|---|
| Physical Titre | qPCR Based DNase Resistant Particle (DRP) | 0.8 × 10\hat{ }12 − 1.5 × 10\hat{ }12 |
| | Assay | |
| Infectious Unit (IU) Titre | Infection of RC32 cells followed by detection of | >1.4 × 10\hat{ }8 IU/mL |
| | AAV2 by qPCR | |
| DRP: IU Ratio- | n /a | <5645:1 |
| Calculation | | |

Potency

| | | |
|---|---|---|
| Potency | Measurement of Construct protein expression | ≥2-fold increase in band density |
| | and substrate following in vitro transduction of | of Construct protein and substrate |
| | HEK293 cells | in transduced cells compared to |
| | | non-transduced cells |

Safety

| | | |
|---|---|---|
| Sterility | Aerobic and Anaerobic culture for bacteria and | Absence of growth |
| | fungus by immersion | |
| Endotoxin | Kinetic Chromogenic (USP/FDA I/E Assay) | <5 EU/mL |

Analytical Procedures: The methods used to control the Drug Product are summarized below.

Appearance (EP 2.2.1, 2.2.2 and 2.9.20 and USP <631>): The product will be inspected visually for transparency, color and the absence/presence of foreign particles. The product will be inspected against white and black backgrounds.

pH (EP 2.2.3 and USP <791>): The pH of the product will be determined using a micro pH electrode with temperature compensation.

Osmolality (EP 2.2.35 and USP <785>): Osmolality is determined by the Freezing Point Depression method.

Physical Titre: Physical titreing on the final product will be performed as described in herein.

Infectious Unit Titre Assay: The infectious titre assay on the final product will be performed as described herein.

Potency: This assay is used to demonstrate that Construct protein is over produced, and is active, in cells transduced with the test article. The cells, HEK293, express endogenous Construct protein at a very low level, allowing for a comparative assessment of increased Construct protein expression due to cell transduction and expression from the AAV2-Construct vector compared to non-transduced cells. Following transduction, cells are lysed and the Construct concentration in the lysates is measured by Western blotting or ELISA. The activity of the expressed Construct protein is measured on the cell lysates using a reaction which measures the incorporation of biotin-labelled GPP into a substrate using Wester blotting or ELISA.

Sterility Test (EP 2.6.1 and USP <71>): This procedure is used to determine if the test article is free from viable bacterial and fungal contamination. The test article is aseptically transferred into Soybean-Casein Digest Medium (SCDM) and Fluid Thioglycollate Medium (FTM). These broths are incubated for 14 days and inspected for evidence of bacterial and fungal growth.

Endotoxin (EP 2.6.14 and USP <85>): This assay is used to determine if bacterial endotoxins are present in the test article. A quantitative procedure is performed by the kinetic-chromogenic method. Known amounts of endotoxin are tested in parallel with the test article for an accurate determination of the level of bacterial endotoxin. The potential for interference by the test article is examined by spiking the test article plus LAL reagent with specified levels of endotoxin. Following the inhibition/enhancement test, the endotoxin content of the test article is then determined.

Validation of Analytical Procedures: Summaries of method validation results, where available, are presented below.

Appearance: This is a compendial method harmonized to meet EP 2.2.1/2.2.2 and USP <631> requirements. Qualification/validation is conducted in accordance with the relevant pharmacopoeial monographs.

pH: This is a compendial method harmonized to meet EP 2.2.3 and USP <791> requirements. Qualification/validation is conducted in accordance with the relevant pharmacopoeial monographs.

Osmolality: This is a compendial method harmonized to meet EP 2.2.35 and USP <785> requirements. Qualification/validation is conducted in accordance with the relevant pharmacopoeial monographs.

Sterility: This is a compendial method harmonized to meet EP 2.6.1 and USP <71> requirements. Qualification/validation is conducted in accordance with the relevant pharmacopoeial monographs.

### Example 13: Clinical Dose Design

Choroideremia is a rare currently incurable X-linked recessive retinal degeneration that presents in late childhood and leads to blindness. This example provides the primary endpoint of a non-randomised, two year, phase 1-2 open label clinical trial assessing retinal gene therapy, using an adeno-associated viral type 2 (AAV2) vector expressing the choroideremia transgene (NCT01461213). Initially 12 patients were recruited, 5 of whom received 1×10¹⁰ genome particles (gp) of AAV.REP1 and 6 received 1×10¹¹ gp. A surgical complication in one patient led to retinal thinning and a reduced vector dose of less than 10¹⁰ gp. The primary outcome measure of safety and efficacy was visual acuity at two years. Retinal sensitivity and anatomical changes were secondary endpoints. Over the entire group of 14 patients, by two years the median visual acuity improved by 4.5 letters in the treated eyes compared with a loss of 1.5 letters in the untreated eyes (p=0.03). For the 12 patients receiving gene therapy without complications, the treated eyes had gained 5.5 letters above their baseline level by two years (p=0.02). Compared to the untreated eyes, this represented a median 4.5 letter gain in favour of the eyes receiving gene therapy by two years (p=0.003). By this time 6 of the 12 treated eyes had gained more than one line of vision (>5 letters), compared with none of the 12 untreated eyes. At the last follow up (range 2-5 years), visual acuity had been maintained or increased in all 12 study eyes that had received the gene therapy as per protocol, compared with only 4 of the 12 untreated eyes. This was equivalent to a mean difference of 15.9 letters (>3 lines) between eyes. No statistically significant differences were seen in secondary endpoints, such as microperimetry, retinal thickness or loss of the outer rim of retina between treated and untreated eyes during the two year follow up. Retinal gene therapy for choroideremia appears safe and may benefit visual acuity.

In this unmasked, non-randomized, prospective interventional gene therapy clinical trial, 14 participants were recruited with informed consent and underwent gene therapy treatment to one eye using the AAV2.REP1 vector. Generally the eye selected for treatment had the worse visual acuity at baseline but in 3 cases the eye with better acuity was chosen because of other factors, such as visual field constriction. All participants were male ranging from 25 to 73 years of age with confirmed null mutations in the CHM gene. The primary objective of the trial was to assess safety in relation to maintaining vision by two years after surgery. Initially 12 patients were to be recruited into two dose cohorts of six patients, each of whom would be monitored for 24 months. Complications in two patients however led to a 24 months delay midway through the trial and a change in protocol relating to the surgical technique and immune suppression regimen used. The ethics committee approved an extension of the trial together with the recruitment of two further patients so that 12 patients in total received the gene therapy treatment as per the protocol without complications.

In the first cohort of 6 patients, a subretinal injection of up to 1×10¹⁰ genome particles (gp) assayed using a supercoiled plasmid vector reference was injected under the retina in a two-step procedure. This comprised an initial detachment of the retina with balanced salt solution delivered through a 41 gauge (G) Teflon cannula (DORC BV, Zuidland, Netherlands) and secondary injection of the AAV.REP1 vector into the newly created subretinal space. In patient six (C1) difficulties in detaching the retina and stretching of the papillomacular bundle resulted in a reduced gene therapy dose of <6×10⁹ and subsequent retinal thinning, but all other patients received either the full low dose of 1×10¹⁰ gp (L1-5) or high dose of 1×10¹¹ gp (C2 and H1-7), as per the protocol. Initially oral prednisolone was administered at 1 mg/kg for 3 days before and 7 days after gene therapy, but C2 developed vitritis and retinitis at 2 weeks post-operatively which adversely affected his vision. The protocol was subsequently amended so that H1-7 received an extension of the prednisolone regime: 0.5 mg/kg (days 8-14), 0.25 mg/kg (days 15-16), then 0.125 mg/kg (days 17-18). An air bubble in the injection system expanded into the subretinal space in H3 and vector administration was deferred because it was felt the vector would be displaced by the subretinal air. The injection system was amended in order to allow more controlled infusion of vector into the subretinal space and reduce the possibility of trapped air bubbles. The protocol allowed for deferred vector administration if indicated for surgical reasons and H3 was brought back for retinal gene therapy at a later date when the full dose of vector was administered without complication.

To aid visualizing the retinal detachment and monitor retinal stretch, intra-operative optical coherence tomography (OCT) imaging was introduced in H3-7. This comprised a vertically aligned Heidelberg Spectralis that was moved in and out of the operating field at various stages to assess the plane of subretinal injection and degree of retinal stretch.

In choroideremia, unlike many other retinal degenerations, loss of the RPE leads to a scarring reaction of the underlying choroid which then becomes firmly adherent to the residual retina. The central island of functional retina however remains intact and a tissue plane for vector administration between RPE and photoreceptors can be identified within it. If however fluid is injected into this plane too quickly, then the developing fluid bleb will form a tight bubble and stretch the central retina, because the peripheral scar tissue will prevent subretinal fluid from propagating outwards into the retinal periphery. This retinal stretch reaction has two negative consequences. First it may directly damage the neurosensory retina—in C1 the papillomacular bundle was subject to stretch as reported previously, because the outer nuclear layer was deficient in this area making it thinner than the central retina and subject to more stretch at a given pressure according to Hooke's Law. Second a higher fluid pressure in the bleb will result in reflux of vector suspension back through the widened retinotomy into the vitreous, thereby reducing the therapeutic dose delivered and increasing the risk of inflammation. Hence following the complication in C1 in which the vector was injected manually from a 1 ml syringe, a new vector administration device was designed and tested, which allowed a precise foot-pedal controlled slow infusion of the vector. This was used successfully in H3-7 in whom vector administration was uncomplicated. A bubble of heavy liquid, perfluoro-n-octane (Bausch & Lomb, Rochester, N.Y., USA) was used to stabilize focal areas of retinal thinning in P10 and P11 during vector infusion.

Visual function was assessed with best-corrected visual acuity (BCVA) according to the Early Treatment for Diabetic Retinopathy Study (ETDRS) protocol. In addition, Pelli-Robson contrast sensitivity testing was performed at baseline, 1 year and 2 years. Microperimetry testing using the MAIA microperimeter (CenterVue SpA, Padova, Italy) followed the protocol described previously [MacLaren et al., 2014] except in H3-7, standard non-customized 20° (38 stimuli) central grids were used. For participants with profound visual field restriction who could achieve barely detectable or 0 dB mean threshold sensitivities on the 20° grids (H4-6), a 10° central grid was used instead. Following subjective reports of improved color vision in L3, color vision assessment using the Farnworth-Munsell 100 hue (FM100) test was added in the protocol change and included in H1-7. While further subjective descriptions of improved color perception in the treated eye were reported by H2, H5, H6 and H7, the test proved difficult to perform in patients with advanced visual field loss. Anatomical assessments included spectral domain optical coherence tomography (OCT) using the Spectralis (Heidelberg Engineering, Heidelberg, Germany) and fundus autofluorescence (AF) imaging (BluePeak, Heidelberg Engineering). Immunology tests to assess T-cell responses to AAV (ELISpot) followed the methodology described previously [MacLaren et al., 2014].

Due to the ceiling effects of including eyes with near maximal visual acuity in the study, the ETDRS scores were found to be skewed (Shapiro-Wilk normality test at 0.05 alpha) and are therefore presented as median values with interquartile ranges (IQR). Changes between treated and control eyes were compared using the Wilcoxon signed rank test. Microperimetry data and anatomical assessments were found to be normally distributed and compared using the paired t-test.

### Results

In total 14 patients were recruited, 13 of whom received either 1×10¹⁰ gp (L1-5) or 1×10¹¹ gp (C2 and H1-7) of the AAV.REP1 vector (Table 59). There were significant adverse events (AEs) relating to vector administration in two patients: C1 and C2. In C1, a surgical complication resulted in retinal thinning and the vector administration was under-dosed. In C2, there was significant retinal inflammation at 2 weeks post-operatively that was most likely related to the vector. In H3 a subretinal air bubble prevented vector administration at the first attempt, but secondary vector delivery was successful without complication at a later date. The change in protocol, particularly with regard to the surgery, has been designed to reduce the chance of these adverse events occurring in the future. The ethics committee approved the recruitment of two further patients, thereby providing 12 patients treated as per the protocol with two year follow up, including 5 at lose-dose (L1-5) and 7 at high-dose (H1-7).

TABLE 59
Demographic characteristics of trial participants.

| ID | Age (yr) | CHM mutation | Predicted protein sequence | Eye treated | Vector dose (vg) |
|---|---|---|---|---|---|
| L1 | 63 | c.940 + 2T > C | splice donor site mutation- | left | 1 × 10¹⁰ |
| | | | intron 7 (+2) | | |
| L2 | 47 | c.189 + 1G > C | splice donor site mutation- | left | 1 × 10¹⁰ |
| | | | intron 3 (+1) | | |
| L3 | 36 | c.492_493delGA | N165Cfs*8 (exon 6) | left | 1 × 10¹⁰ |
| L4 | 55 | c.535_538delGAAA | E179Tfs*17 | left | 1 × 10¹⁰ |
| L5 | 41 | c.529delG | E177Kfs*20 | right | 1 × 10¹⁰ |
| C1 | 57 | c.819 + 1G > T | splice donor site mutation- | left | 6 × 10⁹ |
| | | | intron 6 (+1) | | |
| C2 | 44 | c.130G > T | G44* (exon 3) | left | 1 × 10¹¹ |
| H1 | 38 | c.799C > T | R267* (exon 6) | left | 1 × 10¹¹ |
| H2 | 43 | c.877C > T | R293* (exon 7) | left | 1 × 10¹¹ |
| H3 | 41 | c.1264C > T | Q422* (exon 10) | right | 1 × 10¹¹ |
| H4 | 59 | c.1335_1336insA | R446Tfs*16 | right | 1 × 10¹¹ |
| H5 | 72 | c.757C > T | R253* (exon 6) | right | 1 × 10¹¹ |
| H6 | 55 | c.799C > T | R267* (exon 6) | left* | 1 × 10¹¹ |
| H7 | 24 | c.525_526delAG | E177Kfs*6 | right | 1 × 10¹¹ |

All participants are Caucasian males with genetically confirmed choroideremia and are listed in order of undergoing gene therapy surgery. L1-5 and H1-7 underwent uncomplicated gene therapy at the low dose and high dose, respectively. The surgery in C1 was complicated by retinal stretch resulting in underdosing. C2 developed marked post-operative intra-ocular inflammation, which led to an acute reduction in visual acuity followed by slow rebound once the inflammation had resolved.

Assessments of Visual Acuity

In total, 12 received the AAV.REP1 vector by subretinal injection without complications and 2 were treated off-protocol as a result of adverse events. At the 2 years trial end-point, the median visual acuity in the 14 treated eyes had improved by 4.5 letters (IQR: −2.0 to 8.8) and in the 14 untreated eyes had declined by −1.5 letters (IQR: −5.0 to 0.0). Hence favoring the treated eyes overall, despite significant complications in 2 patients (Wilcoxon Signed rank test, W=68, z=2.12, p=0.034). Hereafter, for the purpose of evaluating efficacy of the investigational medicinal product (IMP), the 12 patients receiving the gene therapy treatment as per protocol (L1-5 and H1-7) are reported separately to the 2 patients with surgical or medical complications (C1 and C2).

Due to the delay midway through the trial to optimize the surgical technique, the first 5 patients (L1-5) have 5 years of follow-up whereas the last 5 (H3-7) have only recently reached the 2 years follow-up, with intermediate follow-up periods in between. The data are therefore presented in two ways: the first is across all 12 patients at 2 years, as per the original end-point defined in the protocol and the second is the last point of follow-up, for which L1-5 and H1-2 extend beyond 2 years.

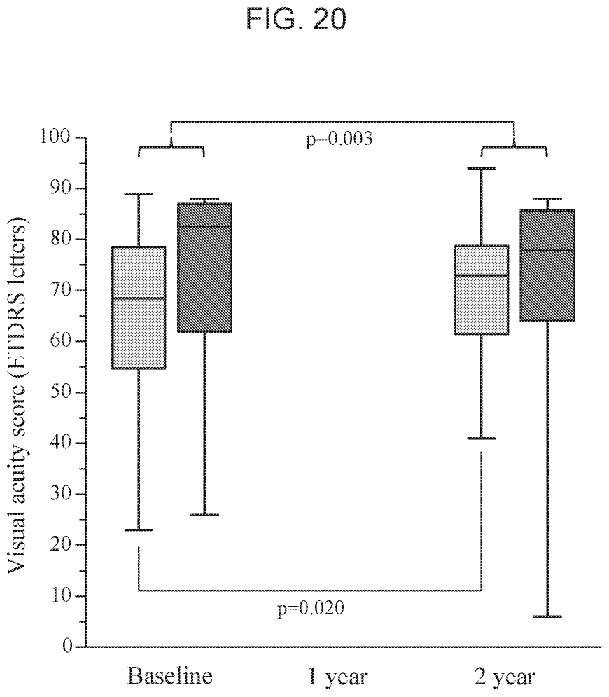

By 2 years, the median visual acuity improved by 5.5 letters (IQR: 2.5 to 9.0) in the 12 treated eyes (Wilcoxon signed rank test, W=60, z=2.33, p=0.020). In contrast, there was a small loss of −1.0 letters (IQR: −5.0 to +1.0) in the untreated eyes over this period (Table 60 and FIG. 20). This represented a median gain of 4.5 letters (IQR: 2.0 to 14.0) in the treated eyes over the untreated fellow eyes (Wilcoxon signed rank test, W=76, z=2.96, p=0.003). At the last follow-up, the treated eyes had gained a median of 6.5 letters (IQR: 3.8 to 10.3), whereas the untreated eyes had lost −2.0 letters (IQR: −5.3 to 0.3). This represented a median gain of 8 letters (IQR: 4.0 to 18.5) in the treated eye over the untreated eye (Wilcoxon signed rank test, W=78, z=3.04, p=0.002).

By the last follow up, visual acuity had been maintained or increased in all 12 treated eyes that had received gene therapy, but 8 of the 12 untreated eyes had deteriorated by variable amounts during this period. Visual acuity is a biological reading that is variable, but testing was performed in a standard manner and none of the untreated eyes had gained more than one line (>5 letters) by 24 months. In contrast, 6 of the 12 treated eyes (50%) gained more than one line by 24 months. Compared with the untreated eye, gains of two lines or more were seen in 4 of 12 patients at 24 months (Table 60: Bold). This increased further to 6 of 12 (50%) by the last follow up with 4 of these having a gain of three lines or greater (≥15 letters) in favor of the treated eyes (Table 60: Bold). At the last follow-up, the visual acuity in the treated eye had improved relative to the untreated eye in every one of the 12 patients (median=8.5 letters, IQR: 4.0 to 18.5), equivalent to a mean gain of 15.9 letters (>3 lines) in favor of the eyes receiving gene therapy

TABLE 60
Visual acuity results (best-corrected ETDRS scores) at 2 years and last
follow-up for all 12 participants.

| | | Lowe dose (1 × 10¹⁰ gp) | | | | | High dose (1 × 10¹¹ gp) | | | | | | | |
|---|---|---|---|---|---|---|---|---|---|---|---|---|---|---|
| | | L1 | L2 | L3 | L4 | L5 | H1 | H2 | H3 | H4 | H5 | H6 | H7 | Med |
| Baseline | TE | 23 | 79 | 89 | 53 | 79 | 77 | 76 | 70 | 61 | 67 | 60 | 39 | 68.5 |
| | CE | 58 | 82 | 85 | 76 | 83 | 87 | 88 | 88 | 57 | 74 | 26 | 87 | 82.5 |
| 24 m | TE | 41 | 73 | 94 | 61 | 76 | 78 | 80 | 79 | 70 | 73 | 63 | 53 | 73.0 |
| | CE | 62 | 77 | 85 | 70 | 78 | 86 | 88 | 87 | 62 | 78 | 6 | 84 | 78.0 |
| Change (24 m) | TE | 18 | −6 | 5 | 8 | −3 | 1 | 4 | 9 | 9 | 6 | 3 | 14 | +5.5 |
| | CE | 4 | −5 | 0 | −6 | −5 | −1 | 0 | −1 | 5 | 4 | −20 | −3 | −1.0 |
| Diff (24 m) | | 14 | 1 | 5 | 14 | 2 | 2 | 4 | 10 | 4 | 2 | 23 | 17 | +4.5 |
| Last FU | TE | 48 | 79 | 93 | 73 | 84 | 80 | 83 | 79 | 70 | 73 | 63 | 53 | 76.0 |
| | CE | 0 | 77 | 86 | 73 | 77 | 86 | 88 | 87 | 62 | 78 | 6 | 84 | 77.5 |
| Change (last FU) | TE | 25 | 0 | 4 | 20 | 5 | 3 | 7 | 9 | 9 | 6 | 3 | 14 | +6.5 |
| | CE | −58 | −5 | 1 | −3 | −6 | −1 | 0 | −1 | 5 | 4 | −20 | −3 | −2.0 |

| | | | | | | | | | | | | | |
|---|---|---|---|---|---|---|---|---|---|---|---|---|---|
| Diff (last FU) | 83 | 5 | 3 | 23 | 11 | 4 | 7 | 10 | 4 | 2 | 23 | 17 | +8.5 |
| Last FU (y) | 5 | 5 | 5 | 5 | 5 | 4 | 4 | 2 | 2 | 2 | 2 | 2 | 4.0 |

gp = genome particles of AAV2.REP1 vector;
Med = median;
TE = treated eye;
CE = control eye;
m = months;
y = years;
DIFF = difference between eyes;
FU = follow-up;
vision changes of 2 lines (10 letters) or higher between eyes are shown in bold.

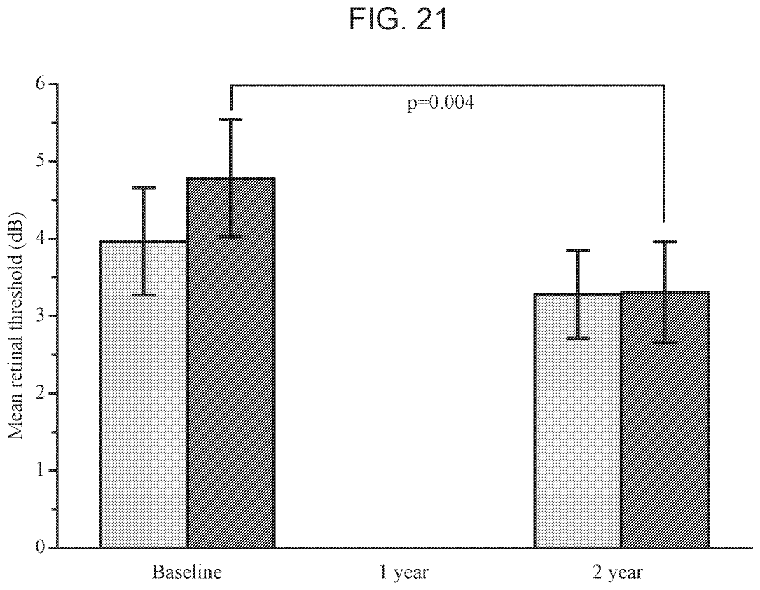

Microperimetry assesses mean retinal sensitivity across a wider area of the macula than visual acuity, but takes longer to perform and requires concentration, particularly for patients with poor fixation [Jolly et al., 2017]. The mean retinal sensitivity in the treated eyes was 4.0±0.7 decibels (dB) at baseline and 3.3±0.6 at 2 years, representing a small statistically insignificant decline (p=0.07). In contrast, the untreated eyes fell significantly from 4.8±0.8 dB at baseline to 3.3±0.7 dB at 2 years (p=0.004). Although there was a relative gain favoring the treated eyes over the untreated eyes of 0.8±0.53 dB (95% CI: −0.3 to 1.8 dB) at the 2 year study endpoint, it was not statistically significant (p=0.17). The data and interim analysis at 1 year are shown in FIG. 21. By this stage the mean retinal sensitivity loss in the treated eyes was less than the untreated eyes by 0.9±0.5 dB (95% CI: 0.0 to 1.8 dB) suggesting that the improvement relative to the untreated eye seen at 2 years may be long-lasting.

Microperimetry also provides useful information about fixation, that is, the retinal locus that has maximal sensitivity (usually the fovea). All patients except L1 still retained some degree of foveal or parafoveal fixation, consistent with the centripetal nature of visual field loss in this disease. It was previously noted that L1 changed his fixation (or preferred retinal locus) to use this treated island, whilst bypassing the untreated island of retina. This was maintained up to 5 years in this patient, consistent with the sustained improvement in visual acuity.

Preservation of retinal structure: Anatomical assessments included optical coherence tomography (OCT), which gives a cross-sectional view to measure retinal thickness and autofluorescence, which generates a map that can be used to estimate the surviving retinal area. The eyes were not randomized, with the more advanced eye in terms of visual acuity selected for gene therapy in 9 of the 12 study patients (Table 59).

OCT was used primarily for safety to assess retinal thinning that might occur, for instance, from surgical trauma, or if there were a long-term toxic effect from REP1 protein over-expression. The mean thickness of the 12 protocol treated eyes at the point of fixation (as indicated by microperimetry) at baseline was 224.8 μm compared with 251.9 μm in the untreated eyes. By 24 months, the treated eyes have reduced to a mean of 207.7 μm and the untreated to 245.6 μm, which was statistically significant (n=12, p=0.04). Further subgroup analysis however revealed that in the first 7 eyes that received manual injection of vector into the subretinal space (L1-5 & H1-2), retinal thinning was greater in the treated eyes (mean 23.1 μm) than the untreated eyes (6.4 μm) (n=7, p=0.02). However in eyes that received automated injection after improvement to the vector delivery system (H3-7), no significant difference was detected (n=5, p=0.72).

For area measurements, the autofluorescence area is correlated to the area of surviving photoreceptors calculated from multiple slices through the ellipsoid zone. Across the whole group of 12 patients, 81.1±3.2% of autofluorescence area was preserved in the treated eyes, whereas 80.8±2.1% was preserved in the untreated eyes by 2 years, which was similar. It should be noted however that AF shrinkage only occurs from the outer most retinal cells at the leading edge of the degeneration and 2 years is insufficient time to assess the long-term effects of retinal gene therapy on the healthier central zones. Only one patient (H5) had no measureable decline (100% preservation) by 2 years and it was observed in his treated eye only. Long-term (5 year) follow-up of 5 patients showed a similar pattern, with 66.1±5.0% remaining autofluorescence in the treated eyes compared with 64.9±3.6% in the untreated eyes (Table S2B). It should however be noted that this was a mixed picture, with the patients with large visual acuity gains doing far better than others. Furthermore, over this time period significant inter-patient differences in the measured rate of degeneration emerged even in the unoperated eyes, which ranged from 58% to 78%. This highlights the need not to over-interpret a limited non-randomized dataset.

Adverse Events

All patients were enrolled without visually significant cataract at baseline because of the need for a clear view of the 41 gauge retinotomy, but cataract formation is a well-known and almost inevitable side effect of vitrectomy surgery. In 2 participants (H5 and H6), cataract surgery had been performed previously. One patient developed visually-significant cataract which was removed at 1 year after gene therapy (H3) but four other patients (L2, L4, L5, H2) had cataract surgery after completion of the 2 years follow-up. Cataract surgery has not yet been performed in the remaining five patients (L1, L3, H1, H4 and H7), although all have signs of cataract progression on LOCS III cataract grading. Across the whole group of 14 patients and even including the two patients with complications, the visual acuity in the treated eyes improved relative to the untreated eyes over this period. Considering only the 12 patients who underwent uncomplicated retinal gene therapy without protocol deviations, by the 2 year endpoint, the visual acuity had improved in the treated eyes compared to their baseline. At the latest time point analyzed which is up to 5 years, all 12 treated eyes had maintained or gained visual acuity above their baseline levels, despite variable losses in visual acuity in 8 of the 12 untreated eyes during this period. The beneficial effects on visual acuity combined with good safety profile warrant further assessment of choroideremia gene therapy as a potential treatment in a fully randomized prospective Phase III clinical trial (see Example 14).

While there was no significant difference in the visual acuity gains between the low dose or high dose cohorts, it should however be noted that this was a very small study group with only 5 patients treated at the lower (1×10¹⁰) dose. Given the heterogeneous nature of the human innate anti-viral immune system, it is likely that a threshold dose may be therapeutic in some, but not all patients. Hence it may be advantageous to treat with a maximal tolerated dose, as long as no toxic effects are seen. The excellent preservation of visual function in the high dose patients over time supports this approach. It is also worth bearing in mind that the patients selected for this study were generally end-stage with very small islands of residual retina and therefore not many remaining retinal cells to treat. The low dose might be insufficient for earlier stage patients in whom a much larger target area would be exposed to vector. It should also be noted that the pharmaceutical formulation used for this study does not comprise a PRE or WPRE element, which may improve therapeutic efficacy.

With the exception of H5, further constriction of the area of autofluorescence in the remaining patients was not consistent with other preclinical models in which improvements in visual function resulting from single gene replacement are invariably associated with slowing of retinal degeneration. Here, however, it is important to bear in mind that the advancement of degeneration measured over the 2 year time frame is only at the extreme edges of the retinal degeneration and this part may be too advanced for rescue. The visual acuity gains in contrast arose mainly from the central retina. Also the retina in choroideremia is difficult to detach beyond the edges of the surviving retina resulting in stretching of the overlying bleb. The concave shape of the subretinal space will increase the height of the bleb centrally, which will therefore be the last part of the retina to reattach as the fluid is reabsorbed. This could potentially provide the central retina with several additional hours of exposure to the vector compared with the extreme periphery. Finally it should be noted that the eyes were not randomized and inter-eye differences in the area of surviving retina seen in some patients at baseline is indicative of asymmetric rates of degeneration. By treating the worse eye, there will by definition be a bias towards treating the eye with the more rapid degeneration.

Whilst visual acuity was maintained or increased in all treated eyes throughout the duration of the study, retinal sensitivity measured with microperimetry showed a trend towards decline and although numerically better than the untreated eyes, there was no statistically significant difference between the two. This is most likely due to the increased variability in performing microperimetry compared with reading single letters on a high contrast eye chart. Nevertheless the tests do have subtle differences in what they measure. Microperimetry assesses retinal function from many points averaged out over 10-30 degrees of central retina, whereas visual acuity is a measurement taken from a single point that generally has maximal sensitivity and is centrally located. Maintaining retinal sensitivity therefore is improved from a successful transduction of the entire area being measured right up to the edges of the surviving retina. For instance, if only 20% of retinal pigment epithelial cells centrally are successfully transduced by the vector, then this might be sufficient to see a visual acuity gain if these cells are beneath the fovea. The remaining 80% of non-transduced cells however would continue to degenerate and this decline would be reflected in a gradual decline in microperimetry over time, but visual acuity would remain stable. If however gene therapy is successful in stopping the degeneration in the areas successfully transduced, then eventually only the transduced cells would remain and the microperimetry readings would then stabilize to remain constant over time. This ‘plateau’ effect is now emerging in L1 who has also maintained his visual acuity gain and who has the longest follow up. Alternatively it might be a simple factor that retinal sensitivity measured by microperimetry is more sensitive to cataract formation than high contrast visual acuity. At the 2 year post-vitrectomy time point, only one of the 10 phakic protocol-treated patients had undergone cataract surgery.

The success of surgery is a critical factor when interpreting outcomes in this and any other gene therapy study that involves delivery of vector under the retina. One potentially adverse event of retinal stretch was related to surgery. The other significant adverse event of inflammation might equally be related to surgery if during the injection there was reflux of vector into the vitreous, which is known to trigger inflammation. Following the protocol change midway through the trial, an automated system of subretinal injection was adopted that allowed precise and controlled delivery of vector into the subretinal space. This was further facilitated by intra-operative retinal scanning (OCT) which provided a real time cross-sectional image of the retina being detached and helped identify the subretinal space in some of the more advanced patients. These additional surgical refinements will improve safety in future gene therapy studies. Nevertheless the results of this Phase I/II clinical trial show that gene therapy for choroideremia is generally safe.

### Example 14: Gemini Study

This is a multi-centre, open-label, prospective, bilateral interventional safety study of AAV2-REP1 in adult male subjects with genetically confirmed CHM.

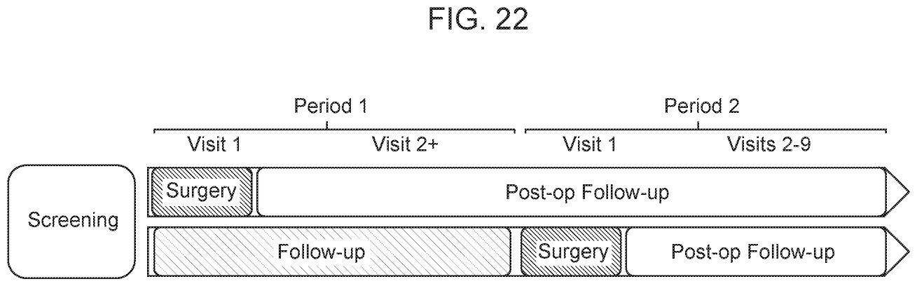

The study will consist of a Screening Visit followed by 2 treatment periods (Period 1 and Period 2) with up to 9 visits per period (FIG. 22). At the Screening Visit, subjects are assessed for eligibility of both eyes. The investigator assigns the order in which the eyes are treated (i.e., study eye 1 [SE1] and study eye 2 [SE2], respectively) with a number assigned to each eye. This assignment is done in collaboration with the subject; however, the worse eye is generally selected for treatment first. The target interval between the surgical procedure in SE1 and SE2 is determined at the Screening Visit. Each study eye is followed for at least 12 months post-treatment, for up to 9 visits per treatment period: Visit 1 (Day 0, Injection Day Visit); Visit 2 (Day 1, post-operative follow up visit); Visit 3 (Day 3); Visit 4 (Day 7); Visit 5 (Day 14); Visit 6 (Month 1); Visit 7 (Month 3); Visit 8 (Month 6) and Visit 9 (Month 12).

At Period 1, Visit 1 (Day 0, Injection Day Visit for SE1), subjects undergo a vitrectomy with retinal detachment and receive a sub-retinal injection of AAV2-REP1 in SE1. Visits 2-9 are conducted according to the Schedule of Study Procedures, unless Period 2 commences during this time. Ophthalmic assessments during Period 1, Visits 2-9 are performed on both eyes.

At Period 2, Visit 1 (Day 0, Injection Day Visit for SE2) subjects undergo a vitrectomy with retinal detachment and sub-retinal injection of AAV2-REP1 in the contralateral, untreated eye (SE2). Subjects no longer follow the Period 1 visit schedule, but instead attend Period 2, Visits 2-9 according to the Schedule of Study Procedures. Ophthalmic assessments scheduled for Period 2, Visits 2-9 will be performed on both eyes.

Subjects are assessed for safety and efficacy throughout the study; assessments are outlined in the Schedule of Study Procedures. Subjects who develop cataracts may undergo cataract surgery if deemed clinically necessary. If cataract surgery is performed, it is carried out at least 4 weeks before Month 12 (Visit 9) for the respective eye.

The primary endpoint is the evaluation of safety following bilateral administration of AAV2-REP1. Secondary endpoints of the study include a change from baseline in best corrected visual acuity (BCVA) as measured by the Early Treatment of Diabetic Retinopathy Study (ETDRS) chart, a change from baseline in autofluorescence (AF), a change from baseline in spectral domain optical coherence tomography (SD-OCT), and a change from baseline in microperimetry.

Inclusion Criteria:

At the Screening Visit, subjects are eligible for study participation if they meet all of the following inclusion criteria. Inclusion criteria: (1)_are willing and able to give informed consent for participation in the study to have both eyes treated, (2) are male and ≥18 years of age, (3) have a genetically-confirmed diagnosis of CHM, (4) have active disease clinically visible within the macular region of both eyes, (5) have a BCVA of: ≥74 ETDRS letters (equivalent to better than or equal to 6/9 or 20/32 Snellen, decimal 0.63, LogMar 0.2) in both eyes, if no prior treatment with AAV2-REP1≥74 ETDRS letters (equivalent to better than or equal to 6/9 or 20/32 Snellen, decimal 0.63, LogMar 0.2) in the untreated eye, if prior treatment with AAV2-REP1 was received in an alternative study. If previously treated with AAV2-REP1 in an alternative study, subjects may be eligible for participation following Sponsor approval.

Exclusion Criteria:

At the Screening Visit, subjects are not eligible for study participation if they meet any of the following exclusion criteria. Exclusion criteria: (1) have a history of amblyopia or inflammatory disorder in either eye, (2) are unwilling to use barrier contraception methods, for a period of 3 months following treatment with AAV2-REP1 in either eye, (3) have had previous intraocular surgery performed within 3 months of the Screening Visit in either eye, (4) have any other significant ocular or non-ocular disease/disorder which, in the opinion of the investigator, may either put the subjects at risk because of participation in the study, or may influence the results of the study, or the subject's ability to participate in the study, and (5) have participated in another research study involving an investigational product in the past 12 weeks or received a gene/cell-based therapy at any time previously, except if treated within another study with AAV2-REP1. This significant ocular or non-ocular disease/disorder which, in the opinion of the investigator, may either put the subjects at risk because of participation in the study, or may influence the results of the study, or the subject's ability to participate in the study includes but is not limited to a potential subject: a) with a contraindication to oral corticosteroid (e.g. prednisolone/prednisone), b) with clinically significant cataract in either eye, c) who, in the clinical opinion of the Investigator, is not an appropriate candidate for sub-retinal surgery.

Test product, dosage, and mode of administration: For each eye, subjects will undergo vitrectomy and retinal detachment and receive a volume of 0.1 mL sub-retinal injection of study drug containing 1×10¹¹ AAV2-REP1 genome particles.

Criteria for Evaluation: The safety evaluation is based on full ophthalmic examination (including intraocular pressure [IOP], slit lamp examination, lens opacity grading and dilated ophthalmoscopy); fundus photography; adverse event (AE) reporting; vector shedding and immunogenicity sampling; and vital signs.

Efficacy: The efficacy evaluation is based on BCVA (as measured by the ETDRS chart), fundus autofluorescence, SD-OCT and microperimetry.

Statistical Methodology: No formal sample size calculation was performed. Continuous variables are summarized over time using descriptive statistics (i.e., mean, standard deviation, 95% confidence interval [CI], median, Q1, Q3, P05, P95, min, and max). Categorical variables are described over time using counts, percentages, and 95% CIs. Summaries are tabulated by visit and eye. No formal statistical testing is performed. AEs are summarized by system organ class, preferred term and eye. Both the number of subjects and the number of eyes experiencing an AE, as well as the number of events, are summarized. Similar summaries will be produced for study drug/procedure-related AEs, AEs leading to discontinuation, and serious AEs. AEs are also summarized by maximum severity, relationship to study drug/procedure, and time to onset and resolution. Vector shedding and immune response profiles will be described. The remaining safety evaluations are analyzed using descriptive statistics.

CHM is incurable and, prior to the development of the pharmaceutical compositions of the disclosure, treatment was supportive at best. The pharmaceutical composition administered to subjects of this trial, AAV2-REP1 is an AAV2 particle encapsulating 1.962 kB complementary deoxyribonucleic acid (cDNA) of the wild-type human REP1 gene. AAV2-REP1 expresses high levels of human REP1 protein, restores REP1 to human CHM fibroblasts, provides functional rescue of human CHM cells, expresses protein in the retina of CHM mice in vivo, and is non-toxic when over-expressed by one log unit. Further, over-expression of the human REP1 protein does not significantly negatively affect retinal function.

Considering that CHM affects both eyes, this study demonstrates bilateral AAV2-REP1 administration. Emerging data from non-clinical and clinical studies with AAV2 vectors demonstrate that AAV2 vectors illicit a minimal immune response, including after bilateral administration. The aim of this study is to provide important insight into the safety and tolerability of sequential, bilateral treatment with AAV2-REP1.

Treatments Administered

Eligible subjects undergo vitrectomy and retinal detachment in each eye. At Period 1, Visit 1 (Day 0, the Injection Day Visit for SE1), subjects receive a volume of 0.1 mL sub-retinal injection of study drug containing 1×10¹¹ AAV2-REP1 gp in SE1. At Period 2, Visit 1 (Day 0, the Injection Day Visit for SE2), subjects receive the same sub-retinal injection of AAV2-REP1 in SE2.

Description of Pharmaceutical Composition

The AAV2 vector contains recombinant human cDNA encoding REP1 (AAV2-REP1). The vector genome (AAV2-CBA-hREP1-WPRE-BGH) is comprised of a strong constitutive expression cassette, a hybrid CBA promoter, the human cDNA encoding REP1, a modified woodchuck hepatitis virus post-transcriptional regulatory element (WPRE) sequence, and a bovine growth hormone polyadenylation (BGH-polyA) sequence flanked by AAV2 inverted terminal repeats. The cDNA fragment was originally isolated from a human retinal cDNA library from unaffected individuals.

The AAV2-REP1 drug product is formulated in a sterile, 20 mM Tris-buffered solution, pH 8.0, and contains 1 mM MgCl2, 200 mM NaCl and 0.001% PF68. The drug product is a clear to slightly opalescent, colorless, sterile-filtered suspension with a target concentration of 1×10¹² gp/mL.

AAV2-REP1 is supplied in sterile vials, stoppered and capped. A total of 0.3 mL vector suspension is supplied for each eye to be treated. Prior to shipment, each vial is placed in a labeled secondary container. The drug product is to be stored at <−60° C. (<−76° F.) in a controlled access, temperature monitored freezer.

Vitrectomy Procedure and Injection of AAV2-REP1

Injection of AAV2-REP1 is performed by an appropriately qualified and experienced retinal surgeon. Due to the complexity and unpredictability of detaching the retina in CHM, in which the retina and choroid can be extremely thin and fused in places, a modification to the technique of sub-retinal gene therapy has been developed. This involves performing the vector delivery in 2 steps after vitrectomy. An advantage of a 2-step procedure is that any unexpected complications of retinal detachment can be managed conservatively, minimizing concerns about the vector escaping into the vitreous. Further, the injection could be deferred until a later date if, for instance, a macular hole was created which required treatment with gas. Also, since the volume of fluid required to detach the fovea is variable, by removing the vector from the first step, a precise consistent dose in terms of genome particles can still be applied into the sub-retinal space.

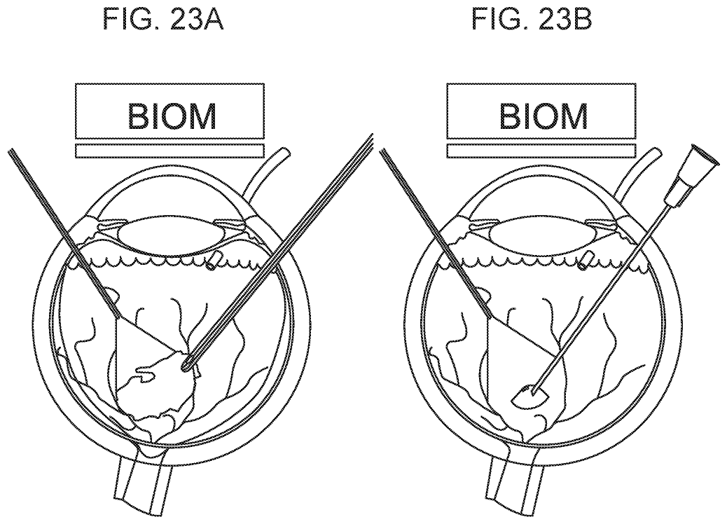

Initially, subjects undergo a standard vitrectomy and detachment of the posterior hyaloid in the respective study eye (FIG. 23). All surgeries will be conducted using the standard BIOM vitrectomy system. A 23-gauge sutured approach is usually favored to avoid any potential risks of wound leakage. The retina will be detached with 0.1-0.5 mL of balanced salt solution (BSS) injected through a 41-gauge sub-retinal cannula connected to a vitreous injection set. A single dose of AAV2-REP1 will be injected into the sub-retinal fluid through the same entry site.

In the second step of the procedure, the BSS cannula is removed from the eye and AAV2-REP1 is prepared for injection. A dose of 1.0×10¹¹ AAV2-REP1 gp is injected into the sub-retinal space through the same entry site. The vector needs to be primed in the 1 mL syringe to avoid formation of air bubbles, and a connector is used so that the 1 mL syringe can be connected to the constant pressure line of the vitrectomy machine. The subretinal injection will target any area of the macula but also include the fovea if possible. In each case, the vector is injected so that the sub-retinal fluid overlies all edge boundaries of the central region that has yet to undergo chorioretinal degeneration, as identified by fundus autofluorescence. After wound closure, care is taken to dispose of all irrigating fluids that may have passed through the eye to limit potential vector spread.

Concomitant Therapy:

Subjects cannot have participated in another research study involving an investigational product in the past 12 weeks or received a gene/cell-based therapy at any time previously, except if treated within another study with AAV2-REP1. Throughout the study, investigators may prescribe any concomitant medications or treatments deemed necessary to provide adequate supportive care. Details of concomitant medications are collected at the Screening Visit and updated at every study visit (including the ET Visit, if applicable). Concomitant medications (including oral corticosteroid) taken during the study are to be recorded in the subject's medical records and eCRF; an exception to this is any medication used in the course of conducting a study procedure (e.g., anaesthesia, dilating eye drops).

To minimize inflammation resulting from surgery and potential or unexpected immune responses to vector/transgene, all subjects will be given a 21-day course of oral prednisone/prednisolone prior to each surgery. Hence, for each surgery, this would be 1 mg/kg/day prednisone/prednisolone for a total of 10 days (beginning 2 days before vector injection, on the day of injection, and then for 7 days); followed by 0.5 mg/kg/day for 7 days; 0.25 mg/kg/day for 2 days; and 0.125 mg/kg/day for 2 days (21 days in total). Details of corticosteroid usage are captured by each subject in a diary card.

Study Procedures

At each study visit, an attempt should be made to perform all procedures in both eyes.

Screening Visit

The investigator explains the study purpose, procedures and subject responsibilities to each potential study subject. Screening procedures will consist of the following (all ophthalmic assessments will be conducted on both eyes): Demography, medical and ocular history, Vital signs, Weight, Vector shedding sampling—blood, tears (both eyes), urine, saliva, Immunogenicity sampling (enzyme-linked immunosorbent assay [ELISA] and enzyme-linked immunospot [ELISPOT]), BCVA, Full ophthalmic examination (including IOP, a slit-lamp examination, lens opacity, and dilated fundus examination), SD-OCT, Autofluorescence, Microperimetry, 7-field color fundus photography (including stereo photographs for fields 1, 2, and 3), Serious AE (SAE) monitoring, Medication review.

Subjects who meet all of the inclusion criteria and none of the exclusion criteria will are assigned a subject number and enrolled into the study. The investigator assigns the order in which the eyes are treated (i.e., SE1 and SE2, respectively). This will be done in collaboration with the subject; however, the worse eye is generally be selected for treatment first. The target interval between the surgical procedure in SE1 and SE2 is determined at this visit.

For each subject, the interval between SE1 and SE2 treatment is expected to range from a few weeks to months. While this interval is decided on a case-by-case basis, an effort should be made to schedule varying treatment intervals (e.g. 1, 3 or 6+ months apart) in order to better characterize the immunological and safety profile of sequential treatment administration.

The next study visit (Period 1, Visit 1) is to be scheduled within 10 weeks of the Screening Visit. Subjects will attend Period 1 Visits 2-9 unless the second surgery visit (Period 2, Visit 1) begins, which will mark the start of study Period 2. Given that the timing of the second surgery visit (Period 2, Visit 1) will vary between subjects, the duration of study Period 1 will also be variable.

Subjects are given two 21-day courses of oral corticosteroid (e.g., prednisolone/prednisone) and instructed to start taking the drug 2 days before their next surgery, at Period 1, Visit 1 for SE1 and Period 2, Visit 1 for SE2. Subjects will be issued a diary card to capture corticosteroid compliance throughout each 21-day period. Subjects are instructed to use barrier contraception for a period of 3 months from the time they are treated.

The schedule of procedures is identical for Periods 1 and 2; during each period, procedures are to be conducted in both SE1 and SE2.

If a subject has previously participated in a clinical trial of AAV2-REP1 for the treatment of CHM and has received AAV2-REP1 in one eye, the untreated eye will be assigned as SE2 at the Screening Visit and the subject will immediately enter Period 2.

Period 1 and 2, Visit 1 (Day 0, Injection Day Visit)

At Visit 1 (Day 0, Injection Day Visit), the following assessments are performed: Full ophthalmic examination (including IOP, a slit-lamp examination, lens opacity, and dilated fundus examination), AE/SAE monitoring, and concomitant medication review (including review of the corticosteroid diary card).

Subjects then undergo vitrectomy and receive a volume of 0.1 mL sub-retinal injection of AAV2-REP1, containing 1×10¹¹ gp in their first (SE1) eye.

Subjects are carefully monitored for the occurrence of AEs during the procedure. Subjects return to the site 1, 3, 7 and 14 days after surgery for post-operative follow-up (Visit 2 [Day 1], Visit 3 [Day 3], Visit 4 [Day 7] and Visit 5 [Day 14], respectively).

To allow accurate characterization of AAV2-REP1 safety and immunogenicity profile, treatment of SE2 should not occur if a previous intraocular surgery was performed on the same eye within 3 months of the planned treatment date. If intraocular surgery has occurred within 3 months, treatment of SE2 should be delayed until such a time that the 3-month interval has elapsed and there is complete post-operative recovery of the eye.

Period 1 and 2, Visit 2 (Post-Operative Visit on Day 1)

For Visit 2, ocular assessments and procedures are performed on each eye. At Visit 2 (Day 1), subjects return to the site for their first post-operative visit for the same eye that underwent surgery at Visit 1 (Period 1 [SE1] or Period 2 [SE2]). The following assessments are performed: Vital signs, Vector shedding sampling—blood, tears (both eyes), urine, saliva, Immunogenicity sampling (ELISA and ELISPOT), BCVA, Full ophthalmic examination (including IOP, a slit-lamp examination, lens opacity, and dilated fundus examination), SD-OCT, AE/SAE monitoring, Medication review (including review of the corticosteroid diary card).

Period 1 and 2, Visit 3 (Day 3)

For Visit 3 (Day 3), ocular assessments and procedures are performed on each eye. At Visit 3 (Day 3), subjects return to the site for their second post-operative visit for the same eye that underwent surgery at Visit 1 (Period 1 [SE1] or Period 2 [SE2]). The following assessments will be performed: Vital signs, Vector shedding sampling—blood, tears (both eyes), urine, saliva, BCVA, Full ophthalmic examination (including IOP, a slit-lamp examination, lens opacity, and dilated fundus examination), AE/SAE monitoring, and Medication review (including review of the corticosteroid diary card).

Period 1 and 2, Visit 4 (Day 7)

For Visit 4 (Day 7±1 day), ocular assessments and procedures are performed on each eye. At Visit 4 (Day 7±1 day), subjects return to the site for their third post-operative visit for the same eye that underwent surgery at Visit 1 (Period 1 [SE1] or Period 2 [SE2]). The following assessments will be performed: Vector shedding sampling—blood, tears (both eyes), urine, saliva, Immunogenicity sampling (ELISA and ELISPOT), BCVA, Full ophthalmic examination (including IOP, a slit-lamp examination, lens opacity, and dilated fundus examination), SD-OCT, Autofluorescence, AE/SAE monitoring, and Medication review (including review of the corticosteroid diary card).

Period 1 and 2, Visit 5 (Day 14)

For Visit 5 (Day 14±3 days), ocular assessments and procedures are performed on each eye. At Visit 5 (Day 14±3 days), subjects return to the site for their fourth postoperative visit for the same eye that underwent surgery at Visit 1 (Period 1 [SE1] or Period 2 [SE2]). The following assessments will be performed: Vector shedding sampling—blood, tears (both eyes), urine, saliva, Immunogenicity sampling (ELISA and ELISPOT), BCVA, Full ophthalmic examination (including IOP, a slit-lamp examination, lens opacity, and dilated fundus examination), SD-OCT, Autofluorescence, AE/SAE monitoring, and Medication review (including review of the corticosteroid diary card)

Period 1 and 2, Visit 6 (Month 1) and Visit 7 (Month 3)

For Visit 6 (Month 1±7 days) and Visit 7 (Month 3±14 days), ocular assessments and procedures will be performed on each eye. The following assessments are performed: Vector shedding sampling—blood, tears (both eyes), urine, saliva, Immunogenicity sampling (ELISA and ELISPOT), BCVA, Full ophthalmic examination (including IOP, a slit-lamp examination, lens opacity, and dilated fundus examination), SD-OCT, Autofluorescence, Microperimetry, AE/SAE monitoring, and Medication review (including review and return of the corticosteroid diary card).

Period 1 and 2, Visit 8 (Month 6) and Visit 9 (Month 12)

For Visit 8 (Month 6±14 days) and Visit 9 (Month 12±14 days), ocular assessments and procedures are performed on each eye. The following assessments are performed: Vital signs (Visit 9, Month 12 only), Immunogenicity sampling (ELISA and ELISPOT), BCVA, Full ophthalmic examination (including IOP, a slit-lamp examination, lens opacity, and dilated fundus examination), SD-OCT, Autofluorescence, Microperimetry, [symbol] -field color fundus photography (including stereo photographs for fields 1, 2, and 3) (Visit 9, Month 12 only), AE/SAE monitoring, and Medication review.

Early Termination

In the event that a subject discontinues the study at any time, the site should use every reasonable effort to ensure that an ET Visit is conducted. The following assessments should be performed (all ophthalmic assessments will be conducted on both eyes): Vital signs, Vector shedding sampling—blood, tears (both eyes), urine, saliva (only if the ET Visit occurs within 3 months post-treatment), Immunogenicity sampling (ELISA and ELISPOT), BCVA, Full ophthalmic examination (including IOP, a slit-lamp examination, lens opacity, and dilated fundus examination), SD-OCT, Autofluorescence, Microperimetry, 7-field color fundus photography (including stereo photographs for fields 1, 2, and 3), AE/SAE monitoring, and Medication review.

Adverse Event

An AE is any untoward medical occurrence in a clinical investigation subject, which does not necessarily have a causal relationship with the study medication/surgical procedure. An AE can therefore be any unfavorable and unintended sign (including an abnormal laboratory finding), symptom, or disease temporally associated with the use of the study medication/surgical procedure, whether or not related to the investigational product or with the surgical procedure described in this protocol. AEs are to also include any pre-existing condition (other than CHM) or illness which worsens during the study (i.e., increases in frequency or intensity).

Serious Adverse Event

An SAE is defined as any untoward medical occurrence that: Results in death, Is life-threatening, Requires inpatient hospitalization or prolongation of existing hospitalization, Results in persistent or significant disability/incapacity, Is a congenital anomaly/birth defect, Results in vision loss or is vision threatening, Is another important medical event(s). The term ‘life-threatening’ in the definition of ‘serious’ refers to an event in which the subject is at risk of death at the time of the event. It does not refer to an event that hypothetically might cause death if it were more severe. Hospitalization for a pre-existing condition, including elective procedures, which has not worsened, does not constitute an SAE. Other events that may not result in death, are not life threatening or do not require hospitalization, may be considered an SAE when, based upon appropriate medical judgment, the event may jeopardize the subject and may require medical or surgical intervention to prevent one of the outcomes listed above.

The following vision loss or vision-threatening events are to be reported as SAEs: Sustained decrease in VA of ≥15 letters on ETDRS chart compared to baseline, except for surgery-related events. Sustained is defined as lasting 48 hours or more until recovery; recovery defined as VA returned to within 5 letters of baseline VA.

Surgery-related events of VA decrease are defined as VA decreases occurring in close temporal association (within <24 hours) with the surgical administration of the study medication, and which are resolving at Day 7 (Period 1/2, Visit 4) post-surgery. These events are not to be reported as an AE or SAE. However, they should be reported as an AE if in the investigator's opinion, their evolution in terms of duration or severity cannot be explained by the procedure. This would include, but not be limited to instances where the abnormal course of post-surgery VA decrease is associated with another complication attributable to the surgery or the study medication, or where the abnormal course of post-surgery VA decrease can be attributed to another identifiable cause. AEs that in the opinion of the investigator, actually or potentially require any surgical or medical intervention to prevent permanent loss of sight.

Laboratory Assessments

Vector Shedding

Blood, tears (both eyes), urine and saliva samples are collected and tested using an appropriate assay for evidence of vector shedding and dispersion.

Immunogenicity

For the evaluation of immunogenicity, blood will be collected at the times indicated in Table 61 (Schedule of Study Procedures). Immunoassays are planned to assess antibody and cell based responses against AAV2-REP1. ELISPOT assays will be used for T-cell mediated immune responses to transgene, and antibody responses will be assayed using ELISA-based methods.

Vital Signs

Vital signs (pulse and systolic and diastolic blood pressure) will be taken at the times indicated in Table 61 (Schedule of Study Procedures). Vital signs should be taken after the subject is seated for at least 5 minutes.

Full Ophthalmic Examination

A full ophthalmic examination will be performed for both eyes at the times indicated in Table 61 (Schedule of Study Procedures). Each ophthalmic examination will include IOP, slit lamp examination, lens opacity grading, and dilated ophthalmoscopy. The same slit lamp machine and lighting conditions should be used across study visits for any given subject. In addition to the parameters listed above, subjects are carefully examined for the presence of intraocular inflammation after vector administration. Cataract can also develop as a result of the vitrectomy procedure and can potentially affect VA. Pre-operative grading of lens opacity and color should therefore be documented. Subjects who develop cataracts may undergo cataract surgery if deemed clinically necessary. If cataract surgery is performed, it should be carried out at least 4 weeks before Month 12 (Visit 9) for the respective eye.

7-Field Color Fundus Photography

Seven-field color fundus photography will be performed for both eyes at the times indicated in Table 61 (Schedule of Study Procedures). Fundus photography will be performed by certified technicians following pupil dilation. Stereo photos should be performed for fields 1, 2 and 3. All fundus photographs will be sent by the sites to the Central Reading Centre (CRC) for review; the CRC will enter the data into the electronic data capture (EDC) system. For complete technical specifications for fundus photography, refer to the Study Operations Manual (which will include procedures from the CRC regarding how measurements are to be taken).

Assessment of Efficacy

Visual Acuity

To evaluate changes in visual acuity (VA) over the study period, BCVA is assessed using the ETDRS VA chart and performed for both eyes at the times indicated in Table 61 (Schedule of Study Procedures). The BCVA test should be performed prior to pupil dilation, and distance refraction should be carried out before BCVA is measured. Initially, letters are read at a distance of 4 meters from the chart. If <20 letters are read at 4 meters, testing at 1 meter should be performed. BCVA is to be reported as number of letters read correctly by the subject. For BCVA, assessors will be appropriately qualified for conducting the assessment.

Fundus Autofluorescence

To assess changes in the area of viable retinal tissue, fundus autofluorescence is performed on both eyes at the times indicated in Table 61 (Schedule of Study Procedures).

Spectral Domain Optical Coherence Tomography (SD-OCT)

SD-OCT are performed on both eyes at the times indicated in Table 61 (Schedule of Study Procedures). SD-OCT is used to quantify integrity of the ellipsoid zone and reduction in the signal from the outer nuclear layer and choroid. In addition, foveal changes are assessed.

Microperimetry

Microperimetry is conducted on both eyes at the times indicated in Table 61 (Schedule of Study Procedures). Microperimetry is conducted to assess changes in retinal sensitivity within the macula.

TABLE 61
Schedule of Study Procedures

| | | Study Period 1/Period 2 | | | | | | | | | | |
|---|---|---|---|---|---|---|---|---|---|---|---|---|
| | | Visit | | | | | | | | | | |
| | | V1 | V2 | V3 | V4 | V5 | V6 | V7 | V8 | V9 | | |
| | Study Day/Month Visit Window | | | | | | | | | | | |
| | | Day 0 | | | | | | | | | | |
| | | Injection | | | | | | | | | | |
| | | Day^{b} | | | | | | | | | | |
| | | (≤10 | Day 1 | | | | | | | | | |
| | | weeks | Post | Day 3 | Day 7 ± | Day 14 ± | M1 ± | M3 ± | M6 ± | M12 ± | ET | Unscheduled |
| | Screening^{a} | Screening) | op | Post op | 1 d | 3 d | 7 d | 14 d | 14 d | 14 d | Visit^{c} | Visit^{d} |
| Assessment/Procedures | | | | | | | | | | | | |
| (All subjects/eyes) | | | | | | | | | | | | |
| Informed Consent | X | | | | | | | | | | | |
| Demography, medical and | X | | | | | | | | | | | |
| ocular history | | | | | | | | | | | | |
| Vital signs (pulse, blood | X | | X | X | | | | | | X | X | |
| pressure) | | | | | | | | | | | | |
| Weight^{e} | X | | | | | | | | | | | |
| Vector shedding sampling^{f} | X | | X | X | X | X | X | X | | | X [symbol] | |
| Immunogenicity sampling | X | | X | | X | X | X | X | X | X | X | |
| (ELISA and ELISPOT) | | | | | | | | | | | | |
| BCVA | X | | X | X | X | X | X | X | X | X | X | X |
| Full ophthalmic exam^{h} | X | X | X | X | X | X | X | X | X | X | X | X |
| SD-OCT | X | | X | | X | X | X | X | X | X | X | X |
| Autofluorescence | X | | | | X | X | X | X | X | X | X | |
| Microperimetry | X | | | | | | X | X | X | X | X | |
| 7-field-colour fundus photosⁱ | X | | | | | | | | | X | X | |
| Study drug/sub-retinal | | X | | | | | | | | | | |
| injection/vitrectomy/retinal | | | | | | | | | | | | |
| detachment | | | | | | | | | | | | |
| AE/SAE monitoring^{j} | X | X | X | X | X | X | X | X | X | X | X | X |
| Concomitant medication^{k} | X | X | X | X | X | X | X | X | X | X | X | X |

[symbol]

indicates data missing or illegible when filed

### Example 15: Star Study

This study is an open label, outcomes-assessor masked, prospective, randomized, parallel controlled group, multi-center, global, interventional study. The study consists of 8 visits with a 12-month evaluation period. At the Screening/Baseline Visit, each subject is assessed for eligibility. For eligible subjects a study eye are assigned, and the subjects are randomized in a 2:1:2 ratio to receive either AAV2-REP1 high dose (1.0×10¹¹ genome particles [gp]), AAV2-REP1 low dose (1.0×10¹⁰ gp) or to enter the untreated Control group.

At the Injection Day Visit (Visit 2, Day 0), subjects in the AAV2-REP1 high and low dose treatment arms undergo vitrectomy and receive a sub-retinal injection of the assigned treatment dose of AAV2-REP1 in their study eye in a double-masked manner; these subjects then return to the site for 2 post-operative follow up visits on Day 1 (Visit 3) and Day 7 (Visit 4). Subjects in the Control group do not undergo surgery, receive any study drug in their study eye (i.e., Control-study eye) or attend the 2 on-site post-operative visits. Instead a telephone contact from the site occurs for the Control group on Day 0 (Visit 2), Day 1 (Visit 3) and Day 7 (Visit 4). All subjects are followed for 12 months from Visit 2 (Day 0).

Study data are collected for both eyes of each subject. Since AAV2-REP1 treatment requires an invasive surgical procedure under general anaesthesia, the sponsor, investigator and the subject are unmasked to the study procedure (i.e., vitrectomy and sub-retinal injection), however within the treated groups, the sponsor, investigator and subject is masked to the assigned dose (1.0×10¹¹ gp or 1.0×10¹⁰ gp). To further minimize potential bias of the treated and non-treated eye evaluations, all subjective ophthalmic assessments at the Screening/Baseline Visit (Visit 1) and from Month 1 (Visit 5) onwards (including the Month 12 Primary Endpoint evaluation) will be conducted by a masked assessor.

Subjects are assessed for efficacy and safety throughout the study as indicated in the Schedule of Study Procedures. Subjects who develop cataracts may undergo cataract surgery if deemed clinically necessary; if surgery is performed, it should be carried out at least 4 weeks before the Month 12 Visit/End of Study (EOS) Visit.

Subjects

Approximately 140 subjects randomized in a 2:1:2 ratio; 56 subjects in the AAV2-REP1 high dose (1.0×10¹¹ gp) group, 28 subjects in the AAV2-REP1 low dose (1.0×10¹⁰ gp) and 56 subjects in the untreated control group.

Inclusion Criteria: Subjects are eligible for study participation if they meet all of the following inclusion criteria: (1) are willing and able to give informed consent for participation in the study, (2) are male and ≥18 years of age, (3) have a genetically-confirmed diagnosis of CHM, (4) have active disease clinically visible within the macular region in the study eye, (5) have a BCVA of 34-73 ETDRS letters (equivalent to worse than or equal to 6/12 or 20/40 Snellen acuity, but better than or equal to 6/60 or 20/200 Snellen acuity) in the study eye.

Exclusion Criteria: Subjects are not eligible for study participation if they meet any of the following exclusion criteria: (1) have a history of amblyopia in the eligible eye, (2) are unwilling to use barrier contraception methods, for a period of 3 months, if treated with AAV2-REP1, (3) previous intraocular surgery performed in the study eye within 3 months of Visit 1, (4) have any other significant ocular or non-ocular disease/disorder which, in the opinion of the investigator, may either put the subjects at risk because of participation in the study, or may influence the results of the study, or the subject's ability to participate in the study (including, but not limited to, a contraindication to oral corticosteroid (e.g. prednisolone/prednisone), with a clinically significant cataract, who, in the clinical opinion of the Investigator, is not an appropriate candidate for sub-retinal surgery), and (5) have participated in another research study involving an investigational product in the past 12 weeks or received a gene/cell-based therapy at any time previously.

Test product, dosage, and mode of administration: All subjects receiving active treatment undergo vitrectomy and receive a volume of 0.1 mL sub-retinal injection of study drug containing either high dose (1.0×10¹¹ gp) AAV2-REP1 or low dose (1.0×10¹⁰ gp) AAV2-REP1 in their study eye.

Criteria for Evaluation:

The primary efficacy endpoint is the proportion of subjects with a ≥15-letter improvement from Baseline in best corrected visual acuity (BCVA) at Month 12 as measured by the Early Treatment of Diabetic Retinopathy Study (ETDRS) chart. The key secondary endpoint is the proportion of subjects with a ≥15-letter improvement from Baseline in BCVA at Month 12 when compared with the change in BCVA over 12 months. Other secondary endpoints include (1) proportion of subjects with a >10 letter increase from Baseline in BCVA at Month 12, (2) mean of the mean change from Baseline in BCVA collected over Months 4, 8 and 12, (3) change from baseline in total area of preserved autofluorescence (AF) at Month 12, (4) change from baseline in the area of preserved ellipsoid zone (spectral domain optical coherence tomography [SD-OCT]) at Month 12, (5) change from baseline in microperimetry at Month 12, (6) change from baseline in contrast sensitivity score at Month 12, (7) change from baseline in color vision at Month 12, (8) change from baseline in reading speed test at Month 12, (9) maintenance of BCVA at Month 12, as measured by the ETDRS chart, (10) change from baseline in the 25-item Visual Function Questionnaire (VFQ-25) at Month 12. An exploratory efficacy endpoint includes tan evaluation of other anatomical and functional outcome measures. A safety endpoint includes an evaluation of safety assessments, including adverse events (AEs).

Efficacy: The efficacy evaluation are based on BCVA, fundus AF, SD-OCT, microperimetry, contrast sensitivity, color vision, reading speed test assessments, VFQ-25 and low luminance visual acuity (LLVA).

Safety: The safety evaluation are based on full ophthalmic examination (including intraocular pressure, slit lamp examination, lens opacity grading, and dilated ophthalmoscopy), fundus photography, AE reporting, immunogenicity, and vital signs. Any safety information collected as a result of the efficacy assessments (e.g., BCVA) is used in the overall safety evaluation, as appropriate.

Study Design

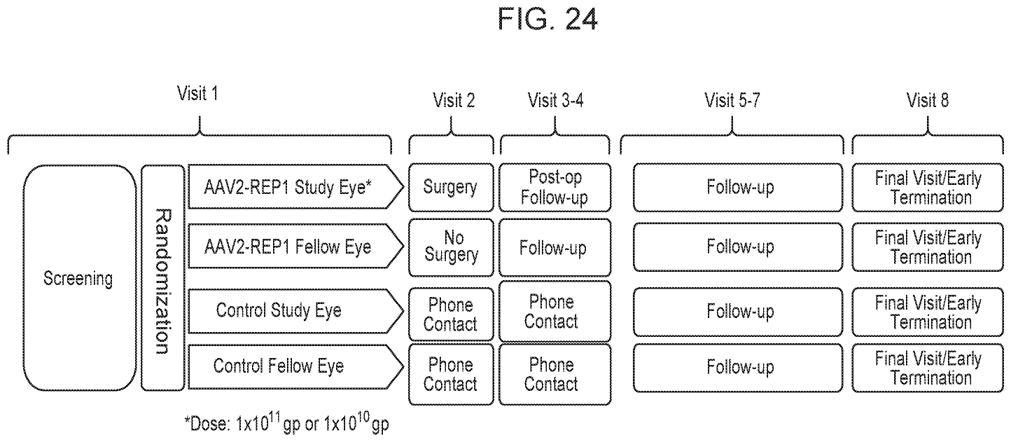

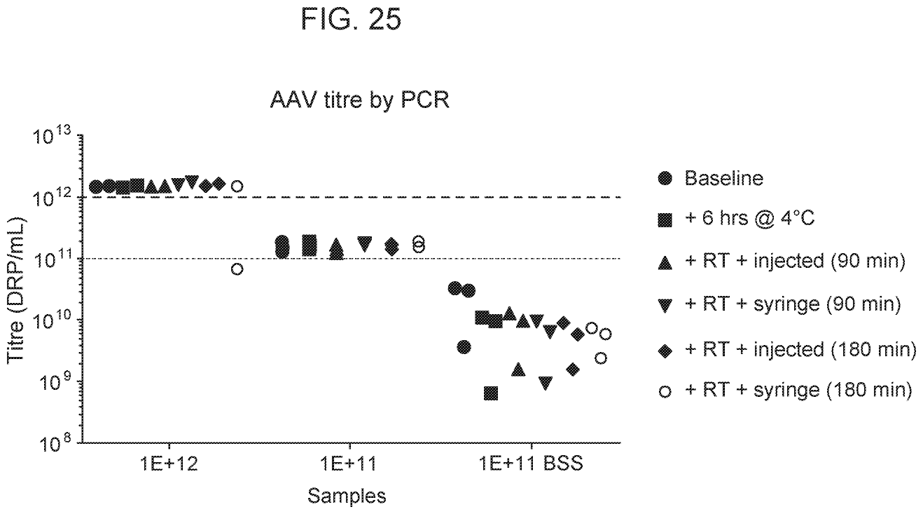

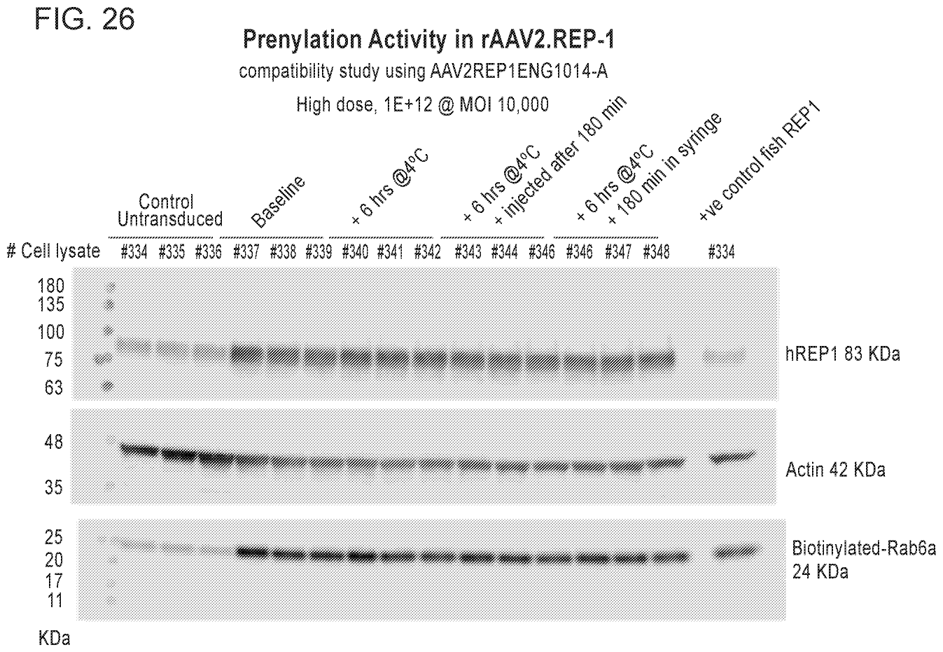

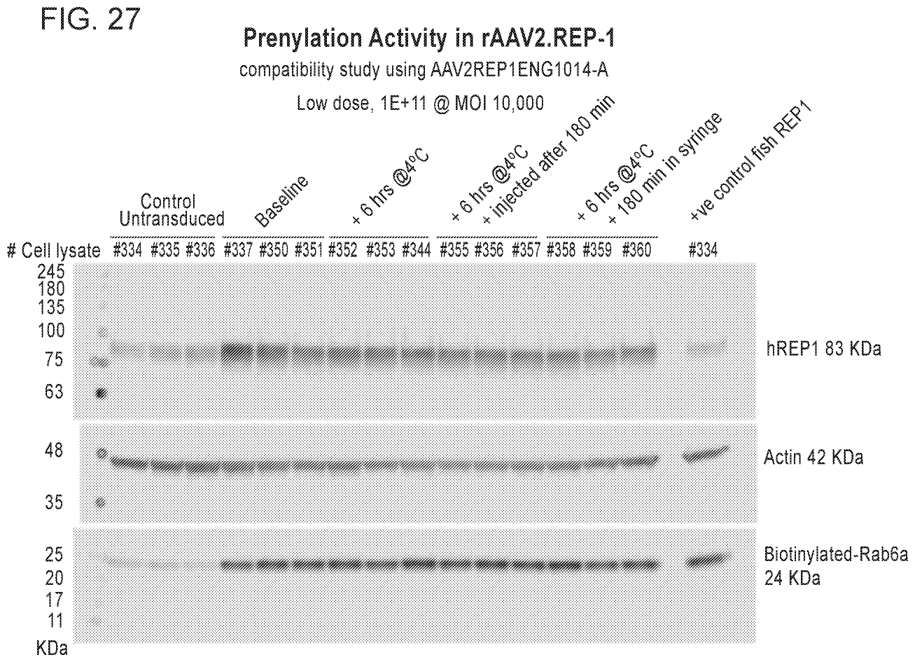

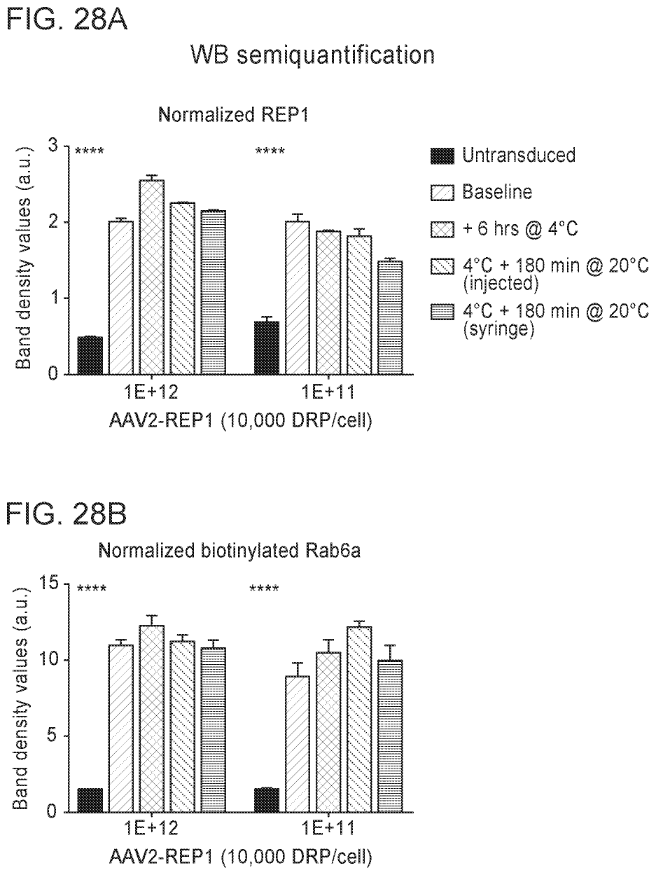

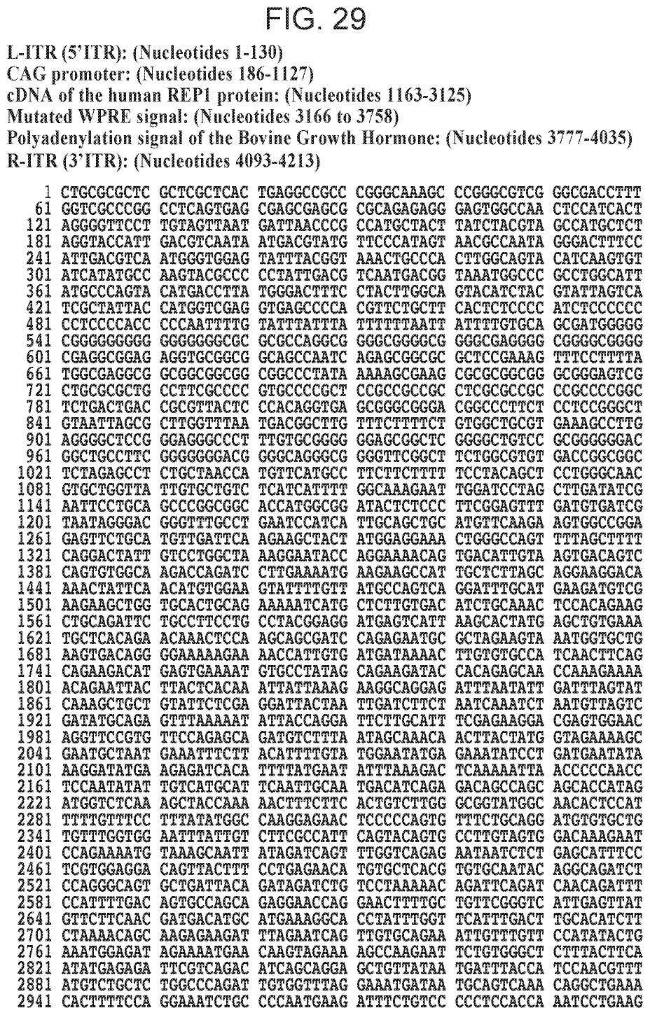

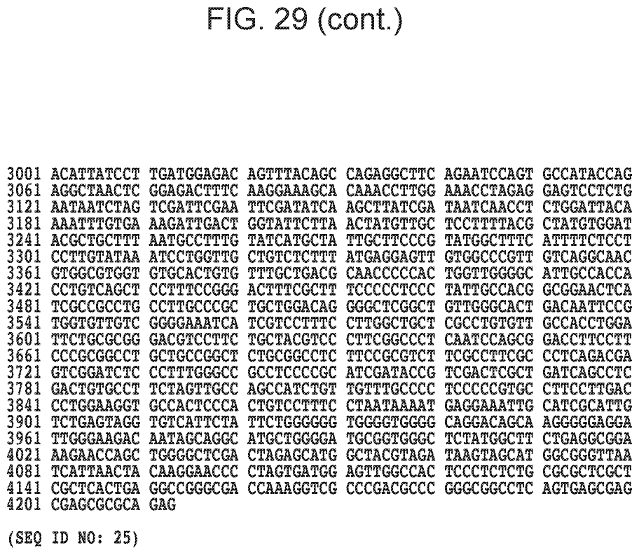

FIG. 24 provides a schematic of the design of this study.

At the Screening/Baseline Visit (Visit 1), each subject is assessed for eligibility of both eyes. If a subject has only 1 eligible eye, that eye is designated as the “study eye” and the subject's other (non-eligible) eye is designated as the “fellow eye.” If a subject has 2 eligible eyes, the selection of the “study eye” is made on clinical grounds and is generally be the worse eye affected. This decision is discussed in detail and agreed with each subject as part of the informed consent process. Subject choice is considered in cases where the degeneration is relatively symmetrical between the two eyes.

At the Screening/Baseline Visit, eligible subjects will be randomized in a 2:1:2 ratio to either the AAV2-REP1 high dose group (1.0×10¹¹ gp), the AAV2-REP1 low dose group (1.0×10¹⁰ gp) or the untreated Control group. To facilitate understanding of the study design, eyes are classified into 4 categories based on treatment group assignment and designation of study/fellow eye: AAV2-REP1-study eye (includes high and low dose); AAV2-REP1-fellow eye (includes high and low dose); Control-study eye; and Control-fellow eye.

At the Injection Day Visit (Visit 2, Day 0), subjects in the AAV2-REP1 treatment groups undergo vitrectomy and retinal detachment before receiving a sub-retinal injection of AAV2-REP1 high dose (1.0×10¹¹ gp) or AAV2-REP1 low dose (1.0×10¹⁰ gp) in their study eye (i.e., AAV2-REP1-study eye) in a double-masked manner; these subjects then return to the site for 2 post-operative follow up visits on Day 1 (Visit 3) and Day 7 (Visit 4). Subjects in the Control group does not undergo surgery, receive study drug or attend the 2 on-site safety post-operative visits. Instead a telephone contact from the site occurs for the Control group on Day 0 (Visit 2), Day 1 (Visit 3) and Day 7 (Visit 4). All subjects are followed for 12 months from Visit 2 (Day 0).

Study data are collected for both eyes of each subject. Since AAV2-REP1 treatment requires an invasive surgical procedure under general anaesthesia, the sponsor, investigator and the subject are unmasked to the study procedure (i.e. vitrectomy and sub-retinal injection), however within the treated groups, the sponsor, investigator and subject are masked (i.e. double-masking) to the assigned dose (1.0×10¹¹ gp or 1.0×10¹⁰ gp). To further minimize potential bias of the treated and non-treated eye evaluations, all subjective ophthalmic assessments at the Screening/Baseline Visit (Visit 1) and from Month 1 (Visit 5) onwards, are conducted by a masked assessor. Subjects are assessed for efficacy and safety throughout the study. The efficacy evaluation is based on BCVA, fundus AF, SD-OCT, microperimetry, contrast sensitivity, color vision, reading speed test, VFQ-25 and low luminance visual acuity (LLVA). The safety evaluation is based on full ophthalmic examination (including intraocular pressure [IOP], slit lamp examination, lens opacity grading, and dilated ophthalmoscopy), fundus photography, adverse event (AE) reporting, laboratory assessments (immunogenicity), and vital signs. Any safety information collected as a result of the efficacy assessments (e.g., BCVA) is used in the overall safety evaluation, as appropriate.

Subjects who develop cataracts may undergo cataract surgery if deemed clinically necessary; if surgery is performed, it should be carried out at least 4 weeks before the Month 12 Visit/End of Study (EOS) Visit (primary endpoint).

A subject is considered to have completed the study if he completes the Month 12 assessments. The end of the trial is the date the last subject completes his Month 12 assessments (or early termination [ET] assessments in the event of premature discontinuation) or the date of last data collection if the last subject is lost to follow-up. After study completion, treated subjects are invited to participate in a long term follow up study which permits continued efficacy and safety monitoring over a period of 5 years post-treatment.

Treatments Administered

At the Screening/Baseline Visit, eligible subjects are randomized in a 2:1:2 ratio to receive either an AAV2-REP1 low dose (1.0×10¹⁰ gp) or an AAV2-REP1 high dose (1.0×10¹¹ gp), while subjects in the untreated Control group receive no sham surgery or study medication. At the Injection Day Visit (Visit 2, Day 0), AAV2-REP1 is administered as a sub-retinal injection after vitrectomy for subjects in the AAV2-REP1 groups.

Description of Pharmaceutical Composition

The AAV2 vector contains recombinant human cDNA encoding REP1 (AAV2-REP1). The vector genome (AAV2-CBA-hREP1-WPRE-BGH) is comprised of a strong constitutive expression cassette, a hybrid CBA promoter, the human cDNA encoding REP1, a modified woodchuck hepatitis virus post-transcriptional regulatory element (WPRE) sequence, and a bovine growth hormone polyadenylation (BGH-polyA) sequence flanked by AAV2 inverted terminal repeats. The cDNA fragment was originally isolated from a human retinal cDNA library from unaffected individuals.

The AAV2-REP1 drug product is formulated in a sterile, 20 mM Tris-buffered solution, pH 8.0, and contains 1 mM MgCl2, 200 mM NaCl and 0.001% PF68. The drug product is a clear to slightly opalescent, colorless, sterile-filtered suspension with a target concentration of 1×10¹² gp/mL.

AAV2-REP1 is supplied in sterile vials, stoppered and capped. A total of 0.3 mL vector suspension is supplied for each eye to be treated. Prior to shipment, each vial is placed in a labeled secondary container. The drug product is to be stored at <−60° C. (<−76° F.) in a controlled access, temperature monitored freezer.

Vitrectomy Procedure and Injection of AAV2-REP1

Injection of AAV2-REP1 is performed by an appropriately qualified and experienced retinal surgeon. Due to the complexity and unpredictability of detaching the retina in CHM, in which the retina and choroid can be extremely thin and fused in places, a modification to the technique of sub-retinal gene therapy has been developed. This involves performing the vector delivery in 2 steps after vitrectomy. An advantage of a 2-step procedure is that any unexpected complications of retinal detachment can be managed conservatively, minimizing concerns about the vector escaping into the vitreous. Further, the injection could be deferred until a later date if, for instance, a macular hole was created which required treatment with gas. Also, since the volume of fluid required to detach the fovea is variable, by removing the vector from the first step, a precise consistent dose in terms of genome particles can still be applied into the sub-retinal space.

Initially, subjects undergo a standard vitrectomy and detachment of the posterior hyaloid in the respective study eye (FIG. 23). All surgeries will be conducted using the standard BIOM vitrectomy system. A 23-gauge sutured approach is usually favored to avoid any potential risks of wound leakage. The retina will be detached with 0.1-0.5 mL of balanced salt solution (BSS) injected through a 41-gauge sub-retinal cannula connected to a vitreous injection set. A single dose of AAV2-REP1 will be injected into the sub-retinal fluid through the same entry site.

In the second step of the procedure, the BSS cannula is removed from the eye and AAV2-REP1 is prepared for injection. A dose of 1.0×10¹¹ AAV2-REP1 gp is injected into the sub-retinal space through the same entry site. The vector needs to be primed in the 1 mL syringe to avoid formation of air bubbles, and a connector is used so that the 1 mL syringe can be connected to the constant pressure line of the vitrectomy machine. The subretinal injection will target any area of the macula but also include the fovea if possible. In each case, the vector is injected so that the sub-retinal fluid overlies all edge boundaries of the central region that has yet to undergo chorioretinal degeneration, as identified by fundus autofluorescence. After wound closure, care is taken to dispose of all irrigating fluids that may have passed through the eye to limit potential vector spread.

Randomization

At the Screening/Baseline Visit all patients are assigned a screening identifier, which includes the center number and subject number. If the subject fulfils all eligibility criteria at the Screening/Baseline Visit, a study eye is assigned and the subject is randomized in a 2:1:2 ratio to receive either AAV2-REP1 high dose (1.0×10¹¹ gp), AAV2-REP1 low dose (1.0×10¹⁰ gp) or to enter the untreated Control group. Randomization is generated using a validated system that automates the random assignment of treatment groups to randomization numbers, and stratified by surgical group.

Concomitant Therapy:

Subjects cannot have participated in another research study involving an investigational product in the past 12 weeks or received a gene/cell-based therapy at any time previously, except if treated within another study with AAV2-REP1. Throughout the study, investigators may prescribe any concomitant medications or treatments deemed necessary to provide adequate supportive care. Details of concomitant medications are collected at the Screening Visit and updated at every study visit (including the ET Visit, if applicable). Concomitant medications (including oral corticosteroid) taken during the study are to be recorded in the subject's medical records and eCRF; an exception to this is any medication used in the course of conducting a study procedure (e.g., anaesthesia, dilating eye drops).

To minimize inflammation resulting from surgery and potential or unexpected immune responses to vector/transgene, all subjects will be given a 21-day course of oral prednisone/prednisolone prior to each surgery. Hence, for each surgery, this would be 1 mg/kg/day prednisone/prednisolone for a total of 10 days (beginning 2 days before vector injection, on the day of injection, and then for 7 days); followed by 0.5 mg/kg/day for 7 days; 0.25 mg/kg/day for 2 days; and 0.125 mg/kg/day for 2 days (21 days in total). Details of corticosteroid usage are captured by each subject in a diary card.

Study Visits and Procedures

The schedule of study procedures is presented in Table 62: Schedule of Study Procedures.

Visit 1 (Screening/Baseline Visit)

The investigator explains the study purpose, procedures, and subject responsibilities to each potential study subject. The subject's willingness and ability to meet the protocol requirements is determined.

After informed consent has been obtained, the subject is allocated a subject identifier number and evaluated to determine eligibility. Screening/baseline procedures consist of the following (assessments can be spread over 2 consecutive days if necessary): Demography, medical and ocular history (Only subjects with a genetically confirmed diagnosis of CHM may enter the study. Genetic confirmation must be available prior to Visit 1); Vital signs; Weight; Immunoassay sampling (enzyme-linked immunosorbent assay [ELISA] and enzyme-linked immunospot [ELISPOT]); BCVA; LLVA; Full ophthalmic examination, including IOP, a slit-lamp examination, lens opacity, and dilated fundus examination; SD-OCT; AF; Microperimetry; Contrast sensitivity test; Color vision test; 7-field color fundus photography (including stereo photographs for fields 1, 2, and 3); Reading speed test (if applicable); VFQ-25; Serious AE (SAE) monitoring; Medication review; and Corticosteroid dispensing (if randomized to AAV2-REP1 groups). In order to capture accurate BCVA values at Visit 1 (Screening/Baseline) the following conditions apply to the BCVA assessment: If the BCVA value at Visit 1 (Screening/Baseline) is ≥±10 letter gain or loss in the study eye compared to the previous NIGHT study visit (if applicable), then BCVA must be repeated an additional 2 times, resulting in a total of 3 BCVA measures at Visit 1. To facilitate the additional BCVA measures this visit should be conducted over 2 days, with BCVA measured twice on Day 1 and once on Day 2 (prior to pupil dilation). All 3 BCVA values must be recorded in the eCRF. The highest score will be used to define subject eligibility. If the BCVA value at Visit 1 (Screening/Baseline) is <±10 letter difference in the study eye compared to the previous NIGHT study visit, then BCVA will be collected once and will not be repeated.

Subjects who meet all of the inclusion criteria and none of the exclusion criteria have a study eye assigned and are enrolled into the study. At this time, subjects are informed of the randomization outcome (i.e., AAV2-REP1 treatment or the Control group) and instructed to not reveal their treatment group assignment to the masked assessors during the study. Subjects randomized to the AAV2-REP1 treatment groups (along with the Investigators and sponsor) remain masked to the assigned dose.

The next study visit (Visit 2) is to be scheduled within 8 weeks of the Screening/Baseline Visit. Subjects randomized to the AAV2-REP1 groups are given a 21-day course of oral corticosteroid (e.g., prednisolone/prednisone) and instructed to start taking the drug 2 days before their next study visit (Visit 2). Subjects randomized to the AAV2-REP1 groups are instructed to use barrier contraception for a period of 3 months from the time they are treated.

If the subject is randomized to the Control group then Visit 2 consists of a telephone call.

Visit 2 (Day 0, Injection Day Visit or Telephone Contact)

At Visit 2 (Day 0), all subjects in the AAV2-REP1 groups will visit the surgical site, and the following assessments are performed: Full ophthalmic examination, including IOP, a slit-lamp examination, lens opacity, and dilated fundus examination; AE/SAE monitoring; Concomitant medication review; and Corticosteroid compliance review.

Subjects in the AAV2-REP1 groups undergo vitrectomy and receive a sub-retinal injection of AAV2-REP1, containing either AAV2-REP1 low dose (1×10¹⁰ vg) or AAV2-REP1 high dose (1×10¹² vg) in their study eye. Subjects are carefully monitored for the occurrence of AEs during the procedure. Subjects return to the site 1 and 7 days after surgery for post-operative follow-up (Visits 3 [Day 1] and 4 [Day 7], respectively).

Subjects in the Control group receive a study visit Telephone Call at the agreed upon date and time. Sites conduct the following assessments during the telephone call: AE/SAE monitoring and concomitant medication review.

Visit 3 (Day 1 Post-Operative Visit)

At Visit 3 (Day 1), subjects in the AAV2-REP1 groups return to the surgical site for a post-operative visit. The following assessments are performed: Vital signs; Immunoassay sampling (ELISA only); BCVA; Full ophthalmic examination, including IOP, a slit-lamp examination, lens opacity, and dilated fundus examination; SD-OCT; AE/SAE monitoring; Medication review; and Corticosteroid compliance review. Subjects are reminded of the requirement to use barrier contraception for a period of 3 months from the time of treatment.

Subjects in the Control group receive a study visit Telephone Call at the agreed upon date and time. Sites conduct the following assessments during the telephone call: AE/SAE monitoring and concomitant medication review.

Visit 4 (Day 7 Post-Operative Visit±3 Days)

At Visit 4 (Day 7±3 days), subjects in the AAV2-REP1 groups return to their host site (i.e. same site where their Visit 1 Screening/Baseline visit was performed) for a second post-operative visit. However, for those subjects that have travelled from a non-surgical site to a surgical site, and in the event of a post-operative complication or for any other safety reason considered appropriate by the study surgeon and surgical site Investigator, Visit 4 may be conducted at the surgical site. In this event post-operative follow-up should continue at the surgical site until the surgeon/surgical site Investigator, agrees to discharge the subject to the care of the non-surgical site. The following assessments are performed: Vital signs; Immunoassay sampling (ELISA and ELISPOT); BCVA; LLVA; Full ophthalmic examination, including IOP, a slit-lamp examination, lens opacity, and dilated fundus examination; SD-OCT; AF; Microperimetry, AE/SAE monitoring; Medication review and Corticosteroid compliance review.

Subjects in the Control group receive a study visit Telephone Call at the agreed upon date and time. Sites conduct the following assessments during the telephone call: AE/SAE monitoring and concomitant medication review.

Visit 5 (Month 1±7 Days)

All subjects attend their host site (i.e. same site where their Visit 1 Screening/Baseline visit was performed) from Visit 5 onwards. At Visit 5 (Month 1±7 days), the following assessments are performed for all subjects (assessments can be spread over 2 consecutive days if necessary): Vital signs; Immunoassay sampling (ELISA and ELISPOT]); BCVA; LLVA; Full ophthalmic examination, including IOP, a slit-lamp examination, lens opacity, and dilated fundus examination; SD-OCT; AF; Microperimetry; AE/SAE monitoring; Medication review; Corticosteroid compliance review

Visit 6 (Month 4±7 Days)

At Visit 6 (Month 4±7 days), the following assessments are performed (assessments can be spread over 2 consecutive days if necessary): Immunoassay sampling (ELISA and ELISPOT); BCVA; LLVA; Full ophthalmic examination, including IOP, a slit-lamp examination, lens opacity, and dilated fundus examination; SD-OCT; AF; Microperimetry; Contrast sensitivity test; Color vision test; AE/SAE monitoring and Medication review.

Visit 7 (Month 8±14 Days)

At Visit 7 (Month 8±14 days), the following assessments are performed (assessments can be spread over 2 consecutive days if necessary): BCVA, LLVA; Full ophthalmic examination, including IOP, a slit-lamp examination, lens opacity, and dilated fundus examination; SD-OCT; AF; Microperimetry; Contrast sensitivity test; Color vision test; AE/SAE monitoring and Medication review.

Visit 8 (Month 12±14 Days, End of Study Visit)

At Visit 8 (Month 12±14 days), the following assessments are performed (assessments can be spread over 2 consecutive days if necessary): Vital signs; BCVA; LLVA; Full ophthalmic examination, including IOP, a slit-lamp examination, lens opacity, and dilated fundus examination; SD-OCT; AF; Microperimetry; Contrast sensitivity test; Color vision test; 7-field color fundus photography (including stereo photographs for fields 1, 2, and 3); Reading speed test (if applicable); VFQ-25; AE/SAE monitoring and Medication review.

Early Termination Visit

In the event that a subject discontinues the study at any time, the site should use every reasonable effort to ensure that an ET Visit is conducted. The following assessments should be performed: Vital signs; Immunoassay sampling (ELISA and ELISPOT) if ET Visit occurs within Month 4; BCVA; LLVA; Full ophthalmic examination, including IOP, a slit-lamp examination, lens opacity, and dilated fundus examination; SD-OCT; AF; Microperimetry; Contrast sensitivity test; Color vision test; 7-field color fundus photography (including stereo photographs for fields 1, 2, and 3); Reading speed test (if applicable); VFQ-25; AE/SAE monitoring and Medication review.

Assessment of Efficacy

For all visits, an attempt should be made to perform all procedures in both eyes.

Best Corrected Visual Acuity

To evaluate changes in visual acuity (VA) over the study period, BCVA is assessed for both eyes using the ETDRS VA chart at the times indicated in Table 62: Schedule of Study Procedures. The BCVA test should be performed prior to pupil dilation, and distance refraction should be carried out before BCVA is measured. Initially, letters are read at a distance of 4 meters from the chart. If <20 letters are read at 4 meters, testing at 1 meter should be performed. BCVA is to be reported as number of letters read correctly by the subject. At the Screening/Baseline Visit, eyes with a BCVA of 34-73 ETDRS letters (equivalent to worse than or equal to 6/12 or 20/40 Snellen acuity, but better than or equal to 6/60 or 20/200 Snellen acuity) will be eligible for the study. If a subject cannot read any letters on the BCVA chart, the subject is tested for finger counting, hand movements or light perception.

Fundus Autofluorescence

To assess changes in the area of viable retinal tissue, fundus AF is performed for both eyes at the times indicated in Table 62: Schedule of Study Procedures. All fundus AF images are performed by certified technicians at the site after dilation of the subject's pupil.

Spectral Domain Optical Coherence Tomography (SD-OCT)

SD-OCT will be performed for both eyes at the times indicated in Table 2: Schedule of Study Procedures. SD-OCT measurements are taken by certified technicians at the site after dilation of the subject's pupil. SD-OCT issued to assess a number of variables, including quantifying the integrity of the ellipsoid zone and reduction in the signal from the outer nuclear layer and choroid. In addition, since progressive foveal thickening has been noted in the early phase of CHM, foveal changes are assessed.

Microperimetry

Microperimetry is conducted for both eyes at the times indicated in Table 62: Schedule of Study Procedures. Microperimetry is conducted by certified technicians to assess changes in retinal sensitivity within the macula.

Contrast Sensitivity

Contrast sensitivity is measured for both eyes at the times indicated in Table 62: Schedule of Study Procedures. Contrast sensitivity is measured prior to pupil dilation using a Pelli-Robson chart.

Color Vision

Color vision is tested for both eyes prior to pupil dilation at the times indicated in Table 62: Schedule of Study Procedures. Eyes are tested separately and in the same order at each assessment.

Low Luminance Visual Acuity

LLVA is measured for both eyes at the times indicated in Table 62: Schedule of Study Procedures. The test should be performed after BCVA testing, prior to pupil dilation, and distance refraction should be carried out before LLVA is measured. LLVA is measured by placing a 2.0-log-unit neutral density filter over the front of each eye and having the subject read the normally illuminated ETDRS chart. Initially, letters are read at a distance of 4 meters from the chart. If <20 letters are read at 4 meters, testing at 1 meter should be performed. LLVA is reported as number of letters read correctly by the subject.

Reading Speed Test

Reading performance will be evaluated prior to pupil dilation at the times indicated in Table 62: Schedule of Study Procedures using International Reading Speed Texts (IReST), which provide standardized assessment of reading performance in 17 languages (Trauzettel-Klosinski et al., 2012).

VFQ-25 Questionnaire

Subjects complete the VFQ-25 at the times indicated in Table 62: Schedule of Study Procedures. This questionnaire measures dimensions of self-reported vision-targeted health status that are most important to persons with eye disease (Mangione et al, 2001). Improvement in the VFQ-25 is evaluated using individual scores, subscale scores, and the overall composite score.

Assessment of Safety

Full Ophthalmic Examination

A full ophthalmic examination is conducted for both eyes at the times indicated in Table 62: Schedule of Study Procedures. For subjects in the AAV2-REP1 groups, the full ophthalmic examination is conducted prior to vitrectomy and administration of study medication at the applicable study visit. The ophthalmic examination will include IOP, slit lamp examination, lens opacity grading, and dilated ophthalmoscopy. The same slit lamp machine and lighting conditions should be used across study visits for any given subject.

In addition to the parameters listed above, relevant subjects are carefully examined for the presence of intraocular inflammation after vector administration. Cataract can also develop as a result of the vitrectomy procedure and can potentially affect VA. Pre-operative grading of lens opacity and color should therefore be documented by the established clinical lens opacities classification system. Cataract surgery is effective in subjects with CHM and without any specific risks. Hence, if clinically indicated, subjects who develop cataracts may undergo cataract surgery. If cataract surgery is performed, it should be carried out at least 4 weeks before the Month 12 Visit/EOS Visit.

Field Color Fundus Photography

To aid in the objective clinical assessment of progressive retinal changes, 7-field color fundus photography is performed for both eyes at the times indicated in Table 62: Schedule of Study Procedures. Fundus photography is performed by certified technicians following pupil dilation. Stereo photos should be performed for fields 1, 2, and 3.

Adverse Event

An AE is any untoward medical occurrence in a clinical investigation subject, which does not necessarily have a causal relationship with the study medication/surgical procedure. An AE can therefore be any unfavorable and unintended sign (including an abnormal laboratory finding), symptom, or disease temporally associated with the use of the study medication/surgical procedure, whether or not related to the investigational product or with the surgical procedure described in this protocol. AEs are to also include any pre-existing condition (other than CHM) or illness which worsens during the study (i.e., increases in frequency or intensity).

Serious Adverse Event

An SAE is defined as any untoward medical occurrence that: Results in death, Is life-threatening, Requires inpatient hospitalization or prolongation of existing hospitalization, Results in persistent or significant disability/incapacity, Is a congenital anomaly/birth defect, Results in vision loss or is vision threatening, Is another important medical event(s). The term ‘life-threatening’ in the definition of ‘serious’ refers to an event in which the subject is at risk of death at the time of the event. It does not refer to an event that hypothetically might cause death if it were more severe. Hospitalization for a pre-existing condition, including elective procedures, which has not worsened, does not constitute an SAE. Other events that may not result in death, are not life threatening or do not require hospitalization, may be considered an SAE when, based upon appropriate medical judgment, the event may jeopardize the subject and may require medical or surgical intervention to prevent one of the outcomes listed above.

The following vision loss or vision-threatening events are to be reported as SAEs: Sustained decrease in VA of ≥15 letters on ETDRS chart compared to baseline, except for surgery-related events. Sustained is defined as lasting 48 hours or more until recovery; recovery defined as VA returned to within 5 letters of baseline VA.

Surgery-related events of VA decrease are defined as VA decreases occurring in close temporal association (within <24 hours) with the surgical administration of the study medication, and which are resolving at Day 7 (Period 1/2, Visit 4) post-surgery. These events are not to be reported as an AE or SAE. However, they should be reported as an AE if in the investigator's opinion, their evolution in terms of duration or severity cannot be explained by the procedure. This would include, but not be limited to instances where the abnormal course of post-surgery VA decrease is associated with another complication attributable to the surgery or the study medication, or where the abnormal course of post-surgery VA decrease can be attributed to another identifiable cause. AEs that in the opinion of the investigator, actually or potentially require any surgical or medical intervention to prevent permanent loss of sight.

Efficacy Analyses

Statistical tests are performed at the alpha level of 0.05 (unless otherwise specified). Statistical tests and 95% CIs will be 2-sided.

Primary Efficacy Endpoint

The primary endpoint are calculated as the proportion of subjects with a ≥15 letter increase from baseline in BCVA at the Month 12 visit. If VA is missing at the Month 12 visit, then the missing value will be imputed using the LOCF method. The primary endpoint is summarized using the summary statistics for categorical data including the 95% CI.

The proportion of successes for the primary endpoint are compared between study arms (high dose vs control, low dose vs control) using the Fisher Exact test.

Key Secondary Efficacy Endpoint

The key secondary endpoint is a paired-sample analysis of the proportion of subjects with a ≥15 letter increase from baseline in BCVA at the Month 12 visit in treated study eyes within the STAR trial, compared to the proportion of subjects with a ≥15 letter BCVA gain from Month 4 (Visit 2) to Month 16 (Visit 5) in the NIGHT study (NSR-CHM-OS1). The analysis of the key secondary endpoint are performed on the Historical Control Full Analysis Set. The key secondary endpoint will be summarized, post-treatment (STAR data) and pre-treatment (NIGHT data), using the summary statistics for categorical data including the 95% CI. The proportion of successes for categorical variables will be compared between pre and post-treatment in each treatment arm separately using the McNemar test.

Other Secondary Efficacy Endpoints

Categorical secondary endpoints are summarized and analyzed using the same procedure as the categorical primary endpoint.

The continuous secondary endpoints are summarized using the summary statistics for continuous data, including the 95% CI. The treatment difference in mean change from baseline and its 95% CI is calculated based on the LSMEANS of the ANCOVA model including Surgery Group, baseline value of the assessment, study arms and the interaction between study arms and Surgery Group. Change from baseline is compared between study arms (high dose vs control, low dose vs control) using the above ANCOVA model. For the Overall Composite Score of VFQ-25, the covariates Age and Race is added to the model.

Safety Analyses

No significance testing is performed for safety analyses. Safety analyses are descriptive, with 95% CIs calculated where appropriate.

Adverse Events

AEs are coded using the Medical Dictionary for Regulatory Affairs. The version of the dictionary current at the time of the database lock will be used. Events are summarized by system organ class, preferred term, and group. Both the number of eyes/subjects experiencing an AE and the number of events is summarized. Similar summaries are produced for study drug/procedure-related AEs, AEs leading to discontinuation, and SAEs. AEs are summarized by maximum severity, relationship to study drug/procedure, and time to onset and resolution.

Full Ophthalmic Examination

IOP and change from Baseline in IOP are summarized by visit, by treatment and by eye. Abnormal slit lamp examination findings and dilated ophthalmoscopy findings, and shift from Baseline are summarized by visit, by treatment and by eye. Lens opacity categories and shift from Baseline are summarized by visit, by treatment and by eye.

Field Color Fundus Photography

Categories of color fundus photography findings and shift from Baseline are be summarized by visit, by treatment and by eye.

| | Visit | | | | | | | | | |
|---|---|---|---|---|---|---|---|---|---|---|
| | Visit 1 ^{a} | Visit 2^{b} | Visit 3^{b} | Visit 4^{b} | Visit 5^{a} | Visit 6^{a} | Visit 7^{a} | Visit 8^{a, c} | | |
| | Study Day/Visit Window | | | | | | | | | |
| | | Day 0 | Day 1 | Day 7 | | | | | | |
| | Screening/ | Injection | Post | Post | Month | Month | Month | Month | Early | Unscheduled |
| | Baseline [symbol] | Day | op | op ± 3 d | 1 ± 7 d | 4 ± 7 d | 8 ± 14 d | 12 ± 14 d | Termination^{d} | Visit^{e} |
| Assessments/Procedures | | | | | | | | | | |
| (All Subjects/Eyes, Unless | | | | | | | | | | |
| Otherwise Specified) | | | | | | | | | | |
| Informed Consent | X | | | | | | | | | |
| Demography, medical and | X | | | | | | | | | |
| ocular history | | | | | | | | | | |
| Vital signs (pulse, blood | X | | X | X | X | | | X | X | |
| pressure) | | | | | | | | | | |
| Weight [symbol] | X | | | | | | | | | |
| Immunoassay blood | X | | X | X | X | X | | | X [symbol] | |
| sampling (ELISA)^{b} | | | | | | | | | | |
| Immunoassay blood | X | | | X | X | X | | | X [symbol] | |
| sampling (ELISPOT)^{b} | | | | | | | | | | |
| BCVA | X [symbol] | | X | X | X | X | X | X | X | X |
| LLVA | X | | | X | X | X | X | X | X | |
| Full ophthalmic exam [symbol] | X | X | X | X | X | X | X | X | X | X |
| SD-OCT | X | | X | X | X | X | X | X | X | X |
| Autofluorescence | X | | | X | X | X | X | X | X | X |
| Microperimetry | X | | | X | X | X | X | X | X | |
| Contrast sensitivity [symbol] | X | | | | | X | X | X | X | |
| Colour vision | X | | | | | X | X | X | X | |
| 7-field colour fundus | X | | | | | | | X | X | |
| photos [symbol] | | | | | | | | X | X | |
| Reading speed test [symbol] | X | | | | | | | X | X | |
| VFQ-25 [symbol] | X | | | | | | | | | |

[symbol]

indicates data missing or illegible when filed

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### Other Embodiments

While particular embodiments of the disclosure have been illustrated and described, various other changes and modifications can be made without departing from the spirit and scope of the disclosure. The scope of the appended claims includes all such changes and modifications that are within the scope of this disclosure.

What is claimed is:

1. A method for the purification of a recombinant AAV (rAAV) particle from a mammalian host cell culture, comprising the steps of:
(a) culturing a plurality of mammalian host cells in a culture media under conditions suitable for the formation of a plurality of rAAV particles, wherein the plurality of mammalian host cells have been transfected with a plasmid vector comprising an exogenous sequence, a helper plasmid vector, and a plasmid vector comprising a sequence encoding a viral Rep protein and a viral Cap protein to produce a plurality of transfected mammalian host cells;
(b) harvesting the culture media comprising the plurality of transfected mammalian host cells;
(c) harvesting a plurality of rAAV particles from the plurality of transfected mammalian host cells;
(d) concentrating the plurality of rAAV particles by tangential flow filtration (TFF) to produce a concentrated plurality of rAAV particles;
(e) enriching the concentrated plurality of rAAV particles for full rAAV particles by density gradient ultracentrifugation to produce an enriched plurality of full rAAV particles;
(f) purifying the enriched plurality of full rAAV particles by anion exchange (AEX) chromatography or affinity chromatography to produce an eluate comprising a purified and enriched plurality of full rAAV particles; and
(g) diafiltering and concentrating the eluate from (f) into a formulation buffer by tangential flow filtration (TFF) to produce a final composition comprising the purified and enriched plurality of full rAAV particles and the formulation buffer.

2. A method for the purification of a recombinant AAV (rAAV) particle from a mammalian host cell culture, comprising the steps of:
(a) culturing a plurality of mammalian host cells in a culture media under conditions suitable for the formation of a plurality of rAAV-REP1 particles, wherein the plurality of mammalian host cells have been transfected with a plasmid vector comprising an exogenous sequence wherein the exogenous sequence comprises a sequence encoding a human Rab escort protein 1 (REP1) protein, a helper plasmid vector, and a plasmid vector comprising a sequence encoding a viral Rep protein and a viral Cap protein to produce a plurality of transfected mammalian host cells;
(b) harvesting the culture media comprising the plurality of transfected mammalian host cells;
(c) harvesting a plurality of rAAV particles from the plurality of transfected mammalian host cells;
(d) concentrating the plurality of rAAV particles by tangential flow filtration (TFF) to produce a concentrated plurality of rAAV particles;
(e) enriching the concentrated plurality of rAAV particles for full rAAV particles by density gradient ultracentrifugation to produce an enriched plurality of full rAAV particles;
(f) purifying the enriched plurality of full rAAV particles by anion exchange (AEX) chromatography or affinity chromatography to produce an eluate comprising a purified and enriched plurality of full rAAV particles; and
(g) diafiltering and concentrating the eluate from (f) into a formulation buffer by tangential flow filtration (TFF) to produce a final composition comprising the purified and enriched plurality of full rAAV particles and the formulation buffer.

3. The method of claim 1 or 2, wherein the culture media comprises a reduced amount of fetal bovine serum.

4. The method of any one of claims 1-3, wherein the culture media does not comprise a reduced amount of fetal bovine serum.

5. The method of any one of claims 1-3, wherein the culture media comprises a serum-free media.

6. The method of any one of claims 1-3, wherein the culture media consists of a serum-free media.

7. The method of any one of claims 1-6, wherein the culture media comprises a protein-free media.

8. The method of any one of claims 1-6, wherein the culture media consists of a protein-free media.

9. The method of any one of claims 1-8, wherein the culture media comprises glycine, L-Arginine hydrochloride, L-Cysteine dihydrochloride, L-Glutamine, L-Histidine hydrochloride-H₂O, L-Isoleucine, L-Leucine, L-Lysine hydrochloride, L-Methionine, L-Phenylalanine, L-Serine, L-Threonine, L-Tryptophan, L-Tyrosine disodium salt dehydrate, L-Valine, Choline chloride, D-Calcium pantothenate, Folic Acid, Niacinamide, Pyridoxine hydrochloride, Riboflavin, Thiamine hydrochloride, i-Inositol, Calcium Chloride (CaCl₂) (anhyd.), Ferric Nitrate (Fe(NO₃)₃″9H2O), Magnesium Sulfate (MgSO₄) (anhyd.), Potassium Chloride (KCl), Sodium Bicarbonate (NaHCO₃), Sodium Chloride (NaCl), Sodium Phosphate monobasic (NaH₂PO4-H₂O), and D-Glucose (Dextrose).

10. The method of any one of claims 1-9, wherein the mammalian cells have been transfected with a composition comprising a PEI transduction reagent.

11. The method of any one of claims 1-10, wherein the plasmid vector comprising an exogenous sequence, further comprises a sequence encoding a 5′ inverted terminal repeat (ITR) and a sequence encoding a 3′ ITR.

12. The method of claim 11, wherein the sequence encoding a 5′ ITR is derived from a sequence encoding a 5′ITR of an AAV of serotype 2 (AAV2).

13. The method of claim 11 or 12, wherein the sequence encoding a 5′ ITR comprises a sequence that is identical to a sequence encoding a 5′ITR of an AAV of serotype 2 (AAV2).

14. The method of claim 11 or 12, wherein the sequence encoding a 5′ ITR comprises a sequence that is not identical to a sequence encoding a 5′ITR of an AAV of serotype 2 (AAV2).

15. The method of any one of claims 11-14, wherein the sequence encoding a 3′ ITR is derived from a sequence encoding a 3′ITR of an AAV of serotype 2 (AAV2).

16. The method of claim 15, wherein the sequence encoding a 3′ ITR comprises a sequence that is identical to a sequence encoding a 3′ITR of an AAV of serotype 2 (AAV2).

17. The method of claim 16, wherein the sequence encoding a 3′ ITR comprises a sequence that is not identical to a sequence encoding a 3′ITR of an AAV of serotype 2 (AAV2).

18. The method of any one of claims 11-17, wherein the sequence encoding a 5′ ITR or the sequence encoding a 3′ ITR comprises 145 base pairs (bp).

19. The method of any one of claims 11-17, wherein the sequence encoding a 5′ ITR or the sequence encoding a 3′ ITR comprises or consists of 134, 135, 136 or 137 (bp).

20. The method of any one of claims 1-19, wherein the plasmid vector comprising an exogenous sequence, the helper plasmid vector or the plasmid vector comprising a sequence encoding a viral Rep protein and a viral Cap protein, further comprises a sequence encoding a selection marker.

21. The method of claim 20, wherein the sequence encoding a selection marker conveys resistance to kanamycin.

22. The method of any one of claims 1-21, wherein the harvesting step (c) comprises a mechanical disruption of the plurality of transfected mammalian cells to release recombinant AAV (rAAV) particles produced by the plurality of transfected mammalian cells.

23. The method of claim 22, wherein the mechanical disruption comprises a microfluidization.

24. The method of any one of claims 1-23, wherein the concentrating step further comprises (1) clarifying the concentrated plurality of rAAV particles by a depth filtration to produce a concentrated and clarified plurality of rAAV particles.

25. The method of claim 24, wherein the concentrating step further comprises (2) freezing the concentrated and clarified plurality of rAAV particles at −80° C. to produce a process intermediate.

26. The method of any one of claims 1-25, wherein the enriching step (e) comprises an iodixanol density gradient ultracentrifugation to produce an enriched plurality of rAAV particles.

27. The method of claim 26, wherein the iodixanol density gradient comprise iodixanol compositions having concentrations of 15%, 25%, 40% and 57%.

28. The method of any one of claims 1-27, wherein the affinity chromatography of the purifying step (f) comprises an AVB Sepharose matrix.

29. The method of any one of claims 1-28, wherein the formulation buffer comprises Tris, MgCl₂, and NaCl.

30. The method of any one of claims 1-27, wherein the formulation buffer comprises 20 mM Tris, 1 mM MgCl₂, and 200 mM NaCl at pH 8.

31. The method of any one of claims 1-28, wherein the formulation buffer comprises 20 mM Tris, 1 mM MgCl₂, 200 mM NaCl at pH 8 and poloxamer 188 at 0.001%.

32. The method of any one of claims 1-31, wherein the AEX Chromatography further comprises the steps of
generating an AEX Chromatogram and
selecting a peak on the AEX Chromatogram containing full rAAV particles.

33. The method of any one of claims 1-32, wherein the TFF of step (d) or step (g) is performed using a 100 kDa hollow fiber filter (HFF).

34. The method of any one of claims 1-33, wherein the TFF of step (d) or step (g) is performed using a 50 kDa HFF.

35. The method of any one of claims 1-34, wherein step (g) the method further comprises a second TFF, wherein the TFF of step (d) and the first TFF of step (g) are performed using a 100 kDa HFF and wherein the second TFF of step (g) is performed using a 50 kDa HFF.

36. The method of any one of claims 2-35, wherein the sequence encoding the human REP1 protein comprises or consists of the nucleic acid sequence of:
(SEQ ID NO: 1)

| | |
|---|---|
| 1 | gatatcgaat tcctgcagcc cggcggcacc atggcggata |
| | ctctcccttc ggagtttgat |
| 61 | gtgatcgtaa tagggacggg tttgcctgaa tccatcattg |
| | cagctgcatg ttcaagaagt |
| 121 | ggccggagag ttctgcatgt tgattcaaga agctactatg |
| | gaggaaactg ggccagtttt |
| 181 | agcttttcag gactattgtc ctggctaaag gaataccagg |
| | aaaacagtga cattgtaagt |
| 241 | gacagtccag tgtggcaaga ccagatcctt gaaaatgaag |
| | aagccattgc tcttagcagg |
| 301 | aaggacaaaa ctattcaaca tgtggaagta ttttgttatg |
| | ccagtcagga tttgcatgaa |
| 361 | gatgtcgaag aagctggtgc actgcagaaa aatcatgctc |
| | ttgtgacatc tgcaaactcc |
| 421 | acagaagctg cagattctgc cttcctgcct acggaggatg |
| | agtcattaag cactatgagc |
| 481 | tgtgaaatgc tcacagaaca aactccaagc agcgatccag |
| | agaatgcgct agaagtaaat |
| 541 | ggtgctgaag tgacagggga aaaagaaaac cattgtgatg |
| | ataaaacttg tgtgccatca |
| 601 | acttcagcag aagacatgag tgaaaatgtg cctatagcag |
| | aagataccac agagcaacca |
| 661 | aagaaaaaca gaattactta ctcacaaatt attaaagaag |
| | gcaggagatt taatattgat |
| 721 | ttagtatcaa agctgctgta ttctcgagga ttactaattg |
| | atcttctaat caaatctaat |
| 781 | gttagtcgat atgcagagtt taaaaatatt accaggattc |
| | ttgcatttcg agaaggacga |
| 841 | gtggaacagg ttccgtgttc cagagcagat gtctttaata |
| | gcaaacaact tactatggta |
| 901 | gaaaagcgaa tgctaatgaa atttcttaca ttttgtatgg |
| | aatatgagaa atatcctgat |
| 961 | gaatataaag gatatgaaga gatcacattt tatgaatatt |
| | taaagactca aaaattaacc |
| 1021 | cccaacctcc aatatattgt catgcattca attgcaatga |
| | catcagagac agccagcagc |
| 1081 | accatagatg gtctcaaagc taccaaaaac tttcttcact |
| | gtcttgggcg gtatggcaac |
| 1141 | actccatttt tgtttccttt atatggccaa ggagaactcc |
| | cccagtgttt ctgcaggatg |
| 1201 | tgtgctgtgt ttggtggaat ttattgtctt cgccattcag |
| | tacagtgcct tgtagtggac |
| 1261 | aaagaatcca gaaaatgtaa agcaattata gatcagtttg |
| | gtcagagaat aatctctgag |
| 1321 | catttcctcg tggaggacag ttactttcct gagaacatgt |
| | gctcacgtgt gcaatacagg |
| 1381 | cagatctcca gggcagtgct gattacagat agatctgtcc |
| | taaaaacaga ttcagatcaa |
| 1441 | cagatttcca ttttgacagt gccagcagag gaaccaggaa |
| | cttttgctgt tcgggtcatt |
| 1501 | gagttatgtt cttcaacgat gacatgcatg aaaggcacct |
| | atttggttca tttgacttgc |
| 1561 | acatcttcta aaacagcaag agaagattta gaatcagttg |
| | tgcagaaatt gtttgttcca |
| 1621 | tatactgaaa tggagataga aaatgaacaa gtagaaaagc |
| | caagaattct gtgggctctt |
| 1681 | tacttcaata tgagagattc gtcagacatc agcaggagct |
| | gttataatga tttaccatcc |
| 1741 | aacgtttatg tctgctctgg cccagattgt ggtttaggaa |
| | atgataatgc agtcaaacag |
| 1801 | gctgaaacac ttttccagga aatctgcccc aatgaagatt |
| | tctgtccccc tccaccaaat |
| 1861 | cctgaagaca ttatccttga tggagacagt ttacagccag |
| | aggcttcaga atccagtgcc |
| 1921 | ataccagagg ctaactcgga gactttcaag gaaagcacaa |
| | accttggaaa cctagaggag |
| 1981 | tcctctqaat aa |

37. The composition of claim 36, wherein the human REP1 protein comprises or consists of the amino acid sequence of
(SEQ ID NO: 2)

| | |
|---|---|
| 1 | MADTLPSEFD VIVIGTGLPE SIIAAACSRS GRRVLHVDSR |
| | SYYGGNWASF SFSGLLSWLK |
| 61 | EYQENSDIVS DSPVWQDQIL ENEEAIALSR KDKTIQHVEV |
| | FCYASQDLHE DVEEAGALQK |
| 121 | NHALVTSANS TEAADSAFLP TEDESLSTMS CEMLTEQTPS |
| | SDPENALEVN GAEVTGEKEN |
| 181 | HCDDKTCVPS TSAEDMSENV PIAEDTTEQP KKNRITYSQI |
| | IKEGRRFNID LVSKLLYSRG |
| 241 | LLIDLLIKSN VSRYAEFKNI TRILAFREGR VEQVPCSRAD |
| | VFNSKQLTMV EKRMLMKFLT |
| 301 | FCMEYEKYPD EYKGYEEITF YEYLKTQKLT PNLQYIVMHS |
| | IAMTSETASS TIDGLKATKN |
| 361 | FLHCLGRYGN TPFLFPLYGQ GELPQCFCRM CAVFGGIYCL |
| | RHSVQCLVVD KESRKCKAII |
| 421 | DQFGQRIISE HFLVEDSYFP ENMCSRVQYR QISRAVLITD |
| | RSVLKTDSDQ QISILTVPAE |
| 481 | EPGTFAVRVI ELCSSTMTCM KGTYLVHLTC TSSKTAREDL |
| | ESVVQKLFVP YTEMEIENEQ |
| 541 | VEKPRILWAL YFNMRDSSDI SRSCYNDLPS NVYVCSGPDC |
| | GLGNDNAVKQ AETLFQEICP |
| 601 | NEDFCPPPPN PEDIILDGDS LQPEASESSA IPEANSETFK |
| | ESTNLGNLEE SSE |

38. A pharmaceutical composition comprising a plurality of rAAV particles produced by the method of any one of claims 1-37.

39. The pharmaceutical composition of claim 38, comprising
(a) between 0.5 and 2.5×10¹² vector genomes (vg)/mL of replication-defective and recombinant adeno-associated virus (rAAV);
(b) less than 50% empty capsids;
(c) less than 4 ng/mL residual host cell protein per 1.0×10¹² vg/mL; and
(d) less than 7×10⁻³ pg/ml residual host cell DNA per 1.0×10¹² vg/mL.

40. The pharmaceutical composition of claim 39, wherein the composition further comprises
(e) a plurality of functional vg/mL, wherein each of functional vector genomes is capable of expressing an exogenous sequence in a cell following transduction.

41. The pharmaceutical composition of claim 40, wherein, following transduction of a cell with the pharmaceutical composition, the plurality of functional vg/mL express the exogenous sequence at a 2-fold increase when compared to a level of expression of a corresponding endogenous sequence in a nontransduced cell.

42. The pharmaceutical composition of claim 40, wherein, following transduction of a cell with the pharmaceutical composition, the plurality of functional vg/mL express the exogenous sequence at a 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, or any other increment fold increase in between, when compared to a level of expression of a corresponding endogenous sequence in a nontransduced cell.

43. The pharmaceutical composition of claim 41 or 42, wherein the exogenous sequence and the corresponding endogenous sequence are identical.

44. The pharmaceutical composition of claim 41 or 42, wherein the exogenous sequence and the corresponding endogenous sequence are not identical.

45. The pharmaceutical composition of claim 41, 42 or 44, wherein the exogenous sequence and the corresponding endogenous sequence are not identical, but a protein encoded by the exogenous sequence and a protein encoded by the endogenous sequence are identical.

46. The pharmaceutical composition of claim 42 or 43, wherein the exogenous sequence and the corresponding endogenous sequence have at least 70%, 75%, 80%, 85%, 90%, 95%, 97%, 99% or any percentage in between of identity.

47. The pharmaceutical composition of any one of claims 37-44, wherein the exogenous sequence is codon-optimized when compared to the endogenous sequence.

48. The pharmaceutical composition of 47, wherein the exogenous sequence and the corresponding endogenous sequence have at least 50%, 55%, 60%, 65%, 70%, 75%, 80%, 85%, 90%, 95%, 97%, 99% or any percentage in between of homology.

49. The pharmaceutical composition of any one of claims 38-48, wherein, following transduction of a cell with a pharmaceutical composition of the disclosure, the exogenous sequence encodes a protein.

50. The pharmaceutical composition of claim 49, wherein the protein encoded by the exogenous sequence has an activity level equal to or greater than an activity level of a protein encoded by a corresponding sequence of a nontransduced cell.

51. The pharmaceutical composition of claim 50, wherein the exogenous sequence and the corresponding endogenous sequence are identical.

52. The pharmaceutical composition of claim 50, wherein the exogenous sequence and the corresponding endogenous sequence are not identical.

53. The pharmaceutical composition of claim 52, wherein the exogenous sequence and the corresponding endogenous sequence have at least 70%, 75%, 80%, 85%, 90%, 95%, 97%, 99% or any percentage in between of identity.

54. The pharmaceutical composition of any one of claims 49-52, wherein the activity comprises binding to, activating, and/or transferring one or more functional groups to a ligand or a substrate.

55. The pharmaceutical composition of any one of claims 38-54, wherein the protein comprises a REP-1 protein and the activity comprises a prenylation of REP-1 substrate.

56. The pharmaceutical composition of any one of claims 38-55, comprising
(a) between 1.0 and 2.0×10¹² vector genomes (vg)/mL of replication-defective and recombinant adeno-associated virus (AAV).

57. The pharmaceutical composition of any one of claims 38-55, comprising
(a) about 1.0×10¹² vector genomes (vg)/mL of replication-defective and recombinant adeno-associated virus (AAV).

58. The pharmaceutical composition of any one of claims 38-55, comprising
(a) 1.0×10¹² vector genomes (vg)/mL of replication-defective and recombinant adeno-associated virus (AAV).

59. The pharmaceutical composition of any one of claims 38-55, comprising
(b) less than 50% empty capsids or less than 30% empty capsids.

60. The pharmaceutical composition of any one of claims 38-59, wherein the replication-defective and recombinant adeno-associated virus (rAAV) contains a sequence isolated or derived from an AAV of serotype 2 (AAV2).

61. The pharmaceutical composition of claim 60, wherein the sequence isolated or derived from an AAV2 comprises a sequence encoding an inverted terminal repeat (ITR).

62. The pharmaceutical composition of claim 60 or 61, wherein the replication-defective and recombinant adeno-associated virus (rAAV) contains a sequence encoding a 5′ ITR and a sequence encoding a 3′ ITR.

63. The pharmaceutical composition of claim 62, wherein the sequence encoding a 5′ ITR and the sequence encoding a 3′ ITR comprise a wild type sequence of an AAV2 ITR.

64. The pharmaceutical composition of any one of claims 38-63, wherein the host cell is isolated or derived from a cultured cell line.

65. The pharmaceutical composition of claim 64, wherein the host cell is an HEK293 cell.

66. The pharmaceutical composition of any one of claims 38-63, wherein the host cell is isolated or derived from a primary cell line.

67. The pharmaceutical composition of claim 66, wherein the host cell is an immortalized cell or a stem cell.

68. The method of any one of claims 1-37, wherein the host cell is isolated or derived from a cultured cell line.

69. The method of claim 68, wherein the host cell is an HEK293 cell.

70. The method of any one of claims 1-37, wherein the host cell is isolated or derived from a primary cell line.

71. The method of claim 70, wherein the host cell is an immortalized cell or a stem cell.

72. The pharmaceutical composition of any one of claims 38-67, wherein each full rAAV of the plurality of full rAAV particles of the final composition further comprises:
a nucleic acid sequence comprising, from 5′ to 3′:
(a) a sequence encoding an AAV2 5′ ITR,
(b) a sequence encoding an early enhancer element,
(c) a sequence encoding a promoter,
(d) a sequence encoding an exon and intron,
(e) a sequence encoding a splice acceptor site,
(f) a sequence encoding a Rab escort protein 1 (REP1) protein,
(g) a sequence encoding a post-transcriptional regulatory element (PRE),
(h) a sequence encoding a polyadenylation (polyA) site, and
(i) a sequence encoding an AAV2 3′ ITR.

73. The pharmaceutical composition of claim 72, wherein the early enhancer element comprises a sequence isolated or derived from a Cytomegalovirus (CMV).

74. The pharmaceutical composition of claim 73, wherein the early enhancer element comprises or consists of the nucleic acid sequence of

| (SEQ ID NO: 3) | |
|---|---|
| 1 | ATTGACGT CAATAATGAC GTATGTTCCC ATAGTAACGC CAATAGGGAC TTTCCATTGA |
| 61 | CGTCAATGGG TGGAGTATTT ACGGTAAACT GCCCACTTGG CAGTACATCA AGTGTATCAT |
| 121 | ATGCCAAGTA CGCCCCCTAT TGACGTCAAT GACGGTAAAT GGCCCGCCTG GCATTATGCC |
| 181 | CAGTACATGA CCTTATGGGA CTTTCCTACT TGGCAGTACA TCTACGTATT AGTCATCGCT |
| 241 | ATTACCATGG. |

75. The pharmaceutical composition of claim 73, wherein the early enhancer element comprises or consists of the nucleic acid sequence of

| (SEQ ID NO: 4) | |
|---|---|
| 1 | CCATTGACGT CAATAATGAC GTATGTTCCC ATAGTAACGC CAATAGGGAC TTTCCATTGA |
| 61 | CGTCAATGGG TGGAGTATTT ACGGTAAACT GCCCACTTGG CAGTACATCA AGTGTATCAT |
| 121 | ATGCCAAGTA CGCCCCCTAT TGACGTCAAT GACGGTAAAT GGCCCGCCTG GCATTATGCC |
| 181 | CAGTACATGA CCTTATGGGA CTTTCCTACT TGGCAGTACA TCTACGTATT AGTCATCGCT |
| 241 | ATTACCATGG. |

76. The pharmaceutical composition of any one of claims 72-75, wherein the sequence encoding the promoter comprises or consists of a sequence isolated or derived from a sequence encoding a chicken beta actin (CBA) gene.

77. The pharmaceutical composition of any one of claims 72-75, wherein the sequence encoding the exon and intron comprises or consists of a sequence isolated or derived from a sequence encoding a chicken beta actin (CBA) gene.

78. The pharmaceutical composition of any one of claims 72-75, wherein the sequence encoding the promoter comprises or consists of the nucleic acid sequence of

| (SEQ ID NO: 5) | |
|---|---|
| 1 | TCGAGGTGAG CCCCACGTTC TGCTTCACTC TCCCCATCTC CCCCCCCTCC CCACCCCCAA |
| 61 | TTTTGTATTT ATTTATTTTT TAATTATTTT GTGCAGCGAT GGGGGCGGGG GGGGGGGGGG |
| 121 | GGCGCGCGCC AGGCGGGGCG GGGCGGGGCG AGGGGCGGGG CGGGGCGAGG CGGAGAGGTG |
| 181 | CGGCGGCAGC CAATCAGAGC GGCGCGCTCC GAAAGTTTCC TTTTATGGCG AGGCGGCGGC |
| 241 | GGCGGCGGCC CTATAAAAAG CGAAGCGCGC GGCGGGCGGG AGTCGCTGCG CGCTGCCTTC |
| 301 | GCCCCGTGCC CCGCTCCGCC GCCGCCTCGC GCCGCCCGCC CCGGCTCTGA CTGACCGCGT |
| 361 | TACTCCCACA GGTGAGCGGG CGGGACGGCC CTTCTCCTCC GGGCTGTAAT TAGCGCTTGG |
| 421 | TTTAATGACG GCTTGTTTCT TTTCTGTGGC TGCGTGAAAG CCTTGAGGGG CTCCGGGAGG |
| 481 | GCCCTTTGTG CGGGGGGAGC GGCTCGGGG. |

79. The pharmaceutical composition of any one of claims 72-78, wherein the sequence encoding the exon and intron comprises or consists of the nucleic acid sequence of

| (SEQ ID NO: 5) | |
|---|---|
| 1 | TCGAGGTGAG CCCCACGTTC TGCTTCACTC TCCCCATCTC CCCCCCCTCC CCACCCCCAA |
| 61 | TTTTGTATTT ATTTATTTTT TAATTATTTT GTGCAGCGAT GGGGGCGGGG GGGGGGGGGG |
| 121 | GGCGCGCGCC AGGCGGGGCG GGGCGGGGCG AGGGGCGGGG CGGGGCGAGG CGGAGAGGTG |
| 181 | CGGCGGCAGC CAATCAGAGC GGCGCGCTCC GAAAGTTTCC TTTTATGGCG AGGCGGCGGC |
| 241 | GGCGGCGGCC CTATAAAAAG CGAAGCGCGC GGCGGGCGGG AGTCGCTGCG CGCTGCCTTC |
| 301 | GCCCCGTGCC CCGCTCCGCC GCCGCCTCGC GCCGCCCGCC CCGGCTCTGA CTGACCGCGT |
| 361 | TACTCCCACA GGTGAGCGGG CGGGACGGCC CTTCTCCTCC GGGCTGTAAT TAGCGCTTGG |
| 421 | TTTAATGACG GCTTGTTTCT TTTCTGTGGC TGCGTGAAAG CCTTGAGGGG CTCCGGGAGG |
| 481 | GCCCTTTGTG CGGGGGGAGC GGCTCGGGG. |

80. The pharmaceutical composition of any one of claims 72-79, wherein the sequence encoding the splice acceptor site comprises a sequence isolated or derived from a sequence encoding an Oryctolagus cuniculus beta globin splice acceptor site.

81. The pharmaceutical composition of claim 80, wherein the sequence encoding the Oryctolagus cuniculus beta globin splice acceptor site comprises or consists of the nucleic acid sequence of

| (SEQ ID NO: 6) | |
|---|---|
| 1 | CTCTGCT AACCATGTTC ATGCCTTCTT CTTTTTCCTA CAGCTCCTGG GCAACGTGCT |
| 61 | GGTTATTGTG CTGTCTCATC ATTTTGGCAA AGAATT |

82. The pharmaceutical composition of any one of claims 72-81, wherein the sequence comprising the early enhancer element, the sequence comprising the promoter, the sequence comprising the intron and exon and the sequence comprising the splice acceptor site, comprise or consist of the nucleic acid sequence of

| (SEQ ID NO: 7) | |
|---|---|
| 1 | TCGAGGTGAG CCCCACGTTC TGCTTCACTC TCCCCATCTC CCCCCCCTCC CCACCCCCAA |
| 61 | TTTTGTATTT ATTTATTTTT TAATTATTTT GTGCAGCGAT GGGGGCGGGG GGGGGGGGGG |
| 121 | GGCGCGCGCC AGGCGGGGCG GGGCGGGGCG AGGGGCGGGG CGGGGCGAGG CGGAGAGGTG |
| 181 | CGGCGGCAGC CAATCAGAGC GGCGCGCTCC GAAAGTTTCC TTTTATGGCG AGGCGGCGGC |
| 241 | GGCGGCGGCC CTATAAAAAG CGAAGCGCGC GGCGGGCGGG AGTCGCTGCG CGCTGCCTTC |
| 301 | GCCCCGTGCC CCGCTCCGCC GCCGCCTCGC GCCGCCCGCC CCGGCTCTGA CTGACCGCGT |
| 361 | TACTCCCACA GGTGAGCGGG CGGGACGGCC CTTCTCCTCC GGGCTGTAAT TAGCGCTTGG |
| 421 | TTTAATGACG GCTTGTTTCT TTTCTGTGGC TGCGTGAAAG CCTTGAGGGG CTCCGGGAGG |
| 481 | GCCCTTTGTG CGGGGGGAGC GGCTCGGGGC TGTCCGCGGG GGGACGGCTG CCTTCGGGGG |
| 541 | GGACGGGGCA GGGCGGGGTT CGGCTTCTGG CGTGTGACCG GCGGCTCTAG AGCCTCTGCT |
| 601 | AACCATGTTC ATGCCTTCTT CTTTTTCCTA CAGCTCCTGG GCAACGTGCT GGTTATTGTG |
| 661 | CTGTCTCATC ATTTTGGCAA AGAATTGGAT C. |

83. The pharmaceutical composition of any one of claims 72-81, wherein the sequence comprising the early enhancer element, the sequence comprising the promoter, the sequence comprising the intron and exon and the sequence comprising the splice acceptor site, comprise or consist of the nucleic acid sequence of

| (SEQ ID NO: 8) | |
|---|---|
| 1 | CCATTGACGT CAATAATGAC GTATGTTCCC ATAGTAACGC CAATAGGGAC TTTCCATTGA |
| 61 | CGTCAATGGG TGGAGTATTT ACGGTAAACT GCCCACTTGG CAGTACATCA AGTGTATCAT |
| 121 | ATGCCAAGTA CGCCCCCTAT TGACGTCAAT GACGGTAAAT GGCCCGCCTG GCATTATGCC |
| 181 | CAGTACATGA CCTTATGGGA CTTTCCTACT TGGCAGTACA TCTACGTATT AGTCATCGCT |
| 241 | ATTACCATGG TCGAGGTGAG CCCCACGTTC TGCTTCACTC TCCCCATCTC CCCCCCCTCC |
| 301 | CCACCCCCAA TTTTGTATTT ATTTATTTTT TAATTATTTT GTGCAGCGAT GGGGGCGGGG |
| 361 | GGGGGGGGGG GGCGCGCGCC AGGCGGGGCG GGGCGGGGCG AGGGGCGGGG CGGGGCGAGG |
| 421 | CGGAGAGGTG CGGCGGCAGC CAATCAGAGC GGCGCGCTCC GAAAGTTTCC TTTTATGGCG |
| 481 | AGGCGGCGGC GGCGGCGGCC CTATAAAAAG CGAAGCGCGC GGCGGGCGGG AGTCGCTGCG |
| 541 | CGCTGCCTTC GCCCCGTGCC CCGCTCCGCC GCCGCCTCGC GCCGCCCGCC CCGGCTCTGA |
| 601 | CTGACCGCGT TACTCCCACA GGTGAGCGGG CGGGACGGCC CTTCTCCTCC GGGCTGTAAT |
| 661 | TAGCGCTTGG TTTAATGACG GCTTGTTTCT TTTCTGTGGC TGCGTGAAAG CCTTGAGGGG |
| 721 | CTCCGGGAGG GCCCTTTGTG CGGGGGGAGC GGCTCGGGGC TGTCCGCGGG GGGACGGCTG |
| 781 | CCTTCGGGGG GGACGGGGCA GGGCGGGGTT CGGCTTCTGG CGTGTGACCG GCGGCTCTAG |
| 841 | AGCCTCTGCT AACCATGTTC ATGCCTTCTT CTTTTTCCTA CAGCTCCTGG GCAACGTGCT |
| 901 | GGTTATTGTG CTGTCTCATC ATTTTGGCAA AGAATTGGAT C. |

84. The pharmaceutical composition of any one of claims 72-83, wherein the sequence encoding the REP1 protein comprises a sequence isolated or derived from a mammalian REP1 sequence.

85. The pharmaceutical composition of claim 84, wherein the mammalian REP1 sequence is isolated or derived from a mouse, a rat, a rabbit, a non-human primate or a human.

86. The pharmaceutical composition of claim 84, wherein the mammalian REP1 sequence is isolated or derived from a human.

87. The pharmaceutical composition of claim 86, wherein the sequence encoding the human REP1 protein comprises or consists of the nucleic acid sequence of

| (SEQ ID NO: 9) | |
|---|---|
| 1 | gatatcgaat tcctgcagcc cggcggcacc atggcggata ctctcccttc ggagtttgat |
| 61 | gtgatcgtaa tagggacggg tttgcctgaa tccatcattg cagctgcatg ttcaagaagt |
| 121 | ggccggagag ttctgcatgt tgattcaaga agctactatg gaggaaactg ggccagtttt |
| 181 | agcttttcag gactattgtc ctggctaaag gaataccagg aaaacagtga cattgtaagt |
| 241 | gacagtccag tgtggcaaga ccagatcctt gaaaatgaag aagccattgc tcttagcagg |
| 301 | aaggacaaaa ctattcaaca tgtggaagta ttttgttatg ccagtcagga tttgcatgaa |
| 361 | gatgtcgaag aagctggtgc actgcagaaa aatcatgctc ttgtgacatc tgcaaactcc |
| 421 | acagaagctg cagattctgc cttcctgcct acggaggatg agtcattaag cactatgagc |
| 481 | tgtgaaatgc tcacagaaca aactccaagc agcgatccag agaatgcgct agaagtaaat |
| 541 | ggtgctgaag tgacagggga aaaagaaaac cattgtgatg ataaaacttg tgtgccatca |
| 601 | acttcagcag aagacatgag tgaaaatgtg cctatagcag aagataccac agagcaacca |
| 661 | aagaaaaaca gaattactta ctcacaaatt attaaagaag gcaggagatt taatattgat |
| 721 | ttagtatcaa agctgctgta ttctcgagga ttactaattg atcttctaat caaatctaat |
| 781 | gttagtcgat atgcagagtt taaaaatatt accaggattc ttgcatttcg agaaggacga |
| 841 | gtggaacagg ttccgtgttc cagagcagat gtctttaata gcaaacaact tactatggta |
| 901 | gaaaagcgaa tgctaatgaa atttcttaca ttttgtatgg aatatgagaa atatcctgat |
| 961 | gaatataaag gatatgaaga gatcacattt tatgaatatt taaagactca aaaattaacc |
| 1021 | cccaacctcc aatatattgt catgcattca attgcaatga catcagagac agccagcagc |
| 1081 | accatagatg gtctcaaagc taccaaaaac tttcttcact gtcttgggcg gtatggcaac |
| 1141 | actccatttt tgtttccttt atatggccaa ggagaactcc cccagtgttt ctgcaggatg |
| 1201 | tgtgctgtgt ttggtggaat ttattgtctt cgccattcag tacagtgcct tgtagtggac |
| 1261 | aaagaatcca gaaaatgtaa agcaattata gatcagtttg gtcagagaat aatctctgag |
| 1321 | catttcctcg tggaggacag ttactttcct gagaacatgt gctcacgtgt gcaatacagg |
| 1381 | cagatctcca gggcagtgct gattacagat agatctgtcc taaaaacaga ttcagatcaa |
| 1441 | cagatttcca ttttgacagt gccagcagag gaaccaggaa cttttgctgt tcgggtcatt |
| 1501 | gagttatgtt cttcaacgat gacatgcatg aaaggcacct atttggttca tttgacttgc |
| 1561 | acatcttcta aaacagcaag agaagattta gaatcagttg tgcagaaatt gtttgttcca |
| 1621 | tatactgaaa tggagataga aaatgaacaa gtagaaaagc caagaattct gtgggctctt |
| 1681 | tacttcaata tgagagattc gtcagacatc agcaggagct gttataatga tttaccatcc |
| 1741 | aacgtttatg tctgctctgg cccagattgt ggtttaggaa atgataatgc agtcaaacag |
| 1801 | gctgaaacac ttttccagga aatctgcccc aatgaagatt tctgtccccc tccaccaaat |
| 1861 | cctgaagaca ttatccttga tggagacagt ttacagccag aggcttcaga atccagtgcc |
| 1921 | ataccagagg ctaactcgga gactttcaag gaaagcacaa accttggaaa cctagaggag |
| 1981 | tcctctgaat aa. |

88. The composition of claim 86 or 87, wherein the human REP1 protein comprises or consists of the amino acid sequence of

| (SEQ ID NO: 10) | |
|---|---|
| 1 | MADTLPSEFD VIVIGTGLPE SIIAAACSRS GRRVLHVDSR SYYGGNWASF SFSGLLSWLK |
| 61 | EYQENSDIVS DSPVWQDQIL ENEEAIALSR KDKTIQHVEV FCYASQDLHE DVEEAGALQK |
| 121 | NHALVTSANS TEAADSAFLP TEDESLSTMS CEMLTEQTPS SDPENALEVN GAEVTGEKEN |
| 181 | HCDDKTCVPS TSAEDMSENV PIAEDTTEQP KKNRITYSQI IKEGRRFNID LVSKLLYSRG |
| 241 | LLIDLLIKSN VSRYAEFKNI TRILAFREGR VEQVPCSRAD VFNSKQLTMV EKRMLMKFLT |
| 301 | FCMEYEKYPD EYKGYEEITF YEYLKTQKLT PNLQYIVMHS IAMTSETASS TIDGLKATKN |
| 361 | FLHCLGRYGN TPFLFPLYGQ GELPQCFCRM CAVFGGIYCL RHSVQCLVVD KESRKCKAII |
| 421 | DQFGQRIISE HFLVEDSYFP ENMCSRVQYR QISRAVLITD RSVLKTDSDQ QISILTVPAE |
| 481 | EPGTFAVRVI ELCSSTMTCM KGTYLVHLTC TSSKTAREDL ESVVQKLFVP YTEMEIENEQ |
| 541 | VEKPRILWAL YFNMRDSSDI SRSCYNDLPS NVYVCSGPDC GLGNDNAVKQ AETLFQEICP |
| 601 | NEDFCPPPPN PEDIILDGDS LQPEASESSA IPEANSETFK ESTNLGNLEE SSE. |

89. The pharmaceutical composition of any one of claims 72-88, wherein the sequence encoding the PRE comprises a sequence isolated or derived from a Woodchuck Hepatitis virus (WPRE).

90. The pharmaceutical composition of claim 89, wherein the sequence encoding the WPRE comprises or consists of a nucleic acid sequence of

| (SEQ ID NO: 11) | |
|---|---|
| 1 | atcaacctct ggattacaaa atttgtgaaa gattgactgg tattcttaac tatgttgctc |
| 61 | cttttacgct atgtggatac gctgctttaa tgcctttgta tcatgctatt gcttcccgta |
| 121 | tggctttcat tttctcctcc ttgtataaat cctggttgct gtctctttat gaggagttgt |
| 181 | ggcccgttgt caggcaacgt ggcgtggtgt gcactgtgtt tgctgacgca acccccactg |
| 241 | gttggggcat tgccaccacc tgtcagctcc tttccgggac tttcgctttc cccctcccta |
| 301 | ttgccacggc ggaactcatc gccgcctgcc ttgcccgctg ctggacaggg gctcggctgt |
| 361 | tgggcactga caattccgtg gtgttgtcgg ggaaatcatc gtcctttcct tggctgctcg |
| 421 | cctgtgttgc cacctggatt ctgcgcggga cgtccttctg ctacgtccct tcggccctca |
| 481 | atccagcgga ccttccttcc cgcggcctgc tgccggctct gcggcctctt ccgcgtcttc |
| 541 | gccttcgccc tcagacgagt cggatctccc tttgggccgc ctccccgc. |

91. The pharmaceutical composition of any one of claims 72-90, wherein the sequence encoding a polyadenylation (polyA) site comprises a sequence isolated or derived from a mammalian gene.

92. The pharmaceutical composition of claim 91, wherein the sequence encoding a polyadenylation (polyA) site comprises a sequence isolated or derived from a bovine growth hormone gene (BGH).

93. The composition of claim 92, wherein the sequence encoding the polyA site comprises or consists of the nucleic acid sequence of

| (SEQ ID NO: 12) | |
|---|---|
| 1 | tcgctgatca gcctcgactg tgccttctag ttgccagcca tctgttgttt gcccctcccc |
| 61 | cgtgccttcc ttgaccctgg aaggtgccac tcccactgtc ctttcctaat aaaatgagga |
| 121 | aattgcatcg cattgtctga gtaggtgtca ttctattctg gggggtgggg tggggcagga |
| 181 | cagcaagggg gaggattggg aagacaatag caggcatgct ggggatgcgg tgggctctat |
| 241 | ggcttctgag gcggaaagaa ccagctgggg. |

94. The pharmaceutical composition of any one of claims 72-93, wherein the sequence encoding the AAV2 5′ITR comprises or consists of the nucleic acid sequence of

| (SEQ ID NO: 13) | |
|---|---|
| 1 | ctgcgcgctc gctcgctcac tgaggccgcc cgggcaaagc ccgggcgtcg ggcgaccttt |
| 61 | ggtcgcccgg cctcagtgag cgagcgagcg cgcagagagg gagtggccaa ctccatcact |
| 121 | aggggttcct tgtagttaat gatt. |

95. The pharmaceutical composition of any one of claims 72-93, wherein the sequence encoding the AAV2 3′ITR comprises or consists of the nucleic acid sequence of

| (SEQ ID NO: 14) | |
|---|---|
| 1 | AGGAACCCCT AGTGATGGAG TTGGCCACTC CCTCTCTGCG CGCTCGCTCG CTCACTGAGG |
| 61 | CCGGGCGACC AAAGGTCGCC CGACGCCCGG GCGGCCTCAG TGAGCGAGCG AGCGCGCAGA |
| 121 | G. |

96. The pharmaceutical composition of any one of claims 72-95, wherein the nucleic acid comprising, from 5′ to 3′, elements (a) through (i) comprises or consists of a DNA sequence.

97. The pharmaceutical composition of claim 96, wherein the nucleic acid comprising, from 5′ to 3′, elements (a) through (i) comprises or consists of a single-stranded DNA sequence.

98. The pharmaceutical composition of any one of claims 72-97, wherein each rAAV of the plurality of full rAAV of the final composition comprises a capsid protein isolated or derived from an AAV2.

99. The pharmaceutical composition of claim 98, wherein the AAV2 capsid protein comprises a sequence having at least 95% identity to the amino acid sequence

| (SEQ ID NO: 15) | |
|---|---|
| 1 | MAADGYLPDW LEDTLSEGIR QWWKLKPGPP PPKPAERHKD DSRGLVLPGY KYLGPFNGLD |
| 61 | KGEPVNEADA AALEHDKAYD RQLDSGDNPY LKYNHADAEF QERLKEDTSF GGNLGRAVFQ |
| 121 | AKKRVLEPLG LVEEPVKTAP GKKRPVEHSP VEPDSSSGTG KAGQQPARKR LNFGQTGDAD |
| 181 | SVPDPQPLGQ PPAAPSGLGT NTMATGSGAP MADNNEGADG VGNSSGNWHC DSTWMGDRVI |
| 241 | TTSTRTWALP TYNNHLYKQI SSQSGASNDN HYFGYSTPWG YFDFNRFHCH FSPRDWQRLI |
| 301 | NNNWGFRPKR LNFKLFNIQV KEVTQNDGTT TIANNLTSTV QVFTDSEYQL PYVLGSAHQG |
| 361 | CLPPFPADVF MVPQYGYLTL NNGSQAVGRS SFYCLEYFPS QMLRTGNNFT FSYTFEDVPF |
| 421 | HSSYAHSQSL DRLMNPLIDQ YLYYLSRTNT PSGTTTQSRL QFSQAGASDI RDQSRNWLPG |
| 481 | PCYRQQRVSK TSADNNNSEY SWTGATKYHL NGRDSLVNPG PAMASHKDDE EKFFPQSGVL |
| 541 | IFGKQGSEKT NVDIEKVMIT DEEEIRTTNP VATEQYGSVS TNLQRGNRQA ATADVNTQGV |
| 601 | LPGMVWQDRD VYLQGPIWAK IPHTDGHFHP SPLMGGFGLK HPPPQILIKN TPVPANPSTT |
| 661 | FSAAKFASFI TQYSTGQVSV EIEWELQKEN SKRWNPEIQY TSNYNKSVNV DFTVDTNGVY |
| 721 | SEPRPIGTRY LTRNL. |

100. The pharmaceutical composition of claim 98, wherein the AAV2 capsid protein comprises the amino acid sequence

| (SEQ ID NO: 15) | |
|---|---|
| 1 | MAADGYLPDW LEDTLSEGIR QWWKLKPGPP PPKPAERHKD DSRGLVLPGY KYLGPFNGLD |
| 61 | KGEPVNEADA AALEHDKAYD RQLDSGDNPY LKYNHADAEF QERLKEDTSF GGNLGRAVFQ |
| 121 | AKKRVLEPLG LVEEPVKTAP GKKRPVEHSP VEPDSSSGTG KAGQQPARKR LNFGQTGDAD |
| 181 | SVPDPQPLGQ PPAAPSGLGT NTMATGSGAP MADNNEGADG VGNSSGNWHC DSTWMGDRVI |
| 241 | TTSTRTWALP TYNNHLYKQI SSQSGASNDN HYFGYSTPWG YFDFNRFHCH FSPRDWQRLI |
| 301 | NNNWGFRPKR LNFKLFNIQV KEVTQNDGTT TIANNLTSTV QVFTDSEYQL PYVLGSAHQG |
| 361 | CLPPFPADVF MVPQYGYLTL NNGSQAVGRS SFYCLEYFPS QMLRTGNNFT FSYTFEDVPF |
| 421 | HSSYAHSQSL DRLMNPLIDQ YLYYLSRTNT PSGTTTQSRL QFSQAGASDI RDQSRNWLPG |
| 481 | PCYRQQRVSK TSADNNNSEY SWTGATKYHL NGRDSLVNPG PAMASHKDDE EKFFPQSGVL |
| 541 | IFGKQGSEKT NVDIEKVMIT DEEEIRTTNP VATEQYGSVS TNLQRGNRQA ATADVNTQGV |
| 601 | LPGMVWQDRD VYLQGPIWAK IPHTDGHFHP SPLMGGFGLK HPPPQILIKN TPVPANPSTT |
| 661 | FSAAKFASFI TQYSTGQVSV EIEWELQKEN SKRWNPEIQY TSNYNKSVNV DFTVDTNGVY |
| 721 | SEPRPIGTRY LTRNL. |

101. The pharmaceutical composition of any one of claims 72-100, wherein the pharmaceutical composition further comprises a formulation buffer.

102. The pharmaceutical composition of claim 101, wherein the formulation buffer comprises Tris, MgCl₂, and NaCl.

103. The pharmaceutical composition of claim 101, wherein the formulation buffer comprises 20 mM Tris, 1 mM MgCl₂, and 200 mM NaCl at pH 8.

104. The pharmaceutical composition of claim 101, wherein the formulation buffer comprises 20 mM Tris, 1 mM MgCl₂, 200 mM NaCl at pH 8 and poloxamer 188 at 0.001%.

105. The pharmaceutical composition of any one of claims 72-104, wherein the plurality of full rAAVs are at a concentration of between 1×10⁸ genome particles (gp)/mL and 1×10¹⁴ gp/mL, inclusive of the endpoints.

106. The pharmaceutical composition of claim 105, wherein the plurality of full rAAVs are at a concentration of between 0.5×10¹⁰ gp/mL and 2.5×10¹² gp/mL, inclusive of the endpoints.

107. The pharmaceutical composition of claim 105, wherein the plurality of full rAAVs are at a concentration of between 1×10¹¹ gp/mL and 5×10¹³ gp/mL inclusive of the endpoints.

108. The pharmaceutical composition of claim 105, wherein the plurality of full rAAVs are at a concentration of between 1×10¹¹ gp/mL and 2×10¹² gp/mL, inclusive of the endpoints.

109. The pharmaceutical composition of claim 105, wherein the plurality of full rAAVs are at a concentration of 1×10¹² gp/mL.

110. The pharmaceutical composition of claim 105, wherein the plurality of full rAAVs are at a concentration of 1×10¹¹ gp/mL.

111. The pharmaceutical composition of any one of claims 72-110, wherein the concentration of the plurality of full rAAVs is measured using qPCR.

112. The pharmaceutical composition of claim 111, wherein the qPCR uses a supercoiled plasmid vector as a standard.

113. The pharmaceutical composition of claim 111, wherein the qPCR uses a linearized plasmid vector as a standard.

114. A delivery device comprising the pharmaceutical composition of any one of claims 72-113.

115. The delivery device of claim 114, wherein the delivery device comprises one or more of a syringe, a catheter and a needle.

116. The delivery device of claim 114, wherein the delivery device is suitable for administering the pharmaceutical composition by injection.

117. The delivery device of claim 114, wherein the delivery device is suitable for administering the pharmaceutical composition by infusion.

118. The delivery device of any one of claims 114-117, wherein the delivery device is suitable for administering the pharmaceutical composition by a subretinal route.

119. The delivery device of any one of claims 114-117, wherein the delivery device is suitable for administering the pharmaceutical composition by a suprachoroidal route.

120. A method of treating a disease or disorder in a subject in need thereof, comprising administering to the subject a therapeutically effective amount of a pharmaceutical composition of any one of claims 38-113.

121. The method of claim 120, wherein the disease or disorder is a retinal disease or disorder.

122. The method of claim 120 or 121, wherein the disease or disorder is Choroideremia.

123. The method of any one of claims 120-122, wherein the therapeutically effective amount comprises an amount between a minimally effective amount and a maximally tolerable amount of the pharmaceutical composition.

124. The method of claim 123, wherein the minimally effective amount comprises an amount of the pharmaceutical composition sufficient to transduce at least one neuron of a retina or a target portion thereof.

125. The method of claim 123, wherein the minimally effective amount comprises an amount of the pharmaceutical composition sufficient to transduce at least 1%, 5%, 10%, 15%, 20%, 25%, 30%, 35%, 40%, 45%, 50%, 55%, 60%, 65%, 70%, 75%, 80%, 85%, 90%, 95%, 97%, 99% or any percentage in between of the neurons of a retina or a target portion thereof.

126. The method of any one of claims 123-125, wherein the minimally effective amount comprises an amount of the pharmaceutical composition sufficient to improve visual acuity of the subject.

127. The method of any one of claims 123-125, wherein the minimally effective amount comprises an amount of the pharmaceutical composition sufficient to reduce a sign or symptom of a retinal disease.

128. The method of claim 127, wherein the retinal disease is Choroideremia.

129. The method of any one of claims 123-128, wherein the maximally tolerable amount comprises an amount of the pharmaceutical composition sufficient to induce an adverse event.

130. The method of claim 129, wherein the adverse effect comprises an immune response to the pharmaceutical composition.

131. The method of claim 130, wherein the immune response comprises inflammation.

132. The method of claim 131, wherein the inflammation is systemic.

133. The method of claim 131, wherein the inflammation is local.

134. The method of any one of claims 129-133, wherein the adverse event is severe.

135. The method of claim 134, wherein the adverse event cannot be prevented, reduced or controlled by administering a secondary medical treatment to the subject.

136. The method of any one of claims 120-135, wherein the therapeutically effective amount of the pharmaceutical composition comprises an amount having a multiplicity of infection (MOI) of between 10⁴ and 10⁶, inclusive of the endpoints.

137. The method of claim 136, wherein the therapeutically effective amount of the pharmaceutical composition comprises an amount having a multiplicity of infection (MOI) of between 10⁴ and 10⁵, inclusive of the endpoints.

138. The method of claim 136, wherein the therapeutically effective amount of the pharmaceutical composition comprises an amount having a multiplicity of infection (MOI) of 10⁵.

139. The method of any one of claims 120-135, wherein the therapeutically effective amount comprises between 1×10⁸ gp and 1×10¹³ gp, inclusive of the endpoints.

140. The method of any one of claims 120-135, wherein the therapeutically effective amount comprises between 6×10⁹ gp and 1×10¹³ gp, inclusive of the endpoints.

141. The method of any one of claims 120-135, wherein the therapeutically effective amount comprises between 6×10⁹ gp and 7×10¹² gp, inclusive of the endpoints.

142. The method of any one of claims 120-135, wherein the therapeutically effective amount comprises between 6×10⁹ gp and 5×10¹² gp, inclusive of the endpoints.

143. The method of any one of claims 120-135, wherein the therapeutically effective amount comprises between 1×10¹⁰ gp and 1×10¹² gp, inclusive of the endpoints.

144. The method of any one of claims 120-135, wherein the therapeutically effective amount comprises or consists of 1×10¹⁰ gp.

145. The method of any one of claims 120-135, wherein the therapeutically effective amount comprises or consists of 1×10¹¹ gp.

146. The method of any one of claims 120-135, wherein the therapeutically effective amount comprises or consists of 1×10¹² gp.

147. The method of any one of claims 120-146, wherein the therapeutically effective amount comprises or consists of a volume between 10 μL and 200 μL, inclusive of the endpoints.

148. The method of claim 147, wherein the therapeutically effective amount comprises or consists of a volume between 10 μL and 50 μL, between 50 μL and 100 μL, between 100 μL and 150 μL or between 150 μL and 200 μL, inclusive of the endpoints, for each range.

149. The method of any one of claims 120-148, wherein the therapeutically effective amount comprises or consists of a volume between 70 μL and 120 μL, inclusive of the endpoints.

150. The method of any one of claims 120-149, wherein the therapeutically effective amount comprises or consists of a volume a volume of 100 μL.

151. The method of any one of claims 120-150, wherein the administering step comprises an injection or an infusion.

152. The method of any one of claims 120-151, wherein the administering step comprises a subretinal, a suprachoroidal or an intravitreal route.

153. The method of any one of claims 120-152, wherein the administering step comprises a subretinal injection or infusion.

154. The method of claim 153, wherein the subretinal injection or infusion comprises a 2 step subretinal injection.

155. The method of any one of claims 120-152, wherein the administering step comprises a suprachoroidal injection or infusion.

156. The method of any one of claims 120-155, wherein the subject is male.

157. The method of claim 156, wherein the subject is at least 18 years of age.

158. The method of any one of claims 120-157, wherein the subject has a genetically confirmed diagnosis of choroideremia.

159. The method of claim 154, wherein the subject has been identified as having a mutation in the REP1 gene.

160. The method of any one of claims 120-159, wherein the subject presents a clinical sign of choroideremia in a macula of at least one eye.

161. The method of any one of claims 120-160, wherein the subject has a Best Corrected Visual Acuity (BCVA) score of 34-73 letters in at least one eye.

162. The method of any one of claims 120-161, wherein the subject has mild or early stage choroideremia.

163. The method of any one of claims 120-162, wherein the subject has advanced or severe choroideremia.

164. The method of any one of claims 120-163, wherein the method comprises treating 10 mm² of a retina of at least one eye.

165. The method of claim 164, wherein the method comprises treating between 5 mm² and 10 mm², inclusive of the endpoints, of a retina of at least one eye.

166. The method of claim 164, wherein the method comprises treating between 2 mm² and 15 mm², inclusive of the endpoints, of a retina of at least one eye.

167. The method of any one of claims 120-166, wherein the pharmaceutical composition is administered to one eye of the subject.

168. The method of any one of claims 120-162167 wherein the pharmaceutical composition is administered to both eyes of the subject.

169. The method of claim 168, wherein the eyes of the subject are treated simultaneously.

170. The method of claim 169, wherein the eyes of the subject are treated sequentially.

171. The method of any one of claims 120-170, wherein at least one eye of the subject had been treated for choroideremia prior to administration of the pharmaceutical composition to the subject.

172. The method of any one of claims 120-171, wherein the method comprises administering between 1 and 12 doses per eye, inclusive of the endpoints.

173. The method of claim 172, wherein the method comprises administering at least one dose at least once per day, once per week, once per month, once every three months, once every 6 months, or once per year.

174. The method of claim 173, wherein the method comprises administering multiple doses and wherein each dose comprises the same amount of the pharmaceutical composition.

175. The method of claim 173, wherein the method comprises administering multiple doses and wherein each dose does not comprise the same amount of the pharmaceutical composition.

176. The method of any one of claims 172-175, wherein the method comprises administering multiple doses and wherein each successive dose comprises a greater number of full rAAV than the previous dose.

177. The method of any one of claims 172-175, wherein the method comprises administering multiple doses and wherein each successive dose comprises a lesser number of full rAAV than the previous dose.

178. The method of any one of claims 172-177, wherein the subject has experienced an adverse event following a dose and wherein the subsequent dose comprises a lesser number of full rAAV than the previous dose that induced the adverse event.

179. The method of claim 178, wherein the subject recovers from the adverse event and wherein a subsequent dose of the pharmaceutical composition is administered to the subject.

180. The method of claim 179, wherein the dose that induced the adverse event and the subsequent dose contain an equal number of full rAAV.

181. The method of claim 179, wherein the dose that induced the adverse event and the subsequent dose do not contain an equal number of full rAAV.

182. The method of any one of claims 120-181, wherein the method further comprises administering an amount of a plurality of placebo rAAVs to the subject prior to administration of a therapeutically effective amount of the pharmaceutical composition, wherein each placebo rAAV is an empty rAAV.

183. The method of claim 182, wherein the empty rAAV does not contain either a promoter to express an exogenous sequence or an exogenous sequence.

184. The method of any one of claims 120-183, wherein administration of the amount of a plurality of placebo rAAVs is systemic.

185. The method of any one of claims 120-183, wherein administration of the amount of a plurality of placebo rAAVs is local.

186. The method of any one of claims 182-185, wherein the method further comprises
(a) determining whether the plurality of placebo rAAVs induced an immune response in the subject and/or
(b) determining whether the subject developed an immune tolerance to the plurality of placebo rAAVs, thereby indicating that administration of a therapeutically effective amount of the pharmaceutical composition should not induce an immune-mediated adverse event in the subject.

187. The method of any one of claims 120-186, wherein the method further comprises administering a suppressant of an immune response.

188. The method of claim 187, wherein the suppressant comprises an anti-inflammatory agent.

189. The method of claim 188, wherein the anti-inflammatory agent comprises a corticosteroid.

190. The method of claim 189, wherein the corticosteroid comprises prednisone or prednisolone.

191. The method of any one of claims 187-190, wherein the administration of the suppressant of an immune response is systemic.

192. The method of claim 191, wherein the suppressant of an immune response is administered orally.

193. The method of any one of claims 187-190, wherein the administration of the suppressant of an immune response is local.

194. The method of claim 193, wherein the suppressant of an immune response is administered to the eye treated with the pharmaceutical composition.

195. The method of any one of claims 187-194, wherein the pharmaceutical composition and the suppressant of an immune response are administered simultaneously.

196. The method of claim 195, wherein the pharmaceutical composition and the suppressant of an immune response are administered on the same day.

197. The method of any one of claims 187-194, wherein the pharmaceutical composition and the suppressant of an immune response are administered sequentially.

198. The method of claim 197, wherein the administration of the suppressant precedes the administration of the pharmaceutical composition by at least one day.

199. The method of claim 193, wherein the administration of the pharmaceutical composition precedes the administration of the suppressant by at least one day.

200. The method of any one of claims 120-199, wherein the method further comprises determining an initial severity of choroideremia-mediated damage in at least one eye of the subject.

201. The method of any one of claims 120-200, wherein the method further comprises determining a subsequent severity of choroideremia-mediated damage in the at least one eye of the subject following administration of the pharmaceutical composition to the at least one eye.

202. The method of claim 200 or 201, wherein the initial or subsequent severity of choroideremia-mediated damage is determined by determining a Best Corrected Visual Acuity (BCVA) test score, measuring an area or a volume of viable retinal tissue, measuring a preserved ellipsoid zone, measuring retinal sensitivity, measuring contrast sensitivity, measuring color vision, measuring low luminance visual acuity, measuring speed reading or any combination thereof.

203. The method of claim 202, wherein the BCVA test utilizes an (Early Treatment of Diabetic Retinopathy Study) ETDRS chart.

204. The method of claim 202 or 203, wherein the BCVA test comprises an assessment of one or more of finger counting, hand movement, light perception and a combination thereof.

205. The method of claim 202, wherein viable retinal tissue comprises fundus autofluorescence and wherein measuring viable retinal tissue comprises detecting a level or pattern of fundus autofluorescence.

206. The method of claim 202, wherein measuring a preserved ellipsoid zone comprises Spectral Domain Optical Coherence Tomography (SD-OCT).

207. The method of claim 202, wherein measuring retinal sensitivity comprises microperimetry.

208. The method of any one of claims 120-207, wherein administration of the therapeutically effective amount of the pharmaceutical composition inhibits or reduces progression of a sign or a symptom of choroideremia.

209. The method of any one of claims 120-207, wherein administration of the therapeutically effective amount of the pharmaceutical composition reduces a sign or a symptom of choroideremia.

210. The method of any one of claims 120-207, wherein a sign or a symptom of choroideremia comprises a loss of photoreceptor cells, a loss of RPE cells, a decreased visual acuity, decreased low luminescence visual acuity, total area of preserved autofluorescence (AF), a low score on a BCVA test, a decreased area of preserved ellipsoid zone, decreased retinal sensitivity, decreased contrast sensitivity, decreased or faded color vision, decreased rates of speed reading or any combination thereof.

211. The method of claim 210, wherein the severity of the sign or symptom of choroideremia is determined relative to a healthy retina.

212. The method of claim 211, wherein the healthy retina belongs to an age-matched control subject.

213. A method of determining a therapeutically effective amount of a pharmaceutical composition of any one of claims 38-212, the method comprising:
(a) measuring an area of a retina of a subject to be treated,
(b) determining whether the area of (a) is in the central 0.5 mm² foveal area or in the macula,
(c) calculating the number of rods, cones and retinal pigment epithelial (RPE) cells within the area of (a), and
(d) multiplying the total number of cells by a multiplicity of infection (MOI) of 1×10⁵ to calculate a number of genome particles (gp) to be included in the therapeutically effective amount,
wherein the maximal area of the retinal to be treated is 10 mm²,
wherein the density of RPE cells in the retina is 5,000 cells per mm²,
wherein the density of rods in the retina is 75,000 rods per mm² exclusive of the central 0.5 mm² foveal area,
wherein the density of cones in the retina is 150,000 cones per mm² in the central 0.5 mm² foveal area and the density of cones in the retina is 25,000 per mm² in the macula outside the central 0.5 mm² foveal area.
